SEQUENCE LISTING (170 sequences)

SEQ ID NO: 1 - DNA, 1536 bp, Homo sapiens
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 2 - DNA, 19 bp, Artificial sequence
  Synthetic oligonucleotide
tgagagtgattcgcgtggg

SEQ ID NO: 3 - DNA, 21 bp, Artificial sequence
  Synthetic oligonucleotide
ccagggtacgaggctttcaat

SEQ ID NO: 4 - DNA, 91 bp, Artificial sequence
  Synthetic oligonucleotide
tgagagtgattcgcgtgggtacccgcaagagccagcttgctcgcatacagacggacagtgtggtggcaacattgaaagcctcgtaccctgg

SEQ ID NO: 5 - DNA, 1261 bp, Homo sapiens
ccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 6 - DNA, 21 bp, Artificial sequence
  Synthetic oligonucleotide
tgacactggcaaaacaatgca

SEQ ID NO: 7 - DNA, 21 bp, Artificial sequence
  Synthetic oligonucleotide
ggtccttttcaccagcaagct

SEQ ID NO: 8 - DNA, 94 bp, Artificial sequence
  Synthetic oligonucleotide
tgacactggcaaaacaatgcagactttgctttccttggtcaggcagtataatccaaagatggtcaaggtcgcaagcttgctggtgaaaaggacc

SEQ ID NO: 9 - DNA, 1261 bp, Homo sapiens
cttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaa

SEQ ID NO: 10 - DNA, 20 bp, Artificial sequence
  Synthetic oligonucleotide
tgcaccaccaactgcttagc

SEQ ID NO: 11 - DNA, 19 bp, Artificial sequence
  Synthetic oligonucleotide
ccatcacgccacagtttcc

SEQ ID NO: 12 - skipped

SEQ ID NO: 13 - DNA, 2650 bp, Homo sapiens
agggacagcccagaggaggcgtggccacgctgccggcggaagtggagccctccgcgagcgcgcgaggccgccggggcaggcggggaaaccggacagtaggggcggggccgggccggcgatggggatgcgggagcactacgcggagctgcacccgtgcccgccggaattggggatgcagagcagcggcagcgggtatggcaggcagccggcgggccggcctccagcgcaggtgcccgagaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtaacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatggatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagccaaaaaaaaaaaaaaaaaaaa

SEQ ID NO: 14 - DNA, 20 bp, Artificial sequence
  Synthetic oligonucleotide
gaggccgtcaccaagaacat

SEQ ID NO: 15 - DNA, 19 bp, Artificial sequence
  Synthetic oligonucleotide
ggacagccggtcagagctc

SEQ ID NO: 16 - DNA, 541 bp, Homo sapiens
cttttcgatccgccatctgcggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaactaaca

SEQ ID NO: 17 - DNA, 20 bp, Artificial sequence
  Synthetic oligonucleotide
ctggcaagcagctggaagat

SEQ ID NO: 18 - DNA, 23 bp, Artificial sequence
  Synthetic oligonucleotide
tttcttagcaccaccacgaagtc

SEQ ID NO: 19 - DNA, 748 bp, Homo sapiens
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 20 - DNA, 23 bp, Artificial sequence
  Synthetic oligonucleotide
tggcaagaagaaggtctggttag

SEQ ID NO: 21 - DNA, 22 bp, Artificial sequence
  Synthetic oligonucleotide
tgatcagcccatctttgatgag

SEQ ID NO: 22 - DNA, 101 bp, Artificial sequence
  Synthetic oligonucleotide
tggcaagaagaaggtctggttagaccccaatgagaccaatgaaatcgccaatgccaactcccgtcagcagatccggaagctcatcaaagatgggctgatca

SEQ ID NO: 23 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 24 - DNA, 20 bp, Artificial sequence
  Synthetic oligonucleotide
cggtttgctgcggtaatcat

SEQ ID NO: 25 - DNA, 21 bp, Artificial sequence
  Synthetic oligonucleotide
tttcttgctgccagtctggac

SEQ ID NO: 26 - DNA, 122 bp, Artificial sequence
  Synthetic oligonucleotide
cggtttgctgcggtaatcatgaggataagagagccacgaaccacggcactgattttcagttctgggaaaatggtgtgcacaggagccaagagtgaagaacagtccagactggcagcaagaaa

SEQ ID NO: 27 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 28 - DNA, 19 bp, Artificial sequence
  Synthetic oligonucleotide
atttgggtcgcggttcttg

SEQ ID NO: 29 - DNA, 21 bp, Artificial sequence
  Synthetic oligonucleotide
tgccttgacattctcgatggt

SEQ ID NO: 30 - DNA, 133 bp, Artificial sequence
  Synthetic oligonucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 31 - DNA, 2277 bp, Homo sapiens
gactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcgcagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtcgatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacagacctggtggagaccctggagctgcagaacctgatgctgtgtgcgctgcagaccatctacggagcagaggcgcggaaggagtcacggggcgcgcatgccagggaagactacaaggtgcggattgatgagtacgattactccaagcccatccaggggcaacagaagaagccctttgaggagcactggaggaagcacaccctgtcctttgtggacgttggcactgggaaggtcactctggaatatagacccgtaatcgacaaaactttgaacgaggctgactgtgccaccatcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgag

SEQ ID NO: 32 - DNA, 20 bp, Artificial sequence
  Synthetic oligonucleotide
tgggaacaagagggcatctg

SEQ ID NO: 33 - DNA, 22 bp, Artificial sequence
  Synthetic oligonucleotide
ccaccactgcatcaaattcatg

SEQ ID NO: 34 - DNA, 86 bp, Artificial sequence
  Synthetic oligonucleotide
tgggaacaagagggcatctgctaaagtttcagattccatttctgctcagtatccagtagtggatcatgaatttgatgcagtggtgg

SEQ ID NO: 35 - DNA, 1720 bp, Homo sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 36 - DNA, 1813 bp, Homo sapiens
ggggagaagcaggcaggagaaccgagtcttggccttaactggctggcaacgtaaaacctgccctccacaaggacaagatgaaaacttggggatttggaggggacaacaaattagactcaatcacaatttcaaacttttggatgtcaatatatccatcaagaaattgaaaagaggcctggcatggtggccgatgcctgtcatcccagcactttgggagaccaacgtcggaggatcacttgaggtcaggagttccagaccagcctgggcaacgtggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 37 - DNA, 1712 bp, Homo sapiens
ggggagaagcaggcaggagaaccgagtcttggccttaactggctggcaacgtaaaacctgccctccacaaggacaagatgaaaacttggggatttggaggggacaacaaattagactcaatcacaatttcaaacttttggatgtcaatatatccatcaagaaattgaaaagaggcctggcatggtggccgatgcctgtcatcccagcactttgggagaccaacgtcggaggatcacttgaggtcaggagttccagaccagcctgggcaacactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 38 - DNA, 1396 bp, Homo sapiens
agaaagcaactagcacagccatgcccaggccaggacccgtgacacagcgatgctctcagatgctgctgatgttacagatgttgttgggggcacccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 39 - DNA, 3148 bp, Homo sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgaggtctgggctgtggtgggacccaggagggggcaggcagaggggcacactcttagcccgccctaaacccatctgtgccgtgtgactccggcaaacctctcacccttgctaagcctagcaactaggttaaggcaccctcaaaccccagtctccccagtctccctaacacttgtaccagcctgaggggctgtgattcctagaggctcagtagtctggaatgtgatgggtttggcactcttcatttctcgggattacacctgtaatcccagcactttaggaggccaaggtgggagcatcgctttaggccagaacattgcagcctgggcagtatagcgagaatccatctctaccaaaaatgtttaaaaaaaattagccagacatgatggtgtgtgcctgtagtctcagttattcaggaggctgaggcgggaggagcacttgaatccaggagattgaggctgcactgagccatgatgcagtggcacgatcttggctcactgcaacctccaccgccccgattcaagccattcttctgcctcagcctcccgagtagctgggatcacaggcgccagccaccatgcccagctaatttttggtagttttagtagaagcgggcgtttcaccatattggccacatatcggccaggctggtcttgaactcctgacctcaagtgatccacctgcatcggcctcccaaagtgctgggattacaggcgtgagccaccgcgcccgcacggagaaatgctcttggtaggggaaataacttgtgcaaaggcgccgccgccgtggtgagatccagcttggcgtgttaggggaaatgggcaccctcgttcctggaaaacggtaggcctgtgggtgaccagcttccctatctgtcttcccacagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 40 - DNA, 2268 bp, Homo sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 41 - DNA, 1925 bp, Homo sapiens
agaaagcaactagcacagccatgcccaggccaggacccgtgacacagcgatgctctcagatgctgctgatgttacagatgttgttgggggcaccctgagggggcaccctgactctgcaggtgagaaccttctggggttcctaggaccctcggacaagcactctgatccggatgactccaggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgaggtctgggctgtggtgggacccaggagggggcaggcagaggggcacactcttagcccgccctaaacccatctgtgccgtgtgactccggcaaacctctcacccttgctaagcctagcaactaggttaaggcaccctcaaaccccagtctccccagtctccctaacacttgtaccagcctgaggggctgtgattcctagaggctcagtagtctggaatgtgatgggtttggcactcttcatttctcgggattacacctgtaatcccagcactttaggaggccaaggtgggagcatcgctttaggccagaacattgcagcctgggcagtatagcgagaatccatctctaccaaaaatgtttaaaaaaaattagccagacatgatggtgtgtgcctgtagtctcagttattcaggaggctgaggcgggaggagcacttgaatccaggagattgaggctgcactgagccatgagtgccccactgcactccagcctgggtaacagcaagaccataataaataaataaataagtgaagcaagtaagtttttgactg

SEQ ID NO: 42 - DNA, 270 bp, Homo sapiens
ggggagaagcaggcaggagaaccgagtcttggccttaactggctggcaacgtaaaacctgccctccacaaggacaagatgaaaacttggggatttggaggggacaacaaattagactcaatcacaatttcaaacttttggatgtcaatatatccatcaagaaattgaaaagaggcctggcatggtggccgatgcctgtcatcccagcactttgggagaccaacgtcggaggatcacttgaggtcaggagttccagaccagcctgggcaac

SEQ ID NO: 43 - DNA, 312 bp, Homo sapiens
actcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcag

SEQ ID NO: 44 - DNA, 238 bp, Homo sapiens
atgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaag

SEQ ID NO: 45 - DNA, 548 bp, Homo sapiens
gtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgag

SEQ ID NO: 46 - DNA, 3 bp, Homo sapiens
gtg

SEQ ID NO: 47 - DNA, 94 bp, Homo sapiens
agaaagcaactagcacagccatgcccaggccaggacccgtgacacagcgatgctctcagatgctgctgatgttacagatgttgttgggggcacc

SEQ ID NO: 48 - DNA, 12 bp, Homo sapiens
ctgagggggcac

SEQ ID NO: 49 - DNA, 74 bp, Homo sapiens
cctgactctgcaggtgagaaccttctggggttcctaggaccctcggacaagcactctgatccggatgactccag

SEQ ID NO: 50 - DNA, 98 bp, Homo sapiens
gtcccaggagttgcagaaacgccaccaggaccttcagagcaccagttagacaaggagagccaggaggagctgggccccaacctcatccaaaagcacag

SEQ ID NO: 51 - protein, 308 aa, Homo sapiens [sequence omitted]

SEQ ID NO: 52 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDDCPLGPGRCCRLHTVRASLEDLGWADWVLSPREVQVTMCIGACPSQFRAANMHAQIKTSLHRLKPDTEPAPCCVPASYNPMVLIQKTDTGVSLQTYDDLLAKDCHCI

SEQ ID NO: 53 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 54 - protein, 308 aa, Homo sapiens
MPGQELRTLNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 55 - protein, 331 aa, Homo sapiens
MTPGPRSCRNATRTFRAPVRQGEPGGAGPQPHPKAQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 56 - protein, 94 aa, Homo sapiens [sequence omitted]

SEQ ID NO: 57 - DNA, 19 bp, Homo sapiens
ctggatccccttgggtctg

SEQ ID NO: 58 - DNA, 20 bp, Homo sapiens
cctggcatccctgtacctca

SEQ ID NO: 59 - DNA, 115 bp, Artificial sequence
  Synthetic oligonucleotide
ctggatccccttgggtctgatggggtagccgatgcctgatttgcacccacaacgtgggaggttataacctgtccccaagttgcaaatggggaaactgaggtacagggatgccagg

SEQ ID NO: 60 - DNA, 3292 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaaagtcctgctctgtcgcccaggctggaatgcagtggcgcgatctcggctcactgcaacctctgcctcccagattcaagcaattctcctgcttcagcctcccaagtagctgggattacaggcacgtgccaccatgcctggctaatttttgtatttttagtagagatggggtttcaccatcttggtcaggctggtctcaaactcctggtgatccacctgcctctgcctcccaaagtgctgggattacagccgtgagccactgcacccagccggcttcatctcttcttgaaatcacttttataccattctatgtggttctcaccatgagcttgagtggtgggctaaagtgcctctccctgctttcagcttcctgctgggaactcactctctcaagttccttccagcaccaccccatagagttcccatcactccacactgtccagtgacaactcccaacatggaagatctgctagttctacagggtgctctctggctgccccagtaacatgtgtttttaaatttttcacatgcatgtttgaccccgactccccgaagtcaggtactgtaactagcagtgtcatttaagaaaaagccctttaacctctctttgccaaaggattcttatcagcaaaacagtgatgaaacaacaatcccataacagctagctggctaccttctcaagcacttattaaatgaggcataatgattttgcttaatcctcaatcctgagaggtgggcgatccctgtggtgatgaggaaaccgaggcttgggggttaatggcttgcctagattcacactgctagccaaggaatgaactggaatttacaccctgaccctgactgcttttcacattttctacacagccttttcaagatccctgccaattctaaaattaaatgattctatgattaactgtgtttcattcttctgcatcagttcccaaaacaaattatatcaagagacagcaaaaatatttgtaaagaaaggatgtccaacaatctgtgtggttgtttttctgtgttcctccaatggtagggcctctgttcaccagtgccgtctcttcttttagctgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 61 - DNA, 2343 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaaagtcctgctctgtcgcccaggctggaatgcagtggcgcgatctcggctcactgcaacctctgcctcccagattcaagcaattctcctgcttcagcctcccaagtagctgggattacagctgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 62 - DNA, 2366 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggtgaggagaggtgacctggtgcccatgctcacctgccctctccctcttcttgcccccacccgtccatccatcccacccatccatctatccctgcggcccccctctgcccgctcctctgaccaacacctgctttgtctgcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 63 - DNA, 3433 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaaagtcctgctctgtcgcccaggctggaatgcagtggcgcgatctcggctcactgcaacctctgcctcccagattcaagcaattctcctgcttcagcctcccaagtagctgggattacaggcacgtgccaccatgcctggctaatttttgtatttttagtagagatggggtttcaccatcttggtcaggctggtctcaaactcctggtgatccacctgcctctgcctcccaaagtgctgggattacagccgtgagccactgcacccagccggcttcatctcttcttgaaatcacttttataccattctatgtggttctcaccatgagcttgagtggtgggctaaagtgcctctccctgctttcagcttcctgctgggaactcactctctcaagttccttccagcaccaccccatagagttcccatcactccacactgtccagtgacaactcccaacatggaagatctgctagttctacagggtgctctctggctgccccagtaacatgtgtttttaaatttttcacatgcatgtttgaccccgactccccgaagtcaggtactgtaactagcagtgtcatttaagaaaaagccctttaacctctctttgccaaaggattcttatcagcaaaacagtgatgaaacaacaatcccataacagctagctggctaccttctcaagcacttattaaatgaggcataatgattttgcttaatcctcaatcctgagaggtgggcgatccctgtggtgatgaggaaaccgaggcttgggggttaatggcttgcctagattcacactgctagccaaggaatgaactggaatttacaccctgaccctgactgcttttcacattttctacacagccttttcaagatccctgccaattctaaaattaaatgattctatgattaactgtgtttcattcttctgcatcagttcccaaaacaaattatatcaagagacagcaaaaatatttgtaaagaaaggatgtccaacaatctgtgtggttgtttttctgtgttcctccaatggtagggcctctgttcaccagtgccgtctcttcttttagctgtaagaaaagcctggagtgcacgaagttgtgcctaccccagattgagaatgttaagggcactgaggactcaggtgaggagaggtgacctggtgcccatgctcacctgccctctccctcttcttgcccccacccgtccatccatcccacccatccatctatccctgcggcccccctctgcccgctcctctgaccaacacctgctttgtctgcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 64 - DNA, 2151 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaagcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 65 - DNA, 2046 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaaagtcctgctctgtcgcccaggctggaatgcagtggcgcgatctcggctcactgcaacctctgcctcccagattcaagcaattctcctgcttcagcctcccaagcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 66 - DNA, 1915 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 67 - DNA, 2250 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaagcctaactctgcggccccattcacaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 68 - DNA, 2227 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaagaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 69 - DNA, 2339 bp, Homo sapiens
ggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 70 - DNA, 1613 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 71 - DNA, 2031 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgg

SEQ ID NO: 72 - DNA, 667 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggtaggggcaagtggaaacggtgaatgccctcaggtctggggtgctgcttctttctctgcttcttccagttgttcttccctaactttgctgtctctcctgggctgggattttctccctccctcctctcctagagacttcagggaatcggccctggctgttgtccctagcatg

SEQ ID NO: 73 - DNA, 171 bp, Homo sapiens
gtaggggcaagtggaaacggtgaatgccctcaggtctggggtgctgcttctttctctgcttcttccagttgttcttccctaactttgctgtctctcctgggctgggattttctccctccctcctctcctagagacttcagggaatcggccctggctgttgtccctagcatg

SEQ ID NO: 74 - DNA, 945 bp, Homo sapiens
gtgccaccatgcctggctaatttttgtatttttagtagagatggggtttcaccatcttggtcaggctggtctcaaactcctggtgatccacctgcctctgcctcccaaagtgctgggattacagccgtgagccactgcacccagccggcttcatctcttcttgaaatcacttttataccattctatgtggttctcaccatgagcttgagtggtgggctaaagtgcctctccctgctttcagcttcctgctgggaactcactctctcaagttccttccagcaccaccccatagagttcccatcactccacactgtccagtgacaactcccaacatggaagatctgctagttctacagggtgctctctggctgccccagtaacatgtgtttttaaatttttcacatgcatgtttgaccccgactccccgaagtcaggtactgtaactagcagtgtcatttaagaaaaagccctttaacctctctttgccaaaggattcttatcagcaaaacagtgatgaaacaacaatcccataacagctagctggctaccttctcaagcacttattaaatgaggcataatgattttgcttaatcctcaatcctgagaggtgggcgatccctgtggtgatgaggaaaccgaggcttgggggttaatggcttgcctagattcacactgctagccaaggaatgaactggaatttacaccctgaccctgactgcttttcacattttctacacagccttttcaagatccctgccaattctaaaattaaatgattctatgattaactgtgtttcattcttctgcatcagttcccaaaacaaattatatcaagagacagcaaaaatatttgtaaagaaaggatgtccaacaatctgtgtggttgtttttctgtgttcctccaatggtagggcctctgttcaccagtgccgtctcttcttttag

SEQ ID NO: 75 - DNA, 141 bp, Homo sapiens
gtgaggagaggtgacctggtgcccatgctcacctgccctctccctcttcttgcccccacccgtccatccatcccacccatccatctatccctgcggcccccctctgcccgctcctctgaccaacacctgctttgtctgcag

SEQ ID NO: 76 - DNA, 69 bp, Homo sapiens
ggcgactgcaccaagaatggcagtgatgttcctgttgagaacctttacccttcaaagtacacacagcag

SEQ ID NO: 77 - DNA, 118 bp, Homo sapiens
gtgtgtattcactcctgcttccaggagagcatgatcaaggagtgtggctgtgcctacatcttctatccgcggccccagaacgtggagtactgtgactacagaaagcacagttcctggg

SEQ ID NO: 78 - DNA, 79 bp, Homo sapiens
ggtactgctactataagctccaggttgacttctcctcagaccacctgggctgtttcaccaagtgccggaagccatgcag

SEQ ID NO: 79 - DNA, 58 bp, Homo sapiens
cgtgaccagctaccagctctctgctggttactcacgatggccctcggtgacatcccag

SEQ ID NO: 80 - DNA, 56 bp, Homo sapiens
gaatgggtcttccagatgctatcgcgacagaacaattacaccgtcaacaacaagag

SEQ ID NO: 81 - DNA, 76 bp, Homo sapiens
aaatggagtggccaaagtcaacatcttcttcaaggagctgaactacaaaaccaattctgagtctccctctgtcacg

SEQ ID NO: 82 - DNA, 6 bp, Homo sapiens
gtgctc

SEQ ID NO: 83 - DNA, 65 bp, Homo sapiens
gtgtccccaaggaaaatatatccaccctcaaaataattcgatttgctgtaccaagtgccacaaag

SEQ ID NO: 84 - DNA, 23 bp, Homo sapiens
cctaactctgcggccccattcac

SEQ ID NO: 85 - DNA, 2 bp, Homo sapiens
ag

SEQ ID NO: 86 - DNA, 3 bp, Homo sapiens
cag

SEQ ID NO: 87 - DNA, 3 bp, Homo sapiens
aac

SEQ ID NO: 88 - DNA, 79 bp, Homo sapiens
gccaggagaaacagaacaccgtgtgcacctgccatgcaggtttctttctaagagaaaacgagtgtgtctcctgtagtaa

SEQ ID NO: 89 - DNA, 102 bp, Homo sapiens
agtcctgctctgtcgcccaggctggaatgcagtggcgcgatctcggctcactgcaacctctgcctcccagattcaagcaattctcctgcttcagcctcccaa

SEQ ID NO: 90 - DNA, 16 bp, Homo sapiens
gtagctgggattacag

SEQ ID NO: 91 - DNA, 4 bp, Homo sapiens
gcac

SEQ ID NO: 92 - DNA, 74 bp, Homo sapiens
ctgtaagaaaagcctggagtgcacgaagttgtgcctaccccagattgagaatgttaagggcactgaggactcag

SEQ ID NO: 93 - DNA, 23 bp, Homo sapiens
gcaccacagtgctgttgcccctg

SEQ ID NO: 94 - DNA, 25 bp, Homo sapiens
gcttcacccccaccctgggcttcag

SEQ ID NO: 95 - DNA, 31 bp, Homo sapiens
agaggtggcaccaccctatcagggggctgac

SEQ ID NO: 96 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 97 - protein, 42 aa, Homo sapiens
TYLYNDCPGPGQDTDCRECESGSFTASENHLRHSLSCSKCRK

SEQ ID NO: 98 - protein, 218 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSKVLLCRPGWNAVARSRLTATSASQIQAILLLQPPK

SEQ ID NO: 99 - protein, 228 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSKHHSAVAPGHFLWSLPFIPPLHWFNVSLPTVEVQALLHCLWEIDT

SEQ ID NO: 100 - protein, 242 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSKVLLCRPGWNAVARSRLTATSASQIQAILLLQPPKLHPHPGLQSRAQFHLHLQLHLYPR

SEQ ID NO: 101 - protein, 247 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSERWHHPIRGLTPSLRQPSPPTPSPTPFRSGRTAPTSHRA

SEQ ID NO: 102 - protein, 594 aa, Homo sapiens
GDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 103 - protein, 251 aa, Homo sapiens
MGLSTVPDLLLPLVLLELLVGIYPSGVIGLVPHLGDREKRDSVCPQGKYIHPQNNSICCTKCHKGTYLYNDCPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 104 - protein, 212 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGEER

SEQ ID NO: 105 - protein, 65 aa, Homo sapiens
MGLSTVPDLLLPLVLLELLVGIYPSGVIGLVPHLGDREKRDSVCPQGKYIHPQNNSICCTKCHKA

SEQ ID NO: 106 - protein, 174 aa, Homo sapiens
MGLSTVPDLLLPLVLLELLVGIYPSGVIGLVPHLGDREKRDSVCPQGKYIHPQNNSICCTKCHKEEPTCTMTVQARGRIRTAGSVRAAPSPLQKTTSDTASAAPNAERKWVRWRSLLAQWTGTPCVAAGRTSTGIIGVKTFSSASIAASASMGPCTSPARRNRTPCAPAMQVSF

SEQ ID NO: 107 - protein, 72 aa, Homo sapiens
MGLSTVPDLLLPLVLLELLVGIYPSGVIGLVPHLGDREKRDSVCPQGKYIHPQNNSICCTKCHKEPVPALLE

SEQ ID NO: 108 - protein, 71 aa, Homo sapiens
MGLSTVPDLLLPLVLLELLVGIYPSGVIGLVPHLGDREKRDSVCPQGKYIHPQNNSICCTKCHKGRGKWKR

SEQ ID NO: 109 - DNA, 6809 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacaggttagcccttcctcagaggggagaggagagggcacaggctagcccttccctacacaggagaggagagggcataggttagcccttccccacaggggagaggagagggcacaggttagccccccacctcgtccacatacatggaaagagcggagtccggcttagcacagagactcacaggaaagaagagcccaggttaacccctccttgtgtagaaaaagccaagaatgcccaggttagccccttaccacagagatagaagagaagagcaaaagttaggccaggacagccacaaggaaaaggacagagctcaggttaaccctcctcaccaaggaaaaatcaggctacactttctttctctaccctcttctgaaaagaaatgaagcccggttaaccctctccacacccaaaagaaaagggaagaggcccgctaagcctggagagccacacacaggcaaagaaaagagcctggctaatctttctctcaggcaaacaaagcaggagggcttgggttaactaccctcacccccaggcagatggaaagaggagagcccaggaatagcccttctccactgaggggttgacaagggcgggtgtgagccgggaaagcaggggtctcacatgccttctccccaccatgtctgggtctagcacagggcctggcacacacctcgagcgctctataaacaccgactgaatggaggggtcagcttccatgcaggatatgggagacaagcccctcccaaagcacacacacaaaacacacacgatcatggggacggggagaggagctggttagtccctccctgctgacctcccccaggccccttatccctctgccctgcctccctgttgtctcagcctcactgggtttcccatcaccccatcgctaggtggcctggcccttaccggcaccgtggccccatttaagccagggatcccctgaatgccaccaatgttcctctgggttaactcctgaactgctgagcccagagggcaccaagcaaaggactgtaggtgcccaaggaggaaaagatggaggctggcaaggcagatggggacctccgagggtaggggtgtcttccaggaatagcaacaacagtaaaaagagcaaattgctggccaggtgcagtggctcacgcctgtaatcccagcactttgggaggccgaggcaggtggatcacctgaggttaggcgttcaagaccagcctgaccaacatggtgaaaccccgtctctactaaaaatacaaaattacctgggcatggtggcaggcgcctgtaatcccagctactccggaggctgaggcaggagaatcacttgaacccgggaggcagaggttgcagtgagtcaggattgtgccattgcactccaacctgggtgacagaatgagactccatctcaaaaaaagtgagtgagttccttcctaagccagccactaggtgctttatgtaattatctcatttaattctcgaaacaacactatgatggaaggactattaccatttccatgttactgaggaaaccgaggcacagagggtttaaggaactagcgcaaggtcacagagcacccagcagtagatctctgcccctccgcagctgactccaggcccagccagagtatttgaggactcaggggtccttctctgctcggtggatggagcccccacttcctccctgggctcagcagcagcaacatgggcttttacaaacacggccgcaagaggaggcattgttgtggagcagggctgggctcaagacccagctctgccacacacctgctgggctggtggcattgggccagtcgcctctctgagccctatttcccccactgcagcagcatggggaccaccgcactggccctgggggctgagatgggagtgagagtgctgggcacagagggctgctaggtgggagcgcctgggaagagtacacaactcaggggttgctgagagaagagaaggcacagctgtcccagccccagttctcctgacaagcttctgtctttctggaagcccctaacccaatccctgagctgagcaggtggagaggaccccaaggaagacagaaggacaaggtcagacagacagagacaaggaggtgggttgacacatagacacagataaagaaatgaagacaccatgcctgtaatcccagcactttgggaggctgaggcgggcgcatcacctgaggtcaggatttcaagaccagcccggccaacatggtaaaaccccgtctctactaaaaatacaaaaattagctgggcgtggtggcacatgcctgtaatcccagctactcgggaggccgaggcaggagaatcacttgaacctgggaggcagaggttgcagtgagccaagatggcgccattgcactccagcctggccaacagagcaagactctgtccaaaaaacaaacaaacagacaaacaaacaaaacaaaaagaaatagagacacagagaagtggaagcggggcggcacagaggggattggagggggggacctgggccccagccacagccacagtagcttggcctgggggagcagggtgcgggcggtggggtggccggggctgaggggtggtgctcagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 110 - DNA, 4525 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcggtgaggccggccggggtccaagctggagaacacagaggggcagccccaaggagggcccatccgttcgctcattcagccactcgacaaacatcacgggcatgctccctgagccgagcggcaacaggacaggtttttctgcccaaaaggagccccgctctgggggagatggaggcagatgcatccagcagatttcctgcgacactgctgtacatgagccatgtccagagtgccgtgggagcactgagcagagagccatccctgtgcctgggggtgatgcctttaggaggctgagaagatgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 111 - DNA, 4332 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatgggtgagcctgctccagggtagggtgggtcggggcagtggggccaggagcccctgtcactgggccctgcctttgcccccgtgctacttgctcttccttctcttgcagaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 112 - DNA, 4521 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtccctcactgcagcctcactgggggtccctctccatagtgatagccatgaggacactgggattctggacttcagctcactgctgaaaaagagagacagtttccggaccccgaggtgagtgcccaacatagcacagcaccctcatcacccctaattctgccaggagcccctccagatccccagcccctcttcagctcccttggcccccagtcctgcctggcctgggacccagagcagctccagctgccccaccagaagggaaggggcagagggacaggggtggctacagctccttggtcctgggcccaggaagccccgcccagggggctgcagtcttgcccccggccacagcctagactgcgggacacagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 113 - DNA, 4417 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgaggtgaggagccctcggggccagggcctgggagatgggaagagcgggcggcagtggggaccctgggctttgctccgttgtcctcggccaagcacctgccctggctgcaagggcacggaccctgccacgcccagatgggcaatagcttccagaaggctgggaggacacagtgacatggcctcctcttctgcagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 114 - DNA, 4220 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttctcaa

SEQ ID NO: 115 - DNA, 3451 bp, Homo sapiens
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatgtaggggtggagatgccaagggcacccaggagaggctctcggcatcaggaagggcctggcccagaga

SEQ ID NO: 116 - DNA, 164 bp, Homo sapiens
ggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacag

SEQ ID NO: 117 - DNA, 2455 bp, Homo sapiens
gttagcccttcctcagaggggagaggagagggcacaggctagcccttccctacacaggagaggagagggcataggttagcccttccccacaggggagaggagagggcacaggttagccccccacctcgtccacatacatggaaagagcggagtccggcttagcacagagactcacaggaaagaagagcccaggttaacccctccttgtgtagaaaaagccaagaatgcccaggttagccccttaccacagagatagaagagaagagcaaaagttaggccaggacagccacaaggaaaaggacagagctcaggttaaccctcctcaccaaggaaaaatcaggctacactttctttctctaccctcttctgaaaagaaatgaagcccggttaaccctctccacacccaaaagaaaagggaagaggcccgctaagcctggagagccacacacaggcaaagaaaagagcctggctaatctttctctcaggcaaacaaagcaggagggcttgggttaactaccctcacccccaggcagatggaaagaggagagcccaggaatagcccttctccactgaggggttgacaagggcgggtgtgagccgggaaagcaggggtctcacatgccttctccccaccatgtctgggtctagcacagggcctggcacacacctcgagcgctctataaacaccgactgaatggaggggtcagcttccatgcaggatatgggagacaagcccctcccaaagcacacacacaaaacacacacgatcatggggacggggagaggagctggttagtccctccctgctgacctcccccaggccccttatccctctgccctgcctccctgttgtctcagcctcactgggtttcccatcaccccatcgctaggtggcctggcccttaccggcaccgtggccccatttaagccagggatcccctgaatgccaccaatgttcctctgggttaactcctgaactgctgagcccagagggcaccaagcaaaggactgtaggtgcccaaggaggaaaagatggaggctggcaaggcagatggggacctccgagggtaggggtgtcttccaggaatagcaacaacagtaaaaagagcaaattgctggccaggtgcagtggctcacgcctgtaatcccagcactttgggaggccgaggcaggtggatcacctgaggttaggcgttcaagaccagcctgaccaacatggtgaaaccccgtctctactaaaaatacaaaattacctgggcatggtggcaggcgcctgtaatcccagctactccggaggctgaggcaggagaatcacttgaacccgggaggcagaggttgcagtgagtcaggattgtgccattgcactccaacctgggtgacagaatgagactccatctcaaaaaaagtgagtgagttccttcctaagccagccactaggtgctttatgtaattatctcatttaattctcgaaacaacactatgatggaaggactattaccatttccatgttactgaggaaaccgaggcacagagggtttaaggaactagcgcaaggtcacagagcacccagcagtagatctctgcccctccgcagctgactccaggcccagccagagtatttgaggactcaggggtccttctctgctcggtggatggagcccccacttcctccctgggctcagcagcagcaacatgggcttttacaaacacggccgcaagaggaggcattgttgtggagcagggctgggctcaagacccagctctgccacacacctgctgggctggtggcattgggccagtcgcctctctgagccctatttcccccactgcagcagcatggggaccaccgcactggccctgggggctgagatgggagtgagagtgctgggcacagagggctgctaggtgggagcgcctgggaagagtacacaactcaggggttgctgagagaagagaaggcacagctgtcccagccccagttctcctgacaagcttctgtctttctggaagcccctaacccaatccctgagctgagcaggtggagaggaccccaaggaagacagaaggacaaggtcagacagacagagacaaggaggtgggttgacacatagacacagataaagaaatgaagacaccatgcctgtaatcccagcactttgggaggctgaggcgggcgcatcacctgaggtcaggatttcaagaccagcccggccaacatggtaaaaccccgtctctactaaaaatacaaaaattagctgggcgtggtggcacatgcctgtaatcccagctactcgggaggccgaggcaggagaatcacttgaacctgggaggcagaggttgcagtgagccaagatggcgccattgcactccagcctggccaacagagcaagactctgtccaaaaaacaaacaaacagacaaacaaacaaaacaaaaagaaatagagacacagagaa

SEQ ID NO: 118 - DNA, 127 bp, Homo sapiens
gtggaagcggggcggcacagaggggattggagggggggacctgggccccagccacagccacagtagcttggcctgggggagcagggtgcgggcggtggggtggccggggctgaggggtggtgctcag

SEQ ID NO: 119 - DNA, 298 bp, Homo sapiens
gtgaggccggccggggtccaagctggagaacacagaggggcagccccaaggagggcccatccgttcgctcattcagccactcgacaaacatcacgggcatgctccctgagccgagcggcaacaggacaggtttttctgcccaaaaggagccccgctctgggggagatggaggcagatgcatccagcagatttcctgcgacactgctgtacatgagccatgtccagagtgccgtgggagcactgagcagagagccatccctgtgcctgggggtgatgcctttaggaggctgagaagatg

SEQ ID NO: 120 - DNA, 190 bp, Homo sapiens
gtgaggagccctcggggccagggcctgggagatgggaagagcgggcggcagtggggaccctgggctttgctccgttgtcctcggccaagcacctgccctggctgcaagggcacggaccctgccacgcccagatgggcaatagcttccagaaggctgggaggacacagtgacatggcctcctcttctgcag

SEQ ID NO: 121 - DNA, 38 bp, Homo sapiens
tccctcactgcagcctcactgggggtccctctccatag

SEQ ID NO: 122 - DNA, 54 bp, Homo sapiens
tgatagccatgaggacactgggattctggacttcagctcactgctgaaaaagag

SEQ ID NO: 123 - DNA, 105 bp, Homo sapiens
gtgagcctgctccagggtagggtgggtcggggcagtggggccaggagcccctgtcactgggccctgcctttgcccccgtgctacttgctcttccttctcttgcag

SEQ ID NO: 124 - DNA, 66 bp, Homo sapiens
taggggtggagatgccaagggcacccaggagaggctctcggcatcaggaagggcctggcccagaga

SEQ ID NO: 125 - DNA, 46 bp, Homo sapiens
ggccaccaacttacagggcgaggcacggtgtgagtgccgcctggag

SEQ ID NO: 126 - DNA, 37 bp, Homo sapiens
tgcctcagtgaccaggctggctcctggggatggccag

SEQ ID NO: 127 - protein, 1274 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 128 - protein, 1274 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFECSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALRELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMQLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 129 - protein, 364 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTG

SEQ ID NO: 130 - protein, 868 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIGEAGRGPSWRTQRGSPKEGPSVRSFSHSTNITGMLPEPSGNRTGFSAQKEPRSGGDGGRCIQQISCDTAVHEPCPECRGSTEQRAIPVPGGDAFRRLRR

SEQ ID NO: 131 - protein, 638 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMGEPAPG

SEQ ID NO: 132 - protein, 311 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISPSLQPHWGSLSIVIAMRTLGFWTSAHC

SEQ ID NO: 133 - protein, 1327 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRGEEPSGPGPGRWEERAAVGTLGFAPLSSAKHLPWLQGHGPCHAQMGNSFQKAGRTQ

SEQ ID NO: 134 - protein, 1327 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLECLSDQAGSWGWPGTTGCQPRARSLEGSWGNPSLLLDVCVTSVSPVLRWGISRAVVGQS

SEQ ID NO: 135 - protein, 1110 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTM

SEQ ID NO: 136 - DNA, 19 bp, Homo sapiens
tccctcactgcagcctcac

SEQ ID NO: 137 - DNA, 26 bp, Homo sapiens
ctctttttcagcagtgagctgaagtc

SEQ ID NO: 138 - DNA, 92 bp, Homo sapiens
tccctcactgcagcctcactgggggtccctctccatagtgatagccatgaggacactgggattctggacttcagctcactgctgaaaaagag

SEQ ID NO: 139 - DNA, 19 bp, Homo sapiens
tgaggcgcgatgagaagaa

SEQ ID NO: 140 - DNA, 21 bp, Homo sapiens
catgtatgtggacgaggtggg

SEQ ID NO: 141 - DNA, 167 bp, Homo sapiens
tgaggcgcgatgagaagaagagcacaggttagcccttcctcagaggggagaggagagggcacaggctagcccttccctacacaggagaggagagggcataggttagcccttccccacaggggagaggagagggcacaggttagccccccacctcgtccacatacatg

SEQ ID NO: 142 - DNA, 19 bp, Homo sapiens
aggcgaggaggtgagcatc

SEQ ID NO: 143 - DNA, 20 bp, Homo sapiens
tcctccatgttctcaatgcg

SEQ ID NO: 144 - DNA, 117 bp, Homo sapiens
aggcgaggaggtgagcatccgcaacagccccacagacaccatcctgttcatccgggccgctcgccgcgtgcattcaggcacttaccaggtgacggtgcgcattgagaacatggagga

SEQ ID NO: 145 - protein, 35 aa, Artificial sequence
  Synthetic peptide
MTPGPRSCRNATRTFRAPVRQGEPGGAGPQPHPKA

SEQ ID NO: 146 - protein, 152 aa, Artificial sequence
  Synthetic peptide
GDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVT

SEQ ID NO: 147 - protein, 99 aa, Artificial sequence
  Synthetic peptide
GEAGRGPSWRTQRGSPKEGPSVRSFSHSTNITGMLPEPSGNRTGFSAQKEPRSGGDGGRCIQQISCDTAVHEPCPECRGSTEQRAIPVPGGDAFRRLRR

SEQ ID NO: 148 - protein, 56 aa, Artificial sequence
  Synthetic peptide
GEEPSGPGPGRWEERAAVGTLGFAPLSSAKHLPWLQGHGPCHAQMGNSFQKAGRTQ

SEQ ID NO: 149 - protein, 58 aa, Artificial sequence
  Synthetic peptide
CLSDQAGSWGWPGTTGCQPRARSLEGSWGNPSLLLDVCVTSVSPVLRWGISRAVVGQS

SEQ ID NO: 150 - DNA, 473 bp, Homo sapiens
cctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttatt

SEQ ID NO: 151 - DNA, 111 bp, Artificial sequence
  Synthetic oligonucleotide
gaggccgtcaccaagaacattcacgagtcctgcatgagccagataggctggaaccgcatcatcgtggagaagcccttcgggagggacctgcagagctctgaccggctgtcc

SEQ ID NO: 152 - DNA, 101 bp, Artificial sequence
  Synthetic oligonucleotide
ctggcaagcagctggaagatggacgtactttgtctgactacaatattcaaaaggagtctactcttcatcttgtgttgagacttcgtggtggtgctaagaaa

SEQ ID NO: 153 - DNA, 6546 bp, Homo sapiens
acacagtactctcagcttgttggtggaagcccctcatctgccttcattctgaaggcagggcccggcagaggaaggatcagagggtcgcggccggagggtcccggccggtggggccaactcagagggagaggaaagggctagagacacgaagaacgcaaaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagaaaaagaaatccaatttcaaaaagaagatgaggacagtggctactaaatcaaaggaaaaatccaaaaagtttacaccccccaaatctgaaaaattttcatccaagaagaagaaaagttatcaagcatcagcaaaagccaaactaggggtaaaggtagcgaagaaaaagcaatcaagatctatcctagataagctggaagatctctgatcatgaatcatagtcttaacccaactagataaatactcacttggctaatactataacccaattaaatcatgatttccatacatttgggatcaccttacatgtttactaattctcttctgaatttaccattaggcaaacatcgttgatgattttcaagaatacaactatggaacaatggaaagttaccagacagaagctcctaggcatgtttctgggacaaatgagccaaatccagttgaagaaatatttactgaagaatatctaacgggagaggattatgattcccagaggaaaaattctgaggatacactatatgaaaacaaagaaatagacggcagggattctgatcttctggtagatggagatttaggcgaatatgatttttatgaatataaagaatatgaagataaaccaacaagcccccctaatgaagaatttggtccaggtgtaccagcagaaactgatattacagaaacaagcataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatacccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagaggggaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccacctgggaaggatgggctgccaggacaccctgggcaacgtggggagactggatttcaaggcaagaccggccctcctgggccagggggagtggttggaccacagggaccaaccggtgagactggtccaataggggaacgtgggcatcctggccctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttaccagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagatggagacaagggtgaaattggtgagccgggacaaaaaggcagcaagggtgacaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtcccatggggccacctggtcctccaggcccaagaggccctcaaggtcccaatggagctgatggaccacaaggacccccagggtctgttggttcagttggtggtgttggagaaaagggtgaacctggagaagcagggaacccagggcctcctggggaagcaggtgtaggcggtcccaaaggagaaagaggagagaaaggggaagctggtccacctggagctgctggacctccaggtgccaaggggccaccaggtgatgatggccctaagggtaacccgggtcctgttggttttcctggagatcctggtcctcctggggaacttggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggcttaccaggtcctcaaggcccaaagggtaacaaaggctctactggacccgctggccagaaaggtgacagtggtcttccagggcctcctgggcctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacatcgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtgcgtccagaaaaggctatgaaaagactgtcattgaaatcaatacaccaaaaattgatcaagtacctattgttgatgtcatgatcaatgactttggtgatcagaatcagaagttcggatttgaagttggtcctgtttgttttcttggctaagattaagacaaagaacatatcaaatcaacagaaaatataccttggtgccaccaacccattttgtgccacatgcaagttttgaataaggatggtatagaaaacaacgctgcatatacaggtaccatttaggaaataccgatgcctttgtgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccatctgactctaattttatgagaattgcagaactctgatggcaataaatatatgtattatgaaaaaataaagttgtaatttctgatgactctaagtccctttctttggttaataataaaatgcctttgtatatattga

SEQ ID NO: 154 - DNA, 186 bp, Homo sapiens
gtagcgaagaaaaagcaatcaagatctatcctagataagctggaagatctctgatcatgaatcatagtcttaacccaactagataaatactcacttggctaatactataacccaattaaatcatgatttccatacatttgggatcaccttacatgtttactaattctcttctgaatttaccattag

SEQ ID NO: 155 - protein, 1806 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGYPGPPGPPGEQGLPGAAGKEGAKGDPGPQGISGKDGPAGLRGFPGERGLPGAQGAPGLKGGEGPQGPPGPVGSPGERGSAGTAGPIGLRGRPGPQGPPGPAGEKGAPGEKGPQGPAGRDGVQGPVGLPGPAGPAGSPGEDGDKGEIGEPGQKGSKGGKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMISDFGDQNQKFGFEVGPVCFLG

SEQ ID NO: 156 - protein, 1806 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGEPGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGSPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCASRKGYEKTVIEINTPKIDQVPIVDVMINDFGDQNQKFGFEVGPVCFLG

SEQ ID NO: 157 - protein, 1818 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEKKKSNFKKKMRTVATKSKEKSKKFTPPKSEKFSSKKKKSYQASAKAKLGVKANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGRMGCPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPWGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMINDFGDQNQKFGFEVGPVCFLG

SEQ ID NO: 158 - protein, 1767 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGRMGCPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPWGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMINDFGDQNQKFGFEVGPVCFLG

SEQ ID NO: 159 - protein, 1818 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEKKKSNFKKKMRTVATKSKEKSKKFTPPKSEKFSSKKKKSYQASAKAKLGVKANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGYPGPPGPPGEQGLPGAAGKEGAKGDPGPQGISGKDGPAGLRGFPGERGLPGAQGAPGLKGGEGPQGPPGPVGSPGERGSAGTAGPIGLRGRPGPQGPPGPAGEKGAPGEKGPQGPAGRDGVQGPVGLPGPAGPAGSPGEDGDKGEIGEPGQKGSKGGKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMISDFGDQNQKFGFEVGPVCFLG

SEQ ID NO: 160 - protein, 1806 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGRMGCPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPWGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMINDFGDQNQKFGFEVGPVCFLG

SEQ ID NO: 161 - protein, 1767 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGYPGPPGPPGEQGLPGAAGKEGAKGDPGPQGISGKDGPAGLRGFPGERGLPGAQGAPGLKGGEGPQGPPGPVGSPGERGSAGTAGPIGLRGRPGPQGPPGPAGEKGAPGEKGPQGPAGRDGVQGPVGLPGPAGPAGSPGEDGDKGEIGEPGQKGSKGGKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMISDFGDQNQKFGFEVGPVCFLG

SEQ ID NO: 162 - protein, 328 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEKKKSNFKKKMRTVATKSKEKSKKFTPPKSEKFSSKKKKSYQASAKAKLGVKVAKKKQSRSILDKLEDL

SEQ ID NO: 163 - DNA, 30 bp, Homo sapiens
aatcaagatctatcctagataagctggaag

SEQ ID NO: 164 - DNA, 30 bp, Homo sapiens
aattgggttatagtattagccaagtgagta

SEQ ID NO: 165 - DNA, 101 bp, Homo sapiens
aatcaagatctatcctagataagctggaagatctctgatcatgaatcatagtcttaacccaactagataaatactcacttggctaatactataacccaatt

SEQ ID NO: 166 - DNA, 50 bp, Homo sapiens
ttcagaactgttaacatcgctgacgggaagtggcatcgggtagcaatcag

SEQ ID NO: 167 - protein, 68 aa, Artificial sequence
  Synthetic peptide
KKKSNFKKKMRTVATKSKEKSKKFTPPKSEKFSSKKKKSYQASAKAKLGVKVAKKKQSRSILDKLEDL

SEQ ID NO: 168 - protein, 17 aa, Artificial sequence
  Synthetic peptide
VAKKKQSRSILDKLEDL

SEQ ID NO: 169 - protein, 6 aa, Homo sapiens
GEPAPG

SEQ ID NO: 170 - protein, 27 aa, Homo sapiens
PSLQPHWGSLSIVIAMRTLGFWTSAHC

(19) United States
(12) Patent Application Publication
(10) Pub. No.: US 2010/0261169 A1
(43) Pub. Date: Oct. 14, 2010

(54) NOVEL NUCLEOTIDE AND AMINO ACID SEQUENCES, AND METHODS OF USE THEREOF FOR DIAGNOSIS

(76) Inventors: Shira Wallach, Hod Hasharon (IL); Shirley Sameah-Greenwald, Kfar Saba (IL); Amit Novik, Tel Mond (IL); Elena Tsypkin, Tel Aviv (IL); Sergey Nemzer, Ra'anana (IL)

Correspondence Address: FENNEMORE CRAIG, 3003 NORTH CENTRAL AVENUE, SUITE 2600, PHOENIX, AZ, 85012, US

(21) Appl. No.: 12/524,770

(22) PCT Filed: Dec. 30, 2007
(86) PCT No.: PCT/IL07/01626

§ 371 Date: Dec. 15, 2009

Related U.S. Application Data
(60) Provisional application No. 60/897,831, filed on Jan. 29, 2007.
(60) Provisional application No. 60/935,205, filed on Jul. 31, 2007.

(51) Int. Cl. C12Q 1/68; C07H 21/04; C07K 14/00; C07K 16/00; G01N 33/68; C12Q 1/00; G01N 33/48; G01N 33/53

(52) U.S. Cl. 435/6; 536/23.1; 530/324; 530/350; 530/387.9; 436/86; 435/29; 436/94; 435/7.92; 435/7.1; 536/24.33

(57) ABSTRACT

The present invention relates to diagnostic markers comprising novel splice variants of known proteins and polynucleotides encoding same, useful in the qualitative and/or quantitative detection of various diseases and/or pathological conditions in a subject, and to the use of known proteins and polynucleotides encoding same for diagnosis. Particularly, the invention relates to the diagnosis of a disease in a sample of body fluid or secretion obtained from the subject, and to the diagnosis of cancer.

### FIELD OF THE INVENTION

The present invention is related to novel nucleotide and protein sequences, and assays and methods of use thereof.

### BACKGROUND OF THE INVENTION

Diagnostic markers are important for early diagnosis of many diseases, as well as for predicting a response to treatment, monitoring treatment progress and determining prognosis of the disease.

Serum markers are examples of diagnostic markers, and are used for diagnosis of many different diseases. Typically, serum markers encompass secreted proteins and/or peptides; however, some serum markers may be released to the blood upon tissue lysis, for example from myocardial infarction (Troponin-I being a specific example). Serum markers can also be used as indicative risk factors of a disease (for example base-line levels of CRP, as a predictor of cardiovascular disease); to monitor disease activity and progression (for example, determination of CRP levels to monitor acute phase inflammatory response); and to predict and monitor drug response (for example, as shedded fragments of the protein Erb-B2).

Immunohistochemistry (IHC) is the study of the distribution of an antigen of choice in a sample based on specific antibody-antigen binding, typically performed on tissue slices. The antibody features a label which can be detected, for example as a stain which is detectable under a microscope. Preparation of the tissue slices for the assay involves fixation; IHC is therefore particularly suitable for antibody-antigen reactions that are not disturbed or destroyed by the process of fixing the tissue slices.

IHC permits determining the localization of the bound antibody-antigen, and hence mapping the presence of the antigen within the tissue and even within different compartments in the cell. Such mapping can provide useful diagnostic information, including:

1) The histological type of the tissue sample
2) The presence of specific cell types within the sample
3) Information regarding the physiological and/or pathological state of cells (e.g. which phase of the cell-cycle they are in)
4) The presence of disease related changes within the sample
5) Differentiation between specific disease subtypes where it is already known that the tissue is diseased (for example, the differentiation between different tumor types when it is already known the sample was taken from cancerous tissue).

IHC information is valuable for more than diagnosis. It can also be used to determine prognosis and progression of a therapy treatment (for example, as in the case of HER-2 in breast cancer) as well as to monitor the disease state.

IHC protein markers could be from any cellular location. Most often these markers are membrane proteins but secreted proteins or intracellular proteins (including intranuclear) can also be used as an IHC marker.

Although widely used as diagnostic tool, the IHC technique has at least two major disadvantages. It is performed on tissue samples and therefore a tissue sample has to be collected from the patient, which most often requires invasive procedures like biopsy associated with pain, discomfort, hospitalization and risk of infection. In addition, the interpretation of the result is observer dependent and therefore subjective. There is no measured value but rather only an estimation (on a scale of 1-4) of how prevalent the antigen is on the target.

Thus, there is a recognized need for, and it would be highly advantageous to have, an alternative diagnostic tool for diagnosing and monitoring diseases.

### SUMMARY OF THE INVENTION

The present invention provides novel nucleic acid and amino acid sequences, which can be used as diagnostic markers.

According to one aspect, the present invention provides a number of novel variants of known proteins which are found in serum and can be used as diagnostic markers. The present invention overcomes the many deficiencies of the background art with regard to the need to obtain tissue samples and subjective interpretations of results. In certain embodiments of the present invention, tissue specific markers are identifiable in serum or plasma. Thus, according to the teachings of the present invention, a simple blood test can provide qualitative and/or quantitative indication of various diseases and/or pathological conditions, according to the expression of certain marker(s).

According to another aspect, the present invention discloses the novel use of known proteins as diagnostic markers. In some embodiments, the markers disclosed can also be used for in-vivo imaging applications.

It is disclosed in the present invention for the first time that the protein variants of the invention are useful as diagnostic markers for various diseases and/or pathological conditions as described in greater detail below. The variants themselves are described by “cluster” or by gene, as these variants are splice variants of known proteins. Therefore, as used in the present invention, the term “marker-detectable disease” refers to a disease that may be detected by a particular marker, with regard to the description of the disease provided herein below. The markers of the present invention, alone or in combination, show a high degree of differential diagnosis between disease and non-disease states.

The present invention further relates to diagnostic assays for detecting a disease, particularly in a sample taken from a subject (patient), preferably a blood sample or a body secretion sample. According to certain embodiments, the diagnostic assays disclosed in the present invention are immunoassays, including, for example, ELISA, RIA, immunohistochemical assay, FACS, radio-imaging assays, slot blots or western blot. According to another embodiments, the diagnostic assays disclosed in the present invention are NAT (nucleic acid amplification technology)-based assays, including, for example, PCR or variations thereof, e.g. real-time PCR. According to other embodiments, the assays encompass nucleic acid hybridization assays. The diagnostic assays can be qualitative or quantitative.

According to certain embodiments, the present invention provides a diagnostic marker comprising a novel splice variant of a known protein or a polynucleotide encoding same, wherein the protein is selected from the group consisting of Growth/differentiation factor 15 precursor (SwissProt accession identifier GDF15_HUMAN; known also according to the synonyms GDF-15; Placental bone morphogenic protein; Placental TGF-beta; Macrophage inhibitory cytokine 1; MIC-1; Prostate differentiation factor; NSAID-regulated protein 1; NRG-1); Tumor necrosis factor receptor superfamily member 1A precursor (SwissProt accession identifier TNR1A_HUMAN; known also according to the synonyms p60; TNF-R1; TNF-RI; TNFR-I; p55; CD120a antigen); Myosin-binding protein C, cardiac-type (SwissProt accession identifier MYPC_HUMAN; known also according to the synonyms Cardiac MyBP-C; C-protein, cardiac muscle isoform and Collagen alpha 1 (SwissProt accession identifier COBA1_HUMAN. According to certain embodiments, the diagnostic marker is found in a body fluid or secretion.

According to one embodiment, the novel splice variant is an isolated polynucleotide comprising a nucleic acid having a nucleic acid sequence as set forth in any one of SEQ ID NOs: 35-41, 60-72, 109-115, 153, or a sequence homologous thereto. According to one embodiment, the isolated polynucleotide is at least 85% homologous to any one of SEQ ID NOs: 35-41, 60-72, 109-115, 153. According to another embodiment, the isolated polynucleotide is at least 95% homologous to any one of SEQ ID NOs: 35-41, 60-72, 109-115, 153.

According to another embodiment, the novel splice variant is an isolated polynucleotide comprising a nucleic acid having a nucleic acid sequence as set forth in any one of SEQ ID NOs: 42-50, 73-95, 116-126, 154 or a sequence homologous thereto. According to one embodiment, the isolated polynucleotide is at least 85% homologous to any one of SEQ ID NOs: 42-50, 73-95, 116-126, 154. According to another embodiment, the isolated polynucleotide is at least 95% homologous to any one of SEQ ID NOs: 42-50, 73-95, 116-126, 154.

According to certain embodiments, the present invention also encompasses isolated polynucleotides having a sequence complementary to any one of the nucleic acid sequences listed herein. According to other embodiments, this invention provides an oligonucleotide of at least about 12 nucleotides, specifically hybridizable with the polynucleotides of this invention. The present invention further provides vectors, cells, liposomes and compositions comprising the isolated polynucleotides of this invention.

According to yet another embodiment, the novel splice variant is an isolated protein or polypeptide having an amino acid sequence as set forth in any one of SEQ ID NOs: 55, 56, 98-108, 129-135, 162, or a sequence homologous thereto. According to one embodiment, the isolated protein or polypeptide is at least 85% homologous to any one of SEQ ID NOs: 55, 56, 98-108, 129-135, 162. According to another embodiment, the isolated polypeptide is at least 95% homologous to any one of SEQ ID NOs: 55, 56, 98-108, 129-135, 162.

According to some embodiments, the sample taken from a subject (patient) to perform the diagnostic assay according to the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cell(s) or tissue(s), wherein the cell or tissue can be obtained from an organ selected from, but not limited to lung, colon, ovarian, prostate, kidney, liver and/or breast tissue; stool or a tissue sample, or any combination thereof. In some embodiments, the term encompasses samples of in vivo cell culture constituents. Prior to be subjected to the diagnostic assay, the sample can optionally be diluted with a suitable eluant.

According to certain embodiments, the present invention now discloses a cluster designated herein D11717, comprising novel amino acid and nucleic acid sequences that are variants of the known GDF15_HUMAN (SEQ ID NO: 51). The novel variant polynucleotides and polypeptides described by the present invention are useful as diagnostic markers, preferably as serum markers.

Surprisingly, the present invention now shows that D11717 variants are expressed specifically in acute and chronic inflammation, and/or cardiovascular and/or cerebrovascular conditions and diseases, and thus can indicate the onset, severity or prognosis of acute and chronic inflammation, and/or cardiovascular and/or cerebrovascular conditions and diseases and can be used for the diagnosis, prognosis, treatment selection, and treatment monitoring and/or assessment of acute and chronic inflammation, and/or cardiovascular and/or cerebrovascular conditions and diseases, as is described in a greater detail below.

Surprisingly, the present invention now shows that D11717 known proteins (SEQ ID NOs: 51-54) and their variants are differentially expressed in cerebrovascular conditions and diseases, and thus can indicate the onset, severity or prognosis of cerebrovascular conditions and diseases and can be used for the diagnosis, prognosis, treatment selection, and treatment monitoring and/or assessment of cerebrovascular conditions and diseases, as is described in a greater detail below.

According to the present invention, the D11717 known proteins (SEQ ID NOs: 51-54) and their variants are useful as diagnostic markers, preferably as serum marker.

The present invention further shows that D11717 variants are differentially expressed in cancerous tissues, particularly in cancerous epithelial malignant tissues, cancerous prostate, cancerous pancreas, cancerous colorectal, cancerous breast, cancerous liver, cancerous skin and cancerous kidney tissues, and thus can be used for the diagnosis, prognosis, treatment selection, and treatment monitoring and/or assessment of cancer, particularly epithelial malignant cancer, prostate cancer, pancreas cancer, colorectal cancer, breast cancer, liver cancer, skin cancer and renal cancer.

Surprisingly, the present invention now shows that D11717 known proteins (SEQ ID NOs: 51-54) and their variants are differentially expressed in cancerous tissues, particularly in cancerous liver and cancerous kidney tissues, and thus can be used for the diagnosis, prognosis, treatment selection, and treatment monitoring and/or assessment of cancer, particularly liver cancer and renal cancer.

In some embodiments, the novel isolated chimeric proteins or polypeptides of the invention comprise an amino acid sequence corresponding to or homologous to SEQ ID NO: 55 and 56.

According to another embodiment, the isolated polypeptide comprises an amino acid sequence as set forth in SEQ ID NO: 55 and 56.

According to the present invention there is provided an isolated chimeric polypeptide, comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), and a second amino acid sequence being at least 90% homologous to amino acids 13-308 of known protein GDF15_HUMAN_V1 (SEQ ID NO:54), which also corresponds to amino acids 36-331 of D11717_P4 (SEQ ID NO:55), wherein said first amino acid sequence and second amino acid sequence are contiguous and in a sequential order.

According to the present invention there is provided an isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

According to the present invention there is provided an isolated chimeric polypeptide, comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), and a second amino acid sequence being at least 90% homologous to amino acids 13-308 of known protein Q9BWA0_HUMAN (SEQ ID NO: 53), which also corresponds to amino acids 36-331 of D11717_P4 (SEQ ID NO:55), wherein said first amino acid sequence and second amino acid sequence are contiguous and in a sequential order.

According to the present invention there is provided an isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

According to the present invention there is provided an isolated chimeric polypeptide, comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), a second amino acid sequence being at least 90% homologous to amino acids 13-201 of known protein(s) NP_{—}004855 (SEQ ID NO: 52), which also corresponds to amino acids 36-224 of D11717_P4 (SEQ ID NO:55), a bridging amino acid H corresponding to amino acid 225 of D11717_P4 (SEQ ID NO:55), a third amino acid sequence being at least 90% homologous to amino acids 203-268 of known protein(s) NP_{—}004855 (SEQ ID NO: 52), which also corresponds to amino acids 226-291 of D11717_P4 (SEQ ID NO:55), a bridging amino acid V corresponding to amino acid 292 of D11717_P4 (SEQ ID NO:55), and a fourth amino acid sequence being at least 90% homologous to amino acids 270-308 of known protein(s) NP_{—}004855 (SEQ ID NO: 52), which also corresponds to amino acids 293-331 of D11717_P4 (SEQ ID NO:55), wherein said first amino acid sequence, second amino acid sequence, bridging amino acid, third amino acid sequence, bridging amino acid and fourth amino acid sequence are contiguous and in a sequential order.

According to the present invention there is provided an isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

According to certain embodiments, the present invention now discloses a cluster designated herein HSTNFR1A, comprising novel amino acid and nucleic acid sequences that are variants of the TNR1A_HUMAN (SEQ ID NO: 96). The novel variant polynucleotides and polypeptides described by the present invention are useful as diagnostic markers, preferably as serum markers.

Surprisingly, the present invention now shows that the HSTNFR1A variants are expressed specifically in acute and chronic inflammation, and/or cardiovascular and/or cerebrovascular conditions and diseases, and thus can indicate the onset, severity or prognosis of acute and chronic inflammation, and/or cardiovascular and/or cerebrovascular conditions and diseases and can be used for the diagnosis, prognosis, treatment selection, and treatment monitoring and/or assessment of acute and chronic inflammation, and/or cardiovascular and/or cerebrovascular conditions and diseases.

Surprisingly, the present invention now shows that HSTNFR1A known proteins (SEQ ID NOs: 96-97) and their variants are differentially expressed in cardiovascular and/or cerebrovascular conditions and diseases and thus can indicate the onset, severity or prognosis of cardiovascular and/or cerebrovascular conditions and diseases and can be used for the diagnosis, prognosis, treatment selection, and treatment monitoring and/or assessment of cardiovascular and/or cerebrovascular conditions and diseases.

According to the present invention, the HSTNFR1A known proteins (SEQ ID NOs: 96-97) and their variants are useful as diagnostic markers, preferably for in-vivo imaging applications.

The present invention further shows that HSTNFR1A variants are differentially expressed in cancerous tissues, particularly in cancerous colorectal tissues and cancerous kidney and thus can be used for the diagnosis, prognosis, treatment selection, and treatment monitoring and/or assessment of cancer, particularly colorectal cancer and renal cancer.

Surprisingly, the present invention now shows that HSTNFR1A known proteins (SEQ ID NOs: 96-97) are differentially expressed in cancerous tissues, particularly in cancerous kidney and thus can be used for the diagnosis, prognosis, treatment selection, and treatment monitoring and/or assessment of cancer, particularly renal cancer.

In some embodiments, the novel isolated chimeric proteins or polypeptides of the invention comprise an amino acid sequence corresponding to or homologous to 98-108.

According to another embodiment, the isolated polypeptide comprises an amino acid sequence as set forth in SEQ ID NO: 98-108.

According to the present invention there is provided an isolated chimeric polypeptide, comprising a first amino acid sequence being at least 70%, optionally at least 80%, preferably at least 85%, more preferably at least 90% and most preferably at least 95%, homologous to a polypeptide having the sequence GDCTKNGSDVPVENLYPSKYTQQVCIHSCFQESMIKECGCAYIFYPRPQNVEYCDYRKHSSWGYCYYKLQ VDFSSDHLGCFTKCRKPCSVTSYQLSAGYSRWPSVTSQEWVFQMLSRQNNYTVNNKRNGVAKVNIFFKE LNYKTNSESPSVT (SEQ ID NO: 146) corresponding to amino acids 1-152 of HSTNFR1A_P26 (SEQ ID NO:102), and a second amino acid sequence being at least 90% homologous to amino acids 14-455 of known protein(s) TNR1A_HUMAN (SEQ ID NO: 96) and NP_{—}001056 (SEQ ID NO: 96), which also corresponds to amino acids 153-594 of HSTNFR1A_P26 (SEQ ID NO:102), wherein said first amino acid sequence and second amino acid sequence are contiguous and in a sequential order.

An isolated polypeptide comprising a head of HSTNFR1A_P26 (SEQ ID NO:102), comprising a polypeptide being at least 70%, optionally at least about 80%, preferably at least about 85%, more preferably at least about 90% and most preferably at least about 95% homologous to the sequence GDCTKNGSDVPVENLYPSKYTQQVCIHSCFQESMIKECGCAYIFYPRPQNVEYCDYRKHSSWGYCYYKLQ VDFSSDHLGCFTKCRKPCSVTSYQLSAGYSRWPSVTSQEWVFQMLSRQNNYTVNNKRNGVAKVNIFFKE LNYKTNSESPSVT (SEQ ID NO: 146) of HSTNFR1A_P26 (SEQ ID NO:102).

According to the present invention there is provided an isolated chimeric polypeptide, comprising a first amino acid sequence being at least 90% homologous to MGLSTVPDLLLPLVLLELLVGIYPSGVIGLVPHLGDREKRDSVCPQGKYIHPQNNSICCTKCHKGTYLYND CP corresponding to amino acids 1-73 of known protein(s) TNR1A HUMAN (SEQ ID NO: 96) and NP_{—}001056 (SEQ ID NO: 96), which also corresponds to amino acids 1-73 of HSTNFR1A_P27 (SEQ ID NO:103), and a second amino acid sequence being at least 90% homologous to amino acids 278-455 of known protein(s) TNR1A_HUMAN (SEQ ID NO: 96) and NP_{—}001056 (SEQ ID NO: 96), which also corresponds to amino acids 74-251 of HSTNFR1A_P27 (SEQ ID NO:103), wherein said first amino acid sequence and second amino acid sequence are contiguous and in a sequential order.

According to the present invention there is provided an isolated chimeric polypeptide comprising an edge portion of HSTNFR1A_P27 (SEQ ID NO:103), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise PG, having a structure as follows: a sequence starting from any of amino acid numbers 73-x to 73; and ending at any of amino acid numbers 74+((n−2)−x), in which x varies from 0 to n−2.

According to certain embodiments, the present invention now discloses a cluster designated herein Z18303, comprising novel amino acid and nucleic acid sequences that are variants of the known MYPC_HUMAN (SEQ ID NO: 127). The novel variant polynucleotides and polypeptides described by the present invention are useful as diagnostic markers, preferably as serum markers.

Surprisingly, the present invention now shows that Z18303 variants are expressed specifically in heart tissue, and thus can be used for the diagnosis, prognosis, treatment selection, and treatment monitoring and/or assessment of cardiovascular disease in a subject, and can be used for the selection of treatment, treatment monitoring, diagnosis or prognosis assessment of any cardiovascular disease, including, inter alia, myocardial infarct, acute coronary syndrome, coronary artery disease, angina pectoris (stable and unstable), cardiomyopathy, myocarditis, congestive heart failure or any type of heart failure, reinfarction, assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof, as is described in a greater detail below.

In some embodiments, the isolated chimeric proteins or polypeptides of the invention comprise an amino acid sequence corresponding to or homologous to SEQ ID NO: 129-135.

According to another embodiment, the isolated polypeptide comprises an amino acid sequence as set forth in SEQ ID NO: 129-135.

According to the present invention there is provided an isolated chimeric polypeptide, comprising a first amino acid sequence being at least 90% homologous to amino acids 1-769 of known protein MYPC_HUMAN (SEQ ID NO: 127) and Q9UM53_HUMAN (SEQ ID NO: 127), which also corresponds to amino acids 1-769 of Z18303_P4 (SEQ ID NO:130), and a second amino acid sequence being at least 70%, optionally at least 80%, preferably at least 85%, more preferably at least 90% and most preferably at least 95% homologous to a polypeptide having the sequence GEAGRGPSWRTQRGSPKEGPSVRSFSHSTNITGMLPEPSGNRTGFSAQKEPRSGGDGGRCIQQISCDTAVH EPCPECRGSTEQRAIPVPGGDAFRRLRR (SEQ ID NO: 147) corresponding to amino acids 770-868 of Z18303_P4 (SEQ ID NO:130), wherein said first amino acid sequence and second amino acid sequence are contiguous and in a sequential order.

According to the present invention there is provided an isolated polypeptide comprising an edge portion of Z18303_P4 (SEQ ID NO:130), comprising an amino acid sequence being at least 70%, optionally at least about 80%, preferably at least about 85%, more preferably at least about 90% and most preferably at least about 95% homologous to the sequence GEAGRGPSWRTQRGSPKEGPSVRSFSHSTNITGMLPEPSGNRTGFSAQKEPRSGGDGGRCIQQISCDTAVH EPCPECRGSTEQRAIPVPGGDAFRRLRR (SEQ ID NO: 147) of Z18303_P4 (SEQ ID NO:130).

According to the present invention there is provided an isolated chimeric polypeptide comprising Z18303_P4 (SEQ ID NO:130), comprising a first amino acid sequence being at least 90% homologous to amino acids 1-247 of known protein NP_{—}000247 (SEQ ID NO: 128), which also corresponds to amino acids 1-247 of Z18303_P4 (SEQ ID NO:130), a bridging amino acid D corresponding to amino acid 248 of Z18303_P4 (SEQ ID NO:130), a second amino acid sequence being at least 90% homologous to amino acids 249-535 of known protein(s) NP_{—}000247 (SEQ ID NO: 128), which also corresponds to amino acids 249-535 of Z18303_P4 (SEQ ID NO:130), a bridging amino acid A corresponding to amino acid 536 of Z18303_P4 (SEQ ID NO:130), a third amino acid sequence being at least 90% homologous to amino acids 537-769 of known protein NP_{—}000247 (SEQ ID NO: 128), which also corresponds to amino acids 537-769 of Z18303_P4 (SEQ ID NO:130), and a fourth amino acid sequence being at least 70%, optionally at least 80%, preferably at least 85%, more preferably at least 90% and most preferably at least 95% homologous to a polypeptide having the sequence GEAGRGPSWRTQRGSPKEGPSVRSFSHSTNITGMLPEPSGNRTGFSAQKEPRSGGDGGRCIQQISCDTAVH EPCPECRGSTEQRAIPVPGGDAFRRLRR (SEQ ID NO: 147) corresponding to amino acids 770-868 of Z18303_P4 (SEQ ID NO:130), wherein said first amino acid sequence, bridging amino acid, second amino acid sequence, bridging amino acid, third amino acid sequence and fourth amino acid sequence are contiguous and in a sequential order.

According to the present invention there is provided an isolated polypeptide comprising an edge portion of Z18303_P4 (SEQ ID NO:130), comprising an amino acid sequence being at least 70%, optionally at least about 80%, preferably at least about 85%, more preferably at least about 90% and most preferably at least about 95%) homologous to the sequence GEAGRGPSWRTQRGSPKEGPSVRSFSHSTNITGMLPEPSGNRTGFSAQKEPRSGGDGGRCIQQISCDTAVH EPCPECRGSTEQRAIPVPGGDAFRRLRR (SEQ ID NO: 147) of Z18303_P4 (SEQ ID NO:130).

According to the present invention there is provided an isolated chimeric polypeptide comprising Z18303_P10 (SEQ ID NO:133), comprising a first amino acid sequence being at least 90% homologous to amino acids 1-1271 of known proteins MYPC_HUMAN (SEQ ID NO: 127) and Q9UM53_HUMAN (SEQ ID NO: 127), which also corresponds to amino acids 1-1271 of Z18303_P10 (SEQ ID NO:133), and a second amino acid sequence being at least 70%, optionally at least 80%, preferably at least 85%, more preferably at least 90% and most preferably at least 95% homologous to a polypeptide having the sequence GEEPSGPGPGRWEERAAVGTLGFAPLSSAKHLPWLQGHGPCHAQMGNSFQKAGRTQ (SEQ ID NO: 148) corresponding to amino acids 1272-1327 of Z18303_P10 (SEQ ID NO:133), wherein said first amino acid sequence and second amino acid sequence are contiguous and in a sequential order.

According to the present invention there is provided an isolated polypeptide comprising an edge portion of Z18303_P10 (SEQ ID NO:133), comprising an amino acid sequence being at least 70%, optionally at least about 80%, preferably at least about 85%, more preferably at least about 90% and most preferably at least about 95% homologous to the sequence GEEPSGPGPGRWEERAAVGTLGFAPLSSAKHLPWLQGHGPCHAQMGNSFQKAGRTQ (SEQ ID NO: 148) of Z18303_P10 (SEQ ID NO:133).

According to the present invention there is provided an isolated chimeric polypeptide, comprising a first amino acid sequence being at least 90% homologous to corresponding to amino acids 1-247 of known protein NP_{—}000247 (SEQ ID NO: 128), which also corresponds to amino acids 1-247 of Z18303_P10 (SEQ ID NO:133), a bridging amino acid D corresponding to amino acid 248 of Z18303_P10 (SEQ ID NO:133), a second amino acid sequence being at least 90% homologous to amino acids 249-535 of known protein NP_{—}000247 (SEQ ID NO: 128), which also corresponds to amino acids 249-535 of Z18303_P10 (SEQ ID NO:133), a bridging amino acid A corresponding to amino acid 536 of Z18303_P10 (SEQ ID NO:133), a third amino acid sequence being at least 90% homologous to amino acids 537-819 of known protein(s) NP_{—}000247 (SEQ ID NO: 128), which also corresponds to amino acids 537-819 of Z18303_P10 (SEQ ID NO:133), a bridging amino acid R corresponding to amino acid 820 of Z18303_P10 (SEQ ID NO:133), a fourth amino acid sequence being at least 90% homologous to amino acids 821-1271 of known protein(s) NP_{—}000247 (SEQ ID NO: 128), which also corresponds to amino acids 821-1271 of Z18303_P10 (SEQ ID NO:133), and a fifth amino acid sequence being at least 70%, optionally at least 80%, preferably at least 85%, more preferably at least 90% and most preferably at least 95% homologous to a polypeptide having the sequence GEEPSGPGPGRWEERAAVGTLGFAPLSSAKHLPWLQGHGPCHAQMGNSFQKAGRTQ (SEQ ID NO: 148) corresponding to amino acids 1272-1327 of Z18303_P10 (SEQ ID NO:133), wherein said first amino acid sequence, bridging amino acid, second amino acid sequence, bridging amino acid, third amino acid sequence, bridging amino acid, fourth amino acid sequence and fifth amino acid sequence are contiguous and in a sequential order.

According to the present invention there is provided an isolated polypeptide comprising an edge portion of Z18303_P10 (SEQ ID NO:133), comprising an amino acid sequence being at least 70%, optionally at least about 80%, preferably at least about 85%, more preferably at least about 90% and most preferably at least about 95% homologous to the sequence GEEPSGPGPGRWEERAAVGTLGFAPLSSAKHLPWLQGHGPCHAQMGNSFQKAGRTQ (SEQ ID NO: 148) of Z18303_P10 (SEQ ID NO:133).

According to the present invention there is provided an isolated chimeric polypeptide, comprising a first amino acid sequence being at least 90% homologous to amino acids 1-1269 of known proteins MYPC_HUMAN (SEQ ID NO: 127) and Q9UM53_HUMAN (SEQ ID NO: 127), which also corresponds to amino acids 1-1269 of Z18303_P12 (SEQ ID NO:134), and a second amino acid sequence being at least 70%, optionally at least 80%, preferably at least 85%, more preferably at least 90% and most preferably at least 95% homologous to a polypeptide having the sequence CLSDQAGSWGWPGTTGCQPRARSLEGSWGNPSLLLDVCVTSVSPVLRWGISRAVVGQS (SEQ ID NO: 149) corresponding to amino acids 1270-1327 of Z18303_P12 (SEQ ID NO:134), wherein said first amino acid sequence and second amino acid sequence are contiguous and in a sequential order.

According to the present invention there is provided an isolated polypeptide comprising an edge portion of Z18303_P12 (SEQ ID NO:134), comprising an amino acid sequence being at least 70%, optionally at least about 80%, preferably at least about 85%, more preferably at least about 90% and most preferably at least about 95% homologous to the sequence CLSDQAGSWGWPGTTGCQPRARSLEGSWGNPSLLLDVCVTSVSPVLRWGISRAVVGQS (SEQ ID NO: 149) of Z18303_P12 (SEQ ID NO:134).

According to the present invention there is provided an isolated chimeric polypeptide comprising a first amino acid sequence being at least 90% homologous to amino acids 1-247 of known protein NP_{—}000247 (SEQ ID NO: 128), which also corresponds to amino acids 1-247 of Z18303_P12 (SEQ ID NO:134), a bridging amino acid D corresponding to amino acid 248 of Z18303_P12 (SEQ ID NO:134), a second amino acid sequence being at least 90% homologous to amino acids 249-535 of known protein NP_{—}000247 (SEQ ID NO: 128), which also corresponds to amino acids 249-535 of Z18303_P12 (SEQ ID NO:134), a bridging amino acid A corresponding to amino acid 536 of Z18303_P12 (SEQ ID NO:134), a third amino acid sequence being at least 90% homologous to amino acids 537-819 of known protein NP_{—}000247 (SEQ ID NO: 128), which also corresponds to amino acids 537-819 of Z18303_P12 (SEQ ID NO:134), a bridging amino acid R corresponding to amino acid 820 of Z18303—P12 (SEQ ID NO:134), a fourth amino acid sequence being at least 90% homologous to amino acids 821-1269 of known protein NP_{—}000247 (SEQ ID NO: 128), which also corresponds to amino acids 821-1269 of Z18303_P12 (SEQ ID NO:134), and a fifth amino acid sequence being at least 70%, optionally at least 80%, preferably at least 85%, more preferably at least 90% and most preferably at least 95% homologous to a polypeptide having the sequence CLSDQAGSWGWPGTTGCQPRARSLEGSWGNPSLLLDVCVTSVSPVLRWGISRAVVGQS (SEQ ID NO: 149) corresponding to amino acids 1270-1327 of Z18303_P12 (SEQ ID NO:134), wherein said first amino acid sequence, bridging amino acid, second amino acid sequence, bridging amino acid, third amino acid sequence, bridging amino acid, fourth amino acid sequence and fifth amino acid sequence are contiguous and in a sequential order.

According to the present invention there is provided an isolated polypeptide comprising an edge portion of Z18303_P12 (SEQ ID NO:134), comprising an amino acid sequence being at least 70%, optionally at least about 80%, preferably at least about 85%, more preferably at least about 90% and most preferably at least about 95% homologous to the sequence CLSDQAGSWGWPGTTGCQPRARSLEGSWGNPSLLLDVCVTSVSPVLRWGISRAVVGQS (SEQ ID NO: 149) of Z18303_P12 (SEQ ID NO:134).

According to the present invention there is provided an isolated chimeric, comprising a amino acid sequence being at least 90% homologous to amino acids 1-1110 of known proteins MYPC_HUMAN (SEQ ID NO: 127) and Q9UM53_HUMAN (SEQ ID NO: 127), which also corresponds to amino acids 1-1110 of Z18303_P19 (SEQ ID NO:135).

According to the present invention there is provided an isolated chimeric polypeptide, comprising a first amino acid sequence being at least 90% homologous to amino acids 1-247 of known protein NP_{—}000247 (SEQ ID NO: 128), which also corresponds to amino acids 1-247 of Z18303_P19 (SEQ ID NO:135), a bridging amino acid D corresponding to amino acid 248 of Z18303_P19 (SEQ ID NO:135), a second amino acid sequence being at least 90% homologous to amino acids 249-535 of known protein NP_{—}000247 (SEQ ID NO: 128), which also corresponds to amino acids 249-535 of Z18303_P19 (SEQ ID NO:135), a bridging amino acid A corresponding to amino acid 536 of Z18303_P19 (SEQ ID NO:135), a third amino acid sequence being at least 90% homologous to amino acids 537-819 of known protein NP_{—}000247 (SEQ ID NO: 128), which also corresponds to amino acids 537-819 of Z18303_P19 (SEQ ID NO:135), a bridging amino acid R corresponding to amino acid 820 of Z18303_P19 (SEQ ID NO:135), and a fourth amino acid sequence being at least 90% homologous to amino acids 821-1110 of known protein(s) NP_{—}000247 (SEQ ID NO: 128), which also corresponds to amino acids 821-1110 of Z18303_P19 (SEQ ID NO:135), wherein said first amino acid sequence, bridging amino acid, second amino acid sequence, bridging amino acid, third amino acid sequence, bridging amino acid and fourth amino acid sequence are contiguous and in a sequential order.

An isolated chimeric polypeptide encoding for Z18303_P3, comprising a first amino acid sequence being at least 90% homologous to amino acids 1-247 of known protein(s) NP_{—}000247, which also corresponds to amino acids 1-247 of Z18303_P3, a bridging amino acid D corresponding to amino acid 248 of Z18303_P3, a second amino acid sequence being at least 90% homologous to amino acids 249-363 of known protein(s) NP_{—}000247, which also corresponds to amino acids 249-363 of Z18303_P3, and a third amino acid G corresponding to amino acid 364 of Z18303_P3, wherein said first amino acid sequence, bridging amino acid, second amino acid sequence and third amino acid are contiguous and in a sequential order.

An isolated chimeric polypeptide encoding for Z18303_P6, comprising a first amino acid sequence being at least 90% homologous to amino acids 1-632 of known protein(s) MYPC_HUMAN and Q9UM53_HUMAN, which also corresponds to amino acids 1-632 of Z18303_P6, and a second amino acid sequence being at least 70%, optionally at least 80%, preferably at least 85%, more preferably at least 90% and most preferably at least 95% homologous to a polypeptide having the sequence GEPAPG (SEQ ID NO: 169) corresponding to amino acids 633-638 of Z18303_P6, wherein said first amino acid sequence and second amino acid sequence are contiguous and in a sequential order.

An isolated polypeptide encoding for an edge portion of Z18303_P6, comprising an amino acid sequence being at least 70%, optionally at least about 80%, preferably at least about 85%, more preferably at least about 90% and most preferably at least about 95% homologous to the sequence GEPAPG (SEQ ID NO: 169).

An isolated chimeric polypeptide encoding for Z18303_P6, comprising a first amino acid sequence being at least 90% homologous to amino acids 1-247 of known protein(s) NP_{—}000247, which also corresponds to amino acids 1-247 of Z18303_P6, a bridging amino acid D corresponding to amino acid 248 of Z18303_P6, a second amino acid sequence being at least 90% homologous to amino acids 249-535 of known protein(s) NP_{—}000247, which also corresponds to amino acids 249-535 of Z18303_P6, a bridging amino acid A corresponding to amino acid 536 of Z18303_P6, a third amino acid sequence being at least 90% homologous to amino acids 537-632 of known protein(s) NP_{—}000247, which also corresponds to amino acids 537-632 of Z18303_P6, and a fourth amino acid sequence being at least 70%, optionally at least 80%, preferably at least 85%, more preferably at least 90% and most preferably at least 95% homologous to a polypeptide having the sequence GEPAPG (SEQ ID NO: 169) corresponding to amino acids 633-638 of Z18303_P6, wherein said first amino acid sequence, bridging amino acid, second amino acid sequence, bridging amino acid, third amino acid sequence and fourth amino acid sequence are contiguous and in a sequential order.

An isolated chimeric polypeptide encoding for Z18303_P7, comprising a first amino acid sequence being at least 90% homologous to amino acids 1-284 of known protein(s) MYPC_HUMAN and Q9UM53_HUMAN, which also corresponds to amino acids 1-284 of Z18303_P7, and a second amino acid sequence being at least 70%, optionally at least 80%, preferably at least 85%, more preferably at least 90% and most preferably at least 95% homologous to a polypeptide having the sequence PSLQPHWGSLSIVIAMRTLGFWTSAHC (SEQ ID NO: 170) corresponding to amino acids 285-311 of Z18303_P7, wherein said first amino acid sequence and second amino acid sequence are contiguous and in a sequential order.

An isolated polypeptide encoding for an edge portion of Z18303_P7, comprising an amino acid sequence being at least 70%, optionally at least about 80%, preferably at least about 85%, more preferably at least about 90% and most preferably at least about 95% homologous to the sequence PSLQPHWGSLSIVIAMRTLGFWTSAHC (SEQ ID NO: 170).

An isolated chimeric polypeptide encoding for Z18303_P7, comprising a first amino acid sequence being at least 90% homologous to amino acids 1-247 of known protein(s) NP_{—}000247, which also corresponds to amino acids 1-247 of Z18303_P7, a bridging amino acid D corresponding to amino acid 248 of Z18303_P7, a second amino acid sequence being at least 90% homologous to amino acids 249-284 of known protein(s) NP_{—}000247, which also corresponds to amino acids 249-284 of Z18303_P7, and a third amino acid sequence being at least 70%, optionally at least 80%, preferably at least 85%, more preferably at least 90% and most preferably at least 95% homologous to a polypeptide having the sequence PSLQPHWGSLSIVIAMRTLGFWTSAHC (SEQ ID NO: 170) corresponding to amino acids 285-311 of Z18303_P7, wherein said first amino acid sequence, bridging amino acid, second amino acid sequence and third amino acid sequence are contiguous and in a sequential order.

According to certain embodiments, the present invention now discloses a cluster designated herein HUMCA1XIA, comprising novel amino acid and nucleic acid sequences that are variants of the known Collagen alpha 1 (SwissProt accession identifier COBA1_HUMAN). The novel variant polynucleotides and polypeptides described by the present invention are useful as diagnostic markers, preferably as serum markers.

Surprisingly, the present invention now shows that HUMCA1XIA variants are overexpressed in cancerous tissues, particularly in cancerous lung, cancerous breast, cancerous ovary and cancerous colon tissues, and thus can be used for the diagnosis, prognosis, treatment selection, and treatment monitoring and/or assessment of cancer, particularly lung cancer, breast cancer, ovarian cancer and colon cancer, as is described in a greater detail below.

In some embodiments, the isolated chimeric proteins or polypeptides of the invention comprise an amino acid sequence corresponding to or homologous to SEQ ID NO:162.

According to another embodiment, the isolated polypeptide comprises an amino acid sequence as set forth in SEQ ID NO:162.

According to the present invention there is provided an isolated chimeric polypeptide, comprising a first amino acid sequence being at least 90% homologous to amino acids 1-260 of COBA1_HUMAN (SEQ ID NO:155), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKMRTVATKSKEKSKKFTPPKSEKFSSKKKKSYQASAKAKLGVKVAKKKQSRSILDKLEDL (SEQ ID NO:167) corresponding to amino acids 261-328 of HUMCA1XIA_P26 (SEQ ID NO:162), wherein said first amino acid sequence and second amino acid sequence are contiguous and in a sequential order.

According to the present invention there is provided an isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167).

According to the present invention there is provided an isolated chimeric polypeptide, comprising a first amino acid sequence being at least 90% homologous to amino acids 1-311 of P12107-2 (SEQ ID NO:159), which also corresponds to amino acids 1-311 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence VAKKKQSRSILDKLEDL (SEQ ID NO:168) corresponding to amino acids 312-328 of HUMCA1XIA_P26 (SEQ ID NO:162), wherein said first amino acid sequence and second amino acid sequence are contiguous and in a sequential order.

According to the present invention there is provided an isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence VAKKKQSRSILDKLEDL (SEQ ID NO:168).

According to certain embodiments, the polypeptides of this invention comprise variants of known proteins, and in other embodiments the polypeptides of this invention comprise splice variants of native proteins expressed in a given subject. In some embodiments, the polypeptides may be obtained through known protein evolution techniques available in the art. In other embodiments, the polypeptides of this invention may be obtained via rational design, based on a particular native polypeptide sequence.

According to another aspect the present invention provides antibodies or antibody fragments specifically interacting with or recognizing a polypeptide of this invention.

According to certain embodiments, the antibody recognizes one or more epitopes (antigen determinants) contained within the polypeptides of this invention, wherein such that binding of the antibody to an epitope distinguish between the splice variants of the present invention and a known polypeptide or protein. Reference to the antibody property of “specific interaction” or “recognition” is to be understood as including covalent and non-covalent associations with a variance of affinity over several orders of magnitude. These terms are to be understood as relative with respect to an index molecule, for which the antibody is thought to have little to no specific interaction or recognition. In one embodiment, the antibodies specifically interact or recognize a particular antigen determinant.

In certain embodiments, the antibodies or antibody fragments of this invention recognize or interact with a polypeptide or protein of the invention, while not substantially recognize or interact with other molecules, even when present in the same sample, for example a biological sample. According to one embodiment, the antibodies of this invention have a specificity such that the specific interaction with or binding to the antigen is at least about 2, or in another embodiment, at least about 5, or in still further embodiment, at least about 10-fold greater than interaction or binding observed under the same reaction conditions with a molecule that does not include the antigenic determinant.

According to certain embodiments, the antibodies are useful in detecting qualitative and/or quantitative changes in the expression of the polypeptides or polynucleotides of this invention. In some embodiments, changes in expression are associated with a particular disease or disorder, such that detection of the changes comprises a diagnostic method of the present invention.

According to other embodiments, the present invention provides an antibody capable of specifically binding to at least one epitope of a polypeptide comprising an amino acid sequence as set forth in any one of SEQ ID NOs: 55, 56, 98-108, 129-135, 162.

According to additional aspect the present invention provides a diagnostic kit for detecting a disease, comprising markers and reagents for detecting qualitative and/or quantitative changes in the expression of a polypeptide or a polynucleotide of this invention.

According to on embodiment, the kit comprises markers and reagents for detecting the changes by employing a NAT-based technology. In one embodiment, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence In Situ Hybridization or Comparative Genomic Hybridization.

According to certain currently preferred embodiments, the kit comprises at least one nucleotide probe or primer. In one embodiment, the kit comprises at least one primer pair capable of selectively hybridizing to a nucleic acid sequence according to the teaching of the present invention. In another embodiment, the kit comprises at least one oligonucleotide capable of selectively hybridizing to a nucleic acid sequence according to the teaching of the present invention.

According to other currently preferred embodiments, the kit comprises an antibody capable of recognizing or interacting with a polypeptide or protein of the present invention. According to certain embodiments, the kit further comprises at least one reagent for performing an ELISA, an RIA, a slot blot, an immunohistochemical assay, FACS, in-vivo imaging, a radio-imaging assay, or a Western blot.

The present invention further provides diagnostic methods for screening for a disease, disorder or conditions, comprising the detection of a polypeptide or polynucleotide of this invention, whereby expression, or relative changes in expression of the polypeptide or polynucleotide herald the onset, severity, or prognosis of an individual with regard to a particular disease, disorder or condition. The detection may comprise detection of the expression of a specific splice variant, or other polypeptide or polynucleotide of this invention, via any means known in the art, and as described herein.

As used herein, the term “screening for a disease” encompasses diagnosing the presence of a disease, its prognosis and/or severity, as well as selecting a treatment and monitoring the treatment of the disease. According to certain currently preferred embodiments, the disease is a marker-detectable disease, wherein the marker is a polynucleotide, polypeptide or protein according to the present invention.

Thus, according to certain aspects, the present invention provides methods for screening for a marker detectable disease, comprising detecting in a subject or in a sample obtained from the subject at least one transcript and/or protein or polypeptide being a member of a cluster selected from the group consisting of cluster D11717, cluster HSTNFR1A, cluster Z18303, cluster HUMCA1XIA, or any combination thereof. According to certain currently preferred embodiments, the method comprises detecting the expression of a splice variant transcript or a product thereof.

According to one aspect, the present invention provide a method for screening for a marker detectable disease in a subject, comprising (a) obtaining a sample from the subject and (b) detecting in the sample at least one polynucleotide and/or polypeptide being a member of a cluster selected from the group consisting of cluster D11717, cluster HSTNFR1A, cluster Z18303, cluster HUMCA1XIA, or any combination thereof. According to one embodiment, the presence of the polynucleotide or polypeptide in the sample is indicative of the presence of the disease and/or its severity and/or its progress. According to another embodiment, a change in the level of the polynucleotide or polypeptide in the sample compared to its level in a sample obtained from a healthy subject is indicative of the presence of the disease and/or its severity. According to another embodiment, a change in the level of the polynucleotide or polypeptide in the sample compared to its level in a sample previously obtained from said subject is indicative of the presence of the disease, its severity and/or the progress of the disease.

According to one embodiment, the present invention provides a method for screening for a cardiovascular disease in a subject, comprising (a) obtaining a sample from the subject and (b) detecting in the sample at least one polypeptide being a member of cluster D11717, cluster HSTNFR1A, cluster 218303, or any combination thereof. According to one embodiment, the presence of the polypeptide in the sample is indicative of the presence of the disease and/or its severity and/or its progress. According to another embodiment, a change in the level of the polypeptide in the sample compared to its level in a sample obtained from a healthy subject is indicative of the presence of the disease and/or its severity. According to another embodiment, a change in the level of the polypeptide in the sample compared to its level in a sample previously obtained from said subject is indicative of the presence of the disease, its severity and/or the progress of the disease. According to currently preferred embodiments, the sample is a serum sample.

According to other embodiments, the cardiovascular disease include inter alia, myocardial infarct, acute coronary syndrome, coronary artery disease, angina pectoris (stable and unstable), cardiomyopathy, myocarditis, congestive heart failure or any type of heart failure and reinfarction. According to other embodiments, the method is useful for the assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof. In further embodiments, the polypeptides of cluster D11717, cluster HSTNFR1A, cluster Z18303, or a combination thereof, are useful in the diagnosis, treatment or assessment of the prognosis of a subject with congestive heart failure (CHF). According to still other embodiments, they are useful in the diagnosis, treatment or assessment of the prognosis of a subject with sudden cardiac death, from arrhythmia or any other heart related reason; rejection of a transplanted heart; conditions that lead to heart failure including but not limited to myocardial infarction, angina, arrhythmias, valvular diseases, atrial and/or ventricular septal defects; conditions that cause atrial and or ventricular wall volume overload, including but not limited to systemic arterial hypertension, pulmonary hypertension and pulmonary embolism; conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

Thus, according to another aspect, the present invention provides a method for screening for a cardiovascular disease in a subject, comprising detecting in the subject at least one polynucleotide and/or polypeptide being a member of cluster Z18303, and/or cluster D11717, and/or cluster HSTNFR1A.

According to another embodiment, the polypeptide or polynucleotide is at least 85% homologous to a secreted splice variant of Z18303 or of D11717, or of HSTNFR1A, or a polynucleotide encoding same, respectively, or a fragment thereof. According to another embodiment, the polypeptide or polynucleotide is at least 95% homologous to a secreted splice variant of Z18303 or of D11717, or of HSTNFR1A, or a polynucleotide encoding same, respectively, or a fragment thereof. According to this embodiment, the method for screening for a cardiovascular disease is performed in vitro with a sample obtained from the subject.

According to another aspect, the present invention provides a method for screening for cardiovascular disease in a subject, comprising detecting in the subject a polypeptide comprising an amino acid sequence at least 85% homologous to the amino acid sequence set forth in any one of SEQ ID NOs: 55, 56, 98-108, 129-135. According to one embodiment, the method comprising detecting polypeptide comprising an amino acid sequence at least 95% homologous to the amino acid sequence set forth in any one of SEQ ID NOs: 55, 56, 98-108, 129-135. According to another embodiment, the method comprises detecting a polypeptide comprising an amino acid sequence as set forth in any one of SEQ ID NOs: 55, 56, 98-108, 129-135. According to another embodiment, the method comprises detecting a polypeptide comprising an amino acid sequence as set forth in any one of SEQ ID NOs:96-97.

According to yet another aspect, the present invention provides a method for screening for cardiovascular disease in a subject, comprising detecting in the subject a polynucleotide comprising a nucleic acid sequence at least 85% homologous to the nucleic acid sequence set forth in any one of SEQ ID NOs: 35-41, 60-72, 109-126. According to one embodiment, the method comprises detecting a polynucleotide comprising a nucleic acid sequence at least 95% homologous to the nucleic acid sequence set forth in any one of SEQ ID NOs: 35-41, 60-72, 109-126. According to another embodiment, the method comprises detecting a polynucleotide comprising a nucleic acid sequence as set forth in any one of SEQ ID NOs: 35-41, 60-72, 109-126.

Each polypeptide of the Z18303 variants, D11717 variants, and HSTNFR1A variants described herein as a marker for cardiovascular conditions, can be used alone or in combination with one or more other variant markers described herein, and/or in combination with known markers for cardiovascular conditions, including but not limited to Heart-type fatty acid binding protein (H-FABP), B-type natriuretic peptide (BNP), Troponin I, Angiotensin, C-reactive protein (CRP), myeloperoxidase (MPO), and/or in combination with the known protein(s) for the variant marker as described herein.

According to one embodiment, the present invention provides a method for screening for acute and chronic inflammation, and/or cerebrovascular diseases in a subject, comprising (a) obtaining a sample from the subject and (b) detecting in the sample at least one polypeptide being a member of a cluster D11711, cluster HSTNFR1A, or any combination thereof. According to one embodiment, the presence of the polypeptide in the sample is indicative of the presence of the disease and/or its severity and/or its progress. According to another embodiment, a change in the level of the polypeptide in the sample compared to its level in a sample obtained from a healthy subject is indicative of the presence of the disease and/or its severity. According to another embodiment, a change in the level of the polypeptide in the sample compared to its level in a sample previously obtained from said subject is indicative of the presence of the disease, its severity and/or the progress of the disease. According to currently preferred embodiments, the sample is a serum sample.

According to other embodiments, the acute and chronic inflammation diseases include a spectrum of diseases where an inflammatory process plays a substantial role. In some embodiments, the polypeptides, polynucleotides and/or methods of this invention may be useful in the diagnosis, treatment or assessment of the prognosis of a subject with hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behcet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute and/or chronic infective processes that involve systemic immune reaction including but not limited to pneumonia, bacteremia, sepsis, pyelonephritis, cellulitis, osteomyelitis, meningitis and viral hepatitis; malignant and idiopathic processes that involve systemic immune reaction and/or proliferation of immune cells including but not limited to granulomatous disorders, Wegener's granulomatosis, lymphomatoid granulomatosis /polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes; conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery; conditions which have similar symptoms, signs and complications as the conditions above and where the differential diagnosis between them and the conditions above is of clinical importance including but not limited to: other (non vascular) causes of heart disease, renal disease and cerebral disease; other (non rheumatic) causes of arthropathy and musculoskeletal pain; other causes of non-specific symptoms and signs such as fever of unknown origin, loss of appetite, weight loss, nonspecific pains, breathing difficulties, anxiety, or any combination thereof, or any disease disorder or condition associated with inflammation. Thus, according to another aspect, the present invention provides a method for screening, diagnosis, or assessment of the prognosis of acute and chronic inflammation, and/or cerebrovascular diseases in a subject, comprising detecting in the subject at least one polynucleotide and/or polypeptide being a member of cluster D11711, cluster HSTNFR1A.

According to another embodiment, the polypeptide or polynucleotide is at least 85% homologous to variant of D11717 or variant of HSTNFR1A or a polynucleotide encoding same, respectively, or a fragment thereof. According to another embodiment, the polypeptide or polynucleotide is at least 95% homologous to a secreted splice variant of D11717 or variant of HSTNFR1A or a polynucleotide encoding same, respectively, or a fragment thereof. According to this embodiment, the method for screening for acute and chronic inflammation, and/or cerebrovascular diseases is performed in vitro with a sample obtained from the subject.

According to another aspect, the present invention provides a method for screening, diagnosis, or assessment of the prognosis of acute and chronic inflammation, and/or cerebrovascular diseases in a subject, comprising detecting in the subject a polypeptide comprising an amino acid sequence at least 85% homologous to the amino acid sequence set forth in any one of SEQ ID NOs: 55, 56, 98-108. According to one embodiment, the method comprising detecting polypeptide comprising an amino acid sequence at least 95% homologous to the amino acid sequence set forth in any one of SEQ ID NOs: 55, 56, 98-108. According to another embodiment, the method comprises detecting a polypeptide comprising an amino acid sequence as set forth in any one of SEQ ID NOs: 55, 56, 98-108). According to another aspect, the present invention provides a method for screening, diagnosis, or assessment of the prognosis of acute and chronic inflammation, and/or cerebrovascular diseases in a subject, comprising detecting in the subject a polypeptide comprising an amino acid sequence as set forth in any one of SEQ ID NOs: 51-54, 96-97.

According to yet another aspect, the present invention provides a method for screening, diagnosis, or assessment of the prognosis of acute and chronic inflammation, and/or cerebrovascular diseases in a subject, comprising detecting in the subject a polynucleotide comprising a nucleic acid sequence at least 85% homologous to the nucleic acid sequence set forth in any one of SEQ ID NOs: 35-41, 60-72. According to one embodiment, the method comprises detecting a polynucleotide comprising a nucleic acid sequence at least 95% homologous to the nucleic acid sequence set forth in any one of SEQ ID NOs: 35-41, 60-72. According to another embodiment, the method comprises detecting a polynucleotide comprising a nucleic acid sequence as set forth in any one of SEQ ID NOs: 35-41, 60-72.

Each variant marker of the present invention described herein as potential marker for cerebrovascular conditions, might optionally be used alone or in combination with one or more other variant markers described herein, and or in combination with known markers for cerebrovascular conditions, including but not limited to CRP, S100b, BNGF, CD40, MCP1, β-amyloid N-Acetyl-Aspartate (NAA), N-methyl-d-aspartate (NMDA) receptor antibodies (NR2Ab), and/or in combination with the known protein(s) for the variant marker as described herein.

The present invention further discloses that surprisingly, detecting in a subject at least one polypeptide or polynucleotide of D11717, variants as disclosed in the present invention, are indicative of cancer, including, but not limited to epithelial malignant tumors, prostate cancer, pancreas cancer, colorectal cancer, breast cancer, renal cancer, skin cancer and liver cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of cancer, including, but not limited to epithelial malignant tumors, prostate cancer, pancreas cancer, colorectal cancer, breast cancer, renal cancer, skin cancer and liver cancer, in said subject. These polynucleotides and polypeptides of cluster D11717 are also useful for treatment selection and treatment monitoring of cancer, including, but not limited to epithelial malignant tumors, prostate cancer, pancreas cancer, colorectal cancer, breast cancer, renal cancer, skin cancer, liver cancer, and/or epithelial malignant tumors, prostate cancer, pancreas cancer, colorectal cancer, breast cancer, renal cancer, skin cancer and liver cancer invasion and metastasis.

Thus, according to another aspect, the present invention provides a method for screening for a cancer in a subject, comprising detecting in the subject at least one polynucleotide and/or polypeptide being a member of cluster D11717.

According to another embodiment, the polypeptide or polynucleotide is at least 85%, and/or 95% homologous to a secreted splice variant of D11717 or a polynucleotide encoding same, respectively, or a fragment thereof. According to this embodiment, the method for screening for a cancer is performed in vitro with a sample obtained from the subject.

According to another aspect, the present invention provides a method for screening for cancer, including, but not limited to epithelial malignant tumors, prostate cancer, pancreas cancer, colorectal cancer, breast cancer, renal cancer, skin cancer, liver cancer, and/or epithelial malignant tumors, prostate cancer, pancreas cancer, colorectal cancer, breast cancer, renal cancer, skin cancer and liver cancer invasion and metastasis, in a subject, comprising detecting in the subject a polypeptide comprising an amino acid sequence at least 85% and/or 95% homologous to the amino acid sequence set forth in any one of SEQ ID NOs: 55, 56. According to one embodiment, the method comprises detecting a polypeptide comprising an amino acid sequence as set forth in any one of SEQ ID NOs: 55, 56.

According to yet another aspect, the present invention provides a method for screening for cancer, including, but not limited to epithelial malignant tumors, prostate cancer, pancreas cancer, colorectal cancer, breast cancer, renal cancer, skin cancer, liver cancer, and/or epithelial malignant tumors, prostate cancer, pancreas cancer, colorectal cancer, breast cancer, renal cancer, skin cancer and liver cancer invasion and metastasis, in a subject, comprising detecting in the subject a polynucleotide comprising a nucleic acid sequence at least 85% and/or 95% homologous to the nucleic acid sequence set forth in any one of SEQ ID NOs: 35-41. According to one embodiment, the method comprises detecting a polynucleotide comprising a nucleic acid sequence as set forth in any one of SEQ ID NOs: 35-41.

According to yet another aspect, the present invention provides a method for screening for cancer, including, but not limited to renal cancer and liver cancer, in a subject, comprising detecting in the subject a polypeptide comprising an amino acid sequence as set forth in any one of SEQ ID NOs: 51-54.

The present invention further discloses that surprisingly, detecting in a subject at least one polypeptide of D11717 variants, as disclosed in the present invention, is indicative of a miscarriage. Detecting the presence of the polypeptide in the woman in lower levels than expected, compared to a standard that is determined based on women who don't miscarry, and/or compared to same woman levels in previous pregnancies/miscarriages, could predict miscarriage.

With regard to miscarriages, the polypeptides of D11717 variants of this invention or markers related thereto may be used for prediction of miscarriages. In some embodiments, the polypeptides of this invention may be used for the prediction of miscarriages, in conjunction with other screening procedures and/or other markers, including but not limited to known MIC-1.

The present invention further discloses that surprisingly, detecting in a subject at least one polypeptide or polynucleotide of HSTNFR1A, variants as disclosed in the present invention, are indicative of cancer, including, but not limited to colorectal cancer and renal cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of cancer, including, but not limited to colorectal cancer and renal cancer, in said subject. These polynucleotides and polypeptides of cluster HSTNFR1A are also useful for treatment selection and treatment monitoring of cancer, including, but not limited to colorectal cancer and renal cancer, and/or colorectal cancer and renal cancer invasion and metastasis.

Thus, according to another aspect, the present invention provides a method for screening for a cancer, including, but not limited to colorectal cancer and renal cancer, in a subject, comprising detecting in the subject at least one polynucleotide and/or polypeptide being a member of cluster HSTNFR1A.

According to another embodiment, the polypeptide or polynucleotide is at least 85%, and/or 95% homologous to a secreted splice variant of HSTNFR1A or a polynucleotide encoding same, respectively, or a fragment thereof. According to this embodiment, the method for screening for a cancer is performed in vitro with a sample obtained from the subject.

According to another aspect, the present invention provides a method for screening for cancer, including, but not limited to colorectal cancer and renal cancer, in a subject, comprising detecting in the subject a polypeptide comprising an amino acid sequence at least 85% and/or 95% homologous to the amino acid sequence set forth in any one of SEQ ID NOs: 98-108. According to one embodiment, the method comprises detecting a polypeptide comprising an amino acid sequence as set forth in any one of SEQ ID NOs: 98-108.

According to yet another aspect, the present invention provides a method for screening for cancer, including, but not limited to colorectal cancer and renal cancer, in a subject, comprising detecting in the subject a polynucleotide comprising a nucleic acid sequence at least 85% and/or 95% homologous to the nucleic acid sequence set forth in any one of SEQ ID NOs: 60-72. According to one embodiment, the method comprises detecting a polynucleotide comprising a nucleic acid sequence as set forth in any one of SEQ ID NOs: 60-72.

According to yet another aspect, the present invention provides a method for screening for cancer, including, but not limited to renal cancer, in a subject, comprising detecting in the subject a polypeptide comprising an amino acid sequence as set forth in any one of SEQ ID NOs:96-97.

The present invention further discloses that surprisingly, detecting in a subject at least one polypeptide or polynucleotide of HUMCA1XIA, variants as disclosed in the present invention, are indicative of cancer, including, but not limited to lung cancer, colon cancer, ovarian cancer and breast cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of cancer, including, but not limited to lung cancer, colon cancer, ovarian cancer and breast cancer, in said subject. These polynucleotides and polypeptides of cluster HUMCA1XIA are also useful for treatment selection and treatment monitoring of cancer, including, but not limited to lung cancer, colon cancer, ovarian cancer, breast cancer, and/or colon, ovarian, breast or lung cancer invasion and metastasis.

Thus, according to another aspect, the present invention provides a method for screening for a cancer in a subject, comprising detecting in the subject at least one polynucleotide and/or polypeptide being a member of cluster HUMCA1XIA.

According to another embodiment, the polypeptide or polynucleotide is at least 85% homologous to a secreted splice variant of HUMCA1XIA or a polynucleotide encoding same, respectively, or a fragment thereof. According to this embodiment, the method for screening for a cancer is performed in vitro with a sample obtained from the subject.

According to another aspect, the present invention provides a method for screening for cancer in a subject, comprising detecting in the subject a polypeptide comprising an amino acid sequence at least 85% homologous to the amino acid sequence set forth in any one of SEQ ID NOs:162. According to one embodiment, the method comprises detecting a polypeptide comprising an amino acid sequence as set forth in any one of SEQ ID NOs:162.

According to yet another aspect, the present invention provides a method for screening for cancer in a subject, comprising detecting in the subject a polynucleotide comprising a nucleic acid sequence at least 85% homologous to the nucleic acid sequence set forth in any one of SEQ ID NOs:153. According to one embodiment, the method comprises detecting a polynucleotide comprising a nucleic acid sequence as set forth in any one of SEQ ID NOs:153.

With regard to lung cancer, the disease is selected from the group consisting of invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affecting treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

The polypeptides and/or polynucleotides of cluster HUMCA1XIA used as markers for lung cancer can be used alone or in combination with one or more alternative polynucleotides or polypeptides described herein, and/or in combination with known markers for lung cancer, including but not limited to CEA, CA15-3, Beta-2-microglobulin, CA19-9, TPA, and/or in combination with the known protein(s) for the variant marker as described herein.

With regard to ovarian cancer, the polypeptides and/or polynucleotide of cluster HUMCA1XIA of the present invention can be used in the diagnosis, treatment or prognostic assessment of invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

The polypeptides and/or polynucleotides of cluster HUMCA1XIA used in the diagnosis, treatment or prognostic assessment of ovarian cancer can be used alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

In another embodiment, the HUMCA1XIA cluster, showing age and/or stage differential diagnostic capability, the present invention provides diagnostic methods, kits and assays for diagnosis, assessment and prognostic indications regarding the indicated disease disorder or condition, according to the age of the subject and/or stage of the condition, as described herein.

With regard to breast cancer, the polypeptides and/or polynucleotides of cluster D11717 and/or cluster HUMCA1XIA are useful in determining a probable outcome in breast cancer; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercalcemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

Each variant marker of the present invention described herein as potential marker for breast cancer can be used alone or in combination with one or more other variant breast cancer described herein, and/or in combination with known markers for breast cancer, including but not limited to Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), and/or in combination with the known protein(s) for the variant marker as described herein. With regard to breast cancer, the disease (and/or diagnostic method to be performed) optionally and preferably comprises one or more of invasive or metastatic breast cancer.

With regard to colon cancer, the disease (and/or diagnostic method to be performed) optionally and preferably comprises one or more of invasive or metastatic colon cancer. Embodiments of markers may be selected from the group consisting of D11717 variants, HUMCA1XIA variants, and HSTNFR1A variants or markers related thereto.

Each marker of the present invention described herein as potential marker for colorectal cancer, might optionally be used alone or in combination with one or more other variant colorectal cancer described herein, and/or in combination with known markers for colorectal cancer, including but not limited to CEA, CA19-9, CA50, and/or in combination with the known protein(s) for the variant marker as described herein.

With regard to prostate cancer, the disease (and/or diagnostic method to be performed) optionally and preferably comprises one or more of invasive or metastatic prostate cancer. Embodiments of markers may be selected from the group consisting of D11717 variants or markers related thereto.

Each marker of the present invention described herein as potential marker for prostate cancer, might optionally be used alone or in combination with one or more other variant prostate cancer described herein, and/or in combination with known markers for prostate cancer, including but not limited to PSA, PAP (prostatic acid phosphatase), CPK-BB, PSMA, PCA3, DD3, and/or in combination with the known protein(s) for the variant marker as described herein.

With regard to renal cancer, the polypeptides and/or polynucleotides of this invention may be used for the diagnosis, treatment selection and monitoring, or assessment of prognosis of renal cancer, including but not limited to renal cell carcinoma. Embodiments of markers may be selected from the group consisting of D11717 variants and HSTNFR1A variants or markers related thereto.

In some embodiments, the polypeptides/polynucleotides of this invention may be used for the diagnosis, treatment selection and monitoring, or assessment of prognosis of renal cancer in conjunction with other screening procedures and/or other markers, including but not limited to urinary protein, creatinine or creatinine clearance, and/or markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

With regard to skin cancer, the polypeptides and/or polynucleotides of this invention may be used for the diagnosis, treatment selection and monitoring, or assessment of prognosis of skin cancer, including but not limited to melanoma. Embodiments of markers may be selected from the group consisting of D11717 variants or markers related thereto. In some embodiments, the polypeptides/polynucleotides of this invention may be used for the diagnosis, treatment selection and monitoring, or assessment of prognosis of skin cancer, specifically of melanoma, in conjunction with other screening procedures and/or other markers, including but not limited to S100-beta, melanoma inhibitory activity (MIA), lactate dehydrogenase (LDH), tyrosinase, 5-S-Cysteinyldopa, L-Dopa/L-tyrosine, VEGF, bFGF, IL-8, ICAM-1, MMPs, IL-6, IL-10, sIL-2R (soluble interleukin-2-receptor), sHLA-DR (soluble HLA-DR), sHLA-class-I (soluble HLA-class I), TuM2-PK, Fas/CD95, sHLA-class-I (soluble HLA-class I), Albumin, TuM2-PK (Tumour pyruvate kinase type M2), sFas/CD95, YKL-40, CYT-MAA (cytoplasmic melanoma-associated antigen), HMW-MAA (high-molecular-weight melanoma-associated antigen), STAT3, STAT1, gp100/HMB45, p16 INK4A, PTEN, pRb (retinoblastoma protein), EGFR, p-Akt, c-Kit, c-myc, AP-2, HDM2, bcl-6, Ki67 (detected by Mib1), Cyclin A, B, D, E, p21CIP1, Geminin, PCNA (proliferating cell nuclear antigen), bcl-2, bax, bak, APAF-1, LYVE-1 (lymphatic vascular endothelial hyaluronan receptor-1), PTN, P-Cadherin, E-Cadherin, Beta-catenin, Integrins beta1 and beta3, MMPs (matrix metalloproteinases), Dysadherin, CEACAM1 (carcinoembryonic-antigen-related cell-adhesion molecule 1), Osteonectin, TA, Melastatin, ALCAM/CD166 (Activated leukocyte cell adhesion molecule), CXCR4, Metallothionein.

With regard to liver cancer, the polypeptides and/or polynucleotides of this invention may be used for the diagnosis, treatment selection and monitoring, or assessment of prognosis of liver cancer, including but not limited to hepatocellular carcinoma (HCC). Embodiments of markers may be selected from the group consisting of D11717 variants or markers related thereto. In some embodiments, the polypeptides/polynucleotides of this invention may be used for the diagnosis, treatment selection and monitoring, or assessment of prognosis of liver cancer, specifically of HCC, in conjunction with other screening procedures and/or other markers, including but not limited to Alpha fetoprotein (AFP), des-gamma-carboxyprothrombin (DCP), Squamous cell carcinoma antigen (SCCA)-immunoglobulin M (IgM), AFP (L3), or fucosylated AFP, GP73 (a golgi protein marker) and its fucosylated form, (TGF)-beta1, HS-GGT, free insulin-like growth factor (IGF)-II.

According to certain embodiments, a combination of anyone of the polynucleotides or polypeptides markers of the present invention with another marker can be used for determining a ratio between a quantitative or semi-quantitative measurement of any marker described herein to any other marker described herein, and/or any other known marker, and/or any other marker. With regard to such a ratio between any marker described herein (or a combination thereof) and a known marker, the known marker preferably comprises the “known protein” as described in greater detail below with regard to each cluster or gene.

It is to be understood that any polynucleotide or polypeptide of this invention may be useful as a marker for a disease, disorder or condition, and such use is to be considered a part of this invention.

According to certain embodiments, detecting the expression of a polynucleotide or polypeptide according to the teaching of the present invention is performed by employing a NAT-based technology (optionally by employing at least one nucleotide probe or primer), or by employing an immunoassay (optionally by employing an antibody according to any of the embodiments described herein), respectively.

In some embodiments, this invention provides a method for screening for a disease in a subject, comprising detecting in the subject or in a sample obtained from said subject at least one polypeptide or polynucleotide selected from the group consisting of:
a. a polypeptide having an amino acid sequence as set forth in any one of SEQ ID NOs: 55, 56, 98-108, 129-135, 51-54, 96-97, 162, or a homologue or a fragment thereof;
b. a polypeptide comprising a bridge, edge portion, tail, or head portion, of any one of SEQ ID NOs: 145-149, 167-170, or a homologue or a fragment thereof;
c. a polynucleotide having a nucleic acid sequence as set forth in any one of SEQ ID NOs: 35-41, 60-72, 109-115, 153, or a homologue or a fragment thereof;
d. a polynucleotide comprising a node having a nucleic acid sequence as set forth in any one of SEQ ID NOs: 42-50, 73-95, 116-126, 154;
e. an oligonucleotide having a nucleic acid sequence as set forth in SEQ ID NOs: 57, 58, 136, 137, 139, 140, 142, 143, 150, 163, 164, 166.

According to one embodiment, detecting the presence of the polypeptide or polynucleotide is indicative of the presence of the disease and/or its severity and/or its progress. According to another embodiment, a change in the expression and/or the level of the polynucleotide or polypeptide compared to its expression and/or level in a healthy subject or a sample obtained therefrom is indicative of the presence of the disease and/or its severity and/or its progress. According to a further embodiment, a change in the expression and/or level of the polynucleotide or polypeptide compared to its level and/or expression in said subject or in a sample obtained therefrom at earlier stage is indicative of the progress of the disease. According to still further embodiment, detecting the presence and/or relative change in the expression and/or level of the polynucleotide or polypeptide is useful for selecting a treatment and/or monitoring a treatment of the disease.

According to one embodiment, detecting a polynucleotide of the invention comprises employing a primer pair, comprising a pair of isolated oligonucleotides capable of specifically hybridizing to at least a portion of a polynucleotide having a nucleic acid sequence as set forth in SEQ ID NOs: 59, 138, 141, 144, 165, or polynucleotides homologous thereto.

According to another embodiment, detecting a polynucleotide of the invention comprises employing a primer pair, comprising a pair of isolated oligonucleotides as set forth in SEQ ID NOs: 57-58, 136-137, 139-140, 142-143, 163-164.

According to further embodiment, detecting a polypeptide of the invention comprises employing an antibody capable of specifically binding to at least one epitope of a polypeptide comprising an amino acid sequence as set forth in any one of SEQ ID NOs: 55, 56, 98-108, 129-135, 162, or of a polypeptide comprising a bridge, edge portion, tail, or head portion of any one of SEQ ID NOs: 145-149, 167-170.

In some embodiments, a method of this invention may make use of a polynucleotide, polypeptide, vector, antibody, biomarker, or combination thereof, as described herein, including any embodiments thereof.

In some embodiments, the methods of this invention are conducted on a whole body. According to other embodiments, the methods of the present invention are conducted with a sample isolated from a subject having, predisposed to, or suspected of having the disease, disorder or condition. According to certain embodiments, the sample is a cell or tissue or a body fluid sample. In some embodiments, the methods are directed to the monitoring of disease progression and/or treatment efficacy and/or relapse of the indicated disease, disorder or condition.

In another embodiment, this invention provides methods for the selection of a particular therapy, or optimization of a given therapy for a disease, disorder or condition, the method comprising quantitatively and/or qualitatively determining or assessing expression of the polypeptides and/or polynucleotides, whereby differences in expression from an index sample, or a sample taken from a subject prior to the initiation of the therapy, or during the course of therapy, is indicative of the efficacy, or optimal activity of the therapy.

According to still further aspect, the present invention provides a method for detecting a splice variant nucleic acid sequence in a biological sample, comprising: hybridizing the isolated splice variant nucleic acid molecules or oligonucleotide fragments thereof of at least about 12 nucleotides to a nucleic acid material of the biological sample and detecting a hybridization complex; wherein the presence of the hybridization complex correlates with the presence of said splice variant nucleic acid sequence in the biological sample.

The nucleic acid sequences and/or amino acid sequences shown herein as embodiments of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide” and “nucleic acid molecule”, or “peptide” and “polypeptide” and “protein”, may optionally be used interchangeably.

All technical and scientific terms used herein should be understood to have the meaning commonly understood by a person skilled in the art to which this invention belongs, as well as any other specified description. The following references provide one of skill in the art with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic description of the cancer biomarker selection engine.

FIG. 2 shows a schematic summary of quantitative real-time PCR analysis.

FIG. 3 shows a graph of cancer and cell-line vs. normal tissue expression for D11717.

FIG. 4 is a histogram showing relative expression of the growth differentiation factor 15 D11717 transcripts which are detectable by amplicon as depicted in sequence name D11717_seg14 (SEQ ID NO: 59) in normal and cancerous Colon tissues.

FIGS. 5A-5F shows histograms, demonstrating the expression of D11717 transcripts using MED discovery engine. FIG. 5A presents the results on colon panel; FIG. 5B presents the results on breast panel; FIG. 5C presents the results on kidney panel; FIG. 5D presents the results on skin panel; FIG. 5E presents the results on prostate panel; FIG. 5F presents the results on liver panel. Series 1: Median of the normalized expression of the chips in the group; Series 2: Standard Deviation

FIG. 6 shows histogram, demonstrating the expression of TNFRFS1A transcripts on kidney panel using MED discovery engine. Series 1: Median of the normalized expression of the chips in the group; Series 2: Standard Deviation.

FIG. 7 shows a graph of cancer and cell-line vs. normal tissue expression for Z18303.

FIG. 8 demonstrates Expression of oligonucleotides in various tissues, prob 208040_s_at.

FIG. 9 is a histogram showing relative expression of the MYBPC3-myosin binding protein C Z18303 transcripts which are detectable by amplicon as depicted in sequence name Z18303_seg19-20 (SEQ ID NO: 138) specifically in heart tissue.

FIG. 10 is a histogram showing relative expression of the MYBPC3-myosin binding protein C Z18303 transcripts which are detectable by amplicon as depicted in sequence name Z18303_seg28-29 (SEQ ID NO:141) specifically in heart tissue.

FIG. 11 is a histogram showing relative expression of the MYBPC3-myosin binding protein C 218303 transcripts which are detectable by amplicon as depicted in sequence name Z18303_seg71F2R2_WT (SEQ ID NO: 144) specifically in heart tissue.

FIG. 12 shows a graph of cancer and cell-line vs. normal tissue expression for HUMCA1XIA.

FIG. 13 is a histogram showing over expression of the Homo sapiens collagen, type XI, alpha 1 (COL11A1) HUMCA1XIA transcripts which are detectable by amplicon as depicted in sequence name HUMCA1XIA_seg15 (SEQ ID NO:165) in normal and cancerous Colon tissues.

FIG. 14 is a histogram showing over expression of Homo sapiens collagen, type XI, alpha 1 (COL11A1) HUMCA1XIA transcripts which are detectable by amplicon as depicted in sequence name HUMCA1XIA_seg15 (SEQ ID NO:165) in normal and cancerous ovary tissues. In FIG. 14A samples are arranged according to the disease stage, and in FIG. 14B samples are arranged according to the patient's age.

FIG. 15 is a histogram showing over expression of Homo sapiens collagen, type XI, alpha 1 (COL11A1) HUMCA1XIA transcripts which are detectable by amplicon as depicted in sequence name HUMCA1XIA_seg15 (SEQ ID NO:165) in normal and cancerous Breast tissues.

FIG. 16 is a histogram showing over expression of Homo sapiens collagen, type XI, alpha 1 (COL11A1) HUMCA1XIA transcripts which are detectable by amplicon as depicted in sequence name HUMCA1XIA_seg15 (SEQ ID NO:165) in normal and cancerous lung tissues.

FIG. 17 is a histogram showing Expression of Homo sapiens collagen, type XI, alpha 1 (COL11A1) HUMCA1XIA transcripts which are detectable by amplicon as depicted in sequence name HUMCA1XIA_seg15 (SEQ ID NO:165) in different normal tissues. FIG. 17A presents relative expression of each sample relative to median of the breast samples. FIG. 17B presents relative expression of each sample relative to median of the colon samples. FIG. 17C presents relative expression of each sample relative to median of the lung samples. FIG. 17D presents relative expression of each sample relative to median of the ovary samples.

### DESCRIPTION OF EMBODIMENTS

The present invention provides polynucleotides, polypeptides, particularly variants of known proteins, and uses thereof, particularly as diagnostic markers.

In some embodiments, the polypeptides and polynucleotides of the present invention are useful as diagnostic markers for certain diseases, and as such the term “marker-detectable” or “variant-detectable” with regard to a disease is to be understood as encompassing use of the described polynucleotides and/or polypeptides for diagnosis.

In some embodiments, certain diseases are associated with differential expression, qualitatively or quantitatively, of the polynucleotides and polypeptides of this invention. Assessment of such expression, in turn, can therefore serve as a marker for a particular disease state, susceptibility to a disease, pathogenesis, etc., including any desired disease-specific event, whose analysis is useful, as will be appreciated by one skilled in the art. In one embodiment, such use as a marker is also referred to herein as the polynucleotides and polypeptides being “variant disease markers”.

The markers of the present invention, alone or in combination, can be used for prognosis, prediction, screening, early diagnosis, staging, therapy selection and treatment monitoring of a marker-detectable disease. For example, optionally and preferably, these markers may be used for staging the disease in patient (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (amino acid and/or nucleic acid sequences) uncovered using the methodology of the present invention and described herein can be efficiently utilized as tissue or pathological markers and/or as drugs or drug targets for treating or preventing a disease.

In some embodiments, these markers are specifically released to the bloodstream under conditions of a particular disease, and/or are otherwise expressed at a much higher level and/or specifically expressed in tissue or cells afflicted with or demonstrating the disease. The measurement of these markers, alone or in combination, in patient samples provides information that the diagnostician can correlate with a probable diagnosis of a particular disease and/or a condition that is indicative of a higher risk for a particular disease.

The present invention provides, in some embodiments, diagnostic assays for a marker-detectable disease and/or an indicative condition, and methods of use of such markers for detection of marker-detectable disease and/or an indicative condition, for example in a sample taken from a subject (patient), which in some embodiments, is a blood sample.

Some embodiments of this invention have been exemplified herein wherein cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm or (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frame shift has occurred. A frame shift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:

model=sw.model

GAPEXT=0

GAPOP=100.0
MATRIX=blosum100

Information is given with regard to overexpression of a cluster in cancer based on ESTs. A key to the p values with regard to the analysis of such overexpression is as follows:
Library-based statistics: P-value without including the level of expression in cell-lines (P1)
Library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to, expression only for ESTs from a particular tissue or cancer.

Some embodiments of this invention have been exemplified herein wherein overexpression of a cluster in cancer was a determination based on microarray use. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number.

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgul33.affx; GeneChip Human Genome U133A 2.0 Array at www.affymetrix.com/products/arrays/specific/hgul33av2.affx; and Human Genome U133 Plus 2.0 Array at www.affymetrix.com/products/arrays/specific/hgul33plus.affx). The probe names follow the Affymetrix naming convention. The data is available from NCBI Gene Expression Omnibus (see www.ncbi.nlm.nih.gov/projects/geo/and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgul33av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below (the first word is the abbreviation while the second word is the full name):

(“BONE”, “bone”);
(“COL”, “colon”);
(“EPI”, “epithelial”);
(“GEN”, “general”);
(“LIVER”, “liver”);
(“LUN”, “lung”);
(“LYMPH”, “lymph nodes”);
(“MARROW”, “bone marrow”);
(“OVA”, “ovary”);
(“PANCREAS”, “pancreas”);
(“PRO”, “prostate”);
(“STOMACH”, “stomach”);
(“TCELL”, “T cells”);

### (“THYROID”, “Thyroid”);

(“MAM”, “breast”);
(“BRAIN”, “brain”);
(“UTERUS”, “uterus”);
(“SKIN”, “skin”);
(“KIDNEY”, “kidney”);
(“MUSCLE”, “muscle”);
(“ADREN”, “adrenal”);
(“HEAD”, “head and neck”);
(“BLADDER”, “bladder”);

The term “homology”, as used herein, refers to a degree of sequence similarity in terms of shared amino acid or nucleotide sequences. There may be partial homology or complete homology (i.e., identity). For amino acid sequence homology amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res. 25:3389-3402). The degree of sequence homology is presented in terms of percentage, e.g. “70% homology”. As used herein, the term “at least” with regard to a certain degree of homology encompasses any degree of homology from the specified percentage up to 100%.

The terms “correspond” or “corresponding to” or “correspondence with” are used herein to indicate identity between two corresponding amino acid or nucleic acid sequences.

In some embodiments, the proteins or polypeptides of this invention comprise chimeric protein or polypeptides.

As used herein, the terms “chimeric protein or polypeptide”, or “chimeric polynucleotide” or “chimera” refers to an assembly or a string of amino acids in a particular sequence, or nucleotides encoding the same, respectively, which does not correspond in their entirety to the sequence of the known (wild type) polypeptide or protein, or the nucleic acid encoding same.

In some embodiments, the variants of this invention are derived from two exons, or an exon and an intron of a known protein, or fragments thereof, or segments having sequences with the indicated homology.

It should be noted that the terms “segment”, “seg” and “node” (abbreviated as “N” in the names of nodes) are used interchangeably in reference to nucleic acid sequences of the present invention, they refer to portions of nucleic acid sequences that were shown to have one or more properties as described herein. They are also the building blocks that were used to construct complete nucleic acid sequences as described in greater detail elsewhere herein. Optionally and preferably, they are examples of oligonucleotides which are embodiments of the present invention, for example as amplicons, hybridization units and/or from which primers and/or complementary oligonucleotides may optionally be derived, and/or for any other use.

In some embodiments, the phrase “disease” refers to its commonly understood meaning, and includes, inter alia, any type of pathology and/or damage, including both chronic and acute damage, as well as a progress from acute to chronic damage.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the term “polypeptide” is to be understood to refer to a molecule comprising from at least 2 to several thousand or more amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to N terminus modification, C terminus modification, peptide bond modification, backbone modifications, residue modification, or others. Inclusion of such peptides within the polypeptides of this invention may produce a polypeptide sharing identity with the polypeptides described herein, for example, those provided in the sequence listing.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the phrase “qualitative” when in reference to differences in expression levels of a polynucleotide, polypeptide or cluster as described herein, refers to the presence versus absence of expression, or in some embodiments, the temporal regulation of expression, or in some embodiments, the timing of expression, or in some embodiments, the variant expressed, or in some embodiments, any post-translational modifications to the expressed molecule, and others, as will be appreciated by one skilled in the art. In some embodiments, the phrase “quantitative” when in reference to differences in expression levels of a polynucleotide, polypeptide or cluster as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, the present invention relates to bridges, tails, heads and/or insertions, and/or analogs, homologs and derivatives of such peptides. Such bridges, tails, heads and/or insertions are described in greater detail below with regard to the Examples.

In some embodiments, the term “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

In some embodiments, the term “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

In some embodiments, the term “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME P1): a sequence starting from any of amino acid numbers 49-x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49-x (for example) is not less than 1, nor 50+((n−2)−x) (for example) greater than the total sequence length.

In another embodiment, this invention provides isolated nucleic acid molecules, which in some embodiments encode for splice variants, having a nucleotide sequence as set forth in any one of the sequences listed herein, being homologous to such sequences, at a percent as described herein, or a sequence complementary thereto. In another embodiment, this invention provides an oligonucleotide of at least about 12 nucleotides, which specifically hybridizes with the nucleic acid molecules of this invention. In another embodiment, this invention provides vectors, cells, liposomes and compositions comprising the isolated nucleic acids or polypeptides of this invention, as appropriate.

In another embodiment, this invention provides a method for detecting the polypeptides of this invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a polynucleotide of this invention in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a the polynucleotide in the biological sample.

In some embodiments of the present invention, the polypeptides/polynucleotides described herein are non-limiting examples of markers for diagnosing marker-detectable disease and/or an indicative condition. Each polypeptide/polynucleotide marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to some embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

In some embodiments of the present invention, there are provided of methods, uses, devices and assays for the diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In some embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In some embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In some embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to some embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In some embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. In one embodiment the confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In an embodiment method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients is measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In an embodiment method, acceptable weighting coefficients may range between zero and one, and an initial weighting coefficient for each marker may be assigned as 0.5. In one embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In some embodiments, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In one embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to embodiments of the present invention, a splice variant protein or a fragment thereof, or a splice variant nucleic acid sequence or a fragment thereof, may be featured as a biomarker for detecting marker-detectable disease and/or an indicative condition, such that a biomarker may optionally comprise any of the above.

According to still other embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention, are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

### Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6×His, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in host bacteria with an impaired capacity to proteolytically cleave the recombinant protein. See, e.g., Gottesman, Gene Expression Technology Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21-codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as 6418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1 SDS and 5×10⁶ cpm ³²P labeled probe, at 65° C., with a final wash solution of 1×SSC and 0.1% SDS and final wash at 50° C.

More generally, hybridization of short nucleic acids (below 200 by in length, e.g. 17-40 by in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies. The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Additional NAT tests are Fluorescence In Situ Hybridization (FISH) and Comparative Genomic Hybridization (CGH). Fluorescence In Situ Hybridization (FISH)— The test uses fluorescent single-stranded DNA probes which are complementary to the DNA sequences that are under examination (genes or chromosomes). These probes hybridize with the complementary DNA and allow the identification of the chromosomal location of genomic sequences of DNA.

Comparative Genomic Hybridization (CGH)—allows a comprehensive analysis of multiple DNA gains and losses in entire genomes. Genomic DNA from the tissue to be investigated and a reference DNA are differentially labeled and simultaneously hybridized in situ to normal metaphase chromosomes. Variations in signal intensities are indicative of differences in the genomic content of the tissue under investigation.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes has 4 to 6 base-pair recognition sequences, and cleaves too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their compositions can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Preferably, nucleic acid sequence homology/identity is determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2—NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Non-conventional or modified amino acids can be incorporated in the polypeptides of this invention as well, as will be known to one skilled in the art.

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

### Antibodies:

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′_{z} fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)₂, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction;

F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

### Antibody Engineering in Phage Display Libraries:

Antibodies of this invention may be prepared through the use of phage display libraries, as is known in the art, for example, as described in PCT Application No. WO 94/18219, U.S. Pat. No. 6,096,551, both of which are hereby fully incorporated by reference, The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immuno-specificities. The method comprises amplifying a CDR portion of an immunoglobulin light chain gene by polymerase chain reaction (PCR) using a PCR primer oligonucleotide. The resultant gene portions are inserted into phagemids for production of a phage display library, wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using Papain produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies, are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin, or fragments thereof may comprise the antibodies of this invention. Humanized antibodies are well known in the art. Methods for humanizing non-human antibodies are well known in the art, for example, as described in Winter and co-workers [Jones et al., Nature, 321:522-525 (1986); Riechmann et al., Nature 332:323-327 (1988); Verhoeyen et al., Science, 239:1534-1536 (1988)], U.S. Pat. No. 4,816,567, Hoogenboom and Winter, J. Mol. Biol., 227:381 (1991); Marks et al., J. Mol. Biol., 222:581 (1991), Cole et al., Monoclonal Antibodies and Cancer Therapy, Alan R. Liss, p. 77 (1985), Boerner et al., J. Immunol., 147(1):86-95 (1991), U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995), all of which are incorporated herein by reference.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C RT al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients'responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

### Monoclonal Antibody Therapy:

In some embodiments, monoclonal antibodies are useful for the identification of cancer cells. In some embodiments, monoclonal antibody therapy is a form of passive immunotherapy useful in cancer treatment. Such antibodies may comprise naked monoclonal antibodies or conjugated monoclonal antibodies—joined to a chemotherapy drug, radioactive particle, or a toxin (a substance that poisons cells). In some embodiments, the former is directly cytotoxic to the target (cancer) cell, or in another embodiment, stimulates or otherwise participates in an immune response ultimately resulting in the lysis of the target cell.

In some embodiments, the conjugated monoclonal antibodies are joined to drugs, toxins, or radioactive atoms. They are used as delivery vehicles to take those substances directly to the cancer cells. The MAb acts as a homing device, circulating in the body until it finds a cancer cell with a matching antigen. It delivers the toxic substance to where it is needed most, minimizing damage to normal cells in other parts of the body. Conjugated

MAbs are also sometimes referred to as “tagged,” “labeled,” or “loaded” antibodies. MAbs with chemotherapy drugs attached are generally referred to as chemolabeled. MAbs with radioactive particles attached are referred to as radiolabeled, and this type of therapy is known as radioimmunotherapy (RIT). MAbs attached to toxins are called immunotoxins.

An illustrative, non-limiting example is provided herein of a method of treatment of a patient with an antibody to a variant as described herein, such that the variant is a target of the antibody. A patient with breast cancer is treated with a radiolabeled humanized antibody against an appropriate breast cancer target as described herein. The patient is optionally treated with a dosage of labeled antibody ranging from 10 to 30 mCi. Of course any type of therapeutic label may optionally be used.

The following sections relate to Candidate Marker Examples. It should be noted that Table numbering is restarted within each Example, which starts with the words “Description for Cluster”.

### CANDIDATE MARKER EXAMPLES SECTION

This Section relates to Examples of sequences according to the present invention, including illustrative methods of selection thereof with regard to cancer; other markers were selected as described below for the individual markers.

### Description of the Methodology Undertaken to Uncover the Biomolecular Sequences of the Present Invention

Human ESTs and cDNAs were obtained from GenBank versions 136 (Jun. 15, 2003 p.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat. Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are differentially expressed in cancer tissues as opposed to non-cancerous. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

Selecting Candidates with Regard to Cancer

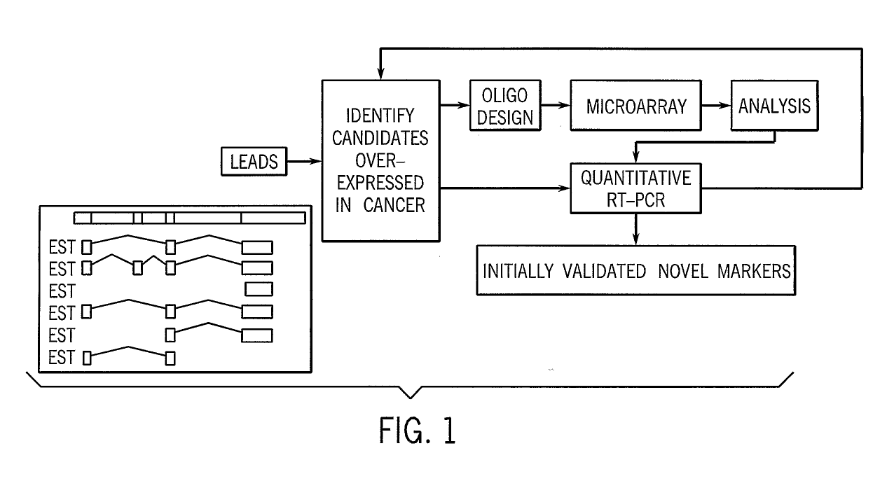

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Example 1

### Identification of Differentially Expressed Gene Products

### Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis

Library annotation—EST libraries are manually classified according to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that are temporally and spacially expressed.

Clusters (genes) having at least five sequences including at least two sequences from the tissue of interest are analyzed.

### Example 2

### Identification of Genes Over Expressed in Cancer

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The algorithm—

Clone counting: For counting EST clones each library protocol class was given a weight based on our belief of how much the protocol reflects actual expression levels:

non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{c + 1}{C} / \frac{n + 1}{N}$$

where:

c—weighted number of “cancer” clones in the cluster.

C— weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 3

### Identification of tissue specific genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 4

### MED Discovery Engine

MED is a platform for collection of public gene-expression data, normalization, annotation and performance of various queries. Expression data from the most widely used Affymetrix microarrays is downloaded from the Gene Expression Omnibus (GEO—www.ncbi.nlm.nih.gov/GEO). Data is multiplicatively normalized by setting the 95 percentile to a constant value (normalized expression=1200), and noise is filtered by setting the lower 30% to 0. Experiments are annotated, first automatically, and then manually, to identify tissue and condition, and chips are grouped according to this annotation. Each probeset in each group is assigned an expression value which is the median of the expressions of that probeset in all chips included in the group. The vector of expression of all probesets within a certain group is the virtual chip of that group, and the collection of all such virtual chips is a virtual panel. The panel (or sub-panels) can be queried to identify probesets with a required behavior (e.g. specific expression in a sub-set of tissues, or differential expression between disease and healthy tissues). These probesets are linked to LEADS contigs and to RefSeqs (http://www.ncbi.nlm.nih.gov/RefSeq/) by probe-level mapping, for further analysis.

The Affymetrix platforms that are downloaded are HG-U95A and the HG-U133 family (A,B, A2.0 and PLUS 2.0). Than three virtual panels were created: U95 and U133 Plus 2.0, based on the corresponding platforms, and U133 which uses the set of common probesets for HG-U133A, HG-U133A2.0 and HG-U133 PLUS 2.0+.

The results of the MED discovery engine are presented in scatter plots. The scatter plot is a compact representation of a given panel (collection of groups). The y-axis is the (normalized) expression and the x-axis describes the groups in the panel. For each group, the median expression is represented by a solid box, and the expression values of the different chips in the group are represented by small segments. The groups are ordered and colored as follows—“Other” groups (e.g. benign, non-cancer diseases, etc.) in orange, Treated cells in black, Normal in blue, Matched in pink, and Cancer in green. The number of chips in each group is also written adjusted to it's name.

### Example 5

### Diseases and Conditions that may be Diagnosed with One or More Variants According to the Present Invention

Acute and Chronic Inflammation and Risk Factors for Cardiovascular and/or Cerebrovascular Diseases

D11717 variants and HSTNFR1A variants are potential markers for inflammation, including a spectrum of diseases where an inflammatory process plays a substantial role. Conditions that may be diagnosed by these markers or variants of them include but are not limited to the presence, risk and/or extent of the following:
1. Conditions that entail an inflammatory process that involves blood vessels including but not limited to hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina.
2. Rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus.
3. Acute and/or chronic infective processes that involve systemic immune reaction including but not limited to pneumonia, bacteremia, sepsis, pyelonephritis, cellulitis, osteomyelitis, meningitis and viral hepatitis.
4. Malignant and idiopathic processes that involve systemic immune reaction and/or proliferation of immune cells including but not limited to granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes.
5. Conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery.
6. Conditions which have similar symptoms, signs and complications as the conditions above and where the differential diagnosis between them and the conditions above is of clinical importance including but not limited to:
   a. Other (non vascular) causes of heart disease, renal disease and cerebral disease.
   b. Other (non rheumatic) causes of arthropathy and musculoskeletal pain.
   c. Other causes of non-specific symptoms and signs such as fever of unknown origin, loss of appetite, weight loss, nonspecific pains, breathing difficulties and anxiety.

### Stroke

Stroke is a manifestation of vascular injury to the brain which is commonly secondary to atherosclerosis or hypertension, and is the third leading cause of death (and the second most common cause of neurologic disability) in the United States. Embodiments of marker(s) for diagnosis of stroke and related conditions as described herein may optionally be selected from the group consisting of D11717 variants and HSTNFR1A variants or markers related thereto.

Specific markers of neural tissue injury are found in the blood or in blood components such as serum and plasma, as well as the CSF of a patient experiencing stroke or TIAs. Furthermore, clearance of the obstructing object in ischemic stroke can cause injury from oxidative insult during reperfusion, and patients with ischemic stroke can sometimes experience hemorrhagic transformation as a result of reperfusion or thrombolytic therapy.

Fibrinolysis is the process of proteolytic clot dissolution. In a manner analogous to coagulation, fibrinolysis is mediated by serine proteinases that are activated from their zymogen form. The serine proteinase plasmin is responsible for the degradation of fibrin into smaller degradation products that are liberated from the clot, resulting in clot dissolution. Fibrinolysis is activated soon after coagulation in order to regulate clot formation. Endogenous serine proteinase inhibitors also function as regulators of fibrinolysis.

The presence of a coagulation or fibrinolysis marker in cerebrospinal fluid would indicate that activation of coagulation or fibrinolysis, depending upon the marker used, coupled with increased permeability of the blood-brain barrier has occurred. In this regard, more definitive conclusions regarding the presence of coagulation or fibrinolysis markers associated with acute stroke may be obtained using cerebrospinal fluid.

Stroke can be categorized into two broad types, “ischemic stroke” and “hemorrhagic stroke.” Additionally, a patient may experience transient ischemic attacks, which are in turn a high risk factor for the future development of a more severe episode.

Ischemic stroke encompasses thrombotic, embolic, lacunar and hypoperfusion types of strokes. Thrombi are occlusions of arteries created in situ within the brain, while emboli are occlusions caused by material from a distant source, such as the heart and major vessels, often dislodged due to myocardial infarct or atrial fibrillation. Less frequently, thrombi may also result from vascular inflammation due to disorders such as meningitis. Thrombi or emboli can result from atherosclerosis or other disorders, for example, arteritis, and lead to physical obstruction of arterial blood supply to the brain. Lacunar stroke refers to an infarct within non-cortical regions of the brain. Hypoperfusion embodies diffuse injury caused by non-localized cerebral ischemia, typically caused by myocardial infarction and arrhythmia.

The onset of ischemic stroke is often abrupt, and can become an “evolving stroke” manifested by neurologic deficits that worsen over a 24-48 hour period. In evolving stroke, “stroke-associated symptom(s)” commonly include unilateral neurologic dysfunction which extends progressively, without producing headache or fever. Evolving stroke may also become a “completed stroke,” in which symptoms develop rapidly and are maximal within a few minutes.

Hemorrhagic stroke is caused by intracerebral or subarachnoid hemorrhage, i.e., bleeding into brain tissue, following blood vessel rupture within the brain. Intracerebral and subarachnoid hemorrhage are subsets of a broader category of hemorrhage referred to as intracranial hemorrhage. Intracerebral hemorrhage is typically due to chronic hypertension, and a resulting rupture of an arteriosclerotic vessel. Stroke-associated symptom(s) of intracerebral hemorrhage are abrupt, with the onset of headache and steadily increasing neurological deficits. Nausea, vomiting, delirium, seizures and loss of consciousness are additional common stroke-associated symptoms.

In contrast, most subarachnoid hemorrhage is caused by head trauma or aneurysm rupture which is accompanied by high pressure blood release which also causes direct cellular trauma. Prior to rupture, aneurysms may be asymptomatic, or occasionally associated with tension or migraine headaches. However, headache typically becomes acute and severe upon rupture, and may be accompanied by varying degrees of neurological deficit, vomiting, dizziness, and altered pulse and respiratory rates.

Transient ischemic attacks (TIAs) have a sudden onset and brief duration, typically 2-30 minutes. Most TIAs are due to emboli from atherosclerotic plaques, often originating in the arteries of the neck, and can result from brief interruptions of blood flow. The symptoms of TIAs are identical to those of stroke, but are only transient. Concomitant with underlying risk factors, patients experiencing TIAs are at a markedly increased risk for stroke.

Current diagnostic methods for stroke include costly and time-consuming procedures such as noncontrast computed tomography (CT) scan, electrocardiogram, magnetic resonance imaging (MRI), and angiography. Determining the immediate cause of stroke and differentiating ischemic from hemorrhagic stroke is difficult. CT scans can detect parenchymal bleeding greater than 1 cm and 95% of all subarachnoid hemorrhages. CT scan often cannot detect ischemic strokes until 6 hours from onset, depending on the infarct size. MRI may be more effective than CT scan in early detection of ischemic stroke, but it is less accurate at differentiating ischemic from hemorrhagic stroke, and is not widely available. An electrocardiogram (ECG) can be used in certain circumstances to identify a cardiac cause of stroke. Angiography is a definitive test to identify stenosis or occlusion of large and small cranial blood vessels, and can locate the cause of subarachnoid hemorrhages, define aneurysms, and detect cerebral vasospasm. It is, however, an invasive procedure that is also limited by cost and availability. Coagulation studies can also be used to rule out a coagulation disorder (coagulopathy) as a cause of hemorrhagic stroke.

Immediate diagnosis and care of a patient experiencing stroke can be critical. For example, tissue plasminogen activator (TPA) given within three hours of symptom onset in ischemic stroke is beneficial for selected acute stroke patients. Alternatively, patients may benefit from anticoagulants (e.g., heparin) if they are not candidates for TPA therapy. In contrast, thrombolytics and anticoagulants are strongly contraindicated in hemorrhagic strokes. Thus, early differentiation of ischemic events from hemorrhagic events is imperative. Moreover, delays in the confirmation of stroke diagnosis and the identification of stroke type limit the number of patients that may benefit from early intervention therapy. Finally, there are currently no diagnostic methods that can identify a TIA, or predict delayed neurological deficits which are often detected at a time after onset concurrent with the presentation of symptoms.

Accordingly, there is a present need in the art for a rapid, sensitive and specific diagnostic assay for stroke and TIA that can also differentiate the stroke type and identify those individuals at risk for delayed neurological deficits. Such a diagnostic assay would greatly increase the number of patients that can receive beneficial stroke treatment and therapy, and reduce the costs associated with incorrect stroke diagnosis.

The present invention relates to the identification and use of diagnostic markers for stroke and neural tissue injury. The methods and compositions described herein can meet the need in the art for rapid, sensitive and specific diagnostic assay to be used in the diagnosis and differentiation of various forms of stroke and TIAs. Moreover, the methods and compositions of the present invention can also be used to facilitate the treatment of stroke patients and the development of additional diagnostic and/or prognostic indicators.

In various aspects, the invention relates to materials and procedures for identifying markers that are associated with the diagnosis, prognosis, or differentiation of stroke and/or TIA in a patient; to using such markers in diagnosing and treating a patient and/or to monitor the course of a treatment regimen; to using such markers to identify subjects at risk for one or more adverse outcomes related to stroke and/or TIA; and for screening compounds and pharmaceutical compositions that might provide a benefit in treating or preventing such conditions.

In a first aspect, the invention discloses methods for determining a diagnosis or prognosis related to stroke, or for differentiating between types of strokes and/or TIA. These methods comprise analyzing a test sample obtained from a subject for the presence or amount of one or more markers for neural tissue injury. These methods can comprise identifying one or more markers, the presence or amount of which is associated with the diagnosis, prognosis, or differentiation of stroke and/or TIA. Once such marker(s) are identified, the level of such marker(s) in a sample obtained from a subject of interest can be measured. In certain embodiments, these markers can be compared to a level that is associated with the diagnosis, prognosis, or differentiation of stroke and/or TIA. By correlating the subject's marker level(s) to the diagnostic marker level(s), the presence or absence of stroke, the probability of future adverse outcomes, etc., in a patient may be rapidly and accurately determined.

In a related aspect, the invention discloses methods for determining the presence or absence of a disease in a subject that is exhibiting a perceptible change in one or more physical characteristics (that is, one or more “symptoms”) that are indicative of a plurality of possible etiologies underlying the observed symptom(s), one of which is stroke. These methods comprise analyzing a test sample obtained from the subject for the presence or amount of one or more markers selected to rule in or out stroke, or one or more types of stroke, as a possible etiology of the observed symptom(s). Etiologies other than stroke that are within the differential diagnosis of the symptom(s) observed are referred to herein as “stroke mimics”, and marker(s) able to differentiate one or more types of stroke from stroke mimics are referred to herein as “stroke differential diagnostic markers”. The presence or amount of such marker(s) in a sample obtained from the subject can be used to rule in or rule out one or more of the following: stroke, thrombotic stroke, embolic stroke, lacunar stroke, hypoperfusion, intracerebral hemorrhage, and subarachnoid hemorrhage, thereby either providing a diagnosis (rule-in) and/or excluding a diagnosis (rule-out).

Obtaining information on the true time of onset can be critical, as early treatments have been reported to be critical for proper treatment. Obtaining this time-of-onset information may be difficult, and is often based upon interviews with companions of the stroke victim. Thus, in various embodiments, markers and marker panels are selected to distinguish the approximate time since stroke onset. For purposes of the present invention, the term “acute stroke” refers to a stroke that has occurred within the prior 12 hours, more preferably within the prior 6 hours, and most preferably within the prior 3 hours; while the term “non-acute stroke” refers to a stroke that has occurred more than 12 hours ago, preferably between 12 and 48 hours ago, and most preferably between 12 and 24 hours ago. Embodiments of markers for differentiating between acute and non-acute strokes, referred to herein as stroke “time of onset markers” are described hereinafter.

For markers appearing in the patent which are already linked to stroke, either ischemic or hemorrhagic, variants could also help to diagnose, directly or by elimination of other conditions including but not limited to:
1. Transient ischemic attack
2. Brain trauma, in case it is unclear whether accompanied by stroke or not
3. Migraine
4. Bleeding in any part of the brain or inside the skull that cause or didn't cause damage to brain tissue
5. Tumor
   In addition, such markers may help determine:
1. The time of stroke
2. The type of stroke
3. The extent of tissue damage as a result of the stroke
4. Response to immediate treatments that are meant to alleviate the extent of stroke and brain damage, when available.

With regard to stroke, according to embodiments of the present invention, the panel may optionally and preferably provide diagnosis of stroke and indication if an ischemic stroke has occurred; diagnosis of stroke and indication if a hemorrhagic stroke has occurred; diagnosis of stroke, indication if an ischemic stroke has occurred, and indication if a hemorrhagic stroke has occurred; diagnosis of stroke and prognosis of a subsequent cerebral vasospasm; and diagnosis of stroke, indication if a hemorrhagic stroke has occurred, and prognosis of a subsequent cerebral vasospasm.

According to other optional embodiments of the present invention, there are provided methods of identifying a patient at risk for cerebral vasospasm. Such methods preferably comprise comparing an amount of one or more marker(s) predictive of a subsequent cerebral vasospasm in a test sample from a patient diagnosed with a subarachnoid hemorrhage. Such markers may be one or more markers related to blood pressure regulation, markers related to inflammation, markers related to apoptosis, and/or specific markers of neural tissue injury. As discussed herein, such marker may be used in panels comprising 2, 3, 4, 5, 6, 7, 8, 9, 10, 15, 20, or more or individual markers. Embodiments of marker(s) may be selected from the group consisting of D11717 variants and HSTNFR1A variants or markers related thereto. The levels of one or more markers may be compared to a predictive level of said marker(s), wherein said patient is identified as being at risk for cerebral vasospasm by a level of said marker(s) equal to or greater than said predictive level. In the alternative, a panel response value for a plurality of such markers may be determined, optionally considering a change in the level of one or more such markers as an additional independent marker.

According to yet other embodiments of the present invention, there are provided methods of differentiating ischemic stroke from hemorrhagic stroke using such marker panels.

### Cardiovascular and Cerebrovascular Conditions

Various examples are listed below for conditions that affect the vascular system, including various cardiovascular and cerebrovascular conditions, for which one or more variants according to the present invention may have a diagnostic utility. Based on these diseases mechanisms and the correlation between the known proteins and the cardiovascular and cerebrovascular conditions, such correlation was predicted also for one or more variants according to the present invention, as described below. Each variant marker of the present invention described herein as potential marker for cardiovascular conditions, might optionally be used alone or in combination with one or more other variant markers described herein, and or in combination with known markers for cardiovascular conditions, including but not limited to Heart-type fatty acid binding protein (H-FABP), B-type natriuretic peptide (BNP), Troponin I, Angiotensin, C-reactive protein (CRP), myeloperoxidase (MPO), and/or in combination with the known protein(s) for the variant marker as described herein. Each variant marker of the present invention described herein as potential marker for cerebrovascular conditions, might optionally be used alone or in combination with one or more other variant markers described herein, and or in combination with known markers for cerebrovascular conditions, including but not limited to CRP, S100b, BNGF, CD40, MCP1, β-amyloid N-Acetyl-Aspartate (NAA), N-methyl-d-aspartate (NMDA) receptor antibodies (NR2Ab), and/or in combination with the known protein(s) for the variant marker as described herein.

### Myocardial Infarction

Z18303 variants, D11717 variants and HSTNFR1A variants are potential markers for myocardial infarction. Other conditions that may be diagnosed by these markers or variants of them include but are not limited to the presence, risk and/or extent of the following:
1. Myocarditis—in myocarditis cardiac muscle cells can go through cell lysis and leakage with the release of intracellular content to the extracellular space and blood, a similar process as happens in myocardial infarction (see also extended description below).
2. Angina—stable or unstable, as the reduction of oxygen delivery to part of the heart often leads to local ischemic conditions that facilitate leakage of intracellular content.
3. Traumatic injury to myocardial tissue—blunt or penetrating, may also result in myocardial cell leakage.
4. Opening an occluded coronary artery following thrombolytic therapy—If such treatment is successful, proteins and other products of the local tissue are washed into the blood and can be detected there.
5. Cardiomyopathy—which is characterized by slow degeneration of the heart muscle (see also extended description below).
6. Myocardial injury after rejection of heart transplant.
7. Congestive heart failure where heart myocytes slowly degenerate (as had been shown for Troponin-I; see also extended description below).
8. Future cardiovascular disease (as a risk factor).
9. Conditions which have similar clinical symptoms as myocardial infarction and where the differential diagnosis between them and myocardial infarction is of clinical importance including but not limited to:
   a. Clinical symptoms resulting from lung related tissue (e.g. Pleuritis, pulmonary embolism)
   b. Musculoskeletal origin of pain
   c. Clinical symptoms resulting from heart related tissue which are not due to myocardial infarction, e.g. acute pericarditis
   d. Upper abdominal pain from abdominal organs including but nor limited to esophagitis, gastro-esophageal reflux, gastritis, gastric ulcer, duodenitis, duodenal ulcer, enteritis, gastroenteritis, cholecystitis, cholelithiasis, cholangiolithiasis, pancreatitis, splenic infarction, splenic trauma, Aortic dissection.

One or more of these markers (variants according to the present invention) may optionally be used a tool to decide on treatment options e.g. anti platelet inhibitors (as has been shown for Troponin-I); as a tool in the assessment of pericardial effusion; and/or as a tool in the assessment of endocarditis and/or rheumatic fever, where progressive damage to the heart muscle may occur.

### Cardiomyopathy and Myocarditis

Cardiomyopathy may be treated with the polynucleotides/polypeptides and/or methods of this invention. Cardiomyopathy is a general diagnostic term designating primary myocardial disease which may progress to heart failure. The disease comprises inflammatory cardiomyopathies, cardiomyopathies resulting from a metabolic disorder such as a nutritional deficiency or by altered endocrine function, exposure to toxic substances, for example from alcohol or exposure to cobalt or lead, infiltration and deposition of abnormal. In some embodiments, the marker(s) for diagnosis of cardiomyopathy and myocarditis, and related conditions as described herein, may optionally be selected from the group consisting of Z18303 variants, D11717 variants and HSTNFR1A variants.

### Congestive Heart Failure (CHF)

Z18303 variants, D11717 variants and HSTNFR1A variants are potential markers for, and may be used to treat, etc., CHF.

The invention provides a means for the identification/prognostication, etc., of a number of conditions including the assessment of the presence, risk and/or extent of the following:
1. A risk factor for sudden cardiac death, from arrhythmia or any other heart related reason.
2. Rejection of a transplanted heart.
3. Conditions that lead to heart failure including but not limited to myocardial infarction, angina, arrhythmias, valvular diseases, atrial and/or ventricular septal defects.
4. Conditions that cause atrial and or ventricular wall volume overload. Wall stretch results in enhanced secretion of cardiac extracellular regulators. Such conditions include but are not limited to systemic arterial hypertension, pulmonary hypertension and pulmonary embolism.
5. Conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

### Cancerous Conditions

Various non-limiting examples are given below of cancerous conditions for which one or more variants according to the present invention may have a diagnostic, or therapeutic utility.

### Breast Cancer

Breast cancer is the most commonly occurring cancer in women, comprising almost a third of all malignancies in females. In one embodiment, the polypeptides and/or polynucleotides of this invention are utilized alone, or in combination with other markers, for the diagnosis, treatment or assessment of prognosis of breast cancer. In one embodiment, the polypeptides and/or polynucleotides serve as markers of disease.

Such markers may be used alone, or in combination with other known markers for breast cancer, including, inter alia, Mucin1 (measured as CA 15-3), CEA (CarcinoEmbryonic Antigen), HER-2, CA125, CA 19-9, PCNA, Ki-67, E-Cadherin, Cathepsin D, TFF1, epidermal growth factor receptor (EGFR), cyclin E, p53, bcl-2, vascular endothelial growth factor, urokinase-type plasminogen activator-1, survivin, or any combination thereof, and includes use of any compound which detects or quantifies the same. ESR (Erythrocyte Sedimentation Rate) values may be obtained, and comprise the marker panel for breast cancer.

In some embodiments, the polypeptides/polynucleotides of this invention serve as prognosticators, in identifying, inter alia, patients at minimal risk of relapse, patients with a worse prognosis, or patients likely to benefit from specific treatments.

There are some non-cancerous pathological conditions which represent an increased risk factor for development breast cancer, and as such, patients with these conditions may be evaluated using the polypeptides/polynucleotides and according to the methods of this invention, for example, as part of the screening methods of this invention, Some of these conditions include, but are not limited to ductal hyperplasia without atypia, atypical hyperplasia, and others.

In some embodiments, the polypeptides/polynucleotides of this invention serve as markers for breast cancer, including, but not limited to: D11717 variants HUMCA1XIA variants or homologues thereof. In some embodiments, the D11717, HUMCA1XIA or polynucleotides encoding the same, can be used alone or in combination with any other desired marker, including, inter alia, Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), or any combinations thereof.

In some embodiments, the polypeptides/polynucleotides of this invention may be useful in, inter alia, assessing the presence, risk and/or extent of the following:
1. The identification of a metastasis of unknown origin which originated from a primary breast cancer tumor.
2. In the assessment of lymphadenopathy, and in particular axillary lymphadenopathy.
3. As a marker to distinguish between different types of breast cancer, therefore potentially affect treatment choice (e.g. as HER-2)
4. As a tool in the assessment of palpable breast mass and in particular in the differential diagnosis between a benign and malignant breast mass.
5. As a tool in the assessment of conditions affecting breast skin (e.g. Paget's disease) and their differentiation from breast cancer.
6. As a tool in the assessment of breast pain or discomfort resulting from either breast cancer or other possible conditions (e.g. Mastitis, Mondors syndrome).
7. Other conditions not mentioned above which have similar symptoms, signs and complications as breast cancer and where the differential diagnosis between them and breast cancer is of clinical importance including but not limited to:
   a. Abnormal mammogram and/or nipple retraction and/or nipple discharge due to causes other than breast cancer. Such causes include but are not limited to benign breast masses, melanoma, trauma and technical and/or anatomical variations.
   b. Any condition suggestive of a malignant tumor including but not limited to anorexia, cachexia, weight loss, fever, hypercalcemia, paraneoplastic syndrome.

Lymphadenopathy, weight loss and other signs and symptoms associated with breast cancer but originate from diseases different from breast cancer including but not limited to other malignancies, infections and autoimmune diseases.
8. Prediction of patient's drug response
9. As surrogate markers for clinical outcome of a treated cancer.
10. Screening for early detection of breast cancer.

### Colorectal Cancer:

Colon and rectal cancers are malignant conditions which occur in the corresponding segments of the large intestine. In one embodiment, the polypeptides and/or polynucleotides of this invention are utilized alone, or in combination with other markers, for the diagnosis, treatment' or assessment of prognosis of colorectal cancer. In some embodiments, the term “colorectal cancers” is to be understood as encompassing adenocarcinomas, carcinoid tumors, for example, found in the appendix and rectum; gastrointestinal stromal tumors for example, found in connective tissue in the wall of the colon and rectum; and lymphomas, which are malignancies of immune cells in the colon, rectum and lymph nodes. In some embodiments, the polypeptides/polynucleotides are useful in diagnosing, treating and/or assessing progression of colorectal pathogenesis, including the maturation of adenomatous polyps, to larger polyps, and all relevant stages in the neoplastic transformation of the tissue.

In some embodiments, the polypeptides/polynucleotides of this invention are utilized in conjunction with other screening procedures, as well as the use of other markers, for the diagnosis, or assessment of prognosis of colorectal cancer in a subject. In some embodiments, such screening procedures may comprise fecal occult blood tests, sigmoidoscopy, barium enema X-ray, digital rectal exam, colonoscopy, detection of carcinoembryonic antigen (CEA) or combinations thereof.

In some embodiments, the polypeptides/polynucleotides are useful in assessing progression of colorectal pathogenesis. Such assessment may reflect the staging of the colorectal cancer. In some embodiments, the polypeptides/polynucleotides are useful in assessing or altering stage progression in a subject with colorectal cancer. When in reference to cancer staging, it is to be understood that any known means or classification system will apply, for any embodiment as described herein. In some embodiments, staging in reference to colorectal cancer may be via the Dukes' system and/or the International Union against Cancer-American Joint Committee on Cancer TNM staging system. Staging will reflect, in some embodiments, the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, and the extent of metastatic invasion into more distant tissues, such as the liver. It is to be understood that the polypeptides/polynucleotides of this invention may be useful both in the identification/assessment of colorectal cancer pathogenesis as a function of stage designation, as well as

In some embodiments, the polypeptides/polynucleotides of this invention may be useful in the diagnosis, treatment and/or assessment of prognosis of colon cancer. According to this aspect and in one embodiment, the polypeptides useful in this context are: D11717 variants, HUMCA1XIA variants, HSTNFR1A variants or homologues thereof, or polynucleotides encoding the same. In some embodiments, these polypeptides/polynucleotides are used alone or in combination with one or more other polypeptides/polynucleotides of this invention, and/or in combination with other markers for colorectal cancer, including but not limited to CEA, CA19-9, CA50, and/or in combination with a native protein associated with the polypeptides of this invention, for example, native proteins of which the polypeptides are variants thereof. In some embodiments, the polypeptides/polynucleotides of this invention may be useful in, inter alia, assessing the presence, risk and/or extent of the following:
1. Early diagnosis, staging, grading, prognosis, monitoring, and treatment of diseases associated with colon cancer, or to indicate a predisposition to such for preventative measures.
2. The identification of a metastasis of unknown origin which originated from a primary colorectal cancer tumor, in particular when the metastasis is located in the liver, lung, bones, supraclavicluar lymph nodes or brain.
3. In the assessment of lymphadenopathy, in particular supraclavicluar or internal abdominal lymphadenopathy.
4. As a marker to distinguish between different types of colorectal tumors including but not limited to nonhereditary carcinoma, Familial Polyposis Coli, Hereditary nonpolyposis colon cancer (Lynch syndrome) and Carcinoid; therefore potentially affect treatment choice.
5. In the assessment of cancer staging, in addition and as a complementary measure to the Dukes system for staging colorectal cancer.
6. As a risk factor to the development of colorectal tumor, and in particular in diseases known to have high incidence of colorectal tumor, including but not limited to Crohn's disease and Ulcerative Colitis.
7. As a tool in the assessment of fecal occult blood or imaging findings suspected for colorectal tumor or abnormal blood tests associated with colorectal cancer including but not limited to elevated CEA level.
8. In the differential diagnosis between malignant and benign colorectal tumors, in particular adenomas and polyps.
9. Other conditions not mentioned above which have similar symptoms, signs and complications as colorectal cancer and where the differential diagnosis between them and colorectal cancer is of clinical importance including but not limited to:
   a. Any condition suggestive of a malignant tumor including but not limited to anorexia, cachexia, weight loss, fever, hypercalcemia, paraneoplastic syndrome.
   b. Lymphadenopathy, weight loss and other signs and symptoms associated with colorectal cancer but originate from diseases different from colorectal cancer including but not limited to other malignancies, infections and autoimmune diseases.
10. Prediction of patient's drug response
11. As surrogate markers for clinical outcome of a treated cancer.

### Ovarian Cancer

Ovarian cancer causes more deaths than any other cancer of the female reproductive system; however, only 25% of ovarian cancers are detected in stage I. No single marker has been shown to be sufficiently sensitive or specific to contribute to the diagnosis of ovarian cancer.

In one embodiment, the markers of this invention are utilized alone, or in combination with other markers, for the diagnosis, treatment or assessment of prognosis of ovarian cancer. Such other markers may comprise CA-125 or mucin 16, CA-50, CA 54-61, CA-195 and CA 19-9, STN and TAG-72, kallikreins, cathepsin L, urine gonadotropin, inhibins, cytokeratins, such as TPA and TPS, members of the Transforming Growth Factors (TGF) beta superfamily, Epidermal Growth Factor, p53 and HER-2 or any combination thereof.

Immunohistochemistry may be used to assess the origin of the tumor and staging as part of the methods of this invention, and as protected uses for the polypeptides of this invention.

In some embodiments, this invention provides polypeptides/polynucleotides which serves as markers for ovarian cancer. In some embodiments, the marker is any polypeptide/polynucleotide as described herein. In some embodiments, the marker is HUMCA1XIA, or variants as described herein or markers related thereto. Each variant marker of the present invention described herein may be used alone or in combination with one or more other variant ovarian cancer described herein, and/or in combination with known markers for ovarian cancer, as described herein. Diagnosis of ovarian cancer and/or of other conditions that may be diagnosed by these markers or variants of them, include but are not limited to the presence, risk and/or extent of the following:
1. The identification of a metastasis of unknown origin which originated from a primary ovarian cancer.
2. As a marker to distinguish between different types of ovarian cancer, therefore potentially affect treatment choice (e.g. discrimination between epithelial tumors and germ cell tumors).
3. As a tool in the assessment of abdominal mass and in particular in the differential diagnosis between a benign and malignant ovarian cysts.
4. As a tool for the assessment of infertility.
5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.
6. Conditions which have similar symptoms, signs and complications as ovarian cancer and where the differential diagnosis between them and ovarian cancer is of clinical importance including but not limited to:
   a. Non-malignant causes of pelvic mass. Including, but not limited to: benign (functional) ovarian cyst, uterine fibroids, endometriosis, benign ovarian neoplasms and inflammatory bowel lesions
   b. Any condition suggestive of a malignant tumor including but not limited to anorexia, cachexia, weight loss, fever, hypercalcemia, skeletal or abdominal pain, paraneoplastic syndrome.
   c. Ascites.
7. Prediction of patient's drug response
8. As surrogate markers for clinical outcome of a treated cancer.
9. Screening for early detection of ovarian cancer.

### Lung Cancer

Lung cancer is the primary cause of cancer death among both men and women in the U.S. In one embodiment, the polypeptides and/or polynucleotides of this invention are utilized alone, or in combination with other markers, for the diagnosis, treatment or assessment of prognosis of lung cancer. In one embodiment, the term “lung cancer” is to be understood as encompassing small cell or non-small cell lung cancers, including adenocarcinomas, bronchoalveolar-alveolar, squamous cell and large cell carcinomas.

In some embodiments, the polypeptides/polynucleotides of this invention are utilized in conjunction with other screening procedures, as well as the use of other markers, for the diagnosis, or assessment of prognosis of lung cancer in a subject. In some embodiments, such screening procedures may comprise the use of chest x-rays, analysis of the type of cells contained in sputum, fiberoptic examination of the bronchial passages, or any combination thereof. Such evaluation in turn may impact the type of treatment regimen pursued, which in turn may reflect the type and stage of the cancer, and include surgery, radiation therapy and/or chemotherapy.

Current radiotherapeutic agents, chemotherapeutic agents and biological toxins are potent cytotoxins, yet do not discriminate between normal and malignant cells, producing adverse effects and dose-limiting toxicities. In some embodiments of this invention, the polypeptides/polynucleotides provide a means for more specific targeting to neoplastic versus normal cells.

In some embodiments, the polypeptides for use in the diagnosis, treatment and/or assessment of progression of lung cancer may comprise: HUMCA1XIA or homologous thereof, or polynucleotides encoding the same. In some embodiments, these polypeptides/polynucleotides may be used alone or in combination with one or more other appropriate markers, including, inter alia, other polypeptides/polynucleotides of this invention. In some embodiments, such use may be in combination with other known markers for lung cancer, including but not limited to CEA, CA15-3, Beta-2-microglobulin, CA19-9, TPA, and/or in combination with native sequences associated with the polypeptides/polynucleotides of this invention, as herein described.

In some embodiments, the polypeptides/polynucleotides of this invention may be useful in, inter alia, assessing the presence, risk and/or extent of the following:
1. The identification of a metastasis of unknown origin which originated from a primary lung cancer.
2. The assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors.
3. Distinguishing between different types of lung cancer, therefore potentially affect treatment choice (e.g. small cell vs. non small cell tumors).
4. Unexplained dyspnea and/or chronic cough and/or hemoptysis, and analysis thereof.
5. Differential diagnosis of the origin of a pleural effusion.
6. Conditions which have similar symptoms, signs and complications as lung cancer and where the differential diagnosis between them and lung cancer is of clinical importance including but not limited to:
   a. Non-malignant causes of lung symptoms and signs. Symptoms and signs include, but are not limited to: lung lesions and infiltrates, wheeze, stridor.
   b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Horner syndrome.
   c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.
7. Prediction of patient's drug response
8. As surrogate markers for clinical outcome of a treated cancer.
9. Screening for early detection of lung cancer.

### Prostate Cancer

Prostate cancer is the most commonly diagnosed malignancy and the second most frequent cause of cancer-related deaths in the western male population. In one embodiment, the polypeptides and/or polynucleotides of this invention are utilized alone, or in combination with other markers, for the diagnosis, treatment or assessment of prognosis of prostate cancer.

In some embodiments, the polypeptides/polynucleotides of this invention are utilized in conjunction with other screening procedures, as well as the use of other markers, for the diagnosis, or assessment of prognosis of colorectal cancer in a subject. In some embodiments, such markers may comprise prostatic acid phosphatase (PAP), prostate-specific antigen (PSA), prostate-specific membrane antigen (PSM), PCA3 DD3 or combinations thereof.

In some embodiments, the polypeptides/polynucleotides of this invention may be useful in the diagnosis, treatment and/or assessment of prognosis of prostate cancer. According to this embodiment, the polypeptides useful in this context are: D11717 variants, homologues thereof, or polynucleotides encoding the same. In some embodiments, these polypeptides/polynucleotides are used alone or in combination with one or more other polypeptides/polynucleotides of this invention, and/or in combination with other markers, including, inter alia, PSA, PAP (prostatic acid phosphatase), CPK-BB, PSMA, PCA3, DD3, and/or a native protein associated with the polypeptides of this invention, for example, native proteins of which the polypeptides are variants thereof. In some embodiments the polypeptides/polynucleotides of this invention are useful in the diagnosis of prostate cancer, which includes, inter alia, the differential diagnosis between prostate cancer and BPH, prostatitis and/or prostatism.

### Renal Cancer

In some embodiments, the polypeptides/polynucleotides of this invention are utilized in conjunction with other screening procedures, as well as the use of other markers, for the diagnosis, or assessment of prognosis of renal cancer in a subject. According to this embodiment, the polypeptides useful in this context are: D11717 variants, HSNFR1A variants or homologues thereof. In some embodiments, these polypeptides/polynucleotides are used alone or in combination with one or more other polypeptides/polynucleotides of this invention, and/or in combination with other markers known to detect renal cancer, and/or screening procedures. In some embodiments, the other markers may comprise markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

### Melanoma

Melanoma is a malignant tumor of melanocytes which are found predominantly in skin but also in the bowel and the eye. It is one of the rarer types of skin cancer but causes the majority of skin cancer related deaths. Melanoma, evolves through various stages from a benign mole, to a primary tumor, and eventually to a metastatic lesion. These stages can be recognized by unique patterns of gene activity, suggesting that melanoma progression can be studied and staged as a series of distinct molecular events.

Significant advances have been made in the field of melanoma biomarkers. Utilization of paraffin-embedded tissue and multiple markers have improved the RT-PCR assays for detection of melanoma cells in lymph node tissue as well as peripheral blood. Lymphangiogenesis has been identified as a novel mechanism for melanoma progression, and candidate markers in the NF-kappaB signaling pathway have been identified to play a key role in melanoma: tumor vasculature interactions. Loss of heterozygosity has been used to identify potential candidates for biochemotherapy.

The serological parameters most widely used for the early detection of a tumor relapse or metastasis in the follow-up of melanoma patients are the melanocyte lineage/differentiation antigens S100-beta and melanoma inhibitory activity (MIA). Both markers have been shown to be useful prognostic markers in melanoma patients with distant metastases (stage 1V, classification system of the American Joint Committee on Cancer, AJCC), but fail to provide prognostic significance in early stages of melanoma, especially in patients who are tumor-free after surgical procedures. Because of the strong correlation of their serum concentrations with the patients' tumor load, S100-beta and MIA are useful markers in the monitoring of therapy response in advanced metastatic melanoma patients (AJCC stage IV).

The strongest prognostic serum biomarker in advanced metastatic melanoma is lactate dehydrogenase (LDH), an unspecific marker indicating high tumor load in a variety of tumor entities, including melanoma.

A variety of other molecules of peripheral blood have been described as markers of tumor load and disease progression in melanoma. These biomarkers are derived from different fields like melanocytic differentiation (e.g. tyrosinase, 5-S-Cysteinyldopa, L-Dopa/L-tyrosine), tumour angiogenesis (e.g. VEGF, bFGF, IL-8), cell adhesion and motility (e.g. ICAM-1, MMPs), cytokines and their receptors (e.g. IL-6, IL-10, sIL-2R (soluble interleukin-2-receptor)), antigen presentation (e.g. HLA molecules: sHLA-DR (soluble HLA-DR), sHLA-class-I (soluble HLA-class I)), tumor cell metabolism (e.g. TuM2-PK), apoptosis (e.g. Fas/CD95) and others: sHLA-class-I (soluble HLA-class I), Albumin, TuM2-PK (Tumour pyruvate kinase type M2), sFas/CD95, YKL-40, CYT-MAA (cytoplasmic melanoma-associated antigen), HMW-MAA (high-molecular-weight melanoma-associated antigen).

Also, STAT3, a protein linked to melanoma progression has been associated with more severe pathologic abnormality of the mole. STAT1, a protein associated with anti-tumor effects, increased 7.8 times and after low-dose interferon it increased 1.4 times over pretreatment levels. In contrast, STAT3 was reduced by 55 percent with high doses of interferon and by 39 percent with low doses.

For the differentiation between benign and malignant melanocytic lesions, the following immunohistochemical markers have been associated with poor prognosis:

| | Association |
|---|---|
| Marker | with poor prognosis |
| Melanocyte lineage/Differentiation antigens | |
| gp100/HMB45 | Increased expression |
| Tumour suppressors/oncogenes/signal | |
| transducers | |
| p16 INK4A | Decreased expression |
| PTEN | Decreased expression |
| pRb (retinoblastoma protein) | Inactivation due to protein |
| | phosphorylation |
| EGFR (epidermal growth factor receptor) | Increased expression |
| p-Akt (activated serine-threonine protein | Increased expression |
| kinase B) | |
| c-Kit | Expression |
| c-myc | Increased expression |
| AP-2 (activator protein-2alpha) transcription | Loss of nuclear AP-2 |
| factor | expression |
| HDM2 (human homologue of murine mdm2) | Increased expression |
| bcl-6 | Expression |
| Cell cycle associated proteins | |
| Ki67 (detected by Mib1) | Increased expression |
| Cyclin A, B, D, E | Increased expression |
| p21CIP1 | Decreased expression |
| Geminin | Increased expression |
| PCNA (proliferating cell nuclear antigen) | Increased expression |
| Regulators of apoptosis | |
| bcl-2 | Increased expression |
| bax | Decreased expression |
| Bak | Decreased expression |
| APAF-1 (Apoptotic protease activating | Decreased expression |
| factor-1) | |
| Surviving | Increased expression |
| Molecules involved in angiogenesis | |
| LYVE-1 (lymphatic vascular endothelial | Increased expression |
| hyaluronan receptor-1) | |
| PTN (pleiotrophin) | Increased expression |
| Molecules involved in cell adhesion and | |
| motility | |
| P-Cadherin | Strong cytoplasmic |
| | expression |
| E-Cadherin | Decreased expression |
| Beta-catenin | Loss of nuclear staining |
| Integrins beta1 and beta3 | Increased expression |
| MMPs (matrix metalloproteinases) | Increased expression |
| Dysadherin | Increased expression |
| CEACAM1 (carcinoembryonic-antigen- | Increased expression |
| related cell-adhesion molecule 1) | |
| Osteonectin (also termed BM40 or SPARC | Increased expression |
| (secreted protein, acidic and rich in | |
| cysteine)) | |
| Others | |
| TA (telomerase activity) | Increased expression |
| Melastatin | Decreased expression |
| ALCAM/CD166 (Activated leukocyte cell | Increased expression |
| adhesion molecule) | |
| CXCR4 receptor | Increased expression |
| Metallothionein | Increased expression |

In some embodiments, the polypeptides/polynucleotides of this invention are utilized in conjunction with other screening procedures, as well as the use of other markers, for the diagnosis, or assessment of prognosis of melanoma in a subject. According to this embodiment, the polypeptides useful in this context are: D11717 variants, or homologues thereof. In some embodiments, these polypeptides/polynucleotides are used alone or in combination with one or more other polypeptides/polynucleotides of this invention, and/or in combination with other markers known to detect melanoma, and/or screening procedures.

### Liver Cancer

While there are other types of liver cancer, the most common form in adults is called hepatocellular carcinoma. Hepatocellular carcinoma (HCC, also called hepatoma) is a primary malignancy (cancer) of the liver. Most cases of HCC are secondary to either a viral hepatitide infection (hepatitis B or C) or cirrhosis (alcoholism being the most common cause of hepatic cirrhosis). In countries where hepatitis is not endemic, most malignant cancers in the liver are not primary HCC but metastasis (spread) of cancer from elsewhere in the body, e.g. the colon

Hepatoma tissues can synthesize various tumor-related proteins, polypeptides, and isoenzymes, such as alpha-fetoprotein (AFP), hepatoma-specific gamma-glutamyl transpeptidase (HS-GGT), etc, and then secrete into blood. The valuable early diagnostic and prognostic biomarkers could predict the development and metastases of HCC.

Alpha fetoprotein (AFP), a protein substance normally produced by liver cells, is widely used as an indicator of HCC because it is found at higher levels in the blood in up to 60 percent of liver cancer patients. But it isn't very specific for cancer because a considerable number of patients with chronic liver disease also have high levels of AFP.

Other biomarkers that have been suggested for HCC: des-gamma-carboxyprothrombin (DCP), a precursor to the protein prothrombin, which is produced by the liver to help blood clot. DCP levels start to rise in patients with liver cancer, and this trend can be monitored through a blood test. Squamous cell carcinoma antigen (SCCA)-D immunoglobulin M (IgM), may be helpful to detect liver cancer. AFP (L3), or fucosylated AFP, a slightly different version of AFP. GP73 (a golgi protein marker) and its fucosylated form was found to correlate with a diagnosis of liver cancer, even if the standard AFP test is negative.

Recently it was shown that circulating hepatoma-specific AFP subtraction, transforming growth factor (TGF)-beta1, HS-GGT, and free insulin-like growth factor (IGF)-II may be more specific markers than total AFP level for early diagnosis for HCC. The circulating genetic markers such as AFP-mRNA, TGF-beta1-mRNA, IGF-II-mRNA, etc from peripheral blood mononuclear cells of HCC patients have been most extensively used in monitoring distal metastasis or postoperative recurrence of HCC.

On the gene expression level, gene expression signatures were analysed, largely in immune cells within the liver microenvironment, which is the area immediately surrounding the tumor. The set of 17 genes included those that encode the mRNAs for cytokines, which are small proteins produced by immune cells that are used to communicate messages between cells in the immune system to either turn up or down the immune response. From the 17-gene set, a unique pattern in the immune cells has been identified and found in normal tissue of the liver microenvironment that could predict the potential for liver tumor metastasis. This metastasis-specific profile included gene activities responsible for increased production of certain cytokines that are associated with an anti-inflammatory response, as well as suppression of immune response. Increased levels of these cytokines are associated with a poor prognosis of cancer.

In some embodiments, the polypeptides/polynucleotides of this invention are utilized in conjunction with other screening procedures, as well as the use of other markers, for the diagnosis, or assessment of prognosis of liver cancer, including but not limited to HCC, in a subject. According to this embodiment, the polypeptides useful in this context are: D11717 variants, or homologues thereof. In some embodiments, these polypeptides/polynucleotides are used alone or in combination with one or more other polypeptides/polynucleotides of this invention, and/or in combination with other markers known to detect liver cancer, including but not limited to HCC, and/or screening procedures.

### Candidate Marker Examples Section

This section relates to examples of sequences according to the present invention, including illustrative methods of selection thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The markers of the present invention were tested with regard to their expression in various cancerous and non-cancerous tissue samples. Unless otherwise noted, all experimental data relates to variants of the present invention, named according to the segment being tested (as expression was tested through RT-PCR as described). A description of the samples used in the ovarian cancer testing panel is provided in Table 1_{—}1 below. A description of the samples used in the lung cancer testing panel is provided in Table 1_{—}2 below. A description of the samples used in the breast cancer testing panel is provided in Table 1_{—}3 below. A description of the samples used in the colon cancer testing panel is provided in Table 1_{—}4 below. A description of the samples used in the normal tissue panel is provided in Table 1_{—}5 below. The keys for the tables 1_{—}1, 1_{—}2, 1_{—}3, and 1_{—}4 are listed in tables 1_{—}1_{—}1, 1_{—}2_{—}1, 1_{—}3_{—}1, and 1_{—}4, respectively. Tests were then performed as described in the “Materials and Experimental Procedures” section below.

TABLE 1_1
Tissue samples in ovarian cancer testing panel

| | | | sample_id | | | | | | | | | | | | | | | | | |
|---|---|---|---|---|---|---|---|---|---|---|---|---|---|---|---|---|---|---|---|---|
| | | | (GCI)/ | | | | | | | | | | | | | | | | | |
| | | | case id | RNA ID | | | | | | | | | | | | | | | | |
| | | | (Asterand)/ | (GCI)/ | | | | | | | | | | | Age | | | | | |
| | | | lot no. | Sample | | | | | | | Meno- | | | | at | | Oral | Oral | | |
| | Source/ | sample | (old | ID | | C | Tumor | | Ethnic | | pausal | Mens | Preg | Preg | first | | Con | Con | Tubal | Recovery |
| Tissue | Delivery | name | samples) | (Asterand) | Diag | Stage | % | age | BG | CA125PRE | Status | Age | Times | Toterm | child | OC | Length | Unit | ligation | Type |
| OVC | Asterand | 1-As-Ser | 23074 | 71900A2 | SA | I | 80 | 49 | CAU | | Pre-M | | 2 | 1 | | | | | | Surg |
| | | SI | | | | | | | | | | | | | | | | | | |
| OVC | Asterand | 2-As-Ser | 22653 | 70270A1 | SA | I | 90 | 69 | WCAU | | Post-M | | 1 | 1 | | | | | | Surg |
| | | SI | | | | | | | | | | | | | | | | | | |
| OVC | Asterand | 3-As-Ser | 18700 | 40771B1 | SA | IB | 100 | 62 | WCAU | | Post-M | | 3 | 3 | | | | | | Surg |
| | | SIB | | | | | | | | | | | | | | | | | | |
| OVC | GOG | 79-(32)- | 93-09-4901 | | SPC | 1B | | 67 | | | | | | | | | | | | |
| | | GO-Ser | | | | | | | | | | | | | | | | | | |
| | | SIB | | | | | | | | | | | | | | | | | | |
| OVC | Asterand | 4-As-Ser | 17646 | 32667B1 | SA | IB | 100 | 68 | W | | Post-M | | 9 | 2 | | | | | | Surg |
| | | SIB | | | | | | | | | | | | | | | | | | |
| OVC | Asterand | 5-As-Ser | 15644 | 22996A1 | SA | IC | 100 | 48 | CAU | | M | | 4 | 2 | | | | | | Surg |
| | | SIC | | | | | | | | | | | | | | | | | | |
| OVC | Asterand | 6-As-Ser | 18701 | 40773C1 | SA | IIA | 100 | 59 | CAU | | Post-M | | 1 | 1 | | | | | | Surg |
| | | SIIA | | | | | | | | | | | | | | | | | | |
| OVC | GCI | 7-GC-Ser | 2O37O | | SA | IIB | 75 | 43 | WCAU | — | Pre-M | 12 | 0 | 0 | 0 | NO | — | | NO | Surg |
| | | SIIB | | | | | | | | | | | | | | | | | | |
| OVC | GCI | 8-GC-Ser | 7B3DP | | SA | IIB | 70 | 70 | WCAU | — | Post-M | 14 | 5 | 3 | 20 | YES | 6 | months | NO | Surg |
| | | SIIB | | | | | | | | | | | | | | | | | | |
| OVC | GOG | 80-(30)- | 2001-08- | | PSC | 3A | | 72 | | | | | | | | | | | | |
| | | GO-Ser | G011 | | | | | | | | | | | | | | | | | |
| | | SIIIA | | | | | | | | | | | | | | | | | | |
| OVC | GOG | 81-(70)- | 95-08-G069 | | PSA | 3B | | 50 | | | | | | | | | | | | |
| | | GO-Ser | | | | | | | | | | | | | | | | | | |
| | | SIIIB | | | | | | | | | | | | | | | | | | |
| OVC | GOG | 82-(5)- | 99-12-G432 | | A | 3C | | 46 | | >500 | | | | | | | | | | |
| | | GO-Ser | | | | | | | | | | | | | | | | | | |
| | | SIIIC | | | | | | | | | | | | | | | | | | |
| OVC | Asterand | 9-As-Ser | 13268 | 19832A1 | SA | IIIC | 90 | 48 | C | | Post-M | | | | | | | | | Surg |
| | | SIIIC | | | | | | | | | | | | | | | | | | |
| OVC | GOG | 83-(29)- | 2001-12- | | SA | 3C | | 50 | | 260 | | | | | | | | | | |
| | | GO-Ser | G035 | | | | | | | | | | | | | | | | | |
| | | SIIIC | | | | | | | | | | | | | | | | | | |
| OVC | GCI | 10-GC- | 3NTIS | | SA | IIIC | 70 | 53 | WCAU | 70 | Post-M | 12 | 1 | 1 | 26 | YES | 3 | months | NO | Surg |
| | | Ser SIIIC | | | | | | | | | | | | | | | | | | |
| OVC | GCI | 11-GC- | CEJUS | | SA | IIIC | 70 | 53 | WCAU | 4814 | Pre-M | — | 2 | 2 | 30 | NO | — | | NO | Surg |
| | | Ser SIIIC | | | | | | | | | | | | | | | | | | |
| OVC | GCI | 12-GC- | 5NCLK | | SA | IIIC | 70 | 54 | WCAU | 209 | Post-M | 13 | 2 | 2 | 21 | YES | 1 | years | NO | Surg |
| | | Ser SIIIC | | | | | | | | | | | | | | | | | | |
| OVC | ABS | 84-(25)- | N0021 | | PSA | 3C | | 55 | CAU | | | | | | | | | | | |
| | | AB-Ser | | | | | | | | | | | | | | | | | | |
| | | SIIIC | | | | | | | | | | | | | | | | | | |
| OVC | GCI | 13-GC- | 1HI5H | | SA | IIIC | 90 | 61 | WCAU | 34 | Post-M | 12 | 6 | 3 | 22 | NO | — | | NO | Surg |
| | | Ser SIIIC | | | | | | | | | | | | | | | | | | |
| OVC | GCI | 14-GC- | 7RMHZ | | SA | IIIC | 80 | 63 | WCAU | — | Post-M | 12 | 2 | 2 | 20 | YES | 10 | years | NO | Surg |
| | | Ser SIIIC | | | | | | | | | | | | | | | | | | |
| OVC | GCI | 15-GC- | 4WAAB | | SA | IIIC | 90 | 63 | WCAU | — | Post-M | 11 | 2 | 1 | 29 | YES | 4 | years | NO | Surg |
| | | Ser SIIIC | | | | | | | | | | | | | | | | | | |
| OVC | GCI | 16-GC- | 79Z67 | | SA | IIIC | 85 | 67 | WCAU | — | Post-M | 12 | 6 | 5 | 24 | YES | 2 | years | YES | Surg |
| | | Ser SIIIC | | | | | | | | | | | | | | | | | | |
| OVC | GOG | 85-(13)- | 94-05-7603 | | APP | 3C | | 67 | | | | | | | | | | | | |
| | | GO-Ser | | | | | | | | | | | | | | | | | | |
| | | SIIIC | | | | | | | | | | | | | | | | | | |
| OVC | GCI | 17-GC- | DDSNL | | SA | IIIC | 70 | 68 | WCAU | — | Post-M | 11 | 4 | 4 | 19 | NO | — | | NO | Surg |
| | | Ser SIIIC | | | | | | | | | | | | | | | | | | |
| OVC | GCI | 18-GC- | DH8PH | | SA | IV | 95 | 70 | WCAU | — | Post-M | 13 | 4 | 3 | 20 | NO | — | | NO | Surg |
| | | Ser SIV | | | | | | | | | | | | | | | | | | |
| OVC | BioChain | 86-(33)- | A503175 | | SPC | | | 41 | Asian | | | | | | | | | | | |
| | | BC-Ser | | | | | | | | | | | | | | | | | | |
| OVC | BioChain | 87-(14)- | A501111 | | A | | | 41 | Asian | | | | | | | | | | | |
| | | Bc-Ser | | | | | | | | | | | | | | | | | | |
| OVC | Biochain | 88-(12)- | A406023 | | A | | | 45 | Asian | | | | | | | | | | | |
| | | Bc-Ser | | | | | | | | | | | | | | | | | | |
| OVC | Biochain | 89-(11)- | A407068 | | A | | | 49 | Asian | | | | | | | | | | | |
| | | Bc-Ser | | | | | | | | | | | | | | | | | | |
| OVC | ABS | 90-(4)- | ILS-7286 | | PC | UN | | 50 | Asian | | | | | | | | | | | |
| | | AB-Ser | | | | | | | | | | | | | | | | | | |
| OVC | ABS | 91-(6)- | A0106 | | A | UN | | 51 | Asian | | | | | | | | | | | |
| | | AB-Ser | | | | | | | | | | | | | | | | | | |
| OVC | ABS | 92-(3)- | ILS-1431 | | PA | UN | | 52 | Asian | | | | | | | | | | | |
| | | AB-Ser | | | | | | | | | | | | | | | | | | |
| OVC | BioChain | 93-(31)- | A503176 | | SPC | | | 52 | Asian | | | | | | | | | | | |
| | | Bc-Ser | | | | | | | | | | | | | | | | | | |
| OVC | ABS | 94-(2)- | ILS-1408 | | PA | UN | | 53 | Asian | | | | | | | | | | | |
| | | AB-Ser | | | | | | | | | | | | | | | | | | |
| OVC | ABS | 95-(7)- | IND-00375 | | A | | | 59 | Asian | | | | | | | | | | | |
| | | AB-Ser | | | | | | | | | | | | | | | | | | |
| OVC | BioChain | 96-(8)- | A501113 | | A | | | 60 | Asian | | | | | | | | | | | |
| | | Bc-Ser | | | | | | | | | | | | | | | | | | |
| OVC | Biochain | 97-(10)- | A407069 | | A | | | 60 | Asian | | | | | | | | | | | |
| | | Bc-Ser | | | | | | | | | | | | | | | | | | |
| OVC | ABS | 98-(1)- | ILS-1406 | | PA | UN | | 73 | Asian | | | | | | | | | | | |
| | | AB-Ser | | | | | | | | | | | | | | | | | | |
| OVC | GCI | 19-GC- | E2WKF | | EA | IA | 70 | 30 | WCAU | — | Pre-M | 12 | 6 | 5 | 17 | YES | 6 | years | NO | Surg |
| | | Endo | | | | | | | | | | | | | | | | | | |
| | | SIA | | | | | | | | | | | | | | | | | | |
| OVC | GCI | 20-GC- | 5895C | | EA | IA | 95 | 39 | WCAU | — | Pre-M | 14 | 2 | 2 | 20 | NO | — | | NO | Surg |
| | | Endo | | | | | | | | | | | | | | | | | | |
| | | SIA | | | | | | | | | | | | | | | | | | |
| OVC | GCI | 21-GC- | 533DX | | EA | IA | 95 | 50 | WCAU | 190 | Pre-M | 11 | 0 | — | — | YES | 2 | years | NO | Surg |
| | | Endo | | | | | | | | | | | | | | | | | | |
| | | SIA | | | | | | | | | | | | | | | | | | |
| OVC | GCI | 22-GC- | HZ2EY | | EA | IA | 90 | 55 | WCAU | 1078 | Pre-M | 13 | 0 | — | — | NO | — | | NO | Surg |
| | | Endo | | | | | | | | | | | | | | | | | | |
| | | SIA | | | | | | | | | | | | | | | | | | |
| OVC | GCI | 23-GC- | RWOIV | | EA | IA | 65 | 47 | WCAU | 1695 | Pre-M | 14 | 0 | — | — | NO | — | | NO | Surg |
| | | Endo | | | | | | | | | | | | | | | | | | |
| | | SIA | | | | | | | | | | | | | | | | | | |
| OVC | GCI | 24-GC- | 1U52X | | EA | IIA | 95 | 61 | WCAU | 275 | | — | — | — | — | | — | | | Surg |
| | | Endo | | | | | | | | | | | | | | | | | | |
| | | SIIA | | | | | | | | | | | | | | | | | | |
| OVC | GCI | 25-GC- | A17WS | | EA | IIB | 70 | 67 | WCAU | 78 | Post-M | 14 | 0 | — | — | NO | — | | NO | Surg |
| | | Endo | | | | | | | | | | | | | | | | | | |
| | | SIIB | | | | | | | | | | | | | | | | | | |
| OVC | GCI | 26-GC- | 1VT3I | | EA | IIIC | 90 | 50 | WCAU | — | Pre-M | 12 | 2 | 2 | 24 | YES | 1 | years | NO | Surg |
| | | Endo | | | | | | | | | | | | | | | | | | |
| | | SIIIC | | | | | | | | | | | | | | | | | | |
| OVC | GCI | 27-GC- | PZQXH | | EA | IIIC | 80 | 52 | WCAU | — | Pre-M | 11 | 0 | — | — | YES | 5 | years | NO | Surg |
| | | Endo | | | | | | | | | | | | | | | | | | |
| | | SIIIC | | | | | | | | | | | | | | | | | | |
| OVC | GCI | 28-GC- | I8VHZ | | EA | IV | 90 | 68 | WCAU | — | Post-M | — | 2 | 2 | 27 | NO | — | | NO | Surg |
| | | Endo | | | | | | | | | | | | | | | | | | |
| | | SIV | | | | | | | | | | | | | | | | | | |
| OVC | GOG | 99-(41)- | 98-03-G803 | | Mixed | 2 | | 38 | | >35 | | | | | | | | | | |
| | | GO-Ser | | | . . . | | | | | | | | | | | | | | | |
| | | Mix SII | | | | | | | | | | | | | | | | | | |
| OVC | GOG | 100-(40)- | 95-11-G006 | — | PS & EC | 3C | | 49 | | | | | | | | | | | | |
| | | GO-Ser | | | | | | | | | | | | | | | | | | |
| | | Mix | | | | | | | | | | | | | | | | | | |
| | | SIIIC | | | | | | | | | | | | | | | | | | |
| OVC | GOG | 101-(37)- | 2002-05- | | MS & EA | 3C | | 56 | | | | | | | | | | | | |
| | | GO-Ser | G513 | | | | | | | | | | | | | | | | | |
| | | Mix | | | | | | | | | | | | | | | | | | |
| | | SIIIC | | | | | | | | | | | | | | | | | | |
| OVC | GOG | 102-(38)- | 2002-05- | | MS & | 3C | | 64 | | | | | | | | | | | | |
| | | GO-Ser | G509 | | EAM | | | | | | | | | | | | | | | |
| | | Mix | | | | | | | | | | | | | | | | | | |
| | | SIIIC | | | | | | | | | | | | | | | | | | |
| OVC | GOG | 103-(34)- | 95-04-2002 | | PEA | 3C | | 68 | | | | | | | | | | | | |
| | | GO-Ser | | | | | | | | | | | | | | | | | | |
| | | Mix | | | | | | | | | | | | | | | | | | |
| | | SIIIC | | | | | | | | | | | | | | | | | | |
| OVC | GOG | 29-(21)- | 95-10-G020 | | MC | IA | | 44 | | | >100 | | | | | | | | | |
| | | GO-Muc | | | | | | | | | | | | | | | | | | |
| | | SIA | | | | | | | | | | | | | | | | | | |
| OVC | GCI | 30-GC- | IMDA1 | | MA | IC | 70 | 41 | WCAU | 50 | Pre-M | 12 | 2 | 1 | 24 | NO | — | | | Surg |
| | | Muc SIC | | | | | | | | | | | | | | | | | | |
| OVC | Asterand | 31-As- | 12742 | 18920A1 | MA | IC | 70 | 61 | C | | Post-M | | 3 | 3 | | | | | | Surg |
| | | Muc SIC | | | | | | | | | | | | | | | | | | |
| OVC | ABS | 32-(22)- | A0139 | | MC | IC | | 72 | Asian | | | | | | | | | | | |
| | | AB-Muc | | | | | | | | | | | | | | | | | | |
| | | SIC | | | | | | | | | | | | | | | | | | |
| OVC | GCI | 33-GC- | NJM4U | | MA | IIA | 80 | 51 | WCAU | | | | | | | | | | | Surg |
| | | Muc | | | | | | | | | | | | | | | | | | |
| | | SIIA | | | | | | | | | | | | | | | | | | |
| OVC | ABS | 34-(20)- | USA-00273 | | PMC | IIIA | | 45 | C | | | | | | | | | | | |
| | | AB-Muc | | | | | | | | | | | | | | | | | | |
| | | SIIIA | | | | | | | | | | | | | | | | | | |
| OVC | GCI | 35-GC- | RAFCW | | MA | IIIA | 75 | 55 | WCAU | 95 | Post-M | 13 | 4 | 3 | 22 | NO | — | | NO | Surg |
| | | Muc | | | | | | | | | | | | | | | | | | |
| | | SIIIA | | | | | | | | | | | | | | | | | | |
| OVC | Asterand | 36-As- | 23177 | 72888A1 | MA | IIIC | 60 | 52 | C | | Pre-M | | | | | | | | | Surg |
| | | Muc | | | | | | | | | | | | | | | | | | |
| | | SIIIC | | | | | | | | | | | | | | | | | | |
| OVC | Asterand | 37-As- | 16103 | 29374B1 | MA | IIIC | 100 | 62 | W | | Post-M | | 1 | 1 | | | | | | Surg |
| | | Muc | | | | | | | | | | | | | | | | | | |
| | | SIIIC | | | | | | | | | | | | | | | | | | |
| OVC | BioChain | 104-(19)- | A504085 | | MA | | | 34 | Asian | | | | | | | | | | | |
| | | Bc-Muc | | | | | | | | | | | | | | | | | | |
| OVC | BioChain | 105-(18)- | A504083 | | MA | | | 45 | Asian | | | | | | | | | | | |
| | | Bc-Muc | | | | | | | | | | | | | | | | | | |
| OVC | BioChain | 106-(17)- | A504084 | | MA | | | 51 | Asian | | | | | | | | | | | |
| | | Bc-Muc | | | | | | | | | | | | | | | | | | |
| OVC | BioChain | 107-(15)- | A407065 | | C | | | 27 | Asian | | | | | | | | | | | |
| | | Bc-Car | | | | | | | | | | | | | | | | | | |
| OVC | Clontech | 108-(16)- | 1090387 | | CNOS | | | 58 | Asian | | | | | | | | | | | |
| | | Cl-Car | | | | | | | | | | | | | | | | | | |
| OVC | GOG | 109-(44)- | 2001-07- | | CCA | 1A | | 73 | | | | | | | | | | | | |
| | | GO- | G084 | | | | | | | | | | | | | | | | | |
| | | Clear cell | | | | | | | | | | | | | | | | | | |
| | | SIA | | | | | | | | | | | | | | | | | | |
| OVC | GOG | 110-(43)- | 2001-10- | | CCA | 3A | | 74 | | slightly | | | | | | | | | | |
| | | GO- | G002 | | | | | | | elevated | | | | | | | | | | |
| | | Clear cell | | | | | | | | | | | | | | | | | | |
| | | SIIIA | | | | | | | | | | | | | | | | | | |
| OV_BT | GCI | 38-GC- | SC656 | | MBT | IA | 75 | 40 | WCAU | 138 | Pre-M | 13 | 2 | 2 | 23 | NO | — | | YES | Surg |
| | | Muc | | | | | | | | | | | | | | | | | | |
| | | Border | | | | | | | | | | | | | | | | | | |
| | | SIA | | | | | | | | | | | | | | | | | | |
| OV_BT | GCI | 39-GC- | 3D5FO | | MBT | IA | 85 | 51 | WCAU | 19 | ? | 15 | 0 | — | — | NO | — | | NO | Surg |
| | | Muc | | | | | | | | | | | | | | | | | | |
| | | Border | | | | | | | | | | | | | | | | | | |
| | | SIA | | | | | | | | | | | | | | | | | | |
| OV_BT | GCI | 40-GC- | 7JP3F | | MBT | IA | 75 | 56 | WCAU | 125 | Post-M | 14 | 3 | 3 | 19 | YES | 5 | years | NO | Surg |
| | | Muc | | | | | | | | | | | | | | | | | | |
| | | Border | | | | | | | | | | | | | | | | | | |
| | | SIA | | | | | | | | | | | | | | | | | | |
| OV_BT | ABS | 111-(23)- | VNM-00187 | | MC Low M | | | 45 | Asian | | | | | | | | | | | |
| | | AB- | | | | | | | | | | | | | | | | | | |
| | | Border | | | | | | | | | | | | | | | | | | |
| OV_BT | GOG | 112-(42)- | 98-08-G001 | | EA of BM | 1A | | 46 | | | | | | | | | | | | |
| | | GO- | | | | | | | | | | | | | | | | | | |
| | | Border | | | | | | | | | | | | | | | | | | |
| | | SIA | | | | | | | | | | | | | | | | | | |
| OVC_B | GOG | 41-(62)- | 99-10-G442 | | BMC | | | 32 | | 6 | | | | | | | | | | |
| | | Go-Ben | | | | | | | | | | | | | | | | | | |
| | | Muc | | | | | | | | | | | | | | | | | | |
| OVC_B | GCI | 43-GC- | QLIKY | | BMC | | 100 | 42 | WCAU | | | | | | | | | | | Surg |
| | | Ben Muc | | | | | | | | | | | | | | | | | | |
| OVC_B | Asterand | 44-As- | 16870 | 30534A1 | BMC | | 100 | 45 | W | | Pre-M | | 2 | 2 | | | | | | Surg |
| | | Ben Muc | | | | | | | | | | | | | | | | | | |
| OVC_B | GOG | 45-(56)- | 99-01-G407 | | BMC | | | 46 | | | | | | | | | | | | |
| | | GO-Ben | | | | | | | | | | | | | | | | | | |
| | | Muc | | | | | | | | | | | | | | | | | | |
| OVC_B | GCI | 46-GC- | 943EC | | BMC | | 75 | 54 | WCAU | | | | | | | | | | | Surg |
| | | Ben Muc | | | | | | | | | | | | | | | | | | |
| OVC_B | GCI | 47-GC- | JO8W7 | | BMC | | 50 | 56 | WCAU | | | | | | | | | | | Surg |
| | | Ben Muc | | | | | | | | | | | | | | | | | | |
| OVC_B | Asterand | 48-As- | 17016 | 30645B1 | BSC | IA | 100 | 38 | C | | Pre-M | | 2 | 2 | | | | | | Surg |
| | | Ben Ser | | | | | | | | | | | | | | | | | | |
| OVC_B | GOG | 49-(64)- | 99-06-G039 | | BSC | | | 57 | | | | | | | | | | | | |
| | | GO-Ben | | | | | | | | | | | | | | | | | | |
| | | Ser | | | | | | | | | | | | | | | | | | |
| OVC_B | GCI | 50-GC- | DQQ2F | | BSCF | | 95 | 68 | WCAU | | | | | | | | | | | Surg |
| | | Ben Ser | | | | | | | | | | | | | | | | | | |
| OVC_B | Asterand | 51-As- | 8786 | 8275A1 | BSC | | 100 | 80 | CAU | | Post-M | | 10 | 9 | | | | | | Surg |
| | | Ben Ser | | | | | | | | | | | | | | | | | | |
| OV_NBM | Asterand | 52-As-N | 15690 | 23054A1 | NO-BM | | | 52 | CAU | | Pre-M | | 10 | 3 | | | | | | Surg |
| | | BM | | | | | | | | | | | | | | | | | | |
| OV_NBM | Asterand | 53-As-N | 16843 | 30488A1 | NO-BM | | | 57 | W | | Post-M | | 4 | 2 | | | | | | Surg |
| | | BM | | | | | | | | | | | | | | | | | | |
| OV_NBM | Asterand | 54-As-N | 16850 | 30496B1 | NO-BM | | | 65 | W | | Post-M | | 2 | 2 | | | | | | Surg |
| | | BM | | | | | | | | | | | | | | | | | | |
| OV_NBM | Asterand | 55-As-N | 16848 | 30499C1 | NO-BM | | | 66 | CAU | | Post-M | | 9 | 2 | | | | | | Surg |
| | | BM | | | | | | | | | | | | | | | | | | |
| OVN | GCI | 56-GC-N | WPU1U | | NO- | | 0 | 32 | WC | | | | | | | | | | | Surg |
| | | PS | | | POSTS | | | | | | | | | | | | | | | |
| OVN | GCI | 57-GC-N | Y9VHI | | NO- | | 0 | 35 | WCAU | | | | | | | | | | | Surg |
| | | PS | | | POSTS | | | | | | | | | | | | | | | |
| OVN | GCI | 58-GC-N | 76VM9 | | NO- | | 0 | 41 | WCAU | | | | | | | | | | | Surg |
| | | PS | | | POSTS | | | | | | | | | | | | | | | |
| OVN | GCI | 59-GC-N | DWHTZ | | NO- | | 0 | 42 | WCAU | | | | | | | | | | | Surg |
| | | PS | | | POSTS | | | | | | | | | | | | | | | |
| OVN | GCI | 60-GC-N | SJ2R2 | | NO- | | 0 | 43 | WCAU | | | | | | | | | | | Surg |
| | | PS | | | POSTS | | | | | | | | | | | | | | | |
| OVN | GCI | 61-GC-N | 9RQMN | | NO- | | 0 | 45 | WCAU | | | | | | | | | | | Surg |
| | | PS | | | POSTS | | | | | | | | | | | | | | | |
| OVN | GCI | 62-GC-N | TOAE5 | | NO- | | 0 | 45 | WCAU | | | | | | | | | | | Surg |
| | | PS | | | POSTS | | | | | | | | | | | | | | | |
| OVN | GCI | 63-GC-N | TW9PM | | NO- | | 0 | 46 | WCAU | | | | | | | | | | | Surg |
| | | PS | | | POSTS | | | | | | | | | | | | | | | |
| OVN | GCI | 64-GC-N | 2VND2 | | NO- | | 0 | 46 | WCAU | | | | | | | | | | | Surg |
| | | PS | | | POSTS | | | | | | | | | | | | | | | |
| OVN | GCI | 65-GC-N | L629F | | NO- | | 0 | 47 | WCAU | | | | | | | | | | | Surg |
| | | PS | | | POSTS | | | | | | | | | | | | | | | |
| OVN | GCI | 66-GC-N | XLB23 | | NO- | | 0 | 47 | WCAU | | | | | | | | | | | Surg |
| | | PS | | | POSTS | | | | | | | | | | | | | | | |
| OVN | GCI | 67-GC-N | IDUVY | | NO- | | 0 | 47 | WCAU | | | | | | | | | | | Surg |
| | | PS | | | POSTS | | | | | | | | | | | | | | | |
| OVN | GCI | 68-GC-N | ZCXAD | | NO- | | 0 | 48 | WCAU | | | | | | | | | | | Surg |
| | | PS | | | POSTS | | | | | | | | | | | | | | | |
| OVN | GCI | 69-GC-N | PEQ6C | | NO- | | 0 | 49 | WCAU | | | | | | | | | | | Surg |
| | | PS | | | POSTS | | | | | | | | | | | | | | | |
| OVN | GCI | 70-GC-N | DD73B | | NO- | | 0 | 49 | WCAU | | | | | | | | | | | Surg |
| | | PS | | | POSTS | | | | | | | | | | | | | | | |
| OVN | GCI | 71-GC-N | E2UF7 | | NO- | | 0 | 53 | WCAU | | | | | | | | | | | Surg |
| | | PS | | | POSTS | | | | | | | | | | | | | | | |
| OVN | GCI | 72-GC-N | GWXUZ | | NO- | | 0 | 53 | WCAU | | | | | | | | | | | Surg |
| | | PS | | | POSTS | | | | | | | | | | | | | | | |
| OVN | GCI | 73-GC-N | 4YG5P | | NO- | | 0 | 55 | WCAU | | | | | | | | | | | Surg |
| | | PS | | | POSTS | | | | | | | | | | | | | | | |
| OVN | GCI | 74-GC-N | FDPL9 | | NO- | | 0 | 56 | WCAU | | | | | | | | | | | Surg |
| | | PS | | | POSTS | | | | | | | | | | | | | | | |
| OVN | BioChain | 75-(45)- | A503274 | | NO-PM | | | 41 | Asian | | | | | | | | | | | |
| | | Bc-N PM | | | | | | | | | | | | | | | | | | |
| OVN | BioChain | 76-(46)- | A504086 | | NO-PM | | | 41 | Asian | | | | | | | | | | | |
| | | Bc-N PM | | | | | | | | | | | | | | | | | | |
| OVN | Ichilov | 77-(71)- | CG-188-7 | | NO-PM | | | 49 | | | | | | | | | | | | |
| | | Ic-N PM | | | | | | | | | | | | | | | | | | |
| OVN | BioChain | 78-(48)- | A504087 | | NO-PM | | | 51 | Asian | | | | | | | | | | | |
| | | Bc-N PM | | | | | | | | | | | | | | | | | | |

TABLE 1_1_2

| Key | Full Name |
|---|---|
| A | Adenocarcinoma |
| APP | Adenocarcinoma from primary peritoneal |
| BMC | BENIGN MUCINOUS CYSTADENOMA |
| BSC | BENIGN SEROUS CYSTADENOMA |
| BSCF | BENIGN SEROUS CYSTADENOFIBROMA |
| C | Carcinoma |
| C Stage | Cancer stage |
| CAU | Caucasian |
| CCA | Clear cell adenocarcinoma |
| CNOS | Carcinoma NOS |
| EA | ENDOMETROID ADENOCARCINOMA |
| EA of BM | Endometroid adenocarcinoma of borderline malignancy |
| HT | Height |
| M | Menopausal |
| MA | MUCINOUS ADENOCARCINOMA |
| MBT | MUCINOUS BORDERLINE TUMOR |
| MC | Mucinous cystadenocarcinoma |
| MC Low M | Mucinous cystadenocarcinoma with low malignant |
| Mens. Age | Menstrual Age |
| Mixed . . . | Mixed epithelial cystadenocarcinoma with mucinous, |
| | endometrioid, squamous and papillary serous |
| MS & EA | Mixed serous and endometrioid adenocarcinoma |
| MS & EAM | Mixed serous and endometrioid adenocarcinoma of |
| | mullerian |
| NO-BM | NORMAL OVARY-Benign Matched |
| NO-PM | NORMAL OVARY-Post Mortum |
| NO-PostS | NORMAL OVARY-Post Surgery |
| OC | Oral Contraceptive |
| OV_BT | Ovary Borderline Tumor |
| OV_NBM | Ovary Normal Benign Matched |
| OVN | Ovary Normal |
| PA | Papillary adenocarcinoma |
| PC | Papillary cystadenocarcinoma |
| PEA | Papillary endometrioid adenocarcinoma |
| PMC | Papillary mucinous cystadenocarcinoma |
| Post-M | Post-menopausal |
| Pre-M | Pre-menopausal |
| PS & EC | Papillary serous and endometrioid cystadenocarcinoma |
| PSA | Papillary serous adenocarcinoma |
| PSC | Papillary serous carcinoma |
| SA | SEROUS ADENOCARCINOMA |
| SPC | Serous papillary cystadenocarcinoma |
| W | White |
| WCAU | WHITE/CAUCASIAN |
| WT | Weight |

TABLE 1_2
Tissue samples in lung cancer testing panel

| | | | Sample id | | | | | | | | | |
|---|---|---|---|---|---|---|---|---|---|---|---|---|
| | | | (GCI)/ | | RNA | | | | | | | |
| | | | case id | TISSUE ID | ID (GCI)/ | | | | | | | |
| | | | (Asterand)/ | (GCI)/specimen | Sample | | | | | | | |
| | Source/ | sample | lot no. (old | ID | ID | | Diag | Specimen | | | | |
| Tissue | Delivery | name | samples) | (Asterand) | (Asterand) | Diag | remarks | location | Gr | TNM | CS | Tum % |
| LC | GCI | 1-GC- | 7Z9V4 | 7Z9V4AYM | | Aden | BAC | | | | IA | 80 |
| | | BAC- | | | | | | | | | | |
| | | SIA | | | | | | | | | | |
| LC | GCI | 2-GC- | ZW2AQ | ZW2AQARP | | Aden | BAC | | | | IB | 70 |
| | | BAC- | | | | | | | | | | |
| | | SIB | | | | | | | | | | |
| LC | Bioch | 72-(44)- | A501123 | | | AC | | | 2 | UN | | |
| | | Bc-BAC | | | | | | | | | | |
| LC | Bioch | 73-(45)- | A501221 | | | AC | | | UN | UN | | |
| | | Bc-BAC | | | | | | | | | | |
| LC | GCI | 4-GC- | 3MOPL | 3MOPLA79 | | Aden | | | | | IA | 60 |
| | | Adeno- | | | | | | | | | | |
| | | SIA | | | | | | | | | | |
| LC | GCI | 5-GC- | KOJXD | KOJXDAV4 | | Aden | | | | | IA | 90 |
| | | Adeno- | | | | | | | | | | |
| | | SIA | | | | | | | | | | |
| LC | GCI | 6-GC- | X2Q44 | X2Q44A79 | | Aden | | | | | IA | 85 |
| | | Adeno- | | | | | | | | | | |
| | | SIA | | | | | | | | | | |
| LC | GCI | 7-GC- | 6BACZ | 6BACZAP5 | | Aden | | | | | IA | 60 |
| | | Adeno- | | | | | | | | | | |
| | | SIA | | | | | | | | | | |
| LC | GCI | 8-GC- | BS9AF | BS9AFA3E | | Aden | | | | | IA | 55 |
| | | Adeno- | | | | | | | | | | |
| | | SIA | | | | | | | | | | |
| LC | GCI | 9-GC- | UCLOA | UCLOAA9L | | Aden | | | | | IA | 80 |
| | | Adeno- | | | | | | | | | | |
| | | SIA | | | | | | | | | | |
| LC | GCI | 10-GC- | BVYK3 | BVYK3A7Z | | Aden | | | | | IA | 60 |
| | | Adeno- | | | | | | | | | | |
| | | SIA | | | | | | | | | | |
| LC | GCI | 11-GC- | U4DM4 | U4DM4AFZ | | Aden | | | | | IB | 65 |
| | | Adeno- | | | | | | | | | | |
| | | SIB | | | | | | | | | | |
| LC | GCI | 12-GC- | OWX5Y | OWX5YA3S | | Aden | | | | | IB | 90 |
| | | Adeno- | | | | | | | | | | |
| | | SIB | | | | | | | | | | |
| LC | GCI | 13-GC- | XYY96 | XYY96A6B | | Aden | | | | | IIA | 70 |
| | | Adeno- | | | | | | | | | | |
| | | SIIA | | | | | | | | | | |
| LC | GCI | 14-GC- | SO7B1 | SO7B1AIJ | | Aden | | | | | IIA | 70 |
| | | Adeno- | | | | | | | | | | |
| | | SIIA | | | | | | | | | | |
| LC | GCI | 15-GC- | QANSY | QANSYACD | | Aden | | | | | IIIA | 65 |
| | | Adeno- | | | | | | | | | | |
| | | SIIIA | | | | | | | | | | |
| LC | Bioch | 16-(95)- | A610063 | | | Aden | | | 1 | UN | | |
| | | BC- | | | | | | | | | | |
| | | Adeno | | | | | | | | | | |
| LC | Bioch | 17-(89)- | A609077 | | | Aden | | | 2-3 | UN | | |
| | | Bc- | | | | | | | | | | |
| | | Adeno | | | | | | | | | | |
| LC | Bioch | 18-(76)- | A609218 | | | Aden | | | 3 | UN | | |
| | | Bc- | | | | | | | | | | |
| | | Adeno | | | | | | | | | | |
| LC | Bioch | 74-(2)- | A504118 | | | Aden | | | 1 | UN | | |
| | | Bc- | | | | | | | | | | |
| | | Adeno | | | | | | | | | | |
| LC | Bioch | 76-(75)- | A609217 | | | Aden | | | 2 | UN | | |
| | | Bc- | | | | | | | | | | |
| | | Adeno | | | | | | | | | | |
| LC | Bioch | 77-(12)- | A504119 | | | Aden | | | 2 | UN | | |
| | | Bc- | | | | | | | | | | |
| | | Adeno | | | | | | | | | | |
| LC | Bioch | 78-(13)- | A504116 | | | Aden | | | 2-3 | UN | | |
| | | Bc- | | | | | | | | | | |
| | | Adeno | | | | | | | | | | |
| LC | Bioch | 79-(94)- | A610118 | | | Aden | | | 3 | UN | | |
| | | Bc- | | | | | | | | | | |
| | | Adeno | | | | | | | | | | |
| LC | Ichilov | 80-(3)- | CG-200 | | | Aden | | | UN | UN | | |
| | | Ic- | | | | | | | | | | |
| | | Adeno | | | | | | | | | | |
| LC | Ichilov | 81-(14)- | CG-111 | | | Aden | | | UN | UN | | |
| | | Ic- | | | | | | | | | | |
| | | Adeno | | | | | | | | | | |
| LC | Aster | 19-As- | 9220 | 9418 | 9418A1 | SQ | | | 1 | TXN0M0 | Occult | 80 |
| | | Sq-S0 | | | | | | | | | | |
| LC | GCI | 20-GC- | U2QHS | U2QHSA2N | | SQ | | | | | IA | 55 |
| | | Sq-SIA | | | | | | | | | | |
| LC | GCI | 21-GC- | TRQR7 | TRQR7ACD | | SQ | | | | | IB | 75 |
| | | Sq-SIB | | | | | | | | | | |
| LC | Aster | 22-As- | 17581 | 32603 | 32603B1 | SQ | | | 3 | T2N0M0 | IB | 90 |
| | | Sq-SIB | | | | | | | | | | |
| LC | Aster | 23-As- | 18309 | 41454 | 41454B1 | SQ | | | 2 | T2N0MX | IB | 100 |
| | | Sq-SIB | | | | | | | | | | |
| LC | Aster | 24-As- | 9217 | 9415 | 9415B1 | SQ | | | 2 | T2N0M0 | IB | 90 |
| | | Sq-SIB | | | | | | | | | | |
| LC | GCI | 25-GC- | RXQ1P | RXQ1PAEA | | SQ | | | | | IIB | 55 |
| | | Sq-SIIB | | | | | | | | | | |
| LC | GCI | 26-GC- | KB5KH | KB5KHA6X | | SQ | | | | | IIB | 65 |
| | | Sq-SIIB | | | | | | | | | | |
| LC | GCI | 27-GC- | LAYMB | LAYMBALF | | SQ | | | | | IIIA | 65 |
| | | Sq-SIIIA | | | | | | | | | | |
| LC | Ichilov | 28-(23)- | CG-109 (1) | | | SQ | | | UN | UN | | |
| | | Ic-Sq | | | | | | | | | | |
| LC | Ichilov | 29-(25)- | CG-204 | | | SQ | | | UN | UN | | |
| | | Ic-Sq | | | | | | | | | | |
| LC | Bioch | 30-(19)- | A408175 | | | SQ | | | 1 | UN | | |
| | | Bc-Sq | | | | | | | | | | |
| LC | Bioch | 31-(78)- | A607125 | | | SQ | | | 2 | UN | | |
| | | Bc-Sq | | | | | | | | | | |
| LC | Bioch | 32-(16)- | A409091 | | | SQ | | | 2 | UN | | |
| | | Bc-Sq | | | | | | | | | | |
| LC | Bioch | 33-(80)- | A609163 | | | SQ | | | 2 | UN | | |
| | | Bc-Sq | | | | | | | | | | |
| LC | Bioch | 34-(18)- | A503387 | | | SQ | | | 2-3 | UN | | |
| | | Bc-Sq | | | | | | | | | | |
| LC | Bioch | 35-(81)- | A609076 | | | SQ | | | 3 | UN | | |
| | | Bc-Sq | | | | | | | | | | |
| LC | Bioch | 82-(21)- | A503187 | | | SQ | | | 2 | UN | | |
| | | Bc-Sq | | | | | | | | | | |
| LC | Bioch | 83-(17)- | A503183 | | | SQ | | | 2 | UN | | |
| | | Bc-Sq | | | | | | | | | | |
| LC | Bioch | 84-(79)- | A609018 | | | SQ | | | 3 | UN | | |
| | | Bc-Sq | | | | | | | | | | |
| LC | Bioch | 85-(22)- | A503386 | | | SQ | | | UN | UN | | |
| | | Bc-Sq | | | | | | | | | | |
| LC | Bioch | 86-(20)- | A501121 | | | SQ | | | UN | UN | | |
| | | Bc-Sq | | | | | | | | | | |
| LC | Bioch | 87-(88)- | A609219 | | | SQ | | | UN | UN | | |
| | | Bc-Sq | | | | | | | | | | |
| LC | Bioch | 88- | A409017 | | | SQ | | | UN | UN | | |
| | | (100)- | | | | | | | | | | |
| | | Bc-Sq | | | | | | | | | | |
| LC | Ichilov | 89-(24)- | CG-123 | | | SQ | | | UN | UN | | |
| | | Ic-Sq | | | | | | | | | | |
| LC | GCI | 36-GC- | AF8AL | AF8ALAAL | | LCC | | | | | IA | 85 |
| | | LCC- | | | | | | | | | | |
| | | SIA | | | | | | | | | | |
| LC | GCI | 37-GC- | O62XU | O62XUA1X | | LCC | | | | | IB | 75 |
| | | LCC- | | | | | | | | | | |
| | | SIB | | | | | | | | | | |
| LC | GCI | 38-GC- | OLOIM | OLOIMAS1 | | LCC | | | | | IB | 70 |
| | | LCC- | | | | | | | | | | |
| | | SIB | | | | | | | | | | |
| LC | GCI | 39-GC- | 1ZWSV | 1ZWSVAB9 | | LCC | | | | | IIB | 50 |
| | | LCC- | | | | | | | | | | |
| | | SIIB | | | | | | | | | | |
| LC | GCI | 40-GC- | 2YHOD | 2YHODA1H | | LCC | NSCC . . . | | | | IIB | 95 |
| | | LCC- | | | | | | | | | | |
| | | SIIB | | | | | | | | | | |
| LC | GCI | 41-GC- | 38B4D | 38B4DAQK | | LCC | | | | | IIB | 90 |
| | | LCC- | | | | | | | | | | |
| | | SIIB | | | | | | | | | | |
| LC | Bioch | 90-(39)- | A504114 | | | LCC | | | UN | UN | | |
| | | Bc-LCC | | | | | | | | | | |
| LC | Bioch | 91-(87)- | A609165 | | | LCC | | | 3 | UN | | |
| | | Bc-LCC | | | | | | | | | | |
| LC | Bioch | 92-(38)- | A504113 | | | LCC | | | UN | UN | | |
| | | Bc-LCC | | | | | | | | | | |
| LC | Bioch | 93-(82)- | A609170 | | | LCNC | | | UN | UN | | |
| | | Bc-LCC | | | | | | | | | | |
| LC | GCI | 42-GC- | QPJQL | QPJQLAF6 | | SCC | NC | | 3 | | IB | 65 |
| | | SCC- | | | | | | | | | | |
| | | SIB | | | | | | | | | | |
| LC | Bioch | 43-(32)- | A501391 | | | SCC | | | | UN | | |
| | | Bc-SCC | | | | | | | | | | |
| LC | Bioch | 44-(30)- | A501389 | | | SCC | | | 3 | UN | | |
| | | Bc-SCC | | | | | | | | | | |
| LC | Bioch | 45-(83)- | A609162 | | | SCC | | | UN | UN | | |
| | | Bc-SCC | | | | | | | | | | |
| LC | Bioch | 46-(86)- | A608032 | | | SCC | | | 3 | UN | | |
| | | Bc-SCC | | | | | | | | | | |
| LC | Bioch | 47-(31)- | A501390 | | | SCC | | | | UN | | |
| | | Bc-SCC | | | | | | | | | | |
| LC | Bioch | 48-(84)- | A609167 | | | SCC | | | UN | UN | | |
| | | Bc-SCC | | | | | | | | | | |
| LC | Bioch | 49-(85)- | A609169 | | | SCC | | | UN | UN | | |
| | | Bc-SCC | | | | | | | | | | |
| LC | Bioch | 50-(33)- | A504115 | | | SCC | | | | UN | | |
| | | Bc-SCC | | | | | | | | | | |
| LN | Aster | 51-As- | 9078 | 9275 | 9275B1 | NL | PS | | | | | |
| | | N-PS | | | | | | | | | | |
| LN | Aster | 52-As- | 8757 | 8100 | 8100B1 | NL | PM | (Right), | | | | |
| | | N-PM | | | | | | Lobe | | | | |
| | | | | | | | | Inferior | | | | |
| LN | Aster | 53-As- | 6692 | 6161 | 6161A1 | NL | PM | | | | | |
| | | N-PM | | | | | | | | | | |
| LN | Aster | 54-As- | 7900 | 7180 | 7180F1 | NL | PM | | | | | |
| | | N-PM | | | | | | | | | | |
| LN | Aster | 55-As- | 8771 | 8163 | 8163A1 | NL | PM | (Left), | | | | |
| | | N-PM | | | | | | Lobe | | | | |
| | | | | | | | | Superior | | | | |
| LN | Aster | 56-As- | 13094 | 19763 | 19763A1 | NL | PM | | | | | |
| | | N-PM | | | | | | | | | | |
| LN | Aster | 57-As- | 19174 | 40654 | 40654A2 | NL | PM | | | | | |
| | | N-PM | | | | | | | | | | |
| LN | Aster | 58-As- | 13128 | 19642 | 19642A1 | NL | PM | | | | | |
| | | N-PM | | | | | | | | | | |
| LN | Aster | 59-As- | 14374 | 20548 | 20548C1 | NL | PM | (Right), | | | | |
| | | N-PM | | | | | | Lobe | | | | |
| | | | | | | | | Superior | | | | |
| LN | Amb | 60-(99)- | 36856 | | | N-PM | PM | | | | | |
| | | Am-N | | | | | | | | | | |
| | | PM | | | | | | | | | | |
| LN | Amb | 61-(96)- | 36853 | | | N-PM | PM | | | | | |
| | | Am-N | | | | | | | | | | |
| | | PM | | | | | | | | | | |
| LN | Amb | 62-(97)- | 36854 | | | N-PM | PM | | | | | |
| | | Am-N | | | | | | | | | | |
| | | PM | | | | | | | | | | |
| LN | Amb | 63-(93)- | 111P0103A | | | N-PM | PM-ICH | | | | | |
| | | Am-N | | | | | | | | | | |
| | | PM | | | | | | | | | | |
| LN | Amb | 64-(98)- | 36855 | | | N-PM | PM | | | | | |
| | | Am-N | | | | | | | | | | |
| | | PM | | | | | | | | | | |
| LN | Bioch | 67-(50)- | A503385 | | | N-PM | PM | | | | | |
| | | Bc-N | | | | | | | | | | |
| | | PM | | | | | | | | | | |
| LN | Bioch | 68-(92)- | A503204 | | | N-PM | PM | | | | | |
| | | Bc-N | | | | | | | | | | |
| | | PM | | | | | | | | | | |
| LN | Bioch | 69-(91)- | A607257 | | | N-P2- | PM | | | | | |
| | | Bc-N | | | | PM | | | | | | |
| | | PM | | | | | | | | | | |
| LN | Bioch | 70-(90)- | A608152 | | | N-P2 | PM | | | | | |
| | | Bc-N | | | | PM | | | | | | |
| | | PM | | | | | | | | | | |
| LN | Bioch | 71-(48)- | A503206 | | | N-PM | PM | | | | | |
| | | Bc-N | | | | | | | | | | |
| | | PM | | | | | | | | | | |

| | | | | | | # of | | | | | | | | |
|---|---|---|---|---|---|---|---|---|---|---|---|---|---|---|
| | | | | | # Cig. | Y. Use | # Y. | | | | | | Cause | |
| | | | | Smoking | Per | of | off | Sm | Sm | Dr | # | Recovery | of | Exc. |
| Tissue | Gen | age | Ethnic B | Status | day | Tobacco | Tobacco | PY? | ppl | Al | Dr | Type | Death | Y. |
| LC | F | 63 | WCAU | Prev U. | 20 | 15 | 27 | N | — | Y | 0 | Surg | | 2001 |
| LC | F | 56 | WCAU | Prev U. | 15 | 28 | 10 | Y | 1 | Y | 6 | Surg | | 2002 |
| LC | F | 61 | | | | | | | | | | | | |
| LC | F | 50 | | | | | | | | | | | | |
| LC | M | 68 | WCAU | Nev U. | — | — | — | N | — | N | — | Surg | | 2001 |
| LC | F | 64 | WCAU | Prev U. | 15 | 40 | 7 | Y | 1 | N | 0 | Surg | | 2003 |
| LC | M | 58 | WCAU | Prev U. | 10 | 47 | 0 | Y | 2 | N | — | Surg | | 2004 |
| LC | F | 65 | WCAU | Curr U. | 6 | 30 | — | Y | 1 | N | — | Surg | | 2004 |
| LC | F | 59 | WCAU | Curr U. | 20 | 40 | — | N | — | N | — | Surg | | 2004 |
| LC | F | 69 | WCAU | Curr U. | 30 | 52 | — | Y | 4 | N | — | Surg | | 2005 |
| LC | F | 60 | WCAU | Curr U. | 40 | 40 | — | N | — | N | — | Surg | | 2002 |
| LC | F | 68 | WCAU | Prev U. | 5 | 4 | 43 | N | — | N | — | Surg | | 2003 |
| LC | M | 69 | WCAU | Curr U. | 10 | — | — | | — | N | — | Surg | | 2002 |
| LC | F | 62 | WCAU | Prev U. | 6 | 40 | 6 | N | — | Y | 0 | Surg | | 2004 |
| LC | M | 56 | WCAU | Curr U. | 30 | 25 | — | Y | 1 | N | — | Surg | | 2001 |
| LC | F | 61 | WCAU | Curr U. | 30 | 36 | — | Y | 1 | N | — | Surg | | 2004 |
| LC | F | 54 | | | | | | | | | | | | |
| LC | M | 62 | | | | | | | | | | | | |
| LC | M | 57 | | | | | | | | | | | | |
| LC | M | 64 | | | | | | | | | | | | |
| LC | M | 65 | | | | | | | | | | | | |
| LC | F | 74 | | | | | | | | | | | | |
| LC | M | 64 | | | | | | | | | | | | |
| LC | M | 68 | | | | | | | | | | | | |
| LC | F | 56 | | | | | | | | | | | | |
| LC | M | 68 | | | | | | | | | | | | |
| LC | M | 67 | CAU | Curr U. | 11-20 | 31-40 | | | | O | | Surg | | 2003 |
| LC | F | 68 | WCAU | Prev U. | 10 | 20 | 0 | N | — | N | — | Surg | | 2004 |
| LC | M | 62 | WCAU | Prev U. | 20 | 50 | 0 | Y | 5 | N | — | Surg | | 2005 |
| LC | M | 73 | CAU | Prev U. | | | | | | O | | Surg | | 2004 |
| LC | M | 66 | CAU | Prev U. | 11-20 | 45 | | | | P | | Surg | | 2005 |
| LC | M | 65 | CAU | Curr U. | 6-10 | 41-50 | | | | O | | Surg | | 2002 |
| LC | F | 44 | WCAU | Prev U. | 20 | 20 | 0 | Y | 2 | N | — | Surg | | 2004 |
| LC | M | 68 | WCAU | Prev U. | 40 | 40 | 0 | Y | 2 | N | — | Surg | | 2004 |
| LC | F | 58 | WCAU | Prev U. | 50 | 40 | 1 | Y | 2 | N | — | Surg | | 2004 |
| LC | M | 65 | | | | | | | | | | | | |
| LC | M | 72 | | | | | | | | | | | | |
| LC | M | 78 | | | | | | | | | | | | |
| LC | M | 62 | | | | | | | | | | | | |
| LC | F | 68 | | | | | | | | | | | | |
| LC | M | 74 | | | | | | | | | | | | |
| LC | M | 63 | | | | | | | | | | | | |
| LC | M | 53 | | | | | | | | | | | | |
| LC | M | 52 | | | | | | | | | | | | |
| LC | M | 57 | | | | | | | | | | | | |
| LC | M | 67 | | | | | | | | | | | | |
| LC | M | 48 | | | | | | | | | | | | |
| LC | M | 64 | | | | | | | | | | | | |
| LC | M | 64 | | | | | | | | | | | | |
| LC | M | 64 | | | | | | | | | | | | |
| LC | M | 76 | | | | | | | | | | | | |
| LC | M | 45 | WCAU | Prev U. | 45 | 33 | 0 | Y | 2 | Y | 28 | Surg | | 2004 |
| LC | F | 60 | WCAU | Prev U. | 30 | 45 | 0 | Y | 3 | N | — | Surg | | 2004 |
| LC | M | 68 | WCAU | Prev U. | — | 55 | — | Y | — | N | — | Surg | | 2001 |
| LC | M | 51 | WCAU | Prev U. | 20 | 12 | 22 | Y | 1 | N | — | Surg | | 2004 |
| LC | M | 62 | WCAU | Prev U. | 40 | 40 | 0 | Y | 2 | Y | 12 | Surg | | 2004 |
| LC | F | 70 | WCAU | Prev U. | 30 | 50 | — | Y | 2 | Y | 13 | Surg | | 2002 |
| LC | F | 35 | | | | | | | | | | | | |
| LC | F | 47 | | | | | | | | | | | | |
| LC | M | 58 | | | | | | | | | | | | |
| LC | M | 68 | | | | | | | | | | | | |
| LC | F | 62 | WCAU | Prev U. | 20 | 35 | 0.15 | Y | 2 | N | — | Surg | | 2003 |
| LC | M | 30 | | | | | | | | | | | | |
| LC | M | 34 | | | | | | | | | | | | |
| LC | F | 47 | | | | | | | | | | | | |
| LC | F | 52 | | | | | | | | | | | | |
| LC | F | 59 | | | | | | | | | | | | |
| LC | F | 59 | | | | | | | | | | | | |
| LC | M | 66 | | | | | | | | | | | | |
| LC | M | | | | | | | | | | | | | |
| LN | M | 22 | CAU | Nev U. | | | | | | NU | | Surg | | 2003 |
| LN | F | 26 | CAU | Nev U. | | | | | | O | | Aut | CA | 2003 |
| LN | M | 37 | CAU | Nev U. | | | | | | C | | Aut | MCE | 2002 |
| LN | F | 76 | CAU | Prev U. | | | | | | | | Aut | CPul A | 2002 |
| LN | M | 81 | CAU | Prev U. | 41 or | 31-40 | | | | O | | Aut | CA | 2003 |
| | | | | | more | | | | | | | | | |
| LN | M | 0 | CAU | Prev U. | 21-40 | 41-50 | | | | P | | Aut | IC | |
| LN | F | 69 | CAU | Curr U. | 21-40 | 31-40 | | | | P | | Aut | CPul A | 2005 |
| LN | F | 75 | CAU | | | | | | | | | Aut | CPul A | 2004 |
| LN | F | 75 | CAU | | | | | | | | | Aut | Cer A | 2004 |
| LN | M | 31 | | | | | | | | | | | | |
| LN | F | 43 | | | | | | | | | | | | |
| LN | M | 46 | | | | | | | | | | | | |
| LN | F | 61 | | | | | | | | | | | | |
| LN | F | 72 | | | | | | | | | | | | |
| LN | M | 28 | | | | | | | | | | | | |
| LN | M | 28 | | | | | | | | | | | | |
| LN | P2 | 24, 29 | | | | | | | | | | | | |
| LN | P2 | 27, 28 | | | | | | | | | | | | |
| LN | M | 44 | | | | | | | | | | | | |

TABLE 1_2_1

| Key | Full Name |
|---|---|
| # Cig. Per day | Number of Cigarettes per day |
| # Dr | Number of Drinks |
| # of Y. Use of Tobacco | Number of Years Using Tobacco |
| # Y. off Tobacco | Number of Years Off Tobacco |
| AC | Alveolus carcinoma |
| Aden | ADENOCARCINOMA |
| Amb | Ambion |
| Aster | Asterand |
| Aut | Autopsy |
| BAC | BRONCHIOLOALVEOLAR |
| | CARCINOMA |
| Bioch | Biochain |
| C | Current Use |
| CA | Cardiac arrest |
| CAU | Caucasian |
| Cer A | Cerebrovascular accident |
| CPul A | Cardiopulmonary arrest |
| CS | Cancer Stage |
| Curr U. | Current Use |
| Diag | Diagnosis |
| Dr Al | Drink Alcohol? |
| Exc Y. | Excision Year |
| Gen | Gender |
| Gr | Grade |
| Height | HT |
| IC | Ischemic cardiomyopathy |
| LC | Lung Cancer |
| LCC | LARGE CELL CARCINOMA |
| LCNC | Large Cell Neuroendocrine |
| | Carcinoma |
| LN | Lung Normal |
| MCE | Massive cerebral edema |
| N | No |
| NC | NEUROENDOCRINE |
| | CARCINOMA |
| Nev. U. | Never Used |
| NL | Normal Lung |
| N-P2-PM | Normal (Pool 2)-Post Mortum |
| N-PM | Normal-Post Mortum |
| NSCC . . . | NON-SMALL CELL CARCINOMA |
| | WITH SARCOMUTOUS |
| | TRANSFORMTAIO |
| NU | Never used |
| O | Occasional Use |
| P | Previous Use |
| P2 | Pool 2 |
| Prev U. | Previous Use |
| SQ | Squamous Cell Carcinoma |
| Sm P Y? | Have people at home smoked in |
| | past 15 yr |
| Sm ppl | If yes, how many? |
| SCC | SMALL CELL CARCINOMA |
| SMOKE_GROWING_UP | Did people smoke at home while |
| | growing up |
| Surg | Surgical |
| Tum % | Tumor Percentage |
| WCAU | White Caucasian |
| Y | Yes |

TABLE 1_3
Tissue samples in Breast cancer testing panel

| | | | sample_id (GCI)/ | | | | | |
|---|---|---|---|---|---|---|---|---|
| | | | case id (Asterand)/ | TISSUE_ID | | | | |
| | Source/ | sample | lot no. (old | (GCI)/specimen | Sample ID | | | |
| Tissue | Delivery | name | samples) | ID (Aster and) | (Asterand) | SampDIAG | Grade | TNM |
| BC_in- | Aster | 1-As- | 19723 | 42509 | 42509A1 | DCIS | High | T1aN0M0 |
| situ | | DCISS0 | | | | | Grade | |
| BC | GCI | 2-GC- | 5IRTK | 5IRTKAXT | | IDC | | |
| | | IDC SI | | | | | | |
| BC | ABS | 3-(42)- | 6005020031T | | | IDC | 3 | T1cN0Mx |
| | | AB-IDC | | | | | | |
| | | SI | | | | | | |
| BC | ABS | 4-(7)-AB- | 7263T | | | IDC | 2 | T1N0M0 |
| | | IDC SI | | | | | | |
| BC | GCI | 5-GC- | DSI52 | DSI52AH3 | | IDC | | |
| | | IDC SI | | | | | | |
| BC | GCI | 6-GC- | S2GBY | S2GBYAGC | | IDC | | |
| | | IDC SI | | | | | | |
| BC | GCI | 7-GC- | POPHP | POPHPAZ4 | | IDC | | |
| | | IDC SI | | | | | | |
| BC | GCI | 8-GC- | I2YLE | I2YLEACP | | IDC | | |
| | | IDC SI | | | | | | |
| BC | Aster | 9-As-IDC | 17959 | 31225 | 31225A1 | IDC | 2 | T1NXM0 |
| | | SI | | | | | | |
| BC | ABS | 10-(12)- | 1432T | | | IDC | 2 | T2N0M0 |
| | | AB-IDC | | | | | | |
| | | SIIA | | | | | | |
| BC | Aster | 11-As- | 17138 | 30697 | 30697A1 | IDC | 3 | T2NXM0 |
| | | IDC | | | | | | |
| | | SIIA | | | | | | |
| BC | GCI | 12-GC- | YSZ67 | YSZ67A48 | | IDC | | |
| | | IDC SIIA | | | | | | |
| BC | ABS | 13-(6)- | 7238T | | | IDC | 1 | T2N0M0 |
| | | AB-IDC | | | | | | |
| | | SIIA | | | | | | |
| BC | ABS | 14-(26)- | 7249T | | | IDC | 3 | T2N0M0 |
| | | AB-IDC | | | | | | |
| | | SIIA | | | | | | |
| BC | GCI | 15-GC- | UT3SE | UT3SEAQY | | IDC | | |
| | | IDC SIIA | | | | | | |
| BC | GCI | 16-GC- | PVSYX | PVSYXA72 | | IDC | | |
| | | IDC SIIA | | | | | | |
| BC | GCI | 17-GC- | GETCV | GETCVAY2 | | IDC | | |
| | | IDC SIIA | | | | | | |
| BC | ABS | 18-(27)- | 4907020072T | | | IDC | 3 | T2N0Mx |
| | | AB-IDC | | | | | | |
| | | SIIA | | | | | | |
| BC | GCI | 19-GC- | SE5BK | SE5BKAEQ | | IDC | | |
| | | IDC SIIB | | | | | | |
| BC | GCI | 20-GC- | OLKL4 | OLKL4AO6 | | IDC | | |
| | | IDC SIIB | | | | | | |
| BC | GCI | 21-GC- | VK1EJ | VK1EJAQE | | IDC | | |
| | | IDC SIIB | | | | | | |
| BC | GCI | 22-GC- | 3Z5Z4 | 3Z5Z4ANH | | IDC | | |
| | | IDC SIIB | | | | | | |
| BC | ABS | 23-(13)- | A0133T | | | IDC | 2 | T2N1aMx |
| | | AB-IDC | | | | | | |
| | | SIIB | | | | | | |
| BC | GCI | 24-GC- | J5MPN | J5MPNA9Q | | IDC | | |
| | | IDC SIIB | | | | | | |
| BC | GCI | 25-GC- | 54NTA | 54NTAAKT | | IDC | | |
| | | IDC SIIB | | | | | | |
| BC | GCI | 27-GC- | RD3F9 | RD3F9AFQ | | IDC | | |
| | | IDC SIIIA | | | | | | |
| BC | ABS | 28-(17)- | 4904020036T | | | IDC | 2-3 | T3N1Mx |
| | | AB-IDC | | | | | | |
| | | SIIIA | | | | | | |
| BC | ABS | 29-(16)- | 4904020032T | | | IDC | 2 | T3N1Mx |
| | | AB-IDC | | | | | | |
| | | IIIA | | | | | | |
| BC | ABS | 30-(15)- | 7259T | | | IDC | 2 | T3N1M0 |
| | | AB-IDC | | | | | | |
| | | SIIIA | | | | | | |
| BC | GCI | 31-GC- | YOLOF | YOLOFARG | | IDC | | |
| | | IDC SIIIA | | | | | | |
| BC | GCI | 32-GC- | 4W2NY | 4W2NYAC1 | | IDC | | |
| | | IDC SIIIB | | | | | | |
| BC | GCI | 33-GC- | YQ1WW | YQ1WWAUV | | IDC | | |
| | | IDC SIIIB | | | | | | |
| BC | GCI | 34-GC- | KIOE7 | KIOE7AI9 | | IDC | | |
| | | IDC SIIIB | | | | | | |
| BC | Bioch | 70-(43)- | A609183 | | | IDC | 2 | |
| | | Bc-IDC | | | | | | |
| BC | Bioch | 71-(54)- | A605353 | | | IDC | 2 | |
| | | Bc-IDC | | | | | | |
| BC | ABS | 72-(55)- | A609179 | | | IDC | 2 | |
| | | Bc-IDC | | | | | | |
| BC | Bioch | 73-(47)- | A609221 | | | IDC | 2 | |
| | | Bc-IDC | | | | | | |
| BC | Bioch | 74-(48)- | A609222 | | | IDC | 2 | |
| | | Bc-IDC | | | | | | |
| BC | Bioch | 75-(53)- | A605151 | | | IDC | 2 | |
| | | Bc-IDC | | | | | | |
| BC | Bioch | 76-(61)- | A610029 | | | IDC | 2 | |
| | | Bc-IDC | | | | | | |
| BC | Bioch | 77-(46)- | A609177 | | | Carc | 2 | |
| | | BC-Carci | | | | | | |
| BC | Ichilov | 78-(62)- | A609194 | | | IDC | 2 | |
| | | Bc-IDC | | | | | | |
| BC | Amb | 79-(32)- | 7116T | | | MC | | T2N0M0 |
| | | AB-Muc | | | | | | |
| | | Carci | | | | | | |
| | | SIIA | | | | | | |
| BC | GCI | 80-(49)- | A609223 | | | IDC | 2 | |
| | | Bc-IDC | | | | | | |
| BC | GCI | 81-(45)- | A609181 | | | IDC | 2 | |
| | | Bc-IDC | | | | | | |
| BC | GCI | 82-(50)- | A609224 | | | IDC | 2 | |
| | | Bc-IDC | | | | | | |
| BC | Bioch | 83-(44)- | A609198 | | | IDC | 2 | |
| | | Bc-IDC | | | | | | |
| BC | Bioch | 84-(51)- | A605361 | | | IDC | 1 | |
| | | Bc-IDC | | | | | | |
| BC | Amb | 85-(31)- | CG-154 | | | IDC | | |
| | | Ic-IDC | | | | | | |
| BC | Aster | 35-As- | 17090 | 30738 | 30738A1 | ILC | | T1cNXM0 |
| | | ILC SI | | | | | | |
| BC | GCI | 36-GC- | I35US | I35USA9G | | ILC | | |
| | | ILC SIIA | | | | | | |
| BC | GCI | 37-GC- | IS84Y | IS84YAAY | | ILC | | |
| | | ILC SIIB | | | | | | |
| BC | Bioch | 38-(52)- | A605360 | | | ILC | 1 | |
| | | Bc-ILC | | | | | | |
| BB | Aster | 39-As- | 11975 | 15478 | 15478B1 | FIBR | | |
| | | Ben | | | | | | |
| BB | GCI | 40-GC- | ZT15M | ZT15MAMR | | FIBR | | |
| | | Ben | | | | | | |
| BB | GCI | 41-GC- | NNP3Q | NNP3QA4V | | FIBR | | |
| | | Ben | | | | | | |
| BB | GCI | 42-GC- | QK8IY | QK8IYALU | | FIBR | | |
| | | Ben | | | | | | |
| BN | GCI | 43-GC-N | 83LO7 | 83LO7NEH | | NB-PS | | |
| | | PS | | | | | | |
| BN | GCI | 45-GC-N | O6JBJ | O6JBJNT1 | | NB-PS | | |
| | | PS | | | | | | |
| BN | GCI | 46-GC-N | E6UDD | E6UDDNCF | | NB-PS | | |
| | | PS | | | | | | |
| BN | GCI | 47-GC-N | DHLR1 | DHLR1NIQ | | NB-PS | | |
| | | PS | | | | | | |
| BN | GCI | 48-GC-N | JHQEH | JHQEHN4D | | NB-PS | | |
| | | PS | | | | | | |
| BN | Amb | 49-(63)- | 26486 | | | NB-PS | | |
| | | Am-N PS | | | | | | |
| BN | GCI | 50-GC-N | ONBFK | ONBFKNO2 | | NB-PS | | |
| | | PS | | | | | | |
| BN | GCI | 51-GC-N | TG6J6 | TG6J6NNA | | NB-PS | | |
| | | PS | | | | | | |
| BN | Aster | 52-GC-N | 14398 | 20021 | 20021D1 | NB-PS | | |
| | | PS | | | | | | |
| BN | GCI | 54-GC-N | AJGXV | AJGXVNFC | | NB-PS | | |
| | | PS | | | | | | |
| BN | GCI | 56-GC-N | HLCZX | HLCZXNLS | | NB-PS | | |
| | | PS | | | | | | |
| BN | GCI | 58-GC-N | FGV8P | FGV8PNQ6 | | NB-PS | | |
| | | PS | | | | | | |
| BN | Aster | 59-As-N | 9264 | 9486 | 9486A1 | NB-PS | | |
| | | PS | | | | | | |
| BN | Bioch | 60-(57)- | A609233 | A609233 | | NB-PM | | |
| | | Bc-N PM | | | | | | |
| BN | Bioch | 61-(59)- | A607155 | A607155 | | NB-PM | | |
| | | Bc-N PM | | | | | | |
| BN | Bioch | 62-(60)- | A609234 | A609234 | | NB-PM | | |
| | | Bc-N PM | | | | | | |
| BN | Amb | 63-(66)- | 36678 | 36678 | | NB-PM | | |
| | | Am-N | | | | | | |
| | | PM | | | | | | |
| BN | Amb | 64-(64)- | 23036 | 23036 | | NB-PM | | |
| | | Am-N | | | | | | |
| | | PM | | | | | | |
| BN | Amb | 65-(65)- | 31410 | 31410 | | NB-PM | | |
| | | Am-N | | | | | | |
| | | PM | | | | | | |
| BN | Amb | 66-(67)- | 073P010602086A | 073P010602086A | | NB-PM | | |
| | | Am-N | | | | | | |
| | | PM | | | | | | |
| BN | Bioch | 67-(58)- | A609232 | A609232 | | NB-PM | | |
| | | Bc-N PM | | | | | | |
| BN | Aster | 68-As-N | 8862 | 8766 | 8766B1 | NB-PM | | |
| | | PM | | | | | | |
| BN | Aster | 69-As-N | 8457 | 7928 | 7928M1 | NB-PM | | |
| | | PM | | | | | | |

| | | | | | | | | | | | Age | | | |
|---|---|---|---|---|---|---|---|---|---|---|---|---|---|---|
| | | | | | | | | | | # | of | | | Year |
| | C | | | | WT | HT | | | # of | Live | first | Br | Recovery | of |
| Tissue | Stage | Tum % | age | MS | (KG) | (CM) | BMI | Ethnic B | Preg. | Bir | child | child | Type | birth |
| BC_in- | 0 | 100 | 39 | Pre-M | 102 | 157 | 41.4 | CAU | 2 | 1 | | | Surg | |
| situ | | | | | | | | | | | | | | |
| BC | I | 75 | 39 | Pre-M | 48.1 | 147 | 22.2 | WCAU | 1 | 0 | — | — | Surg | 1962 |
| BC | I | | 42 | | | | | | | | | | | |
| BC | I | | 43 | | | | | | | | | | | |
| BC | I | 50 | 50 | Post-M | 113.4 | 175 | 36.9 | WCAU | 2 | 2 | 17 | 0 | Surg | 1951 |
| BC | I | 55 | 56 | Post-M | 57.6 | 168 | 20.5 | WCAU | 0 | — | — | — | Surg | 1945 |
| BC | I | 65 | 57 | Post-M | 76.2 | 165 | 28.0 | WCAU | 2 | 2 | 17 | 0 | Surg | 1944 |
| BC | I | 65 | 60 | Post-M | 68 | 140 | 34.8 | WCAU | 1 | 1 | 23 | 0 | Surg | 1942 |
| BC | I | 90 | 65 | Post-M | 70 | 168 | 24.8 | CAU | 3 | 2 | | | Aut | |
| BC | IIA | | 46 | | | | | | | | | | | |
| BC | IIA | 90 | 46 | Pre-M | 69 | 174 | 22.8 | CAU | 1 | 1 | | | Surg | |
| BC | IIA | 70 | 46 | Pre-M | 76.2 | 165 | 28.0 | WCAU | 1 | 0 | 0 | 0 | Surg | 1956 |
| BC | IIA | | 60 | | | | | | | | | | | |
| BC | IIA | | 60 | | | | | | | | | | | |
| BC | IIA | 80 | 67 | Post-M | 113.4 | 168 | 40.4 | WCAU | 3 | 1 | 34 | 0 | Surg | 1938 |
| BC | IIA | 65 | 70 | Pre-M | 79.4 | 163 | 30.0 | WCAU | 2 | 2 | 20 | 0 | Surg | 1932 |
| BC | IIA | 55 | 70 | Post-M | 72.6 | 163 | 27.5 | WCAU | 2 | 2 | 21 | 0 | Surg | 1931 |
| BC | IIA | | 91 | | | | | | | | | | | |
| BC | IIB | 55 | 41 | Pre-M | 61.2 | 165 | 22.5 | WCAU | 0 | — | — | — | Surg | 1960 |
| BC | IIB | 60 | 46 | Pre-M | 111.1 | 168 | 39.5 | WCAU | 2 | 2 | 24 | 2 | Surg | 1955 |
| BC | IIB | 60 | 54 | Post-M | 72.6 | 168 | 25.8 | WCAU | 0 | — | — | — | Surg | 1947 |
| BC | IIB | 85 | 60 | Post-M | 80 | 163 | 30.3 | WCAU | 1 | 1 | 22 | 0 | Surg | 1942 |
| BC | IIB | | 63 | | | | | | | | | | | |
| BC | IIB | 55 | 64 | Post-M | 68.5 | 157 | 27.6 | WCAU | 4 | 3 | 25 | 3 | Surg | 1938 |
| BC | IIB | 70 | 67 | Post-M | 56.7 | 160 | 22.1 | WCAU | 5 | 5 | 20 | 0 | Surg | 1934 |
| BC | IIIA | 90 | 41 | ? | 63.5 | 157 | 25.6 | WCAU | 1 | 1 | 25 | 0 | Surg | 1962 |
| BC | IIIA | | 42 | | | | | | | | | | | |
| BC | IIIA | | 49 | | | | | | | | | | | |
| BC | IIIA | | 59 | | | | | | | | | | | |
| BC | IIIA | 85 | 62 | Post-M | 82.6 | 160 | 32.2 | WCAU | 3 | 3 | 20 | 0 | Surg | 1943 |
| BC | IIIB | 50 | 39 | Pre-M | 54.4 | 163 | 20.6 | WCAU | 2 | 2 | 26 | 0 | Surg | 1962 |
| BC | IIIB | 60 | 62 | Pre-M | 78 | 163 | 29.5 | WCAU | 0 | — | — | — | Surg | 1940 |
| BC | IIIB | 55 | 65 | Post-M | 73.5 | 157 | 29.6 | WCAU | 2 | 2 | 18 | 2 | Surg | 1939 |
| BC | | | 40 | | | | | | | | | | | |
| BC | | | 41 | | | | | | | | | | | |
| BC | | | 42 | | | | | | | | | | | |
| BC | | | 42 | | | | | | | | | | | |
| BC | | | 44 | | | | | | | | | | | |
| BC | | | 44 | | | | | | | | | | | |
| BC | | | 46 | | | | | | | | | | | |
| BC | | | 48 | | | | | | | | | | | |
| BC | | | 51 | | | | | | | | | | | |
| BC | IIA | | 54 | | | | | | | | | | | |
| BC | | | 54 | | | | | | | | | | | |
| BC | | | 58 | | | | | | | | | | | |
| BC | | | 69 | | | | | | | | | | | |
| BC | | | 77 | | | | | | | | | | | |
| BC | | | 79 | | | | | | | | | | | |
| BC | | | 83 | | | | | | | | | | | |
| BC | I | 100 | 50 | | 94 | 170 | 32.5 | W | 2 | 2 | | | Surg | |
| BC | IIA | 60 | 70 | | 77.1 | 178 | 24.4 | WCAU | — | — | — | — | Surg | 1932 |
| BC | IIB | 65 | 67 | Post-M | 62.6 | 163 | 23.7 | WCAU | 2 | 2 | 16 | 0 | Surg | 1934 |
| BC | | | 60 | | | | | | | | | | | |
| BB | | 100 | 24 | Pre-M | 80 | 164 | 29.7 | CAU | 2 | 0 | | | Surg | |
| BB | | 100 | 34 | | 57.6 | 165 | 21.1 | WCAU | | | | | Surg | 1967 |
| BB | | 95 | 54 | | 56.7 | 157 | 22.9 | WCAU | | | | | Surg | 1948 |
| BB | | 100 | 41 | | 59 | 173 | 19.7 | WCAU | | | | | Surg | 1960 |
| BN | | | 32 | | 78.5 | 155 | 32.7 | WCAU | | | | | Surg | 1969 |
| BN | | | 38 | | 67.1 | 173 | 22.5 | WCAU | | | | | Surg | 1963 |
| BN | | | 40 | | 99.8 | 170 | 34.5 | WCAU | | | | | Surg | 1961 |
| BN | | | 40 | | 90.7 | 168 | 32.3 | WCAU | | | | | Surg | 1961 |
| BN | | | 41 | | 65.3 | 157 | 26.3 | WCAU | | | | | Surg | 1960 |
| BN | | | 43 | | | | | | | | | | | |
| BN | | | 45 | | 81.6 | 165 | 30.0 | WCAU | | | | | Surg | 1956 |
| BN | | | 46 | | 90.7 | 173 | 30.4 | WCAU | | | | | Surg | 1955 |
| BN | | | 49 | Pre-M | 68 | 165 | 25.0 | CAU | 3 | 3 | | | Surg | |
| BN | | | 52 | | 70 | 168 | 24.8 | WCAU | | | | | Surg | 1949 |
| BN | | | 54 | | 67 | 163 | 25.2 | WCAU | | | | | Surg | 1947 |
| BN | | | 61 | | 106.6 | 168 | 37.9 | WCAU | | | | | Surg | 1940 |
| BN | | | 0 | | 0 | 0 | 0.0 | | | | | | | |
| BN | | | 34 | | | | | | | | | | Aut | |
| BN | | | 35 | | | | | | | | | | Aut | |
| BN | | | 36 | | | | | | | | | | Aut | |
| BN | | | 45 | | | | | | | | | | Aut | |
| BN | | | 57 | | | | | | | | | | Aut | |
| BN | | | 63 | | | | | | | | | | Aut | |
| BN | | | 64 | | | | | | | | | | Aut | |
| BN | | | 65 | | | | | | | | | | Aut | |
| BN | | | 74 | | 64 | 157 | 26.0 | CAU | | | | | Aut | |
| BN | | | 87 | | 59 | 165 | 21.7 | CAU | | | | | Aut | |

TABLE 1_3_1

| Key | Full Name |
|---|---|
| # Live Bir | # Live Births |
| # of Preg | Number of Pregnancies |
| Amb | Ambion |
| Aster | Asterand |
| Aut | Autopsy |
| BB | Breast Benign |
| BC | Breast Cancer |
| Bioch | Biochain |
| BN | Breast Normal |
| Breastfeed child | Br Child |
| C Stage | Cancer Stage |
| Carc | Carcinoma |
| CAU | Caucasian |
| DCIS | Ductal Carcinoma In Situ |
| Ethnic B | Ethnic Background |
| FIBR | FIBROADENOMA |
| IDC | INFILTRATING DUCTAL CARCINOMA |
| ILC | INFILTRATING LOBULAR CARCINOMA |
| M | Menopausal |
| MC | Mucinous carcinoma |
| MS | Menopausal Status |
| NB-PM | NORMAL BREAST-PM |
| NB-PS | NORMAL BREAST-PS |
| Post-M | Post-Menopausal |
| Pre-M | Pre-Menopausal |
| Samp DIAG | Sample Diagnosis |
| Surg | Surgical |
| Tum % | Percentage of Tumor |
| W | White |
| WCAU | White Caucasian |

TABLE 1_4
Tissue samples in colon cancer testing panel

| | | | | TISSUE ID | | | | | |
|---|---|---|---|---|---|---|---|---|---|
| | | | sample_id (GCI)/ | (GCI)/ | | | | | |
| | | | case id (Asterand)/ | specimen | | | | | |
| | | sample | lot no. (old | ID | Sample ID | | Diag | Specimen | |
| Tissue | Source/Delivery | name | samples | (Asterand) | (Asterand) | Diag | remarks | location | Gr |
| CC | Asterand | 1-As- | 18036 | 31312 | 31312B1 | Aden | | Cec | 3 |
| | | AdenS0 | | | | | | | |
| CC | GCI | 2-GC- | 4QDH8 | 4QDH8ADT | | Aden | | Dis C | |
| | | AdenoSI | | | | | | | |
| CC | Ichilov | 3-(7)-Ic- | CG-235 | | | AI | | Rectum | UN |
| | | AdenoSI | | | | | | | |
| CC | GCI | 4-GC- | NTAI8 | NTAI8AOU | | Aden | | Cec | |
| | | AdenoSI | | | | | | | |
| CC | GCI | 5-GC- | ARA7P | ARA7PAQA | | Aden | | Ret, Low | |
| | | AdenoSI | | | | | | Ant | |
| CC | Ichilov | 6-(20)-Ic- | CG-249 | | | UA | | | 3 |
| | | AdenoSIIA | | | | | | | |
| CC | GCI | 7-GC- | AFTS6 | AFTS6AP6 | | Aden | | | |
| | | AdenoSIIA | | | | | | | |
| CC | GCI | 8-GC- | 5CYDK | 5CYDKACS | | Aden | | | |
| | | AdenoSIIA | | | | | | | |
| CC | GCI | 9-GC- | XKSLS | XKSLSAF7 | | Aden | | | |
| | | AdenoSIIA | | | | | | | |
| CC | GCI | 10-GC- | B4RU8 | B4RU8A8Q | | Aden | | | |
| | | AdenoSIIA | | | | | | | |
| CC | GCI | 11-GC- | HB8EY | HB8EYA8I | | Aden | | | |
| | | AdenoSIIA | | | | | | | |
| CC | Ichilov | 12-(22)- | CG-229C | | | Aden | | | 2 |
| | | AdenoSII | | | | | | | |
| CC | GCI | 13-GC- | X8C7X | X8C7XATL | | Aden | | | |
| | | AdenoSIIA | | | | | | | |
| CC | GCI | 14-GC- | HCP6K | HCP6KA8Z | | Aden | | | |
| | | AdenoSIIA | | | | | | | |
| CC | Asterand | 16-As- | 17915 | 31176 | 31176A1 | Aden | | | 2-3 |
| | | AdenoSIIA | | | | | | | |
| CC | Ichilov | 17-(1)-Ic- | CG-335 | | | Aden | | Cec | 2 |
| | | AdenoSIIA | | | | | | | |
| CC | Asterand | 19-As- | 12772 | 18885 | 18885A1 | Aden | | rectum | 2 |
| | | AdenoSIIA | | | | | | | |
| CC | GCI | 20-GC- | JFYXP | JFYXPAMP | | Aden | | | |
| | | AdenoSIIA | | | | | | | |
| CC | GCI | 21-GC- | OJXW9 | OJXW9ASR | | Aden | | | |
| | | AdenoSIIA | | | | | | | |
| CC | Ichilov | 22-(28)-Ic- | CG-284 | | | Aden | | sigma | 2 |
| | | AdenoSIIA | | | | | | | |
| CC | Ichilov | 23-(10)-Ic- | CG-311 | | | Aden | | Sig Col | 1-2 |
| | | AdenoSIIA | | | | | | | |
| CC | Ichilov | 24-(14)-Ic- | CG-222 (2) | | | WP | | Rectum | |
| | | AdenoSIII | | | | Aden | | | |
| CC | Ichilov | 25-(23)-Ic- | CG-282 | | | MA | | sigma | UN |
| | | AdenoSIII | | | | | | | |
| CC | GCI | 26-GC- | OTPI7 | OTPI7AWY | | Aden | | | |
| | | AdenoSIII | | | | | | | |
| CC | GCI | 27-GC- | IG9NK | IG9NKAD3 | | MA | | | |
| | | AdenoSIII | | | | | | | |
| CC | GCI | 28-GC- | 53OM7 | 53OM7AGL | | Aden | | | |
| | | AdenoSIII | | | | | | | |
| CC | GCI | 29-GC- | BLUW6 | BLUW6A6Y | | Aden | | | |
| | | AdenoSIII | | | | | | | |
| CC | GCI | 30-GC- | VZ6QA | VZ6QAAFA | | Aden | | RECTUM | |
| | | AdenoSIII | | | | | | | |
| CC | Ichilov | 31-(6)-Ic- | CG-303 (3) | | | Aden | | | 1-2 |
| | | AdenoSIII | | | | | | | |
| CC | Ichilov | 32-(2)-Ic- | CG-307 | | | Aden | | Cecum | 2 |
| | | AdenoSIII | | | | | | | |
| CC | Ichilov | 33-(11)-Ic- | CG-337 | | | Aden | | | 1-2 |
| | | AdenoSIII | | | | | | | |
| CC | Asterand | 34-As- | 18462 | 40971 | 40971A1 | TA | | Sig Col | 2 |
| | | AdenoSIIIC | | | | | | | |
| CC | Ichilov | 35-(13)-Ic- | CG-290 | | | Aden | | Rect Col | 2 |
| | | AdenoSIV | | | | | | | |
| CC | GCI | 36-GC- | 7D7QV | 7D7QVAE6 | | Aden | | | |
| | | AdenoSIV | | | | | | | |
| CC | GCI | 37-GC- | 38U4V | 38U4VAA4 | | Aden | | | |
| | | AdenoSIV | | | | | | | |
| CC | Ichilov | 38-(9)-Ic- | CG-297 | | | Aden | | Rectum | 2 |
| | | AdenoSIV | | | | | | | |
| CC | Ichilov | 71-(16)-Ic- | CG-278C | | | Aden | | | 2 |
| | | Adeno | | | | | | | |
| CC | Ichilov | 72-(4)-Ic- | CG-276 | | | Carc | | | 3 |
| | | Adeno | | | | | | | |
| CC | Ichilov | 73-(17)-Ic- | CG-163 | | | Aden | | Rectum | 2 |
| | | Adeno | | | | | | | |
| CC | Ichilov | 74-(5)-Ic- | CG-308 | | | Aden | | Col Sig | 2 |
| | | Adeno | | | | | | | |
| CC | Ichilov | 75-(72)-Ic- | CG-309 | | | Aden | | | 3 |
| | | Adeno | | | | | | | |
| CC | Ichilov | 76-(18)-Ic- | CG-22C | | | Aden | | | UN |
| | | Adeno | | | | | | | |
| CC | Ichilov | 78-(21)-Ic- | CG-18C | | | Aden | | | UN |
| | | Adeno | | | | | | | |
| CC | Ichilov | 79-(24)-Ic- | CG-12 | | | Aden | | | UN |
| | | Adeno | | | | | | | |
| CC | Ichilov | 80-(25)-Ic- | CG-2 | | | Aden | | | UN |
| | | Adeno | | | | | | | |
| CC | biochain | 82-(61)-Bc- | A606258 | | | Aden, | | | 2 |
| | | Adeno | | | | Ulcer | | | |
| CC | biochain | 83-(57)-Bc- | A609150 | | | Aden | | | 3 |
| | | Adeno | | | | | | | |
| CC | biochain | 84-(56)-Bc- | A609148 | | | Aden | | | 2 |
| | | Adeno | | | | | | | |
| CC | biochain | 85-(53)-Bc- | A609161 | | | Aden | | | 3 |
| | | Adeno | | | | | | | |
| CC | biochain | 86-(54)-Bc- | A609142 | | | Aden | | | 3 |
| | | Adeno | | | | | | | |
| CC | biochain | 87-(59)-Bc- | A609059 | | | Aden, | | | 1 |
| | | Adeno | | | | Ulcer | | | |
| CC | biochain | 88-(60)-Bc- | A609058 | | | Aden, | | | 2 |
| | | Adeno | | | | Ulcer | | | |
| CC | biochain | 89-(55)-Bc- | A609144 | | | Aden | | | 3 |
| | | Adeno | | | | | | | |
| CC | biochain | 90-(58)-Bc- | A609152 | | | Aden | | | 1 |
| | | Adeno | | | | | | | |
| CB | GCI | 40-GC-Ben | IG3OY | IG3OYN7S | | TS | | RT Col | |
| | | | | | | Aden | | | |
| CB | GCI | 41-GC-Ben | GKIEY | GKIEYAV4 | | TS | | Prox T | |
| | | | | | | Aden | | Col | |
| | | | | | | HGD | | | |
| CN | GCI | 42-GC-N | AGVTC | AGVTCNK7 | | NC | DIV | | |
| | | PS | | | | | | | |
| CN | Asterand | 43-As-N PS | 8956 | 9153 | 9153B1 | NC | | | |
| CN | GCI | 44-GC-N | IG3OY | IG3OYN7S | | NC | | RT Col | |
| | | PS | | | | | | | |
| CN | GCI | 45-GC-N | K9OYX | K9OYXN4F | | NC | Divs | LT Col | |
| | | PS | | | | | w/F DIV | | |
| CN | Asterand | 46-As-N PS | 23024 | 74445 | 74445B1 | NC | Chr | | |
| | | | | | | | Divs | | |
| CN | Asterand | 47-As-N PS | 23049 | 71410 | 71410B2 | NC | Chr | | |
| | | | | | | | Divs | | |
| CN | GCI | 48-GC-N | G7JJX | G7JJXAX7 | | NC | Divs | Sig Col | |
| | | PS | | | | | w/ | | |
| | | | | | | | DIV . . . | | |
| CN | Asterand | 49-As-N PS | 22900 | 74446 | 74446B1 | NC | AD | | |
| | | | | | | | w/AF | | |
| CN | GCI | 50-GC-N | XVPZ2 | XVPZ2NDD | | NC | Div | | |
| | | PS | | | | | | | |
| CN | GCI | 51-GC-N | CDSUV | CDSUVNR3 | | NC | CU | | |
| | | PS | | | | | | | |
| CN | GCI | 52-GC-N | GP5KH | GP5KHAOC | | NC | Div | | |
| | | PS | | | | | | | |
| CN | GCI | 53-GC-N | YUZNR | YUZNRNDN | | NC | Divs | Sig Col | |
| | | PS | | | | | | | |
| CN | GCI | 54-GC-N | 28QN6 | 28QN6NI1 | | NC | TS | RT Col | |
| | | PS | | | | | Aden | | |
| CN | GCI | 55-GC-N | GV6N8 | GV6N8NG9 | | NC | Divs, | | |
| | | PS | | | | | PA | | |
| CN | GCI | 56-GC-N | ZJ17R | ZJ17RNIH | | NC | Tub | RT Col | |
| | | PS | | | | | Aden | | |
| CN | GCI | 57-GC-N | 2EEBJ | 2EEBJN2Q | | NC | Div/Chr | | |
| | | PS | | | | | Infl | | |
| CN | GCI | 58-GC-N | 68IX5 | 68IX5N1H | | NC | Chr Div | LT Col | |
| | | PS | | | | | | | |
| CN | GCI | 59-GC-N | 9GEGL | 9GEGLN1V | | NC | Ext Divs | Sig Col | |
| | | PS | | | | | | | |
| CN | GCI | 60-GC-N | PKU8O | PKU8OAJ3 | | NC | Divs, | Sig Col | |
| | | PS | | | | | Chr | | |
| | | | | | | | Div . . . | | |
| CN | Asterand | 61-As-N PS | 22903 | 74452 | 74452B1 | NC | MU | | |
| | | | | | | | w/MI | | |
| CN | Asterand | 62-As-N PS | 16364 | 31802 | 31802B1 | NC | UC | | |
| CN | biochain | 63-(65)-Bc- | A607115 | | | NC- | PM | | |
| | | N PM | | | | PM | | | |
| CN | Ambion | 64-(71)- | 071P10B | | | NC- | PM | | |
| | | Am-N PM | | | | PM | | | |
| CN | biochain | 65-(66)-Bc- | A609262 | | | NC- | PM | | |
| | | N PM | | | | PM | | | |
| CN | biochain | 66-(63)-Bc- | A609260 | | | NC- | PM | | |
| | | N PM | | | | PM | | | |
| CN | biochain | 67-(62)-Bc- | A608273 | | | NC- | PM | | |
| | | N PM | | | | PM | | | |
| CN | biochain | 68-(64)-Bc- | A609261 | | | NC- | PM | | |
| | | N PM | | | | PM | | | |
| CN | biochain | 69-(41)-Bc- | A501156 | | | NC- | PM | | |
| | | N PM | | | | PM | | | |
| CN | biochain | 70-(67)-Bc- | A406029 + A411078 | | | NC- | PM | | |
| | | N PM | | | | PM | | | |
| | | | | | | P10 | | | |

| | | | | | | | | Alcohol | Dr. per | Alc. | Recovery | Exc. |
|---|---|---|---|---|---|---|---|---|---|---|---|---|
| Tissue | TNM | CS | CS2 | Tumor % | Gender | age | Ethnic B | Status | day | Dur. | Type | Y. |
| CC | TXN0M0 | 0 | | 80 | F | 43 | CAU | NU | | | Auto | 2004 |
| CC | | I | Duke A | 85 | F | 44 | WCAU | Y | 4 | | Surg | |
| CC | | I | Duke A | | F | 66 | | | | | | |
| CC | | I | Duke | 80 | M | 53 | WCAU | Y | — | | Surg | |
| | | | B1 | | | | | | | | | |
| CC | | I | Duke | 70 | F | 70 | WCAU | Y | 0 | | Surg | |
| | | | B1 | | | | | | | | | |
| CC | | IIA | Duke | | M | 36 | | | | | | |
| | | | B2 | | | | | | | | | |
| CC | | IIA | Duke | 75 | M | 39 | WCAU | N | 0 | | Surg | |
| | | | B2 | | | | | | | | | |
| CC | | IIA | Duke | 65 | M | 44 | WCAU | N | — | | Surg | |
| | | | B2 | | | | | | | | | |
| CC | | IIA | Duke | 65 | M | 48 | WCAU | Y | 10 | | Surg | |
| | | | B2 | | | | | | | | | |
| CC | | IIA | Duke | 65 | F | 50 | WCAU | N | — | | Surg | |
| | | | B2 | | | | | | | | | |
| CC | | IIA | Duke | 65 | M | 53 | WCAU | N | — | | Surg | |
| | | | B2 | | | | | | | | | |
| CC | | II | Duke B | | F | 55 | | | | | | |
| CC | | IIA | Duke | 90 | M | 56 | WCAU | N | — | | Surg | |
| | | | B2 | | | | | | | | | |
| CC | | IIA | Duke | 80 | M | 58 | WCAU | Y | 4 | | Surg | |
| | | | B2. | | | | | | | | | |
| CC | T3N0M0 | IIA | Duke | 60 | F | 64 | CAU | occ | 1 | 21-30 | Auto | 2004 |
| | | | B2 | | | | | | drink/week | years | | |
| CC | | IIA | Duke | | F | 66 | | | | | | |
| | | | B2 | | | | | | | | | |
| CC | T3NXM0 | IIA | Duke | 60 | F | 67 | CAU | NU | | | Surg | 2004 |
| | | | B2 | | | | | | | | | |
| CC | | IIA | Duke | 60 | F | 68 | WCAU | Y | — | | Surg | |
| | | | B2 | | | | | | | | | |
| CC | | IIA | Duke | 90 | F | 69 | WCAU | N | — | | Surg | |
| | | | B2 | | | | | | | | | |
| CC | | IIA | Duke | | F | 72 | | | | | | |
| | | | B2 | | | | | | | | | |
| CC | | e | Duke | | M | 88 | | | | | | |
| | | IIA | B2 | | | | | | | | | |
| CC | | III | Duke C | | F | 49 | | | | | | |
| CC | | III | Duke C | | M | 51 | | | | | | |
| CC | | III | Duke | 70 | F | 54 | WCAU | N | — | | Surg | |
| | | | C2 | | | | | | | | | |
| CC | | III | Duke | 90 | F | 54 | WCAU | N | — | | Surg | |
| | | | C2 | | | | | | | | | |
| CC | | III | Duke | 75 | F | 61 | WCAU | N | — | | Surg | |
| | | | C2 | | | | | | | | | |
| CC | | III | Duke | 85 | F | 64 | WCAU | N | — | | Surg | |
| | | | C2 | | | | | | | | | |
| CC | | III | Duke | 60 | M | 67 | WCAU | Y | 14 | | Surg | |
| | | | C2 | | | | | | | | | |
| CC | | III | Duke | | F | 77 | | | | | | |
| | | | C2. | | | | | | | | | |
| CC | | III | Duke | | F | 89 | | | — | | | |
| | | | C2. | | | | | | | | | |
| CC | | III | Duke | | NA | NA | | | | | | |
| | | | C2. | | | | | | | | | |
| CC | TXN2M0 | IIIC | | 76 | F | 68 | CAU | NU | | | Surg | 2005 |
| CC | | IV | Duke | | M | 47 | | | | | | |
| | | | D. | | | | | | | | | |
| CC | | IV | Duke D | 80 | F | 52 | WCAU | Y | 3 | | Surg | |
| CC | | IV | Duke D | 85 | F | 53 | WCAU | | — | | Surg | |
| CC | | IV | Duke | | M | 62 | | | | | | |
| | | | D. | | | | | | | | | |
| CC | | | UN | 50 | F | 60 | | | | | | |
| CC | | | UN | 75 | M | 64 | | | | | | |
| CC | | | UN | | M | 73 | | | | | | |
| CC | | | UN | | F | 80 | | | | | | |
| CC | | | UN | | F | 88 | | | | | | |
| CC | | | UN | | NA | NA | | | | | | |
| CC | | | UN | | NA | NA | | | | | | |
| CC | | | UN | | NA | NA | | | | | | |
| CC | | | UN | | NA | NA | | | | | | |
| CC | | | UN | | M | 41 | | | | | | |
| CC | | | UN | | F | 45 | | | | | | |
| CC | | | UN | 40 | F | 48 | | | | | | |
| CC | | | UN | | F | 53 | | | | | | |
| CC | | | UN | | M | 53 | | | | | | |
| CC | | | UN | | M | 58 | | | | | | |
| CC | | | UN | | M | 67 | | | | | | |
| CC | | | UN | | M | 68 | | | | | | |
| CC | | | UN | | M | 73 | | | | | | |
| CB | | | | | F | 48 | WCAU | Y | 1 | | Surg | |
| CB | | | | | F | 75 | WCAU | N | — | | Surg | |
| CN | | | | 0 | M | 45 | WCAU | N | — | | Surg | |
| CN | | | | 0 | F | 46 | CAU | NU | | | Surg | 2002 |
| CN | | | | 0 | F | 48 | WCAU | Y | 1 | | Surg | |
| CN | | | | 0 | F | 50 | WCAU | N | — | | Surg | |
| CN | | | | 0 | F | 52 | CAU | Occ | | | Surg | 2005 |
| CN | | | | 0 | F | 52 | CAU | occ | | | Surg | 2005 |
| CN | | | | 0 | M | 52 | WCAU | N | — | | Surg | |
| CN | | | | 0 | M | 54 | CAU | Cur U | | | Surg | 2005 |
| CN | | | | 0 | F | 55 | WCAU | N | — | | Surg | |
| CN | | | | 0 | M | 55 | WCAU | N | — | | Surg | |
| CN | | | | 0 | F | 57 | WCAU | Y | 6 | | Surg | |
| CN | | | | 0 | F | 57 | WCAU | Y | 1 | | Surg | |
| CN | | | | 0 | M | 59 | WCAU | Y | 42 | | Surg | |
| CN | | | | 0 | F | 61 | WCAU | Y | 3 | | Surg | |
| CN | | | | 0 | M | 61 | WCAU | Y | — | | Surg | |
| CN | | | | 0 | F | 66 | WCAU | Y | 4 | | Surg | |
| CN | | | | 0 | F | 66 | WCAU | N | — | | Surg | |
| CN | | | | 0 | M | 68 | WCAU | N | — | | Surg | |
| CN | | | | 0 | F | 69 | WCAU | N | — | | Surg | |
| CN | | | | 0 | M | 71 | CAU | Occ | | | Surg | 2005 |
| CN | | | | 0 | F | 74 | WCAU | Occ | | | Surg | 2004 |
| CN | | | | | M | 24 | | | | | | |
| CN | | | | | F | 34 | | | | | | |
| CN | | | | | M | 58 | | | | | | |
| CN | | | | | M | 61 | | | | | | |
| CN | | | | | M | 66 | | | | | | |
| CN | | | | | F | 68 | | | | | | |
| CN | | | | | M | 78 | | | | | | |
| CN | | | | | F&M | M | | | | | | |
| | | | | | | (26-78) | | | | | | |
| | | | | | | &F | | | | | | |
| | | | | | | (53-77). | | | | | | |

TABLE 1_4_1

| Key | Full Name |
|---|---|
| CC | Colon Cancer |
| CB | Colon Benign |
| CN | Colon Normal |
| WT | Weight |
| HT | Height |
| Aden | Adenocarcinoma |
| AI | Adenocarcinoma intramucosal |
| UA | Ulcerated adenocarcinoma |
| WP Aden | Well polypoid adeocarcinoma |
| MA | Mucinus adenocarcinoma |
| TA | Tubular adenocarcinoma |
| Carc | Carcinoma |
| TS Aden | TUBULOVILLOUS ADENOMA |
| TS Aden HGD | TUBULOVILLOUS ADENOMA with HIGH |
| | GRADE DYSPLASIA |
| NC | Normal Colon |
| NC-PM | Normal PM |
| NC-PM P10 | Normal PM (Pool 10) |
| Diag | Diagnosis |
| Div | DIVERTICULITIS |
| Divs w/F DIV | Diverticulosis with Focal DIVERTICULITIS |
| Chr Divs | Chronic diverticulosis |
| Divs w/DIV . . . | DIVERTICULOSIS WITH DIVERTICULITIS AND |
| | FOCAL ABSCESS FORMATION; NO |
| | MALIGNANCY |
| AD w/AF | Acute diverticulitis with abscess formation |
| CU | CECAL ULCERATION |
| Divs, PA | DIVERTICULOSIS AND PERICOLIC ABSCESS |
| Tub Aden | TUBULAR ADENOMA |
| Div/Chr Infl | DIVERTICULOSIS/CHRONIC INFLAMMATION |
| Chr Div | CHRONIC DIVERTICULITIS |
| Ext Divs | EXTENSIVE DIVERTICULOSIS |
| Divs, Chr Div . . . | DIVERTICULOSIS AND CHRONIC |
| | DIVERTICULITIS, SEROSAL FIBROSIS AND |
| | CHRONIC SEROSITIS |
| MU w/MI | Mucosal ulceration with mural inflammation |
| UC | Ulcerative colitis |
| Cec | cecum |
| Dis C | DISTAL COLON |
| Ret, Low Ant | RETROSIGMOID, LOW ANTERIOR |
| Rect Col | Rectosigmoidal colon |
| Sig col | Sigmod colon |
| Col Sig | Colon Sigma |
| RT Col | RIGHT COLON |
| Prox T Col | PROXIMAL TRANSVERSE COLON |
| LT Col | Left Colon |
| Gr | Grade |
| CS | Cancer Stage |
| Ethnic B | Ethnic background |
| NU | Never Used |
| Occ | Occasion |
| Cur U | Current use |
| Dr. per day | Drinks per day |
| Alc. Dur. | Alcohol Duration |
| Auto. | Autopsy |
| Surg. | Surgical |
| Exc. Y. | Excision Year |

TABLE 1_5
Tissue samples in normal panel:

| | | Sample | | |
|---|---|---|---|---|
| | | id(GCI)/case | | |
| | | id | Tissue id | Sample id |
| | | (Asterand) | (GCI)/Specimen | (Asterand)/RNA |
| sample name | Source | Lot no. | id (Asternd) | id (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowl | GCI | V9L7D | V9L7DN6Z | |
| 7-GC-Small bowl | GCI | M3GVT | M3GVTN5R | |
| 8-GC-Small bowl | GCI | 196S2 | 196S2AJN | |
| 9-(9)-Am-Stomach | Ambion | 110P04A | | |
| 10-(10)-Bc-Stomach | Biochain | A501159 | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 15-(48)-Ic-Liver | Ichilov | CG-93-3 | | |
| 16-As-Liver | Asterand | 7916 | 7203 | 7203B1 |
| 17-(28)-Am-Bladder | Ambion | 071P02C | | |
| 18-(29)-Bc-Bladder | Biochain | A504088 | | |
| 19-(64)-Am-Kidney | Ambion | 111P0101B | | |
| 20-(65)-Cl-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 24-(42)-Ic-Adrenal | Ichilov | CG-184-10 | | |
| 25-(43)-Bc-Adrenal | Biochain | A610374 | | |
| 26-(16)-Am-Lung | Ambion | 111P0103A | | |
| 27-(17)-Bc-Lung | Biochain | A503204 | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 35-(21)-Am-Cerix | Ambion | 101P0101A | | |
| 36-GC-cervix | GCI | E2P2N | E2P2NAP4 | |
| 37-(24)-Bc-Uterus | Biochain | A411074 | | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am-Prostate | Ambion | 25955 | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 48-GC-Artery | GCI | 7FUUP | 7FUUPAMP | |
| 49-GC-Artery | GCI | YGTVY | YGTVYAIN | |
| 50-Th-Blood-PBMC | Tel- | 52497 | | |
| | Hashomer | | | |
| 51-Th-Blood-PBMC | Tel- | 31055 | | |
| | Hashomer | | | |
| 52-Th-Blood-PBMC | Tel- | 31058 | | |
| | Hashomer | | | |
| 53-(54)-Ic-Spleen | Ichilov | CG-267 | | |
| 54-(55)-Am-Spleen | Ambion | 111P0106B | | |
| 55-(57)-Ic-Thymus | Ichilov | CG-98-7 | | |
| 56-(58)-Am-Thymus | Ambion | 101P0101A | | |
| 57-(60)-Bc-Thyroid | Biochain | A610287 | | |
| 58-(62)-Ic-Thyroid | Ichilov | CG-119-2 | | |
| 59-Gc-Sali gland | GCI | NNSMV | NNSMVNJC | |
| 60-(67)-Ic-Cerebellum | Ichilov | CG-183-5 | | |
| 61-(68)-Ic-Cerebellum | Ichilov | CG-212-5 | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

### Materials and Experimental Procedures

RNA preparation—RNA was obtained from ABS (Wilmington, Del. 19801, USA, absbioreagents.com), BioChain Inst. Inc. (Hayward, Calif. 94545 USA biochain.com), GOG for ovary samples—Pediatric Cooperative Human Tissue Network, Gynecologic Oncology Group Tissue Bank, Children Hospital of Columbus (Columbus Ohio 43205 USA), Clontech (Franklin Lakes, N.J. USA 07417, clontech.com), Ambion (Austin, Tex. 78744 USA, ambion.com), Asternad (Detroit, Mich. 48202-3420, USA, asterand.com), and from Genomics Collaborative Inc., a Division of Seracare (Cambridge, Mass. 02139, USA, .genomicsinc.com). Alternatively, RNA was generated from tissue samples using TR1—Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

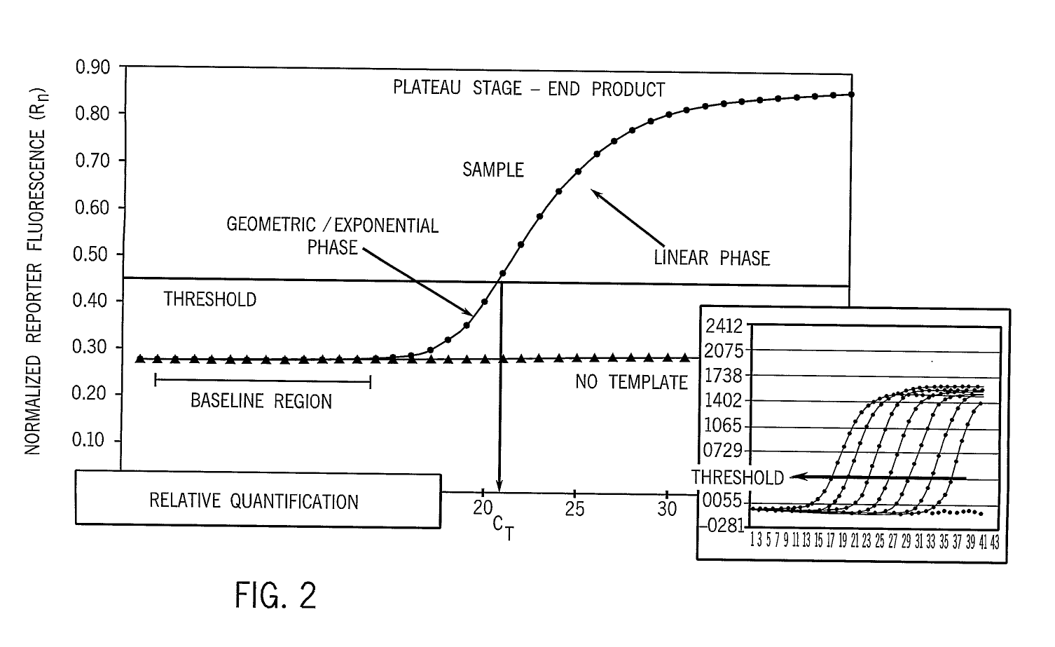

Real-Time RT-PCR analysis—cDNA (50), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. Non-detected samples were assigned Ct value of 41 and were calculated accordingly. The relative quantity was calculated using the equation Q=efficiencŷ^{-Ct}. The efficiency of the PCR reaction was calculated from a standard curve, created by using serial dilutions of several reverse transcription (RT) reactions. To minimize inherent differences in the RT reaction, the resulting relative quantities were normalized to normalization factor calculated as follows: the expression of several housekeeping (HSKP) genes was checked on every panel. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 2. As shown, the x-axis shows the cycle number. The C_{T}=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

Real-Time RT-PCR analysis using TaqMan® probes—cDNA (50), prepared as described above, was used as a template in Real-Time PCR reactions using the TaqMan Universal PCR Master mix (PE Applied Biosystem) with specific primers and specific TaqMan® MGB probes. The primers were used at a concentration of 500 nM and the probes at a concentration of 200 nM. The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=2̂−Ct. To minimize inherent differences in the RT reaction, the resulting relative quantities were normalized using normalization factor calculated as follows: The expression of several housekeeping (HSKP) genes was checked on the RT panel by qRT-PCR using SYBR Green detection. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 2. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples on colon cancer tissue testing panel were as follows:

PBGD (GenBank Accession No. BC019323 (SEQ ID NO:
1)),
PBGD Forward primer (SEQ ID NO: 2):
TGAGAGTGATTCGCGTGGG
PBGD Reverse primer (SEQ ID NO: 3):
CCAGGGTACGAGGCTTTCAAT
PBGD-amplicon (SEQ ID NO: 4):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194 (SEQ ID NO:
5)),
HPRT1 Forward primer (SEQ ID NO: 6):
TGACACTGGCAAAACAATGCA
HPRT1 Reverse primer (SEQ ID NO: 7):
GGTCCTTTTCACCAGCAAGCT
HPRT1-amplicon (SEQ ID NO: 8):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
G6PD (GenBank Accession No. NM_000402 (SEQ ID NO:
13))
G6PD Forward primer (SEQ ID NO: 14):
gaggccgtcaccaagaacat
G6PD Reverse primer (SEQ ID NO: 15):
ggacagccggtcagagctc
G6PD-amplicon (SEQ ID NO: 151):
gaggccgtcaccaagaacattcacgagtcctgcatgagccagataggctg
gaaccgcatcatcgtggagaagcccttcgggagggacctgcagagctctg
accggctgtcc
RPS27A (GenBank Accession No. NM_002954 (SEQ ID
NO: 16))
RPS27A Forward primer (SEQ ID NO: 17):
CTGGCAAGCAGCTGGAAGAT
Reverse primer (SEQ ID NO: 18):
TTTCTTAGCACCACCACGAAGTC
RPS27A-amplicon (SEQ ID NO: 152):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples on normal tissue samples panel were as follows:

RPL19 (GenBank Accession No. NM_000981 (SEQ ID NO:
19))
RPL19Forwar primer (SEQ ID NO: 20):
TGGCAAGAAGAAGGTCTGGTTAG
RPL19Reverse primer (SEQ ID NO: 21):
TGATCAGCCCATCTTTGATGAG
RPL19-amplicon (SEQ ID NO: 22):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A
TATA box (GenBank Accession No. NM_003194 (SEQ ID
NO: 23)),
TATA box Forward primer (SEQ ID NO: 24):
CGGTTTGCTGCGGTAATCAT
TATA box Reverse primer (SEQ ID NO: 25):
TTTCTTGCTGCCAGTCTGGAC
TATA box-amplicon (SEQ ID NO: 26):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA
Ubiquitin (GenBank Accession No. BC000449 (SEQ ID
NO: 27))
Ubiquitn Forward primer (SEQ ID NO: 28):
ATTTGGGTCGCGGTTCTTG
Ubiquitin Reverse primer (SEQ ID NO: 29):
TGCCTTGACATTCTCGATGGT
Ubiquitin-amplicon (SEQ ID NO: 30)
ATTTGGGTCGCGGTTCTTGTTTGTGGATCGCTGTGATCGTCACTTGACAA
TGCAGATCTTCGTGAAGACTCTGACTGGTAAGACCATCACCCTCGAGG
TTGAGCCCAGTGACACCATCGAGAATGTCAAGGCA
SDHA (GenBank Accession No. NM_004168 (SEQ ID NO:
31))
SDHA Forward primer (SEQ ID NO: 32):
TGGGAACAAGAGGGCATCTG
SDHA Reverse primer (SEQ ID NO: 33):
CCACCACTGCATCAAATTCATG
SDHA-amplicon (SEQ ID NO: 34):
TGGGAACAAGAGGGCATCTGCTAAAGTTTCAGATTCCATTTCTGCTCAGT
ATCCAGTAGTGGATCATGAATTTGATGCAGTGGTGG

### Actual Marker Examples

The following examples relate to specific actual marker examples. It should be noted that Table numbering is restarted within each example related to a particular Cluster, as indicated by the titles below.

### Description for Cluster D11717

Cluster D11717 (internal ID 71276133) features 7 transcript(s) and 9 segment(s) of interest, the names for which are given in Tables 2 and 3. The selected protein variants are given in table 4.

TABLE 2
Transcripts of interest
Transcript Name

D11717_T0 (SEQ ID NO: 35)
D11717_T2 (SEQ ID NO: 36)
D11717_T3 (SEQ ID NO: 37)
D11717_T4 (SEQ ID NO: 38)
D11717T14 (SEQ ID NO: 39)
D11717_T17 (SEQ ID NO: 40)
D11717_T21 (SEQ ID NO: 41)

TABLE 3
Segments of interest
Segment Name

D11717_N0 (SEQ ID NO: 42)
D11717_N13 (SEQ ID NO: 43)
D11717_N14 (SEQ ID NO: 44)
D11717_N15 (SEQ ID NO: 45)
D11717_N1 (SEQ ID NO: 46)
D11717_N3 (SEQ ID NO: 47)
D11717_N4 (SEQ ID NO: 48)
D11717_N5 (SEQ ID NO: 49)
D11717_N6 (SEQ ID NO: 50)

TABLE 4
Proteins of interest

| Protein Name | Corresponding Transcript(s) |
|---|---|
| D11717_P4 (SEQ ID NO: 55) | D11717_T4 (SEQ ID NO: 38) |
| D11717_P18 (SEQ ID NO: 56) | D11717_T14 (SEQ ID NO: 39); |
| | D11717_T17 (SEQ ID NO: 40); |
| | D11717_T21 (SEQ ID NO: 41) |

These sequences are variants of the known protein Growth/differentiation factor 15 precursor (SEQ ID NO:51) (SwissProt accession identifier GDF15_HUMAN (SEQ ID NO: 51); known also according to the synonyms GDF-15; Placental bone morphogenic protein; Placental TGF-beta; Macrophage inhibitory cytokine 1; MIC-1; Prostate differentiation factor; NSAID-regulated protein 1; NRG-1), referred to herein as the previously known protein. Known polymorphisms for this sequence are as shown in Table 5.

TABLE 5
Amino acid mutations for Known Protein

| SNP position(s) on amino | |
|---|---|
| acid sequence | Comment |
| 9 | L -> V |
| 269 | V -> E |
| 288 | T -> A |
| 48 | T -> S (in dbSNP: 1059369). /FTId = |
| | VAR_010386 |
| 202 | H -> D |

Protein Growth/differentiation factor 15 precursor (SEQ ID NO:51) localization is believed to be Secreted protein (Probable).

Growth differentiation factor 15 (GDF15), a 308 amino acid protein, is a member of the bone morphogenetic protein (BMP) family and the TGF-beta superfamily, is important in regulating inflammation. It has been implicated in a variety of functions directly related to tumorigenicity including antiproliferative and pro-apoptotic effects through its involvement in inflammation. Specifically, inflammation of the prostate has been suggested to favor tumor development. BMP proteins are secreted growth factors that are characterized by seven conserved cysteine residues. In general, they are regulators of cell growth and differentiation in both embryonic and adult tissues. GDF15 is an important downstream mediator of DNA damage signaling and a transcriptional target of p53.

GDF-15 is highly expressed in placenta, with lower levels in prostate and colon and some expression in kidney. mRNA is most abundant in the liver, with lower levels seen in some other tissues. GDF-15 induction after organ injury is a hallmark of many tissues. Its expression in liver can be significantly up-regulated during injury of organs such as liver, kidney, heart and lung. It may therefore serve as an early mediator of the injury response and might regulate inflammation, cell survival, proliferation, and apoptosis in a variety of injured tissues and disease processes.

It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Unidentified pharmacological activity. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anticancer, other.

The following GO Annotation(s) apply to the previously known protein. The following annotation(s) were found: transforming growth factor beta receptor signaling pathway; signal transduction; cell-cell signaling, which are annotation(s) related to Biological Process; cytokine activity, which are annotation(s) related to Molecular Function; and extracellular region, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As indicated below, table 6, this protein might have the potential to serve as a diagnostic marker for various types of inflammatory conditions, cancers and cardiovascular diseases.

TABLE 6
Diagnostic utility for Cluster D11717

| Diagnostic | | |
|---|---|---|
| Utility | Reason | reference |
| Diagnosis of | 1) MIC-1 was overexpressed in local and metastatic PC whereas | Clin Cancer Res. |
| Prostate Cancer | peritumoral interstitial staing for MIC-1 identified lower grade tumor | 2006 Jan; 1: 12(1): |
| (PC) | destined for recurrence. It was also found to be an independent predictor | 89-96. |
| | of the presence of PC with a Gleason sum ≧ 7. Also, it showed an | |
| | improved specificity for diagnostic blood testing for PC over percentage | |
| | of free PSA antigen, potentially reducing unnecessary biopsies by 27%. | |
| | 2) Neuropeptide Y (NPY) & MIC-1 immunostaining was higher in low- | Cancer Epidemiol |
| | grade intraepithelial neoplasia (PIN) compared with other benign tissues | Biomarkers Prev. |
| | (both P < 0.0001) and was equivalent to immunostaining in high grade PIN. | 2006 |
| | MIC-1 immunostaining (20% categories) was associated with pathologic | Apr: 15(4): 711-6. |
| | stage >2C after adjusting for predictors of pathologic stage. | |
| | 3) MIC-1 gene overexpression in cancerous tissues was observed in 88% | Br. J. Cancer. 2003 |
| | of cases, compared to noncancerous tissue (P < 0.001). The expression | Apr; 7: 88(7): 1101-4. |
| | level of MIC-1 in cancerous tissue was significantly higher than in | |
| | noncancerous tissue (P < 0.001), especially in more aggressive forms of the | |
| | disease (Gleason score > 5. | |
| Diagnosis of | A comparison of MIC-1 to other biomarkers, in relation to PC bone | Cancer Epidemiol |
| bone metastases | metastases, revealed that | Biomarkers Prev. |
| in patients with | it was the best predictor for the presence of baseline bone metastases. It | 2007 |
| PC | was significantly better than carboxy terminal telopeptide of type I | Mar: 16(3): 532-7. |
| | collagen, PSA & amino-terminal propeptide of type I procollagen (PINP). | |
| | Patients who experienced bone relapse had significantly higher levels of | |
| | baseline MIC-1 compared with patients who did not (P = 0.003). | |
| Biomarker of the risk | When GDF-15 levels were determined by immunoradiometric | Circulation. 2007 |
| for death in patients | assay, about ⅔ of patients (acute chest pain & either ST-segment | Feb 27; 115(8): 962-71. |
| with non-ST-elevation | depression or troponin elevation) had GDF-15 levels > the upper | |
| acute coronary | limit of normal in healthy controls (1200 ng/L). ⅓ presented with | |
| syndrome. | levels >1800 ng/L. Increased GDF-15 levels have been associated | |
| | with increased risk for death at 1 year. By multiple Cox | |
| | regression analysis, only the levels of GDF-15 & N-terminal pro- | |
| | B-type natriuretic acid peptide contributed independently to 1-year | |
| | mortality risk. Roc analysis further illustrated that GDF-15 is a | |
| | strong marker of 1-year mortality risk (area under curve, 0.757: | |
| | best cutoff, 1808 ng/L). At this cutoff value, GDF-15 added | |
| | significant prognostic information in patient subgroups defined by | |
| | age, gender, time from symptoms to admission, cardiovascular | |
| | risk factors, previous cardiovascular disease, and the risk markers: | |
| | ST-segment depression, troponin T, N-terminal pro-B-type | |
| | natriuretic acid, C reactive protein & creatinin clearance. | |
| Diagnosis of pancreatic | A comparison of the diagnostic utility of a panel of candidate | Clin Cancer Res. |
| cancer. | serum markers of pancreatic cancer (CA19-9, MIC-1, osteopontin, | 2006 Jan 15; 12(2): |
| | tissue inhibitor of metalloproteinase 1, hepatocarcinoma-intestine- | 442-6. |
| | pancreas protein level) revealed that by logistic regression that | |
| | MIC-1 and CA19-9 were significant independent predictors of | |
| | diagnosis. Roc analysis revealed that MIC-1 was significantly | |
| | better than CA19-9 in differentiating patients with pancreatic | |
| | cancer from healthy controls (area under the curve was 0.99 and | |
| | 0.78 respectively, P = 0.003). | |
| Prediction of a | MIC-1 concentrations in pregnant women who subsequently | Lancet 2004 Jan 10: |
| miscarriage | miscarried, were a third of those who had ongoing pregnancies. | 363(9403): 129-30. |
| | Multiples of the median for miscarriage was 0.32 versus 1.00 for | |
| | ongoing pregnancies; P < 0.0001. Concentrations were just as low | |
| | 3 weeks before diagnosis as on the day of diagnosis. | |
| Determination of | MIC-1 was expressed in CRC tissue and the cancer cell line | Clin Cancer Res. |
| progress and prognosis | CaCo2. There was a progressive increase in serum MIC-1 levels | 2003 Jul; 9(7): 2642-50. |
| for colorectal | between normal individuals (mean: 495 ± 210), those with | |
| carcinoma (CRC) | adenomatous polys (681 ± 410) and those with CRC (783 ± 491). | |
| | Serum MIC-1 level was correlated with the extent of disease so | |
| | that the levels were higher in patients with higher Tumor-Node- | |
| | Metastasis stage. There were significant differences in time to | |
| | relapse and overall survival between subjects with different MIC- | |
| | 1 levels and genotypes. | |
| Risk assessment for | MIC-1 serum levels were higher at baseline in women who later | Lancer. 2002 June |
| cardiovascular event in | had cardiovascular events. Levels >90th percentile were | 22: |
| women | associated with a 2.7x greater risk and independent of traditional | 359(9324): 2159-63. |
| | cardiovascular risk factors and at least additive to that of CRP | |

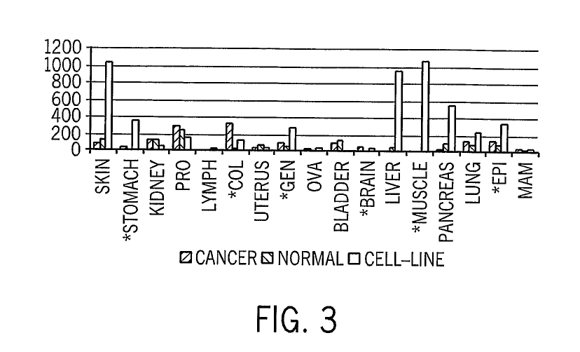

Cluster D11717 can be used as a diagnostic marker according to overexpression of transcripts of this cluster in cancer. Expression of such transcripts in normal tissues is also given according to the previously described methods. The term “number” in the right hand column of the table and the numbers on the y-axis of FIG. 3 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 3 and Table 7. This cluster is overexpressed (at least at a minimum level) in the following pathological conditions colorectal cancer, a mixture of malignant tumors from different tissues and epithelial malignant tumors.

TABLE 7
Normal tissue distribution

| Name of Tissue | Number |
|---|---|
| skin | 127 |
| stomach | 0 |
| kidney | 118 |
| prostate | 250 |
| lymph nodes | 0 |
| colon | 12 |
| uterus | 53 |
| general | 51 |
| ovary | 0 |
| bladder | 123 |
| brain | 2 |
| liver | 48 |
| muscle | 0 |
| pancreas | 84 |
| lung | 77 |
| epithelial | 79 |
| breast | 17 |

As noted above, cluster D11717 features 7 transcript(s), which were listed in Table 2 above. These transcript(s) encode for protein(s) which are variant(s) of protein Growth/differentiation factor 15 precursor (SEQ ID NO:51). A description of each variant protein according to the present invention is now provided.

Variant protein D11717_P4 (SEQ ID NO:55) according to the present invention is encoded by transcript(s) D11717_T4 (SEQ ID NO:38). An alignment is given to the known protein (Growth/differentiation factor 15 precursor (SEQ ID NO:51)) at the end of the application. One or more alignments to one or more previously published protein sequences are given at the end of the application. A brief description of the relationship of the variant protein according to the present invention to each such aligned protein is as follows:

### 1. Comparison Report Between D11717_P4 (SEQ ID NO:55) and Known Protein(s) GDF15_HUMAN_V1 (SEQ ID NO:54):

A. An isolated chimeric polypeptide comprising D11717_P4 (SEQ ID NO:55), comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), and a second amino acid sequence being at least 90% homologous to QMLLVLLVLSWLPHGGALSLAEASRASFPGPSELHSEDSRFRELRKRYEDLLTRLRANQSWEDSNTDLVPA PAVRILTPEVRLGSGGHLHLRISRAALPEGLPEASRLHRALFRLSPTASRSWDVTRPLRRQLSLARPQAPALH LRLSPPPSQSDQLLAESSSARPQLELHLRPQAARGRRRARARNGDHCPLGPGRCCRLHTVRASLEDLGWAD WVLSPREVQVTMCIGACPSQFRAANMHAQIKTSLHRLKPDTVPAPCCVPASYNPMVLIQKTDTGVSLQTY DDLLAKDCHCI corresponding to amino acids 13-308 of known protein(s) GDF15_HUMAN_V1 (SEQ ID NO:54), which also corresponds to amino acids 36-331 of D11717_P4 (SEQ ID NO:55), wherein said first amino acid sequence and second amino acid sequence are contiguous and in a sequential order.

B. An isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

It should be noted that the known protein(s) sequence (GDF15_HUMAN (SEQ ID NO: 51)) has one or more changes than the sequence given in SEQ ID NO:51, and named as being the amino acid sequence for GDF15_HUMAN_V1 (SEQ ID NO:54). These changes were previously known to occur and are listed in table 8 below.

TABLE 8
Changes to GDF15_HUMAN_V1 (SEQ ID NO: 54)

| SNP position on amino | |
|---|---|
| acid sequence | Type of change |
| 48 | variant |

### 2. Comparison Report Between D11717_P4 (SEQ ID NO:55) and Known Protein(s) Q9BWA0_HUMAN (SEQ ID NO: 53):

A. An isolated chimeric polypeptide comprising D11717_P4 (SEQ ID NO:55), comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), and a second amino acid sequence being at least 90% homologous to QMLLVLLVLSWLPHGGALSLAEASRASFPGPSELHSEDSRFRELRKRYEDLLTRLRANQSWEDSNTDLVPA PAVRILTPEVRLGSGGHLHLRISRAALPEGLPEASRLHRALFRLSPTASRSWDVTRPLRRQLSLARPQAPALH LRLSPPPSQSDQLLAESSSARPQLELHLRPQAARGRRRARARNGDHCPLGPGRCCRLHTVRASLEDLGWAD WVLSPREVQVTMCIGACPSQFRAANMHAQIKTSLHRLKPDTVPAPCCVPASYNPMVLIQKTDTGVSLQTY DDLLAKDCHCI corresponding to amino acids 13-308 of known protein(s) Q9BWA0_HUMAN (SEQ ID NO: 53), which also corresponds to amino acids 36-331 of D11717_P4 (SEQ ID NO:55), wherein said first amino acid sequence and second amino acid sequence are contiguous and in a sequential order.

B. An isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

3. Comparison Report Between D11717_P4 (SEQ ID NO:55) and known protein(s) NP_{—}004855 (SEQ ID NO: 52):

A. An isolated chimeric polypeptide comprising D11717_P4 (SEQ ID NO:55), comprising a first amino acid sequence being at least 70%, optionally at least 80%, preferably at least 85%, more preferably at least 90% and most preferably at least 95%, homologous to a polypeptide having the sequence MTPGPRSCRNATRTFRAPVRQGEPGGAGPQPHPKA (SEQ ID NO: 145) corresponding to amino acids 1-35 of D11717_P4 (SEQ ID NO:55), a second amino acid sequence being at least 90% homologous to QMLLVLLVLSWLPHGGALSLAEASRASFPGPSELHSEDSRFRELRKRYEDLLTRLRANQSWEDSNTDLVPA PAVRILTPEVRLGSGGHLHLRISRAALPEGLPEASRLHRALFRLSPTASRSWDVTRPLRRQLSLARPQAPALH LRLSPPPSQSDQLLAESSSARPQLELHLRPQAARGRRRARARNGD corresponding to amino acids 13-201 of known protein(s) NP_{—}004855 (SEQ ID NO: 52), which also corresponds to amino acids 36-224 of D11717_P4 (SEQ ID NO:55), a bridging amino acid H corresponding to amino acid 225 of D11717_P4 (SEQ ID NO:55), a third amino acid sequence being at least 90% homologous to CPLGPGRCCRLHTVRASLEDLGWADWVLSPREVQVTMCIGACPSQFRAANMHAQIKTSLHRLKPDT corresponding to amino acids 203-268 of known protein(s) NP_{—}004855 (SEQ ID NO: 52), which also corresponds to amino acids 226-291 of D11717_P4 (SEQ ID NO:55), a bridging amino acid V corresponding to amino acid 292 of D11717_P4 (SEQ ID NO:55), and a fourth amino acid sequence being at least 90% homologous to PAPCCVPASYNPMVLIQKTDTGVSLQTYDDLLAKDCHCI corresponding to amino acids 270-308 of known protein(s) NP_{—}004855 (SEQ ID NO: 52), which also corresponds to amino acids 293-331 of D11717_P4 (SEQ ID NO:55), wherein said first amino acid sequence, second amino acid sequence, bridging amino acid, third amino acid sequence, bridging amino acid and fourth amino acid sequence are contiguous and in a sequential order.

B. An isolated polypeptide comprising a head of D11717_P4 (SEQ ID NO:55), comprising a polypeptide being at least 70%, optionally at least about 80%, preferably at least about 85%, more preferably at least about 90% and most preferably at least about 95% homologous to the sequence MTPGPRSCRNATRTFRAPVRQGEPGGAGPQPHPKA (SEQ ID NO: 145) of D11717_P4 (SEQ ID NO:55).

The localization of the variant protein was determined according to results from a number of different software programs and analyses, including analyses from SignalP and other specialized programs. The variant protein is believed to be located as follows with regard to the cell: secreted.

Variant protein D11717_P4 (SEQ ID NO:55) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein D11717_P4 (SEQ ID NO:55) sequence provides support for the deduced sequence of this variant protein according to the present invention).

TABLE 9
Amino acid mutations

| SNP position(s) on amino acid | |
|---|---|
| sequence | Alternative amino acid(s) |
| 69 | L -> * |
| 69 | L -> W |
| 70 | H -> Q |
| 134 | P -> L |
| 163 | P -> L |
| 163 | P -> R |
| 202 | Q -> |
| 224 | D -> E |
| 239 | V -> G |
| 249 | A -> |
| 254 | S -> A |
| 254 | S -> P |
| 268 | P -> |
| 271 | F -> L |
| 295 | P -> T |
| 297 | C -> |

The glycosylation sites of variant protein D11717_P4 (SEQ ID NO:55), as compared to the known protein Growth/differentiation factor 15 precursor (SEQ ID NO:51), are described in Table 10 (given according to their position(s) on the amino acid sequence in the first column; the second column indicates whether the glycosylation site is present in the variant protein; and the last column indicates whether the position is different on the variant protein).

TABLE 10
Glycosylation site(s)

| Position(s) on known amino acid | Present in | Position(s) on |
|---|---|---|
| sequence | variant protein? | variant protein |
| 105 | Yes | 128 |

Variant protein D11717_P4 (SEQ ID NO:55) is encoded by the following transcript(s): D11717_T4 (SEQ ID NO:38).The coding portion of transcript D11717_T4 (SEQ ID NO:38) starts at position 159 and ends at position 1151. The transcript also has the following SNPs as listed in Table 11 (given according to their position on the nucleotide sequence, with the alternative nucleic acid listed; the last column indicates whether the SNP is known or not; the presence of known SNPs in variant protein D11717_P4 (SEQ ID NO:55) sequence provides support for the deduced sequence of this variant protein according to the present invention).

TABLE 11
Nucleic acid SNPs

| Polymorphism | SNP position(s) on nucleotide sequence |
|---|---|
| C -> | 96, 762, 905, 961 |
| T -> A | 364 |
| T -> G | 364, 874, 918 |
| C -> A | 368, 830, 1041 |
| C -> T | 368, 417, 559, 560, 646 |
| A -> T | 389 |
| A -> G | 389, 1192 |
| C -> G | 646, 830, 905, 971 |
| G -> T | 647, 1266 |
| -> A | 663, 823, 1019, 1026, 1056 |
| G -> A | 698, 797, 1185, 1188 |
| G -> C | 698, 1185, 1223, 1224, 1266 |
| -> T | 823, 1169 |
| T -> C | 918, 1267 |
| -> G | 1019 |
| G -> | 1048 |
| -> C | 1056, 1159, 1169 |
| A -> C | 1192 |

Variant protein D11717_P18 (SEQ ID NO:56) according to the present invention is encoded by transcript(s) D11717_T14 (SEQ ID NO:39), D11717_T17 (SEQ ID NO:40) and D11717_T21 (SEQ ID NO:41).

The localization of the variant protein was determined according to results from a number of different software programs and analyses, including analyses from SignalP and other specialized programs. The variant protein is believed to be located as follows with regard to the cell: secreted.

Variant protein D11717_P18 (SEQ ID NO:56) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein D11717_P18 (SEQ ID NO:56) sequence provides support for the deduced sequence of this variant protein according to the present invention).

TABLE 12
Amino acid mutations

| SNP position(s) on amino acid | |
|---|---|
| sequence | Alternative amino acid(s) |
| 9 | V -> L |
| 46 | L -> * |
| 46 | L -> W |
| 47 | H -> Q |

The glycosylation sites of variant protein D11717_P18 (SEQ ID NO:56), as compared to the known protein Growth/differentiation factor 15 precursor (SEQ ID NO:51), are described in Table 13 (given according to their position(s) on the amino acid sequence in the first column; the second column indicates whether the glycosylation site is present in the variant protein; and the last column indicates whether the position is different on the variant protein).

TABLE 13
Glycosylation site(s)

| Position(s) on known amino acid | Present | Position(s) |
|---|---|---|
| sequence | in variant protein? | on variant protein |
| 70 | Yes | 70 |

Variant protein D11717_P18 (SEQ ID NO:56) is encoded by the following transcript(s): D11717_T14 (SEQ ID NO:39), D11717_T17 (SEQ ID NO:40) and D11717_T21 (SEQ ID NO:41).

The coding portion of transcript D11717_T14 (SEQ ID NO:39) starts at position 552 and ends at position 833. The transcript also has the following SNPs as listed in Table 14 (given according to their position on the nucleotide sequence, with the alternative nucleic acid listed; the last column indicates whether the SNP is known or not; the presence of known SNPs in variant protein D11717_P18 (SEQ ID NO:56) sequence provides support for the deduced sequence of this variant protein according to the present invention).

TABLE 14
Nucleic acid SNPs

| Polymorphism | SNP position(s) on nucleotide sequence |
|---|---|
| C -> | 108, 2514, 2657, 2713 |
| C -> T | 400, 541, 692, 741, 2311, 2312, 2398 |
| C -> G | 541, 1797, 1878, 2398, 2582, 2657, 2723 |
| G -> A | 572, 2450, 2549, 2937, 2940 |
| G -> C | 575, 576, 2450, 2937, 2975, 2976, 3018 |
| T -> A | 688, 1454 |
| T -> G | 688, 1454, 2626, 2670 |
| C -> A | 692, 1797, 1878, 2582, 2793 |
| A -> T | 713, 1968 |
| A -> G | 713, 1968, 1977, 2944 |
| G -> T | 2399, 3018 |
| -> A | 2415, 2575, 2771, 2778, 2808 |
| -> T | 2575, 2921 |
| T -> C | 2670, 3019 |
| -> G | 2771 |
| G -> | 2800 |
| -> C | 2808, 2911, 2921 |
| A -> C | 2944 |

The coding portion of transcript D11717_T17 (SEQ ID NO:40) starts at position 552 and ends at position 833. The transcript also has the following SNPs as listed in Table 15 (given according to their position on the nucleotide sequence, with the alternative nucleic acid listed; the last column indicates whether the SNP is known or not; the presence of known SNPs in variant protein D11717_P18 (SEQ ID NO:56) sequence provides support for the deduced sequence of this variant protein according to the present invention).

TABLE 15
Nucleic acid SNPs

| Polymorphism | SNP position(s) on nucleotide sequence |
|---|---|
| C -> | 108, 1634, 1777, 1833 |
| C -> T | 400, 541, 692, 741, 1431, 1432, 1518 |
| C -> G | 541, 1518, 1702, 1777, 1843 |
| G -> A | 572, 1570, 1669, 2057, 2060 |
| G -> C | 575, 576, 1570, 2057, 2095, 2096, 2138 |
| T -> A | 688 |
| T -> G | 688, 1746, 1790 |
| C -> A | 692, 1702, 1913 |
| A -> T | 713 |
| A -> G | 713, 2064 |
| G -> T | 1519, 2138 |
| -> A | 1535, 1695, 1891, 1898, 1928 |
| -> T | 1695, 2041 |
| T -> C | 1790, 2139 |
| -> G | 1891 |
| G -> | 1920 |
| -> C | 1928, 2031, 2041 |
| A -> C | 2064 |

The coding portion of transcript D11717_T21 (SEQ ID NO:41) starts at position 552 and ends at position 833. The transcript also has the following SNPs as listed in Table 16 (given according to their position on the nucleotide sequence, with the alternative nucleic acid listed; the last column indicates whether the SNP is known or not; the presence of known SNPs in variant protein D11717_P18 (SEQ ID NO:56) sequence provides support for the deduced sequence of this variant protein according to the present invention).

TABLE 16
Nucleic acid SNPs

| Polymorphism | SNP position(s) on nucleotide sequence |
|---|---|
| C -> | 108 |
| C -> T | 400, 541, 692, 741 |
| C -> G | 541, 1797 |
| G -> A | 572 |
| G -> C | 575, 576 |
| T -> A | 688, 1454 |
| T -> G | 688, 1454 |
| C -> A | 692, 1797 |
| A -> T | 713 |
| A -> G | 713 |

As noted above, cluster D11717 features 9 segment(s), which were listed in Table 3 above. These segment(s) are portions of nucleic acid sequence(s) which are described herein separately because they are of particular interest. A description of each segment according to the present invention is now provided.

Segment cluster D11717_N0 (SEQ ID NO:42) according to the present invention is supported by 2 libraries. The number of libraries was determined as previously described. This segment can be found in the following transcript(s): D11717_T2 (SEQ ID NO:36) and D11717_T3 (SEQ ID NO:37). Table 17 below describes the starting and ending position of this segment on each transcript.

TABLE 17
Segment location on transcripts

| | Segment | Segment |
|---|---|---|
| Transcript name | starting position | ending position |
| D11717_T2 (SEQ ID NO: 36) | 1 | 270 |
| D11717_T3 (SEQ ID NO: 37) | 1 | 270 |

Segment cluster D11717_N13 (SEQ ID NO:43) according to the present invention is supported by 186 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40), D11717_T2 (SEQ ID NO:36), D11717_T21 (SEQ ID NO:41) and D11717_T3 (SEQ ID NO:37). Table 18 below describes the starting and ending position of this segment on each transcript.

TABLE 18
Segment location on transcripts

| | Segment | Segment |
|---|---|---|
| Transcript name | starting position | ending position |
| D11717_T0 (SEQ ID NO: 35) | 279 | 590 |
| D11717_T14 (SEQ ID NO: 39) | 279 | 590 |
| D11717_T17 (SEQ ID NO: 40) | 279 | 590 |
| D11717_T2 (SEQ ID NO: 36) | 372 | 683 |
| D11717_T21 (SEQ ID NO: 41) | 279 | 590 |
| D11717_T3 (SEQ ID NO: 37) | 271 | 582 |

Segment cluster D11717_N14 (SEQ ID NO:44) according to the present invention is supported by 213 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40), D11717_T2 (SEQ ID NO:36), D11717_T21 (SEQ ID NO:41), D11717_T3 (SEQ ID NO:37) and D11717_T4 (SEQ ID NO:38). Table 19 below describes the starting and ending position of this segment on each transcript.

TABLE 19
Segment location on transcripts

| | Segment | Segment |
|---|---|---|
| Transcript name | starting position | ending position |
| D11717_T0 (SEQ ID NO: 35) | 591 | 828 |
| D11717_T14 (SEQ ID NO: 39) | 591 | 828 |
| D11717_T17 (SEQ ID NO: 40) | 591 | 828 |
| D11717_T2 (SEQ ID NO: 36) | 684 | 921 |
| D11717_T21 (SEQ ID NO: 41) | 591 | 828 |
| D11717_T3 (SEQ ID NO: 37) | 583 | 820 |
| D11717_T4 (SEQ ID NO: 38) | 267 | 504 |

Segment cluster D11717_N15 (SEQ ID NO:45) according to the present invention is supported by 15 libraries. The number of libraries was determined as previously described. This segment can be found in the following transcript(s): D11717_T14 (SEQ ID NO:39), D11717_T17 (SEQ ID NO:40) and D11717_T21 (SEQ ID NO:41). Table 20 below describes the starting and ending position of this segment on each transcript.

TABLE 20
Segment location on transcripts

| | Segment | Segment |
|---|---|---|
| Transcript name | starting position | ending position |
| D11717_T14 (SEQ ID NO: 39) | 829 | 1376 |
| D11717_T17 (SEQ ID NO: 40) | 829 | 1376 |
| D11717_T21 (SEQ ID NO: 41) | 829 | 1376 |

According to an optional embodiment of the present invention, short segments related to the above cluster are also provided. These segments are up to about 120 by in length, and so are included in a separate description.

Segment cluster D11717_N1 (SEQ ID NO:46) according to the present invention is supported by 1 libraries. The number of libraries was determined as previously described. This segment can be found in the following transcript(s): D11717_T2 (SEQ ID NO:36). Table 21 below describes the starting and ending position of this segment on each transcript.

TABLE 21
Segment location on transcripts

| | Segment | Segment |
|---|---|---|
| Transcript name | starting position | ending position |
| D11717_T2 (SEQ ID NO: 36) | 271 | 273 |

Segment cluster D11717_N3 (SEQ ID NO:47) according to the present invention is supported by 6 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40), D11717_T21 (SEQ ID NO:41) and D11717_T4 (SEQ ID NO:38). Table 22 below describes the starting and ending position of this segment on each transcript.

TABLE 22
Segment location on transcripts

| | Segment | Segment |
|---|---|---|
| Transcript name | starting position | ending position |
| D11717_T0 (SEQ ID NO: 35) | 1 | 94 |
| D11717_T14 (SEQ ID NO: 39) | 1 | 94 |
| D11717_T17 (SEQ ID NO: 40) | 1 | 94 |
| D11717_T21 (SEQ ID NO: 41) | 1 | 94 |
| D11717_T4 (SEQ ID NO: 38) | 1 | 94 |

Segment cluster D11717_N4 (SEQ ID NO:48) according to the present invention is supported by 4 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40) and D11717_T21 (SEQ ID NO:41). Table 23 below describes the starting and ending position of this segment on each transcript.

TABLE 23
Segment location on transcripts

| | Segment | Segment |
|---|---|---|
| Transcript name | starting position | ending position |
| D11717_T0 (SEQ ID NO: 35) | 95 | 106 |
| D11717_T14 (SEQ ID NO: 39) | 95 | 106 |
| D11717_T17 (SEQ ID NO: 40) | 95 | 106 |
| D11717_T21 (SEQ ID NO: 41) | 95 | 106 |

Segment cluster D11717_N5 (SEQ ID NO:49) according to the present invention is supported by 6 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40), D11717_T21 (SEQ ID NO:41) and D11717_T4 (SEQ ID NO:38). Table 24 below describes the starting and ending position of this segment on each transcript.

TABLE 24
Segment location on transcripts

| | Segment | Segment |
|---|---|---|
| Transcript name | starting position | ending position |
| D11717_T0 (SEQ ID NO: 35) | 107 | 180 |
| D11717_T14 (SEQ ID NO: 39) | 107 | 180 |
| D11717_T17 (SEQ ID NO: 40) | 107 | 180 |
| D11717_T21 (SEQ ID NO: 41) | 107 | 180 |
| D11717_T4 (SEQ ID NO: 38) | 95 | 168 |

Segment cluster D11717_N6 (SEQ ID NO:50) according to the present invention is supported by 7 libraries. The number of libraries was determined as previously described. This segment can be found in the following transcript(s): D11717_T0 (SEQ ID NO:35), D11717_T14 (SEQ ID NO:39), D11717_T17 (SEQ ID NO:40), D11717_T2 (SEQ ID NO:36), D11717_T21 (SEQ ID NO:41) and D11717_T4 (SEQ ID NO:38). Table 25 below describes the starting and ending position of this segment on each transcript.

TABLE 25
Segment location on transcripts

| | Segment | Segment |
|---|---|---|
| Transcript name | starting position | ending position |
| D11717_T0 (SEQ ID N: 35) | 181 | 278 |
| D11717_T14 (EQ ID NO: 39) | 181 | 278 |
| D11717_T17 (SEQ ID NO: 40) | 181 | 278 |
| D11717_T2 (SEQ ID NO: 36) | 274 | 371 |
| D11717_T21 (SEQ ID NO: 41) | 181 | 278 |
| D11717_T4 (SEQ ID NO: 38) | 169 | 266 |

### Variant Protein Alignment to the Previously Known Protein:

Alignment of: D11717_P4 (SEQ ID NO:55)×known protein(s) GDF15_HUMAN_V1 (SEQ ID NO:54):
Total length: 343
Matching length: 296

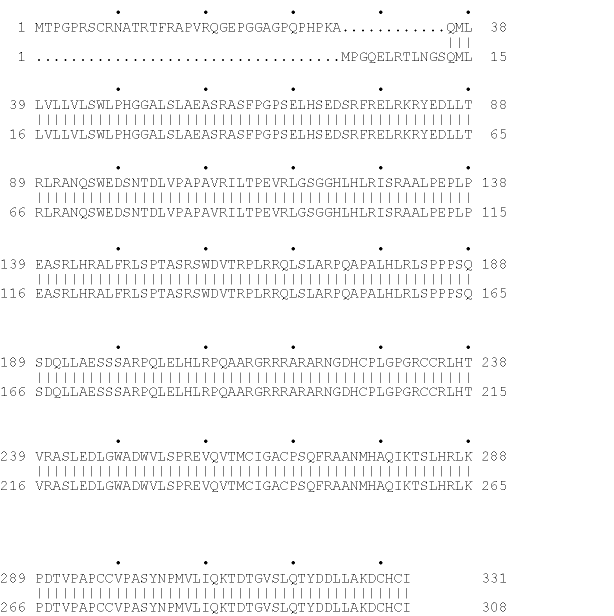

Alignment of: D11717_P4 (SEQ ID NO:55)×known protein(s) Q9BWA0_HUMAN (SEQ ID NO: 53):
Total length: 343
Matching length: 296

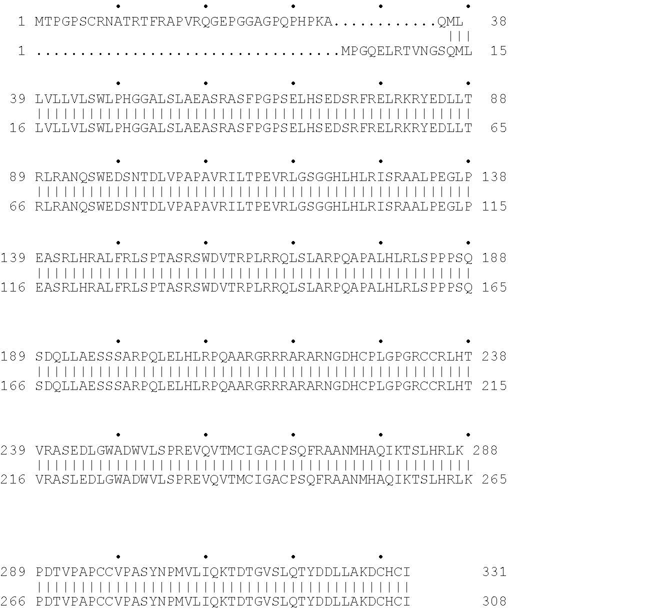

Alignment of: D11717_P4 (SEQ ID NO:55)×known protein(s) NP_{—}004855 (SEQ ID NO: 52):
Total length: 343
Matching length: 296

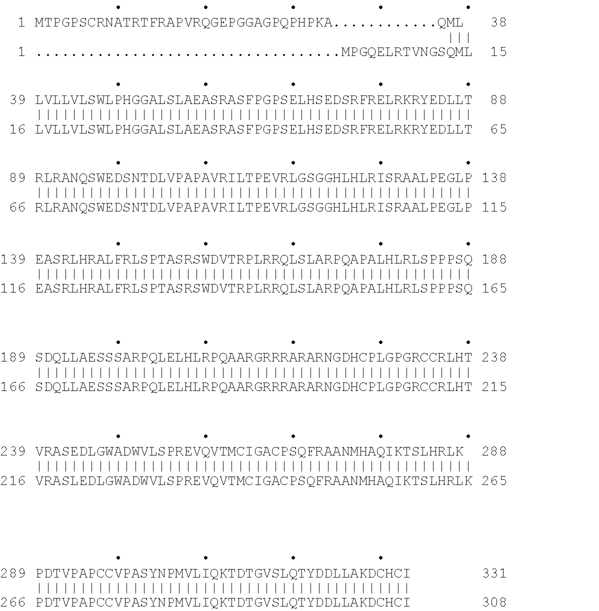

Expression of Growth Differentiation Factor 15 D11717 Transcripts which are Detectable by Amplicon as Depicted in Sequence Name D11717_seg14 (SEQ ID NO: 59) in Normal and Cancerous Colon Tissues

Expression of growth differentiation factor 15 transcripts detectable by or according to seg14-D11717_seg14 (SEQ ID NO: 59) amplicon and primers D11717_seg14F (SEQ ID NO:57) and D11717_seg14R SEQ ID NO:58) was measured by real time PCR. In parallel the expression of several housekeeping genes—HPRT1 (GenBank Accession No. NM_{—}000194 (SEQ ID NO: 5); amplicon—HPRT1—amplicon (SEQ ID NO: 8)), PBGD (GenBank Accession No. BC019323 (SEQ ID NO: 1); amplicon—PBGD-amplicon (SEQ ID NO: 4)), and G6PD (GenBank Accession No. NM_{—}000402 (SEQ ID NO: 13); G6PD amplicon (SEQ ID NO: 151)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42-70, Table 1_{—}4 above), to obtain a value of fold up-regulation for each sample relative to median of the normal samples.

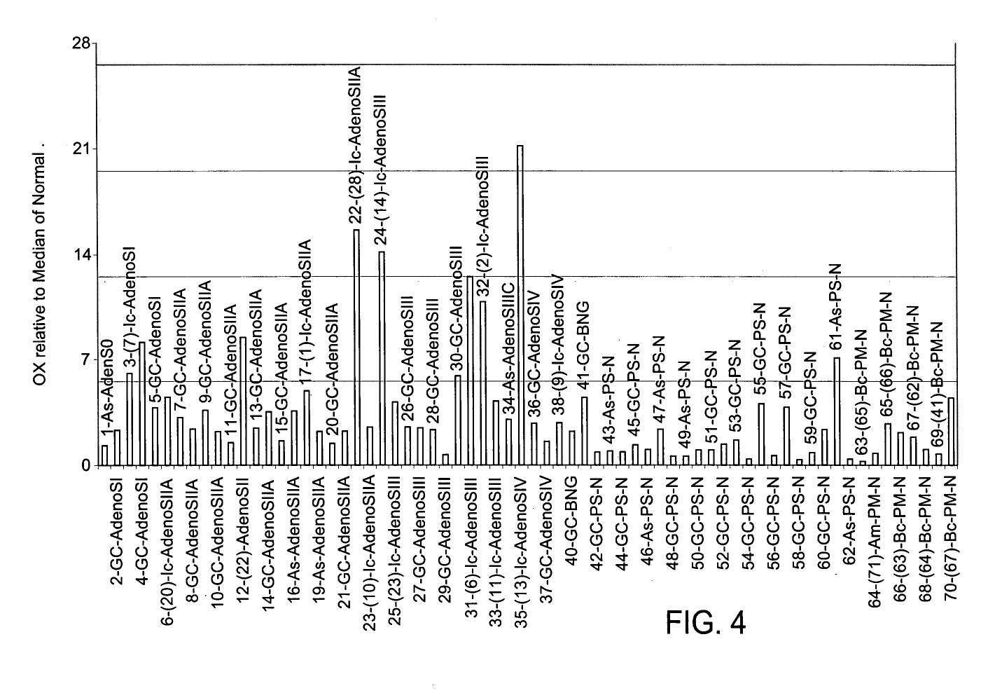

FIG. 4 is a histogram showing over expression of the above-indicated growth differentiation factor 15 transcripts in cancerous Colon samples relative to the normal samples.

As is evident from FIG. 4, the expression of growth differentiation factor 15 transcripts detectable by the above amplicon in cancer samples was higher than in the non-cancerous samples (sample numbers 42-70, Table 1_{—}4 above). Notably an over-expression of at least 7 fold was found in 7 out of 37 adenocarcinoma samples.

Statistical analysis was applied to verify the significance of these results, as described below.

The P value for the difference in the expression levels of growth differentiation factor 15 transcripts detectable by the above amplicon in Colon cancer samples versus the normal tissue samples was determined by T test as 1.27e-004.

The above values demonstrate statistical significance of the results.

Primer pairs are also optionally and preferably encompassed within the present invention; for example, for the above experiment, the following primer pair was used as a non-limiting illustrative example only of a suitable primer pair: D11717_seg14F (SEQ ID NO:57) forward primer; and D11717_seg14R (SEQ ID NO:58) reverse primer.

The present invention also preferably encompasses any amplicon obtained through the use of any suitable primer pair; for example, for the above experiment, the following amplicon was obtained as a non-limiting illustrative example only of a suitable amplicon: D11717_seg14 (SEQ ID NO: 59).

Forward Primer (D11717_seg14F (SEQ ID NO: 57)):
>D11717_seg14F (SEQ ID NO: 57):
CTGGATCCCCTTGGGTCTG
Reverse Primer (D11717_seg14R (SEQ ID NO: 58)):
>D11717_seg14R (SEQ ID NO: 58):
CCTGGCATCCCTGTACCTCA
>D11717_seg14 (SEQ ID NO: 59) amplicon:
CTGGATCCCCTTGGGTCTGATGGGGTAGCCGATGCCTGATTTGCACCCAC
AACGTGGGAGGTTATAACCTGTCCCCAAGTTGCAAATGGGGAAACTGAGG
TACAGGGATGCCAGG

Expression of Homo sapiens GDF-15 Cluster Using MED Discovery Engine:

MED discovery engine, described in Example 4 herein, was used to assess the expression of growth differentiation factor 15 (cluster D11717).

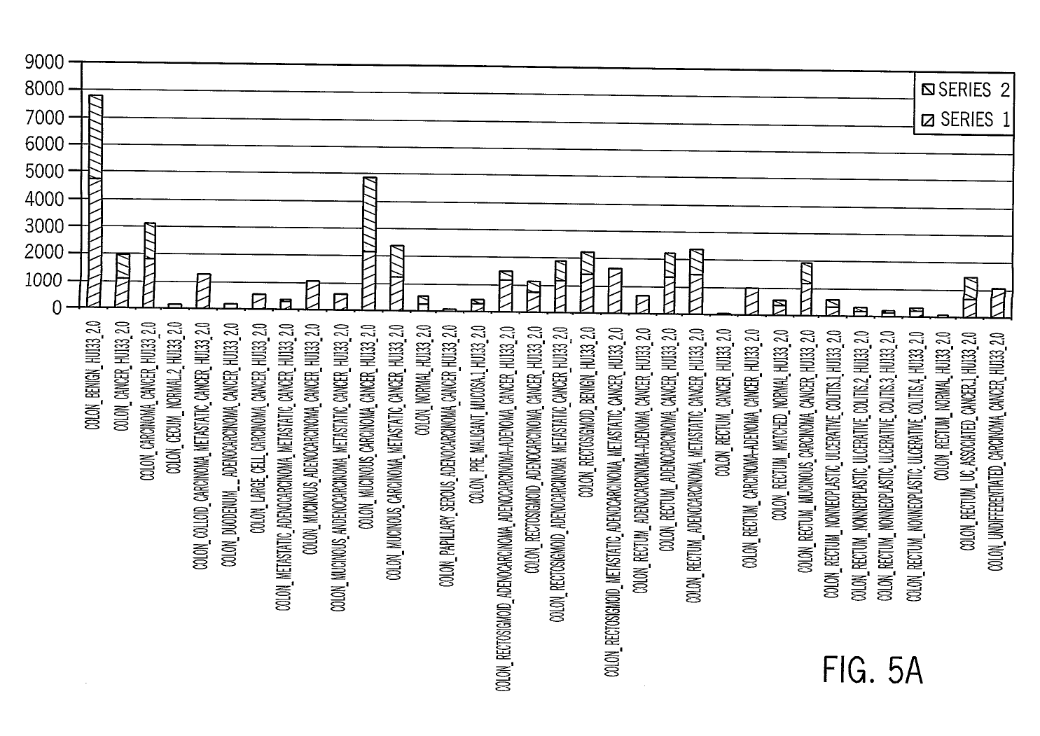

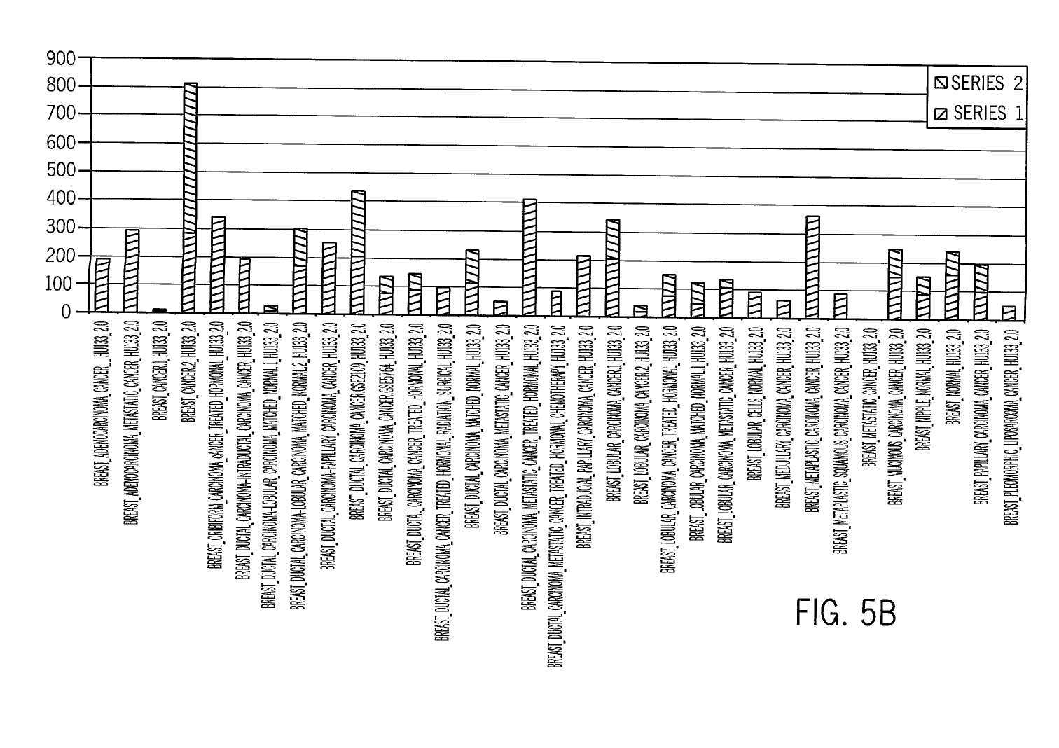

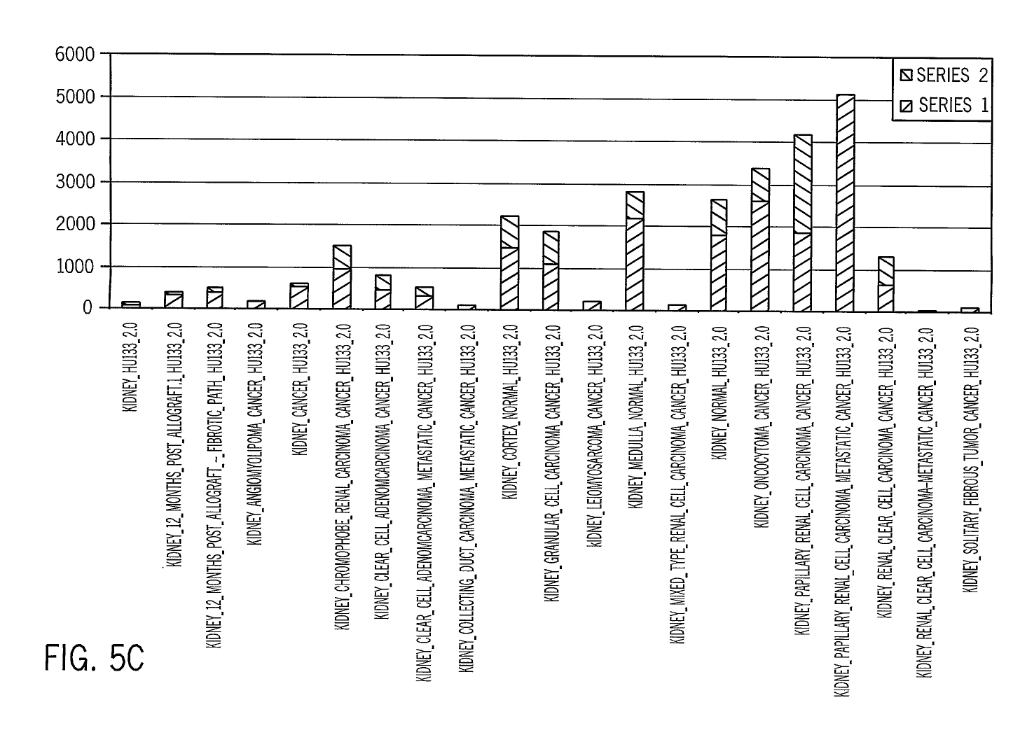

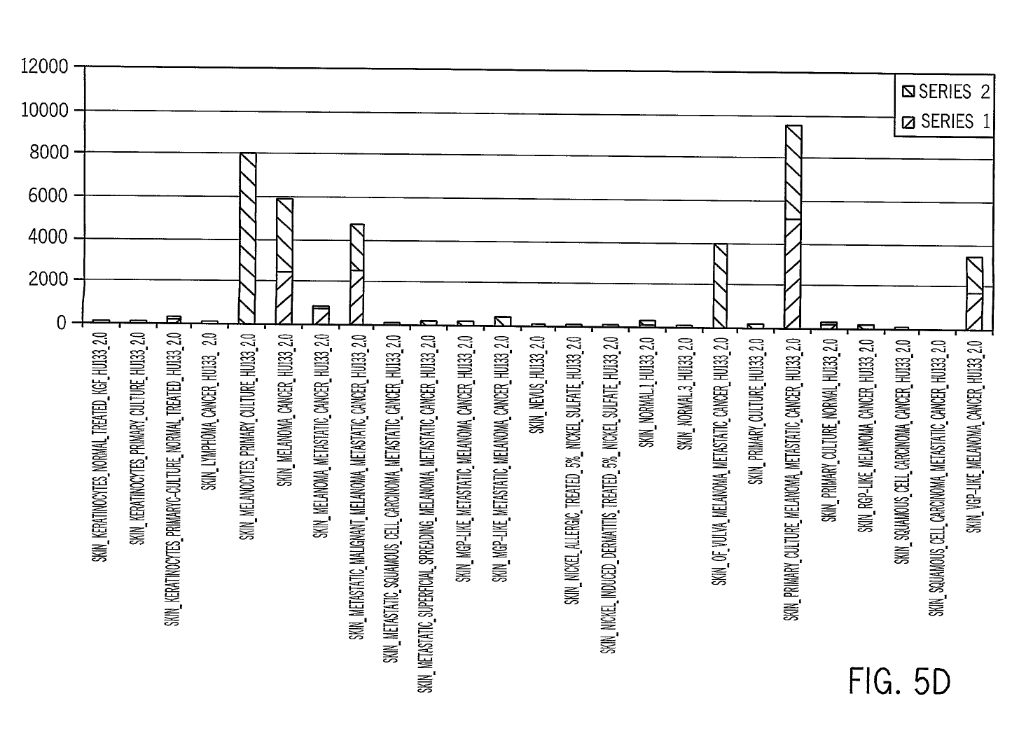

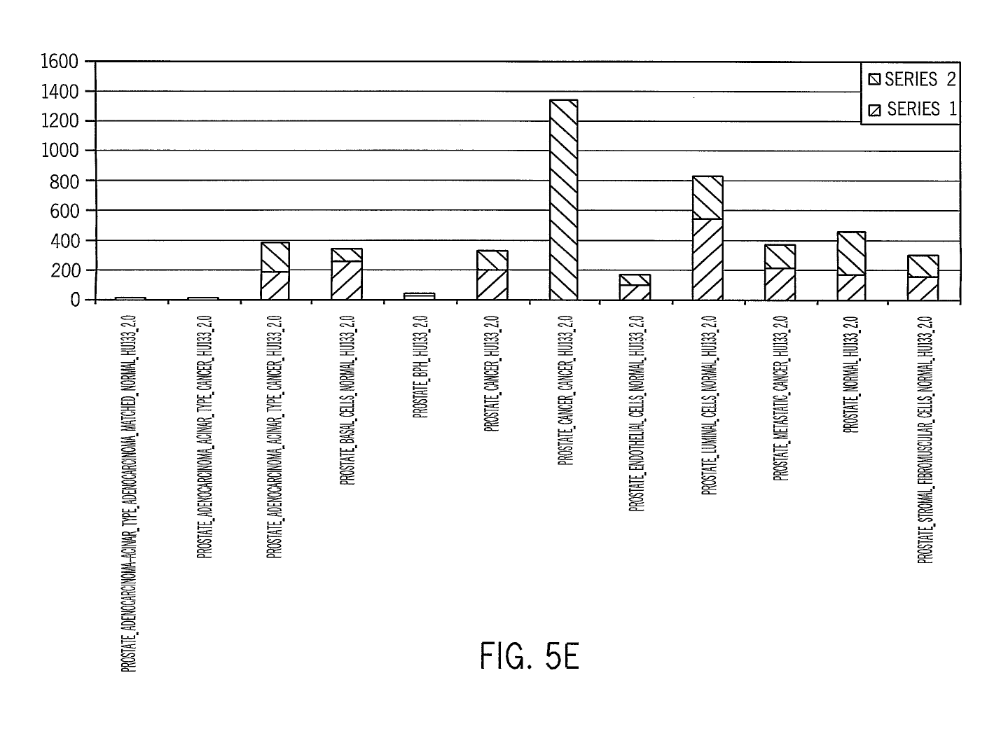

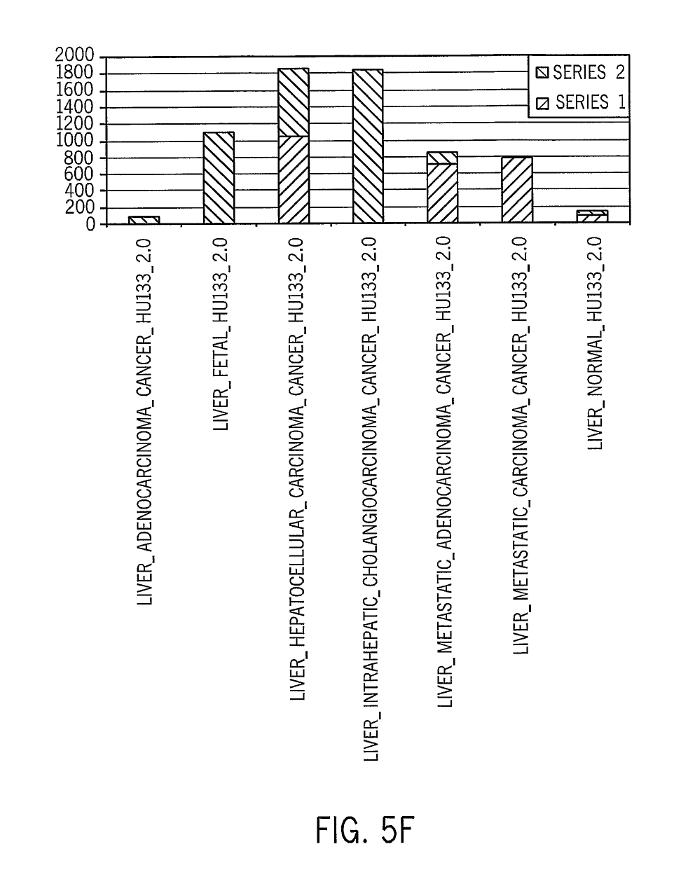

Expression data for Affymetrix probe set 221577_x_at representing GDF-15 from HG-U133 family data is shown in FIGS. 5A-5F.

As is evident from the scatter plots, shown in FIGS. 5A-5F, the expression of D11717 transcripts detectable with the above probe set in cancer samples was higher than in the normal samples. FIG. 5A presents the results on colon panel, showing over expression of cluster D11717 in colon cancer. FIG. 5B presents the results on breast panel, showing over expression of cluster D11717 in breast cancer. FIG. 5C presents the results on kidney panel, showing over expression of cluster D11717 in Kidney papillary renal cell carcinoma. FIG. 5D presents the results on skin panel, showing over expression of cluster D11717 in melanoma. FIG. 5E presents the results on prostate panel, showing over expression of cluster D11717 in prostate cancer. FIG. 5F presents the results on liver panel, showing over expression of cluster D11717 in liver cancer.

### Description for Cluster HSTNFR1A

Cluster HSTNFR1A (internal ID 70365395) features 13 transcript(s) and 23 segment(s) of interest, the names for which are given in Tables 26 and 27. The selected protein variants are given in table 28.

TABLE 26
Transcripts of interest
Transcript Name

HSTNFR1A_T5 (SEQ ID NO: 60)
HSTNFR1A_T6 (SEQ ID NO: 61)
HSTNFR1A_T8 (SEQ ID NO: 62)
HSTNFR1A_T12 (SEQ ID NO: 63)
HSTNFR1A_T14 (SEQ ID NO: 64)
HSTNFR1A_T19 (SEQ ID NO: 65)
HSTNFR1A_T20 (SEQ ID NO: 66)
HSTNFR1A_T23 (SEQ ID NO: 67)
HSTNFR1A_T24 (SEQ ID NO: 68)
HSTNFR1A_T35 (SEQ ID NO: 69)
HSTNFR1A_T38 (SEQ ID NO: 70)
HSTNFR1A_T41 (SEQ ID NO: 71)
HSTNFR1A_T44 (SEQ ID NO: 72)

TABLE 27
Segments of interest
Segment Name

HSTNFR1A_N37 (SEQ ID NO: 73)
HSTNFR1A_N62 (SEQ ID NO: 74)
HSTNFR1A_N64 (SEQ ID NO: 75)
HSTNFR1A_N2 (SEQ ID NO: 76)
HSTNFR1A_N4 (SEQ ID NO: 77)
HSTNFR1A_N6 (SEQ ID NO: 78)
HSTNFR1A_N8 (SEQ ID NO: 79)
HSTNFR1A_N10 (SEQ ID NO: 80)
HSTNFR1A_N12 (SEQ ID NO: 81)
HSTNFR1A_N31 (SEQ ID NO: 82)
HSTNFR1A_N36 (SEQ ID NO: 83)
HSTNFR1A_N39 (SEQ ID NO: 84)
HSTNFR1A_N40 (SEQ ID NO: 85)
HSTNFR1A_N43 (SEQ ID NO: 86)
HSTNFR1A_N53 (SEQ ID NO: 87)
HSTNFR1A_N56 (SEQ ID NO: 88)
HSTNFR1A_N59 (SEQ ID NO: 89)
HSTNFR1A_N60 (SEQ ID NO: 90)
HSTNFR1A_N61 (SEQ ID NO: 91)
HSTNFR1A_N63 (SEQ ID NO: 92)
HSTNFR1A_N65 (SEQ ID NO: 93)
HSTNFR1A_N75 (SEQ ID NO: 94)
HSTNFR1A_N82 (SEQ ID NO: 95)

TABLE 28
Proteins of interest

| Protein Name | Corresponding Transcript(s) |
|---|---|
| HSTNFR1A_P10 (SEQ ID NO: 98) | HSTNFR1A_T12 (SEQ ID NO: 63); HSTNFR1A_T5 |
| | (SEQ ID NO: 60); HSTNFR1A_T6 (SEQ ID NO: 61) |
| HSTNFR1A_P11 (SEQ ID NO: 99) | HSTNFR1A_T14 (SEQ ID NO: 64) |
| HSTNFR1A_P14 (SEQ ID NO: 100) | HSTNFR1A_T19 (SEQ ID NO: 65) |
| HSTNFR1A_P15 (SEQ ID NO: 101) | HSTNFR1A_T20 (SEQ ID NO: 66) |
| HSTNFR1A_P26 (SEQ ID NO: 102) | HSTNFR1A_T35 (SEQ ID NO: 69) |
| HSTNFR1A_P27 (SEQ ID NO: 103) | HSTNFR1A_T38 (SEQ ID NO: 70) |
| HSTNFR1A_P36 (SEQ ID NO: 104) | HSTNFR1A_T8 (SEQ ID NO: 62) |
| HSTNFR1A_P37 (SEQ ID NO: 105) | HSTNFR1A_T23 (SEQ ID NO: 67) |
| HSTNFR1A_P38 (SEQ ID NO: 106) | HSTNFR1A_T24 (SEQ ID NO: 68) |
| HSTNFR1A_P43 (SEQ ID NO: 107) | HSTNFR1A_T41 (SEQ ID NO: 71) |
| HSTNFR1A_P44 (SEQ ID NO: 108) | HSTNFR1A_T44 (SEQ ID NO: 72) |

These sequences are variants of the known protein Tumor necrosis factor receptor superfamily member 1A precursor (SEQ ID NO:96) (SwissProt accession identifier TNR1A_HUMAN (SEQ ID NO: 96); known also according to the synonyms p60; TNF-R1; TNF-RI; TNFR-I; p55; CD120a antigen), referred to herein as the previously known protein.

Protein Tumor necrosis factor receptor superfamily member 1A precursor (SEQ ID NO:96) is known or believed to have the following function(s): Receptor for TNFSF2/TNF-alpha and homotrimeric TNFSF1/lymphotoxin-alpha. The adapter molecule FADD recruits caspase-8 to the activated receptor. The resulting death-inducing signaling complex (DISC) performs caspase-8 proteolytic activation which initiates the subsequent cascade of caspases (aspartate-specific cysteine proteases) mediating apoptosis. Contributes to the induction of noncytocidal TNF effects including anti-viral state and activation of the acid sphingomyelinase. Known polymorphisms for this sequence are as shown in Table 29.

TABLE 29
Amino acid mutations for Known Protein

| SNP position(s) on amino | |
|---|---|
| acid sequence | Comment |
| 115 | S -> G (in FHF). /FTId = VAR_019331 |
| 79 | T -> M (in FHF). /FTId = VAR_013412 |
| 117 | C -> Y (in FHF). /FTId = VAR_013415 |
| 51 | H -> Q (in FHF). /FTId = VAR_019329 |
| 99 | C -> S (in FHF). /FTId = VAR_019304 |
| 121 | R -> P (in FHF). /FTId = VAR_019305 |
| 443-446 | GPAA -> APP |
| 121 | R -> Q (in FHF; may be a polymorphism; |
| | dbSNP: 4149584). |
| | /FTId = VAR_019332 |
| 59 | C -> S (in FHF). /FTId = VAR_019302 |
| 62 | C -> G (in FHF). /FTId = VAR_019303 |
| 62 | C -> Y (in FHF). /FTId = VAR_013411 |
| 305 | P -> T (in dbSNP: 1804532). |
| | /FTId = VAR_011813 |
| 75 | P -> L (in FHF; may be a polymorphism; |
| | dbSNP: 4149637). |
| | /FTId = VAR_019330 |
| 117 | C -> R (in FHF). /FTId = VAR_013414 |
| 412 | Missing |
| 59 | C -> R (in FHF). /FTId = VAR_013410 |
| 81 | C -> F (in FHF). /FTId = VAR_013413 |

Protein Tumor necrosis factor receptor superfamily member 1A precursor (SEQ ID NO:96) localization is believed to be Cell membrane; single-pass type I membrane protein Secreted protein.

The previously known protein also has the following indication(s) and/or potential therapeutic use(s): Fibrosis, pulmonary; Arthritis, psoriatic; Ankylosing spondylitis; Multiple sclerosis, general; Arthritis, rheumatoid; Psoriasis; Uveitis; Chronic obstructive pulmonary disease; Sepsis; Wegener's granulomatosis; Infection, hepatitis-C virus; Asthma. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Tumour necrosis factor agonist; Tumour necrosis factor alpha antagonist; Tumour necrosis factor antagonist; Tumour necrosis factor alpha agonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anticancer, immunological; Respiratory; COPD treatment; Formulation, conjugate, pegylated; Immunoconjugate, other; Monoclonal antibody, other; Cytokine; Septic shock treatment; Antiarthritic, other; Antipsoriasis; Recombinant, other; Antiarthritic, immunological; Antiasthma; Gene therapy; Immunosuppressant; Opthalmological; Multiple sclerosis treatment; Cardiovascular; Immunomodulator, anti-infective.

The following GO Annotation(s) apply to the previously known protein. The following annotation(s) were found: positive regulation of inflammatory response; cytokine and chemokine mediated signaling pathway; prostaglandin metabolism; positive regulation of I-kappaB kinase/NF-kappaB cascade; inflammatory response; positive regulation of transcription from Pol II promoter, which are annotation(s) related to Biological Process; signal transducer activity; tumor necrosis factor receptor activity; protein binding, which are annotation(s) related to Molecular Function; and integral to plasma membrane; extracellular region, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

The present invention provides a number of different novel amino acid and nucleic acid sequences of known TNR1A_HUMAN protein, which may optionally be used as diagnostic markers, preferably as serum markers.

Variants HSTNFR1A_P10 (SEQ ID NO:98), HSTNFR1A_P11 (SEQ ID NO:99), HSTNFR1A_P14 (SEQ ID NO:100) and HSTNFR1A_P15 (SEQ ID NO:101) were previously disclosed by the inventors in U.S. application Ser. No. 11/043,770, hereby incorporated by reference as if fully set forth herein, but have now been shown to have novel and surprising diagnostic uses as described herein for other variants of cluster HSTNFR1A.

According to optional but preferred embodiments of the present invention, variants of this cluster according to the present invention (amino acid of HSTNFR1A) may optionally have one or more of the following utilities, as described with regard to the Table 30 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 30
Utilities for variants of the HSTNFR1A related to various types of cancers and various inflammatory
disorders

| Utility | Reason | reference |
|---|---|---|
| Indicator for prognosis in | In a cohort of 56 formalin-fixed and paraffin-embedded | Anticancer Res. |
| Duke's stage C colorectal | specimens with Duke's stage C colorectal cancer, 66% had a | 2003 Jan-Feb; |
| cancer | high expression and 34% had a low expression of TNFR-1A. | 23(1A): 85-9. |
| | Expression of TNFR-1A had no significant correlation with any | |
| | of the clinopathological factors studies except for tumor | |
| | differentiation status. Patients with a high TNFR-1A expression | |
| | had a significantly better disease-specific survival rate than | |
| | those with a low TNFR-1A (77.8% vs. 60.3%, respectively. | |
| | P = 0.0159 log rank test). Also, in the multivariable analysis, | |
| | TNFR-1A expression was an independent prognostic factor. | |
| Differentiating tumor | Serum levels of soluble tumor necrosis factor super family 1A | Circulation. |
| necrosis factor receptor | (TNFRS1A), TNF-alpha, Interleukin (IL)-6, and C-reactive | 2007 Feb 27; |
| associated periodic syndrome | protein (CRP) were measured in two siblings with TRAPS who | 115(8): 962-71. |
| (TRAPS) from systemic | initially have been diagnosed with JIA and in JIA patients. Both | |
| juvenile idiopathic arthritis | siblings had serum levels of soluble TNFRSF1A that were | |
| (JIA). | below the normal reference range, and did not reach a level | |
| | corresponding to that of systemic JIA. On TNFRSF1A gene | |
| | analysis, a single missence mutation resulting in C30Y was | |
| | found in both siblings. Based on the clinical features and the | |
| | TNFRSF1A mutation, both siblings were given a diagnosis of | |
| | TRAPS. The serum levels of soluble TNFRSF1A, measured | |
| | along with the CRP level, may be a useful screening marker for | |
| | differentiating TRAPS from systemic JIA. | |
| Prognostic value of soluble | For MS patients treated with IFN for a period of 15 | Eur Neurol. 2001; |
| tumor necrosis factor receptors | months, a cranial MRI was performed every 6 months and | 46(4): 210-4. |
| 1 (sTNF-R1) and 2 (sTNF-R2) | blood was drawn every 6 months to determine cytokine | |
| in patients with multiple | receptor levels. In the treatment group, the MRI | |
| sclerosis (MS) treated with | responders, had a significantly larger mean values for the | |
| interferon beta-1b (IFN). | area under the concentration time curve of sTNF-R1 and | |
| | sTNF-R2 when compared to the nonresponders group | |
| | during the 15-month observation period. For the MRI | |
| | responders group, there was an increase in sTNF-R1 and | |
| | sTNF-R2 of more than 20% in the first 3 months of | |
| | treatment: sensitivity: 33% and 58%, specificity: 90% and | |
| | 60%, PPV: 80% and 64% respectively. Assessment of | |
| | sTNF-Rs may contribute to the identification of | |
| | subgroups of patients who are likely to response better | |
| | than others to treatment with INF. | |
| TNF-receptor-associated | The TNF-receptor periodic syndrome (TRAPS) is an | Clin Rheumatol. |
| periodic syndrome (TRAPS): | autosomal dominant auto-inflamatory disorder | 2006 |
| autosomal dominant | characterized by recurrent febrile attacks and localized | Nov; 25(6); 773-7. |
| multisystem disorder. | inflammation. TRAPS is caused by mutations in the gene | |
| | encoding the TNF receptor Super Family 1A | |
| | (TNFRSF1A) on chromosme 12p13. Some low- | |
| | penetrance TNFRSF1A variants contribute to atypical | |
| | inflammatory responses in other autoimmune diseases. | |
| An association between TNF- | A novel mutation, a heterozygous C to T transition in | Rheunatology |
| receptor-associated periodic | exon 3 which substitutes an isoleucine for a threonine at | (Oxford). 2004. |
| syndrome (TRAPS) and | position 61 (T611) was detected in the TNFRSF1A gene | oct; 43(10): 1292-9. |
| systemic lupos erythematous | derived from the genomic DNA of a Japanese female | |
| (SLE). | patient with TRAPS complicated with SLE. This | |
| | mutation was identified in 8.3% of SLE patients and 4.2% | |
| | among healthy individuals. High titers of serum TNF and | |
| | soluble TNFRSF1B were observed in this patient but low | |
| | titers of soluble TNFRSF1A protein were detected. | |
| Association o the R92Q | The frequency of this mutation in patients with | Arthritis Rheum. |
| TNFRSF1A mutation and | Behcet's disease was significantly higher than than in | 2005 Feb; 52(2): 608-11. |
| extracranial deep vein | controls. Deep vein thrombosis was significantly | |
| thrombosis in patients with | associated with the R92Q mutation. Among 20 patients | |
| Behcet's disease. | with Behcet's disease who had extracranial deep vein | |
| | thrombosis, 6 had the mutation. The R92Q mutation in | |
| | patients with Behcet's disease is associated with an | |
| | increased risk of extracranial venous thrombosis. | |
| Tumor necrosis factor receptor | Among 205 CD patients who were genotyped for | Am. J. |
| gene polymorphism in Crohn's | polymorphism in TNFRSF1A (position +36, −609), | Gastroenterol. 2005 |
| disease (CD): association with | TNFRSF1B (position +196, −1466) and 3 common | May; 100(5): 1126-33. |
| clinical phenotypes. | CARD15 variants, TNFRSF1A +36 and TNFRSF1 +196 | |
| | SNP's were associated with CD. These findings may | |
| | help understand the role of TNFR mutation in | |
| | determining clinical heterogeneity in CD. | |

As noted above, cluster HSTNFR1A features 13 transcript(s), which were listed in Table 26 above. These transcript(s) encode for protein(s) which are variant(s) of protein Tumor necrosis factor receptor superfamily member 1A precursor (SEQ ID NO:96). A description of each variant protein according to the present invention is now provided.

Variant protein HSTNFR1A P10 (SEQ ID NO:98) according to the present invention is encoded by transcript(s) HSTNFR1A_T12 (SEQ ID NO:63), HSTNFR1A_T5 (SEQ ID NO:60) and HSTNFR1A_T6 (SEQ ID NO:61).

The localization of the variant protein was determined according to results from a number of different software programs and analyses, including analyses from SignalP and other specialized programs. The variant protein is believed to be located as follows with regard to the cell: secreted.

Variant protein HSTNFR1A_P10 (SEQ ID NO:98) also has the following non-silent SNPs (Single Nucleotide Polymorphisms) as listed in Table 31 (given according to their position(s) on the amino acid sequence, with the alternative amino acid(s) listed; the last column indicates whether the SNP is known or not; the presence of known SNPs in variant protein HSTNFR1A_P10 (SEQ ID NO:98) sequence provides support for the deduced sequence of this variant protein according to the present invention).

TABLE 31
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 75 | P -> L |
| 116 | S -> |
| 157 | S -> F |
| 171 | G -> V |

The glycosylation sites of variant protein HSTNFR1A_P10 (SEQ ID NO:98), as compared to the known protein Tumor necrosis factor receptor superfamily member 1A precursor (SEQ ID NO:96), are described in Table 32 (given according to their position(s) on the amino acid sequence in the first column; the second column indicates whether the glycosylation site is present in the variant protein; and the last column indicates whether the position is different on the variant protein).

TABLE 32
Glycosylation site(s)

| Position(s) on known amino acid | Present in | Position(s) on |
|---|---|---|
| sequence | variant protein? | variant protein |
| 54 | Yes | 54 |
| 145 | Yes | 145 |
| 151 | Yes | 151 |

The variant protein has the following domains, as determined by using InterPro. The domains are described in Table 33:

TABLE 33
InterPro domain(s)

| Domain description | Analysis type | Position(s) on protein |
|---|---|---|
| TNFR | HMMPfam | 44-81, 84-125, 127-166, 168-196 |
| TNFR | HMMSmart | 44-81, 84-125, 127-166, 168-196 |
| TNFR | ProfileScan | 43-81, 83-125, 126-166 |
| EGF-like | ScanRegExp | 166-179 |
| TNFR | ScanRegExp | 44-81, 84-125, 127-166 |
| Cytochrome c | ScanRegExp | 59-64 |
| heme-binding site | | |

Variant protein HSTNFR1A_P10 (SEQ ID NO:98) is encoded by the following transcript(s): HSTNFR1A_T12 (SEQ ID NO:63), HSTNFR1A_T5 (SEQ ID NO:60) and HSTNFR1A_T6 (SEQ ID NO:61).

The coding portion of transcript HSTNFR1A_T12 (SEQ ID NO:63) starts at position 304 and ends at position 957. The transcript also has the following SNPs as listed in Table 34 (given according to their position on the nucleotide sequence, with the alternative nucleic acid listed; the last column indicates whether the SNP is known or not; the presence of known SNPs in variant protein HSTNFR1A_P10 (SEQ ID NO:98) sequence provides support for the deduced sequence of this variant protein according to the present invention).

TABLE 34
Nucleic acid SNPs

| Polymorphism | SNP position(s) on nucleotide sequence |
|---|---|
| A -> G | 339, 3075 |
| C -> A | 340, 1330, 2425, 3213 |
| C -> G | 340, 1330, 2887, 3213 |
| C -> T | 436, 527, 573, 773, 2109, 2852, 2902, 2923, 2945, 2980, |
| | 3027, 3189 |
| T -> | 487 |
| G -> T | 528, 815, 2911, 3415 |
| G -> C | 528 |
| C -> | 650, 2307, 2496, 2850, 2887, 3066, 3289 |
| G -> A | 666, 2005, 2006, 3415 |
| T -> A | 1507 |
| -> G | 1994 |
| G -> | 2734, 3065 |
| T -> C | 2812 |
| A -> C | 3361 |

The coding portion of transcript HSTNFR1A_T5 (SEQ ID NO:60) starts at position 304 and ends at position 957. The transcript also has the following SNPs as listed in Table 35 (given according to their position on the nucleotide sequence, with the alternative nucleic acid listed; the last column indicates whether the SNP is known or not; the presence of known SNPs in variant protein HSTNFR1A_P10 (SEQ ID NO:98) sequence provides support for the deduced sequence of this variant protein according to the present invention).

TABLE 35
Nucleic acid SNPs

| Polymorphism | SNP position(s) on nucleotide sequence |
|---|---|
| A -> G | 339, 2934 |
| C -> A | 340, 1330, 2284, 3072 |
| C -> G | 340, 1330, 2746, 3072 |
| C -> T | 436, 527, 573, 773, 2711, 2761, 2782, 2804, |
| | 2839, 2886, 3048 |
| T -> | 487 |
| G -> T | 528, 815, 2770, 3274 |
| G -> C | 528 |
| C -> | 650, 2166, 2355, 2709, 2746, 2925, 3148 |
| G -> A | 666, 3274 |
| T -> A | 1507 |
| -> G | 1994 |
| G -> | 2593, 2924 |
| T -> C | 2671 |
| A -> C | 3220 |

The coding portion of transcript HSTNFR1A_T6 (SEQ ID NO:61) starts at position 304 and ends at position 957. The transcript also has the following SNPs as listed in Table 36 (given according to their position on the nucleotide sequence, with the alternative nucleic acid listed; the last column indicates whether the SNP is known or not; the presence of known SNPs in variant protein HSTNFR1A_P10 (SEQ ID NO:98) sequence provides support for the deduced sequence of this variant protein according to the present invention).

TABLE 36
Nucleic acid SNPs

| Polymorphism | SNP position(s) on nucleotide sequence |
|---|---|
| A -> G | 339, 1985 |
| C -> A | 340, 1335, 2123 |
| C -> G | 340, 1797, 2123 |
| C -> T | 436, 527, 573, 773, 1762, 1812, 1833, 1855, 1890, 1937, |
| | 2099 |
| T -> | 487 |
| G -> T | 528, 815, 1821, 2325 |
| G -> C | 528 |
| C -> | 650, 1217, 1406, 1760, 1797, 1976, 2199 |
| G -> A | 666, 2325 |
| -> G | 1045 |
| G -> | 1644, 1975 |
| T -> C | 1722 |
| A -> C | 2271 |

Variant protein HSTNFR1A_P11 (SEQ ID NO:99) according to the present invention is encoded by transcript(s) HSTNFR1A_T14 (SEQ ID NO:64). The localization of the variant protein was determined according to results from a number of different software programs and analyses, including analyses from SignalP and other specialized programs. The variant protein is believed to be located as follows with regard to the cell: secreted.

Variant protein HSTNFR1A_P11 (SEQ ID NO:99) also has the following non-silent SNPs (Single Nucleotide Polymorphisms) as listed in Table 37, (given according to their position(s) on the amino acid sequence, with the alternative amino acid(s) listed; the last column indicates whether the SNP is known or not; the presence of known SNPs in variant protein HSTNFR1A_P11 (SEQ ID NO:99) sequence provides support for the deduced sequence of this variant protein according to the present invention).

TABLE 37
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 75 | P -> L |
| 116 | S -> |
| 157 | S -> F |
| 171 | G -> V |

The glycosylation sites of variant protein HSTNFR1A_P11 (SEQ ID NO:99), as compared to the known protein Tumor necrosis factor receptor superfamily member 1A precursor (SEQ ID NO:96), are described in Table 38 (given according to their position(s) on the amino acid sequence in the first column; the second column indicates whether the glycosylation site is present in the variant protein; and the last column indicates whether the position is different on the variant protein).

TABLE 38
Glycosylation site(s)

| Position(s) on known amino acid | Present in | Position(s) on |
|---|---|---|
| sequence | variant protein? | variant protein |
| 54 | Yes | 54 |
| 145 | Yes | 145 |
| 151 | Yes | 151 |

The variant protein has the following domains, as determined by using InterPro. The domains are described in Table 39:

TABLE 39
InterPro domain(s)

| Domain description | Analysis type | Position(s) on protein |
|---|---|---|
| TNFR | HMMSmart | 44-81, 84-125, 127-166 |
| TNFR | ProfileScan | 43-81, 83-125, 126-166 |
| TNFR | ScanRegExp | 44-81, 84-125, 127-166 |
| EGF-like | ScanRegExp | 166-179 |
| TNFR | HMMPfam | 44-81, 84-125, 127-166 |
| Cytochrome c heme-binding | ScanRegExp | 59-64 |
| site | | |

Variant protein HSTNFR1A_P11 (SEQ ID NO:99) is encoded by the following transcript(s): HSTNFR1A_T14 (SEQ ID NO:64). The coding portion of transcript HSTNFR1A_T14 (SEQ ID NO:64) starts at position 304 and ends at position 987. The transcript also has the following SNPs as listed in Table 40 (given according to their position on the nucleotide sequence, with the alternative nucleic acid listed; the last column indicates whether the SNP is known or not; the presence of known SNPs in variant protein HSTNFR1A_P11 (SEQ ID NO:99) sequence provides support for the deduced sequence of this variant protein according to the present invention).

TABLE 40
Nucleic acid SNPs

| Polymorphism | SNP position(s) on nucleotide sequence |
|---|---|
| A -> G | 339, 1793 |
| C -> A | 340, 1143, 1931 |
| C -> G | 340, 1605, 1931 |
| C -> T | 436, 527, 573, 773, 1570, 1620, 1641, 1663, |
| | 1698, 1745, 1907 |
| T -> | 487 |
| G -> T | 528, 815, 1629, 2133 |
| G -> C | 528 |
| C -> | 650, 1025, 1214, 1568, 1605, 1784, 2007 |
| G -> A | 666, 2133 |
| G -> | 1452, 1783 |
| T -> C | 1530 |
| A -> C | 2079 |

Variant protein HSTNFR1A_P14 (SEQ ID NO:100) according to the present invention is encoded by transcript(s) HSTNFR1A_T19 (SEQ ID NO:65).

The localization of the variant protein was determined according to results from a number of different software programs and analyses, including analyses from SignalP and other specialized programs. The variant protein is believed to be located as follows with regard to the cell: secreted.

Variant protein HSTNFR1A_P14 (SEQ ID NO:100) also has the following non-silent SNPs (Single Nucleotide Polymorphisms) as listed in Table 41, (given according to their position(s) on the amino acid sequence, with the alternative amino acid(s) listed; the last column indicates whether the SNP is known or not; the presence of known SNPs in variant protein HSTNFR1A_P14 (SEQ ID NO:100) sequence provides support for the deduced sequence of this variant protein according to the present invention).

TABLE 41
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 75 | P -> L |
| 116 | S -> |
| 157 | S -> F |
| 171 | G -> V |

The glycosylation sites of variant protein HSTNFR1A_P14 (SEQ ID NO:100), as compared to the known protein Tumor necrosis factor receptor superfamily member 1A precursor (SEQ ID NO:96), are described in Table 42 (given according to their position(s) on the amino acid sequence in the first column; the second column indicates whether the glycosylation site is present in the variant protein; and the last column indicates whether the position is different on the variant protein).

TABLE 42
Glycosylation site(s)

| Position(s) on known | Present in variant | Position(s) on |
|---|---|---|
| amino acid sequence | protein? | variant protein |
| 54 | Yes | 54 |
| 145 | Yes | 145 |
| 151 | Yes | 151 |

The variant protein has the following domains, as determined by using InterPro. The domains are described in Table 43:

TABLE 43
InterPro domain(s)

| Domain description | Analysis type | Position(s) on protein |
|---|---|---|
| TNFR | HMMSmart | 44-81, 84-125, 127-166, 168-196 |
| TNFR | ProfileScan | 43-81, 83-125, 126-166 |
| TNFR | HMMPfam | 44-81, 84-125, 127-166, 168-196 |
| Cytochrome c | ScanRegExp | 59-64 |
| heme-binding site | | |
| TNFR | ScanRegExp | 44-81, 84-125, 127-166 |
| EGF-like | ScanRegExp | 166-179 |

Variant protein HSTNFR1A_P14 (SEQ ID NO:100) is encoded by the following transcript(s): HSTNFR1A_T19 (SEQ ID NO:65). The coding portion of transcript HSTNFR1A_T19 (SEQ ID NO:65) starts at position 304 and ends at position 1029. The transcript also has the following SNPs as listed in Table 44 (given according to their position on the nucleotide sequence, with the alternative nucleic acid listed; the last column indicates whether the SNP is known or not; the presence of known SNPs in variant protein HSTNFR1A_P14 (SEQ ID NO:100) sequence provides support for the deduced sequence of this variant protein according to the present invention).

TABLE 44
Nucleic acid SNPs

| Polymorphism | SNP position(s) on nucleotide sequence |
|---|---|
| A -> G | 339, 1688 |
| C -> A | 340, 1038, 1826 |
| C -> G | 340, 1500, 1826 |
| C -> T | 436, 527, 573, 773, 1465, 1515, 1536, 1558, 1593, |
| | 1640, 1802 |
| T -> | 487 |
| G -> T | 528, 815, 1524, 2028 |
| G -> C | 528 |
| C -> | 650, 1109, 1463, 1500, 1679, 1902 |
| G -> A | 666, 2028 |
| G -> | 1347, 1678 |
| T -> C | 1425 |
| A -> C | 1974 |

Variant protein HSTNFR1A_P15 (SEQ ID NO:101) according to the present invention is encoded by transcript(s) HSTNFR1A_T20 (SEQ ID NO:66).

The localization of the variant protein was determined according to results from a number of different software programs and analyses, including analyses from SignalP and other specialized programs. The variant protein is believed to be located as follows with regard to the cell: secreted.

Variant protein HSTNFR1A_P15 (SEQ ID NO:101) also has the following non-silent SNPs (Single Nucleotide Polymorphisms) as listed in Table 45, (given according to their position(s) on the amino acid sequence, with the alternative amino acid(s) listed; the last column indicates whether the SNP is known or not; the presence of known SNPs in variant protein HSTNFR1A_P15 (SEQ ID NO:101) sequence provides support for the deduced sequence of this variant protein according to the present invention).

TABLE 45
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 75 | P -> L |
| 116 | S -> |
| 157 | S -> F |
| 171 | G -> V |
| 225 | P -> |

The glycosylation sites of variant protein HSTNFR1A_P15 (SEQ ID NO:101), as compared to the known protein Tumor necrosis factor receptor superfamily member 1A precursor (SEQ ID NO:96), are described in Table 46 (given according to their position(s) on the amino acid sequence in the first column; the second column indicates whether the glycosylation site is present in the variant protein; and the last column indicates whether the position is different on the variant protein).

TABLE 46
Glycosylation site(s)

| Position(s) on known | Present in | Position(s) on |
|---|---|---|
| amino acid sequence | variant protein? | variant protein |
| 54 | Yes | 54 |
| 145 | Yes | 145 |
| 151 | Yes | 151 |

The variant protein has the following domains, as determined by using InterPro. The domains are described in Table 47:

TABLE 47
InterPro domain(s)

| Domain description | Analysis type | Position(s) on protein |
|---|---|---|
| TNFR | HMMPfam | 44-81, 84-125, 127-166, 168-195 |
| TNFR | HMMSmart | 44-81, 84-125, 127-166, 168-195 |
| TNFR | ScanRegExp | 44-81, 84-125, 127-166 |
| TNFR | ProfileScan | 43-81, 83-125, 126-166 |
| EGF-like | ScanRegExp | 166-179 |
| Cytochrome c | ScanRegExp | 59-64 |
| heme-binding site | | |

Variant protein HSTNFR1A_P15 (SEQ ID NO:101) is encoded by the following transcript(s): HSTNFR1A_T20 (SEQ ID NO:66). The coding portion of transcript HSTNFR1A_T20 (SEQ ID NO:66) starts at position 304 and ends at position 1044. The transcript also has the following SNPs as listed in Table 48 (given according to their position on the nucleotide sequence, with the alternative nucleic acid listed; the last column indicates whether the SNP is known or not; the presence of known SNPs in variant protein HSTNFR1A_P15 (SEQ ID NO:101) sequence provides support for the deduced sequence of this variant protein according to the present invention).

TABLE 48
Nucleic acid SNPs

| Polymorphism | SNP position(s) on nucleotide sequence |
|---|---|
| A -> G | 339, 1557 |
| C -> A | 340, 1695 |
| C -> G | 340, 1369, 1695 |
| C -> T | 436, 527, 573, 773, 1334, 1384, 1405, 1427, 1462, |
| | 1509, 1671 |
| T -> | 487 |
| G -> T | 528, 815, 1393, 1897 |
| G -> C | 528 |
| C -> | 650, 978, 1332, 1369, 1548, 1771 |
| G -> A | 666, 1897 |
| -> G | 927 |
| G -> | 1216, 1547 |
| T -> C | 1294 |
| A -> C | 1843 |

Variant protein HSTNFR1A_P26 (SEQ ID NO:102) according to the present invention is encoded by transcript(s) HSTNFR1A_T35 (SEQ ID NO:69). An alignment is given to the known protein (Tumor necrosis factor receptor superfamily member 1A precursor (SEQ ID NO:96)) at the end of the application. One or more alignments to one or more previously published protein sequences are given at the end of the application. A brief description of the relationship of the variant protein according to the present invention to each such aligned protein is as follows: Comparison report between HSTNFR1A_P26 (SEQ ID NO:102) and known protein(s) TNR1A HUMAN (SEQ ID NO: 96) and NP_{—}001056 (SEQ ID NO: 96):

A. An isolated chimeric polypeptide comprising HSTNFR1A_P26 (SEQ ID NO:102), comprising a first amino acid sequence being at least 70%, optionally at least 80%, preferably at least 85%, more preferably at least 90% and most preferably at least 95%, homologous to a polypeptide having the sequence GDCTKNGSDVPVENLYPSKYTQQVCIHSCFQESMIKECGCAYIFYPRPQNVEYCDYRKHSSWGYCYYKLQ VDFSSDHLGCFTKCRKPCSVTSYQLSAGYSRWPSVTSQEWVFQMLSRQNNYTVNNKRNGVAKVNIFFKEL NYKTNSESPSVT (SEQ ID NO: 146) corresponding to amino acids 1-152 of HSTNFR1A_P26 (SEQ ID NO:102), and a second amino acid sequence being at least 90% homologous to VLLELLVGIYPSGVIGLVPHLGDREKRDSVCPQGKYIHPQNNSICCTKCHKGTYLYNDCPGPGQDTDCREC ESGSFTASENHLRHCLSCSKCRKEMGQVEISSCTVDRDTVCGCRKNQYRHYWSENLFQCFNCSLCLNGTV HLSCQEKQNTVCTCHAGFFLRENECVSCSNCKKSLECTKLCLPQIENVKGTEDSGTTVLLPLVIFFGLCLLSL LFIGLMYRYQRWKSKLYSIVCGKSTPEKEGELEGTTTKPLAPNPSFSPTPGFTPTLGFSPVPSSTFTSSSTYTP GDCPNFAAPRREVAPPYQGADPILATALASDPIPNPLQKWEDSAHKPQSLDTDDPATLYAVVENVPPLRWK EFVRRLGLSDHEIDRLELQNGRCLREAQYSMLATWRRRTPRREATLELLGRVLRDMDLLGCLEDIEEALCG PAALPPAPSLLR corresponding to amino acids 14-455 of known protein(s) TNR1A_HUMAN (SEQ ID NO: 96) and NP_{—}001056 (SEQ ID NO: 96), which also corresponds to amino acids 153-594 of HSTNFR1A_P26 (SEQ ID NO:102), wherein said first amino acid sequence and second amino acid sequence are contiguous and in a sequential order.

B. An isolated polypeptide comprising a head of HSTNFR1A_P26 (SEQ ID NO:102), comprising a polypeptide being at least 70%, optionally at least about 80%, preferably at least about 85%, more preferably at least about 90% and most preferably at least about 95% homologous to the sequence GDCTKNGSDVPVENLYPSKYTQQVCIHSCFQESMIKECGCAYIFYPRPQNVEYCDYRKHSSWGYCYYKLQ VDFSSDHLGCFTKCRKPCSVTSYQLSAGYSRWPSVTSQEWVFQMLSRQNNYTVNNKRNGVAKVNIFFKEL NYKTNSESPSVT (SEQ ID NO: 146) of HSTNFR1A_P26 (SEQ ID NO:102).

The localization of the variant protein was determined according to results from a number of different software programs and analyses, including analyses from SignalP and other specialized programs. The variant protein is believed to be located as follows with regard to the cell: membrane.

Variant protein HSTNFR1A_P26 (SEQ ID NO:102) also has the following non-silent SNPs (Single Nucleotide Polymorphisms) as listed in Table 49, (given according to their position(s) on the amino acid sequence, with the alternative amino acid(s) listed; the last column indicates whether the SNP is known or not; the presence of known SNPs in variant protein HSTNFR1A_P26 (SEQ ID NO:102) sequence provides support for the deduced sequence of this variant protein according to the present invention).

TABLE 49
Amino acid mutations

| SNP position(s) on amino acid | |
|---|---|
| sequence | Alternative amino acid(s) |
| 102 | W -> L |
| 102 | W -> S |
| 105 | V -> L |
| 184 | P -> S |
| 201 | C -> |
| 214 | P -> L |
| 255 | S -> |
| 296 | S -> F |
| 310 | G -> V |
| 405 | P -> |
| 444 | P -> H |
| 468 | L -> |
| 547 | R -> |
| 573 | L -> P |
| 586 | L -> |

The glycosylation sites of variant protein HSTNFR1A_P26 (SEQ ID NO:102), as compared to the known protein Tumor necrosis factor receptor superfamily member 1A precursor (SEQ ID NO:96), are described in Table 50 (given according to their position(s) on the amino acid sequence in the first column; the second column indicates whether the glycosylation site is present in the variant protein; and the last column indicates whether the position is different on the variant protein).

TABLE 50
Glycosylation site(s)

| Position(s) on known | Present in | Position(s) on |
|---|---|---|
| amino acid sequence | variant protein? | variant protein |
| 206 | Yes | 345 |
| 297 | Yes | 436 |
| 303 | Yes | 442 |

The variant protein has the following domains, as determined by using InterPro. The domains are described in Table 51:

TABLE 51
InterPro domain(s)

| | Analysis | |
|---|---|---|
| Domain description | type | Position(s) on protein |
| TNFR | HMMPfam | 183-220, 223-264, 266-305, 307-334 |
| Death | HMMPfam | 496-580 |
| TNFR | HMMSmart | 183-220, 223-264, 266-305, 307-334 |
| TNFR | ProfileScan | 182-220, 222-264, 265-305 |
| TNFR | ScanRegExp | 183-220, 223-264, 266-305 |
| EGF-like | ScanRegExp | 305-318 |
| Na+ channel, | FPrintScan | 20-31, 31-48, 84-104, 127-147 |
| amiloride-sensitive | | |
| Death | HMMSmart | 484-580 |
| Na+ channel, | ScanRegExp | 20-40 |
| amiloride-sensitive | | |
| Death | ProfileScan | 495-580 |
| Cytochrome c | ScanRegExp | 198-203 |
| heme-binding site | | |

Variant protein HSTNFR1A_P26 (SEQ ID NO:102) is encoded by the following transcript(s): HSTNFR1A_T35 (SEQ ID NO:69). The coding portion of transcript HSTNFR1A_T35 (SEQ ID NO:69) starts at position 1 and ends at position 1782. The transcript also has the following SNPs as listed in Table 52 (given according to their position on the nucleotide sequence, with the alternative nucleic acid listed; the last column indicates whether the SNP is known or not; the presence of known SNPs in variant protein HSTNFR1A_P26 (SEQ ID NO:102) sequence provides support for the deduced sequence of this variant protein according to the present invention).

TABLE 52
Nucleic acid SNPs

| Polymorphism | SNP position(s) on nucleotide sequence |
|---|---|
| T -> A | 33 |
| T -> C | 33, 1718 |
| G -> T | 305, 313, 642, 929, 1817, 2321 |
| G -> C | 305, 642 |
| C -> T | 550, 641, 687, 887, 1758, 1808, 1829, 1851, 1886, |
| | 1933, 2095 |
| T -> | 601 |
| C -> | 764, 1213, 1402, 1756, 1793, 1972, 2195 |
| G -> A | 780, 2321 |
| -> G | 1041 |
| C -> A | 1331, 2119 |
| G -> | 1640, 1971 |
| C -> G | 1793, 2119 |
| A -> G | 1981 |
| A -> C | 2267 |

Variant protein HSTNFR1A_P27 (SEQ ID NO:103) according to the present invention is encoded by transcript(s) HSTNFR1A_T38 (SEQ ID NO:70). An alignment is given to the known protein (Tumor necrosis factor receptor superfamily member 1A precursor (SEQ ID NO:96)) at the end of the application. One or more alignments to one or more previously published protein sequences are given at the end of the application. A brief description of the relationship of the variant protein according to the present invention to each such aligned protein is as follows:

### Comparison Report Between HSTNFR1A_P27 (SEQ ID NO:103) and Known Protein(s) TNR1A_HUMAN (SEQ ID NO: 96) and NP_{—}001056 (SEQ ID NO: 96):

A. An isolated chimeric polypeptide comprising HSTNFR1A_P27 (SEQ ID NO:103), comprising a first amino acid sequence being at least 90% homologous to MGLSTVPDLLLPLVLLELLVGIYPSGVIGLVPHLGDREKRDSVCPQGKYIHPQNNSICCTKCHKGTYLYNDC P corresponding to amino acids 1-73 of known protein(s) TNR1A_HUMAN (SEQ ID NO: 96) and NP_{—}001056 (SEQ ID NO: 96), which also corresponds to amino acids 1-73 of HSTNFR1A_P27 (SEQ ID NO:103), and a second amino acid sequence being at least 90% homologous to GFTPTLGFSPVPSSTFTSSSTYTPGDCPNFAAPRREVAPPYQGADPILATALASDPIPNPLQKWEDSAHKPQS LDTDDPATLYAVVENVPPLRWKEFVRRLGLSDHEIDRLELQNGRCLREAQYSMLATWRRRTPRREATLEL LGRVLRDMDLLGCLEDIEEALCGPAALPPAPSLLR corresponding to amino acids 278-455 of known protein(s) TNR1A_HUMAN (SEQ ID NO: 96) and NP_{—}001056 (SEQ ID NO: 96), which also corresponds to amino acids 74-251 of HSTNFR1A_P27 (SEQ ID NO:103), wherein said first amino acid sequence and second amino acid sequence are contiguous and in a sequential order.

B. An isolated chimeric polypeptide comprising an edge portion of HSTNFR1A_P27 (SEQ ID NO:103), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise PG, having a structure as follows: a sequence starting from any of amino acid numbers 73-x to 73; and ending at any of amino acid numbers 74+((n−2)−x), in which x varies from 0 to n−2.

The localization of the variant protein was determined according to results from a number of different software programs and analyses, including analyses from SignalP and other specialized programs. The variant protein is believed to be located as follows with regard to the cell: secreted.

Variant protein HSTNFR1A_P27 (SEQ ID NO:103) also has the following non-silent SNPs (Single Nucleotide Polymorphisms) as listed in Table 53 (given according to their position(s) on the amino acid sequence, with the alternative amino acid(s) listed; the last column indicates whether the SNP is known or not; the presence of known SNPs in variant protein HSTNFR1A_P27 (SEQ ID NO:103) sequence provides support for the deduced sequence of this variant protein according to the present invention).

TABLE 53
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 101 | P -> H |
| 125 | L -> |
| 204 | R -> |
| 230 | L -> P |
| 243 | L -> |

The glycosylation sites of variant protein HSTNFR1A_P27 (SEQ ID NO:103), as compared to the known protein Tumor necrosis factor receptor superfamily member 1A precursor (SEQ ID NO:96), are described in Table 54 (given according to their position(s) on the amino acid sequence in the first column; the second column indicates whether the glycosylation site is present in the variant protein; and the last column indicates whether the position is different on the variant protein).

TABLE 54
Glycosylation site(s)

| Position(s) on known | Present in variant | |
|---|---|---|
| amino acid sequence | protein? | Position(s) on variant protein |
| 54 | Yes | 54 |
| 145 | No | |
| 151 | No | |

The variant protein has the following domains, as determined by using InterPro. The domains are described in Table 55:

TABLE 55
InterPro domain(s)

| Domain description | Analysis type | Position(s) on protein |
|---|---|---|
| Cytochrome c heme-binding site | ScanRegExp | 59-64 |
| Death | HMMPfam | 153-237 |
| TNFR | HMMPfam | 44-80 |
| Death | HMMSmart | 141-237 |
| Death | ProfileScan | 152-237 |

Variant protein HSTNFR1A_P27 (SEQ ID NO:103) is encoded by the following transcript(s): HSTNFR1A_T38 (SEQ ID NO:70). The coding portion of transcript HSTNFR1A_T38 (SEQ ID NO:70) starts at position 304 and ends at position 1056. The transcript also has the following SNPs as listed in Table 56 (given according to their position on the nucleotide sequence, with the alternative nucleic acid listed; the last column indicates whether the SNP is known or not; the presence of known SNPs in variant protein HSTNFR1A_P27 (SEQ ID NO:103) sequence provides support for the deduced sequence of this variant protein according to the present invention).

TABLE 56
Nucleic acid SNPs

| Polymorphism | SNP position(s) on nucleotide sequence |
|---|---|
| A -> G | 339, 1255 |
| C -> A | 340, 605, 1393 |
| C -> G | 340, 1067, 1393 |
| C -> T | 436, 1032, 1082, 1103, 1125, 1160, 1207, 1369 |
| T -> | 487 |
| C -> | 676, 1030, 1067, 1246, 1469 |
| G -> | 914, 1245 |
| T -> C | 992 |
| G -> T | 1091, 1595 |
| A -> C | 1541 |
| G -> A | 1595 |

Variant protein HSTNFR1A_P36 (SEQ ID NO:104) according to the present invention is encoded by transcript(s) HSTNFR1A_T8 (SEQ ID NO:62).

The localization of the variant protein was determined according to results from a number of different software programs and analyses, including analyses from SignalP and other specialized programs. The variant protein is believed to be located as follows with regard to the cell: secreted.

Variant protein HSTNFR1A_P36 (SEQ ID NO:104) also has the following non-silent SNPs (Single Nucleotide Polymorphisms) as listed in Table 57, (given according to their position(s) on the amino acid sequence, with the alternative amino acid(s) listed; the last column indicates whether the SNP is known or not; the presence of known SNPs in variant protein HSTNFR1A_P36 (SEQ ID NO:104) sequence provides support for the deduced sequence of this variant protein according to the present invention).

TABLE 57
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 75 | P -> L |
| 116 | S -> |
| 157 | S -> F |
| 171 | G -> V |
| 212 | R -> K |

The glycosylation sites of variant protein HSTNFR1A_P36 (SEQ ID NO:104), as compared to the known protein Tumor necrosis factor receptor superfamily member 1A precursor (SEQ ID NO:96), are described in Table 58 (given according to their position(s) on the amino acid sequence in the first column; the second column indicates whether the glycosylation site is present in the variant protein; and the last column indicates whether the position is different on the variant protein).

TABLE 58
Glycosylation site(s)

| Position(s) on known amino acid | Present in | Position(s) |
|---|---|---|
| sequence | variant protein? | on variant protein |
| 54 | Yes | 54 |
| 145 | Yes | 145 |
| 151 | Yes | 151 |

The variant protein has the following domains, as determined by using InterPro. The domains are described in Table 59:

TABLE 59
InterPro domain(s)

| Domain description | Analysis type | Position(s) on protein |
|---|---|---|
| TNFR | HMMPfam | 44-81, 84-125, 127-166, 168-195 |
| Cytochrome c | ScanRegExp | 59-64 |
| heme-binding site | | |
| TNFR | ScanRegExp | 44-81, 84-125, 127-166 |
| TNFR | ProfileScan | 43-81, 83-125, 126-166 |
| TNFR | HMMSmart | 44-81, 84-125, 127-166, 168-195 |
| EGF-like | ScanRegExp | 166-179 |

Variant protein HSTNFR1A_P36 (SEQ ID NO:104) is encoded by the following transcript(s): HSTNFR1A_T8 (SEQ ID NO:62). The coding portion of transcript HSTNFR1A_T8 (SEQ ID NO:62) starts at position 304 and ends at position 939. The transcript also has the following SNPs as listed in Table 60 (given according to their position on the nucleotide sequence, with the alternative nucleic acid listed; the last column indicates whether the SNP is known or not; the presence of known SNPs in variant protein HSTNFR1A_P36 (SEQ ID NO:104) sequence provides support for the deduced sequence of this variant protein according to the present invention).

TABLE 60
Nucleic acid SNPs

| Polymorphism | SNP position(s) on nucleotide sequence |
|---|---|
| A -> G | 339, 2008 |
| C -> A | 340, 1358, 2146 |
| C -> G | 340, 1820, 2146 |
| C -> T | 436, 527, 573, 773, 1042, 1785, 1835, |
| | 1856, 1878, 1913, 1960, 2122 |
| T -> | 487 |
| G -> T | 528, 815, 1844, 2348 |
| G -> C | 528 |
| C -> | 650, 1240, 1429, 1783, 1820, 1999, 2222 |
| G -> A | 666, 938, 939, 2348 |
| -> G | 927 |
| G -> | 1667, 1998 |
| T -> C | 1745 |
| A -> C | 2294 |

Variant protein HSTNFR1A_P37 (SEQ ID NO:105) according to the present invention is encoded by transcript(s) HSTNFR1A_T23 (SEQ ID NO:67).

The localization of the variant protein was determined according to results from a number of different software programs and analyses, including analyses from SignalP and other specialized programs. The variant protein is believed to be located as follows with regard to the cell: secreted.

Variant protein HSTNFR1A_P37 (SEQ ID NO:105) also has the following non-silent SNPs (Single Nucleotide Polymorphisms) as listed in Table 61, (given according to their position(s) on the amino acid sequence, with the alternative amino acid(s) listed; the last column indicates whether the SNP is known or not; the presence of known SNPs in variant protein HSTNFR1A_P37 (SEQ ID NO:105) sequence provides support for the deduced sequence of this variant protein according to the present invention).

TABLE 61
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |

The glycosylation sites of variant protein HSTNFR1A_P37 (SEQ ID NO:105), as compared to the known protein Tumor necrosis factor receptor superfamily member 1A precursor (SEQ ID NO:96), are described in Table 62 (given according to their position(s) on the amino acid sequence in the first column; the second column indicates whether the glycosylation site is present in the variant protein; and the last column indicates whether the position is different on the variant protein).

TABLE 62
Glycosylation site(s)

| Position(s) on known | Present in | Position(s) on |
|---|---|---|
| amino acid sequence | variant protein? | variant protein |
| 54 | Yes | 54 |
| 145 | No | |
| 151 | No | |

The variant protein has the following domains, as determined by using InterPro. The domains are described in Table 63:

TABLE 63
InterPro domain(s)

| Domain description | Analysis type | Position(s) on protein |
|---|---|---|
| Cytochrome c heme-binding site | ScanRegExp | 59-64 |

Variant protein HSTNFR1A_P37 (SEQ ID NO:105) is encoded by the following transcript(s): HSTNFR1A_T23 (SEQ ID NO:67). The coding portion of transcript HSTNFR1A_T23 (SEQ ID NO:67) starts at position 304 and ends at position 498. The transcript also has the following SNPs as listed in Table 64 (given according to their position on the nucleotide sequence, with the alternative nucleic acid listed; the last column indicates whether the SNP is known or not; the presence of known SNPs in variant protein HSTNFR1A_P37 (SEQ ID NO:105) sequence provides support for the deduced sequence of this variant protein according to the present invention).

TABLE 64
Nucleic acid SNPs

| Polymorphism | SNP position(s) on nucleotide sequence |
|---|---|
| A -> G | 339, 1892 |
| C -> A | 340, 1242, 2030 |
| C -> G | 340, 1704, 2030 |
| C -> T | 436, 552, 598, 798, 1669, 1719, 1740, 1762, 1797, 1844, |
| | 2006 |
| T -> | 487 |
| G -> T | 553, 840, 1728, 2232 |
| G -> C | 553 |
| C -> | 675, 1124, 1313, 1667, 1704, 1883, 2106 |
| G -> A | 691, 2232 |
| -> G | 952 |
| G -> | 1551, 1882 |
| T -> C | 1629 |
| A -> C | 2178 |

Variant protein HSTNFR1A_P38 (SEQ ID NO:106) according to the present invention is encoded by transcript(s) HSTNFR1A_T24 (SEQ ID NO:68).

The localization of the variant protein was determined according to results from a number of different software programs and analyses, including analyses from SignalP and other specialized programs. The variant protein is believed to be located as follows with regard to the cell: secreted.

Variant protein HSTNFR1A_P38 (SEQ ID NO:106) also has the following non-silent SNPs (Single Nucleotide Polymorphisms) as listed in Table 65, (given according to their position(s) on the amino acid sequence, with the alternative amino acid(s) listed; the last column indicates whether the SNP is known or not; the presence of known SNPs in variant protein HSTNFR1A_P38 (SEQ ID NO:106) sequence provides support for the deduced sequence of this variant protein according to the present invention).

TABLE 65
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |
| 76 | R -> L |
| 76 | R -> P |
| 76 | R -> W |
| 91 | P -> L |
| 117 | L -> |
| 122 | G -> E |
| 158 | P -> S |
| 172 | V -> F |

The glycosylation sites of variant protein HSTNFR1A_P38 (SEQ ID NO:106), as compared to the known protein Tumor necrosis factor receptor superfamily member 1A precursor (SEQ ID NO:96), are described in Table 66 (given according to their position(s) on the amino acid sequence in the first column; the second column indicates whether the glycosylation site is present in the variant protein; and the last column indicates whether the position is different on the variant protein).

TABLE 66
Glycosylation site(s)

| Position(s) on known | Present in | Position(s) |
|---|---|---|
| amino acid sequence | variant protein? | on variant protein |
| 54 | Yes | 54 |
| 145 | No | |
| 151 | No | |

The variant protein has the following domains, as determined by using InterPro. The domains are described in Table 67:

TABLE 67
InterPro domain(s)

| Domain description | Analysis type | Position(s) on protein |
|---|---|---|
| Cytochrome c heme-binding site | ScanRegExp | 59-64 |

Variant protein HSTNFR1A_P38 (SEQ ID NO:106) is encoded by the following transcript(s): HSTNFR1A_T24 (SEQ ID NO:68). The coding portion of transcript HSTNFR1A_T24 (SEQ ID NO:68) starts at position 304 and ends at position 825. The transcript also has the following SNPs as listed in Table 68 (given according to their position on the nucleotide sequence, with the alternative nucleic acid listed; the last column indicates whether the SNP is known or not; the presence of known SNPs in variant protein HSTNFR1A_P38 (SEQ ID NO:106) sequence provides support for the deduced sequence of this variant protein according to the present invention).

TABLE 68
Nucleic acid SNPs

| Polymorphism | SNP position(s) on nucleotide sequence |
|---|---|
| A -> G | 339, 1869 |
| C -> A | 340, 1219, 2007 |
| C -> G | 340, 1681, 2007 |
| C -> T | 436, 529, 575, 775, 1646, 1696, 1717, 1739, 1774, |
| | 1821, 1983 |
| T -> | 487 |
| G -> T | 530, 817, 1705, 2209 |
| G -> C | 530 |
| C -> | 652, 1101, 1290, 1644, 1681, 1860, 2083 |
| G -> A | 668, 2209 |
| -> G | 929 |
| G -> | 1528, 1859 |
| T -> C | 1606 |
| A -> C | 2155 |

Variant protein HSTNFR1A_P43 (SEQ ID NO:107) according to the present invention is encoded by transcript(s) HSTNFR1A_T41 (SEQ ID NO:71).

The localization of the variant protein was determined according to results from a number of different software programs and analyses, including analyses from SignalP and other specialized programs. The variant protein is believed to be located as follows with regard to the cell: secreted.

Variant protein HSTNFR1A_P43 (SEQ ID NO:107) also has the following non-silent SNPs (Single Nucleotide Polymorphisms) as listed in Table 69, (given according to their position(s) on the amino acid sequence, with the alternative amino acid(s) listed; the last column indicates whether the SNP is known or not; the presence of known SNPs in variant protein HSTNFR1A_P43 (SEQ ID NO:107) sequence provides support for the deduced sequence of this variant protein according to the present invention).

TABLE 69
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |

The glycosylation sites of variant protein HSTNFR1A_P43 (SEQ ID NO:107), as compared to the known protein Tumor necrosis factor receptor superfamily member 1A precursor (SEQ ID NO:96), are described in Table 70 (given according to their position(s) on the amino acid sequence in the first column; the second column indicates whether the glycosylation site is present in the variant protein; and the last column indicates whether the position is different on the variant protein).

TABLE 70
Glycosylation site(s)

| Position(s) on known | Present | Position(s) |
|---|---|---|
| amino acid sequence | in variant protein? | on variant protein |
| 54 | Yes | 54 |
| 145 | No | |
| 151 | No | |

The variant protein has the following domains, as determined by using InterPro. The domains are described in Table 71:

TABLE 71
InterPro domain(s)

| Domain description | Analysis type | Position(s) on protein |
|---|---|---|
| Cytochrome c heme-binding site | ScanRegExp | 59-64 |

Variant protein HSTNFR1A_P43 (SEQ ID NO:107) is encoded by the following transcript(s): HSTNFR1A_T41 (SEQ ID NO:71). The coding portion of transcript HSTNFR1A_T41 (SEQ ID NO:71) starts at position 304 and ends at position 519. The transcript also has the following SNPs as listed in Table 72 (given according to their position on the nucleotide sequence, with the alternative nucleic acid listed; the last column indicates whether the SNP is known or not; the presence of known SNPs in variant protein HSTNFR1A_P43 (SEQ ID NO:107) sequence provides support for the deduced sequence of this variant protein according to the present invention).

TABLE 72
Nucleic acid SNPs

| Polymorphism | SNP position(s) on nucleotide sequence |
|---|---|
| A -> G | 339, 1673 |
| C -> A | 340, 1023, 1811 |
| C -> G | 340, 1485, 1811 |
| C -> T | 436, 579, 1450, 1500, 1521, 1543, 1578, 1625, 1787 |
| T -> | 487 |
| G -> T | 621, 1509, 2013 |
| -> G | 733 |
| C -> | 905, 1094, 1448, 1485, 1664, 1887 |
| G -> | 1332, 1663 |
| T -> C | 1410 |
| A -> C | 1959 |
| G -> A | 2013 |

Variant protein HSTNFR1A_P44 (SEQ ID NO:108) according to the present invention is encoded by transcript(s) HSTNFR1A_T44 (SEQ ID NO:72).

The localization of the variant protein was determined according to results from a number of different software programs and analyses, including analyses from SignalP and other specialized programs. The variant protein is believed to be located as follows with regard to the cell: secreted.

Variant protein HSTNFR1A_P44 (SEQ ID NO:108) also has the following non-silent SNPs (Single Nucleotide Polymorphisms) as listed in Table 73, (given according to their position(s) on the amino acid sequence, with the alternative amino acid(s) listed; the last column indicates whether the SNP is known or not; the presence of known SNPs in variant protein HSTNFR1A_P44 (SEQ ID NO:108) sequence provides support for the deduced sequence of this variant protein according to the present invention).

TABLE 73
Amino acid mutations

| SNP position(s) on amino acid | |
|---|---|
| sequence | Alternative amino acid(s) |
| 13 | L -> M |
| 13 | L -> V |
| 45 | P -> S |
| 62 | C -> |

The glycosylation sites of variant protein HSTNFR1A_P44 (SEQ ID NO:108), as compared to the known protein Tumor necrosis factor receptor superfamily member 1A precursor (SEQ ID NO:96), are described in Table 74 (given according to their position(s) on the amino acid sequence in the first column; the second column indicates whether the glycosylation site is present in the variant protein; and the last column indicates whether the position is different on the variant protein).

TABLE 74
Glycosylation site(s)

| Position(s) on known | Present in | Position(s) on |
|---|---|---|
| amino acid sequence | variant protein? | variant protein |
| 54 | Yes | 54 |
| 145 | No | |
| 151 | No | |

The variant protein has the following domains, as determined by using InterPro. The domains are described in Table 75:

TABLE 75
InterPro domain(s)

| Domain description | Analysis type | Position(s) on protein |
|---|---|---|
| Cytochrome c heme-binding site | ScanRegExp | 59-64 |

Variant protein HSTNFR1A_P44 (SEQ ID NO:108) is encoded by the following transcript(s): HSTNFR1A_T44 (SEQ ID NO:72). The coding portion of transcript HSTNFR1A_T44 (SEQ ID NO:72) starts at position 304 and ends at position 516. The transcript also has the following SNPs as listed in Table 76 (given according to their position on the nucleotide sequence, with the alternative nucleic acid listed; the last column indicates whether the SNP is known or not; the presence of known SNPs in variant protein HSTNFR1A_P44 (SEQ ID NO:108) sequence provides support for the deduced sequence of this variant protein according to the present invention).

TABLE 76
Nucleic acid SNPs

| Polymorphism | SNP position(s) on nucleotide sequence |
|---|---|
| A -> G | 339 |
| C -> A | 340 |
| C -> G | 340 |
| C -> T | 436 |
| T -> | 487 |
| G -> A | 516 |

As noted above, cluster HSTNFR1A features 23 segment(s), which were listed in Table 27. These segment(s) are portions of nucleic acid sequence(s) which are described herein separately because they are of particular interest. A description of each segment according to the present invention is now provided.

Segment cluster HSTNFR1A_N37 (SEQ ID NO:73) according to the present invention is supported by 2 libraries. The number of libraries was determined as previously described. This segment can be found in the following transcript(s): HSTNFR1A_T44 (SEQ ID NO:72). Table 77 below describes the starting and ending position of this segment on each transcript.

TABLE 77
Segment location on transcripts

| | Segment | Segment |
|---|---|---|
| Transcript name | starting position | ending position |
| HSTNFR1A_T44 (SEQ ID NO: 72) | 497 | 667 |

Segment cluster HSTNFR1A_N62 (SEQ ID NO:74) according to the present invention is supported by 17 libraries. The number of libraries was determined as previously described. This segment can be found in the following transcript(s): HSTNFR1A_T12 (SEQ ID NO:63) and HSTNFR1A_T5 (SEQ ID NO:60). Table 78 below describes the starting and ending position of this segment on each transcript.

TABLE 78
Segment location on transcripts

| | Segment | Segment |
|---|---|---|
| Transcript name | starting position | ending position |
| HSTNFR1A_T12 (SEQ ID NO: 63) | 977 | 1921 |
| HSTNFR1A_T5 (SEQ ID NO: 60) | 977 | 1921 |

Segment cluster HSTNFR1A_N64 (SEQ ID NO:75) according to the present invention is supported by 4 libraries. The number of libraries was determined as previously described. This segment can be found in the following transcript(s): HSTNFR1A T12 (SEQ ID NO:63) and HSTNFR1A_T8 (SEQ ID NO:62). Table 79 below describes the starting and ending position of this segment on each transcript.

TABLE 79
Segment location on transcripts

| | Segment | Segment |
|---|---|---|
| Transcript name | starting position | ending position |
| HSTNFR1A_T12 (SEQ ID NO: 63) | 1996 | 2136 |
| HSTNFR1A_T8 (SEQ ID NO: 62) | 929 | 1069 |

According to an optional embodiment of the present invention, short segments related to the above cluster are also provided. These segments are up to about 120 by in length, and so are included in a separate description.

Segment cluster HSTNFR1A_N2 (SEQ ID NO:76) according to the present invention is supported by 2 libraries. The number of libraries was determined as previously described. This segment can be found in the following transcript(s): HSTNFR1A_T35 (SEQ ID NO:69). Table 80 below describes the starting and ending position of this segment on each transcript.

TABLE 80
Segment location on transcripts

| | Segment | Segment |
|---|---|---|
| Transcript name | starting position | ending position |
| HSTNFR1A_T35 (SEQ ID NO: 69) | 1 | 69 |

Segment cluster HSTNFR1A_N4 (SEQ ID NO:77) according to the present invention is supported by 2 libraries. The number of libraries was determined as previously described. This segment can be found in the following transcript(s): HSTNFR1A_T35 (SEQ ID NO:69). Table 81 below describes the starting and ending position of this segment on each transcript.

TABLE 81
Segment location on transcripts

| | Segment | Segment |
|---|---|---|
| Transcript name | starting position | ending position |
| HSTNFR1A_T35 (SEQ ID NO: 69) | 70 | 187 |

Segment cluster HSTNFR1A_N6 (SEQ ID NO:78) according to the present invention is supported by 2 libraries. The number of libraries was determined as previously described. This segment can be found in the following transcript(s): HSTNFR1A_T35 (SEQ ID NO:69). Table 82 below describes the starting and ending position of this segment on each transcript.

TABLE 82
Segment location on transcripts

| | Segment | Segment |
|---|---|---|
| Transcript name | starting position | ending position |
| HSTNFR1A_T35 (SEQ ID NO: 69) | 188 | 266 |

Segment cluster HSTNFR1A_N8 (SEQ ID NO:79) according to the present invention is supported by 2 libraries. The number of libraries was determined as previously described. This segment can be found in the following transcript(s): HSTNFR1A_T35 (SEQ ID NO:69). Table 83 below describes the starting and ending position of this segment on each transcript.

TABLE 83
Segment location on transcripts

| | Segment | Segment |
|---|---|---|
| Transcript name | starting position | ending position |
| HSTNFR1A_T35 (SEQ ID NO: 69) | 267 | 324 |

Segment cluster HSTNFR1A_N10 (SEQ ID NO:80) according to the present invention is supported by 3 libraries. The number of libraries was determined as previously described. This segment can be found in the following transcript(s): HSTNFR1A_T35 (SEQ ID NO:69). Table 84 below describes the starting and ending position of this segment on each transcript.

TABLE 84
Segment location on transcripts

| | Segment | Segment |
|---|---|---|
| Transcript name | starting position | ending position |
| HSTNFR1A_T35 (SEQ ID NO: 69) | 325 | 380 |

Segment cluster HSTNFR1A_N12 (SEQ ID NO:81) according to the present invention is supported by 3 libraries. The number of libraries was determined as previously described. This segment can be found in the following transcript(s): HSTNFR1A_T35 (SEQ ID NO:69). Table 85 below describes the starting and ending position of this segment on each transcript.

TABLE 85
Segment location on transcripts

| | Segment | Segment |
|---|---|---|
| Transcript name | starting position | ending position |
| HSTNFR1A_T35 (SEQ ID NO: 69) | 381 | 456 |

Segment cluster HSTNFR1A_N31 (SEQ ID NO:82) according to the present invention is supported by 285 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T38 (SEQ ID NO:70), HSTNFR1A_T41 (SEQ ID NO:71), HSTNFR1A_T44 (SEQ ID NO:72), HSTNFR1A_T5 (SEQ ID NO:60), HSTNFR1A_T6 (SEQ ID NO:61) and HSTNFR1A_T8 (SEQ ID NO:62). Table 86 below describes the starting and ending position of this segment on each transcript.

TABLE 86
Segment location on transcripts

| | Segment | Segment |
|---|---|---|
| Transcript name | starting position | ending position |
| HSTNFR1A_T12 (SEQ ID NO: 63) | 343 | 348 |
| HSTNFR1A_T14 (SEQ ID NO: 64) | 343 | 348 |
| HSTNFR1A_T19 (SEQ ID NO: 65) | 343 | 348 |
| HSTNFR1A_T20 (SEQ ID NO: 66) | 343 | 348 |
| HSTNFR1A_T23 (SEQ ID NO: 67) | 343 | 348 |
| HSTNFR1A_T24 (SEQ ID NO: 68) | 343 | 348 |
| HSTNFR1A_T35 (SEQ ID NO: 69) | 457 | 462 |
| HSTNFR1A_T38 (SEQ ID NO: 70) | 343 | 348 |
| HSTNFR1A_T41 (SEQ ID NO: 71) | 343 | 348 |
| HSTNFR1A_T44 (SEQ ID NO: 72) | 343 | 348 |
| HSTNFR1A_T5 (SEQ ID NO: 60) | 343 | 348 |
| HSTNFR1A_T6 (SEQ ID NO: 61) | 343 | 348 |
| HSTNFR1A_T8 (SEQ ID NO: 62) | 343 | 348 |

Segment cluster HSTNFR1A_N36 (SEQ ID NO:83) according to the present invention is supported by 273 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T38 (SEQ ID NO:70), HSTNFR1A_T41 (SEQ ID NO:71), HSTNFR1A_T44 (SEQ ID NO:72), HSTNFR1A_T5 (SEQ ID NO:60), HSTNFR1A_T6 (SEQ ID NO:61) and HSTNFR1A_T8 (SEQ ID NO:62). Table 87 below describes the starting and ending position of this segment on each transcript.

TABLE 87
Segment location on transcripts

| | Segment | Segment |
|---|---|---|
| Transcript name | starting position | ending position |
| HSTNFR1A_T12 (SEQ ID NO: 63) | 432 | 496 |
| HSTNFR1A_T14 (SEQ ID NO: 64) | 432 | 496 |
| HSTNFR1A_T19 (SEQ ID NO: 65) | 432 | 496 |
| HSTNFR1A_T20 (SEQ ID NO: 66) | 432 | 496 |
| HSTNFR1A_T23 (SEQ ID NO: 67) | 432 | 496 |
| HSTNFR1A_T24 (SEQ ID NO: 68) | 432 | 496 |
| HSTNFR1A_T35 (SEQ ID NO: 69) | 546 | 610 |
| HSTNFR1A_T38 (SEQ ID NO: 70) | 432 | 496 |
| HSTNFR1A_T41 (SEQ ID NO: 71) | 432 | 496 |
| HSTNFR1A_T44 (SEQ ID NO: 72) | 432 | 496 |
| HSTNFR1A_T5 (SEQ ID NO: 60) | 432 | 496 |
| HSTNFR1A_T6 (SEQ ID NO: 61) | 432 | 496 |
| HSTNFR1A_T8 (SEQ ID NO: 62) | 432 | 496 |

Segment cluster HSTNFR1A_N39 (SEQ ID NO:84) according to the present invention is supported by 13 libraries. The number of libraries was determined as previously described. This segment can be found in the following transcript(s): HSTNFR1A_T23 (SEQ ID NO:67). Table 88 below describes the starting and ending position of this segment on each transcript.

TABLE 88
Segment location on transcripts

| | Segment | Segment |
|---|---|---|
| Transcript name | starting position | ending position |
| HSTNFR1A_T23 (SEQ ID NO: 67) | 497 | 519 |

Segment cluster HSTNFR1A_N40 (SEQ ID NO:85) according to the present invention is supported by 24 libraries. The number of libraries was determined as previously described. This segment can be found in the following transcript(s): HSTNFR1A_T23 (SEQ ID NO:67) and HSTNFR1A_T24 (SEQ ID NO:68). Table 89 below describes the starting and ending position of this segment on each transcript.

TABLE 89
Segment location on transcripts

| | Segment | Segment |
|---|---|---|
| Transcript name | starting position | ending position |
| HSTNFR1A_T23 (SEQ ID NO: 67) | 520 | 521 |
| HSTNFR1A_T24 (SEQ ID NO: 68) | 497 | 498 |

Segment cluster HSTNFR1A_N43 (SEQ ID NO:86) according to the present invention is supported by 266 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T38 (SEQ ID NO:70), HSTNFR1A_T5 (SEQ ID NO:60), HSTNFR1A_T6 (SEQ ID NO:61) and HSTNFR1A_T8 (SEQ ID NO:62). Table 90 below describes the starting and ending position of this segment on each transcript.

TABLE 90
Segment location on transcripts

| | Segment | Segment |
|---|---|---|
| Transcript name | starting position | ending position |
| HSTNFR1A_T12 (SEQ ID NO: 63) | 521 | 523 |
| HSTNFR1A_T14 (SEQ ID NO: 64) | 521 | 523 |
| HSTNFR1A_T19 (SEQ ID NO: 65) | 521 | 523 |
| HSTNFR1A_T20 (SEQ ID NO: 66) | 521 | 523 |
| HSTNFR1A_T23 (SEQ ID NO: 67) | 546 | 548 |
| HSTNFR1A_T24 (SEQ ID NO: 68) | 523 | 525 |
| HSTNFR1A_T35 (SEQ ID NO: 69) | 635 | 637 |
| HSTNFR1A_T38 (SEQ ID NO: 70) | 521 | 523 |
| HSTNFR1A_T5 (SEQ ID NO: 60) | 521 | 523 |
| HSTNFR1A_T6 (SEQ ID NO: 61) | 521 | 523 |
| HSTNFR1A_T8 (SEQ ID NO: 62) | 521 | 523 |

Segment cluster HSTNFR1A_N53 (SEQ ID NO:87) according to the present invention is supported by 179 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 91 below describes the starting and ending position of this segment on each transcript.

TABLE 91
Segment location on transcripts

| | Segment | Segment |
|---|---|---|
| Transcript name | starting position | ending position |
| HSTNFR1A_T12 (SEQ ID NO: 63) | 691 | 693 |
| HSTNFR1A_T14 (SEQ ID NO: 64) | 691 | 693 |
| HSTNFR1A_T19 (SEQ ID NO: 65) | 691 | 693 |
| HSTNFR1A_T20 (SEQ ID NO: 66) | 691 | 693 |
| HSTNFR1A_T23 (SEQ ID NO: 67) | 716 | 718 |
| HSTNFR1A_T24 (SEQ ID NO: 68) | 693 | 695 |
| HSTNFR1A_T35 (SEQ ID NO: 69) | 805 | 807 |
| HSTNFR1A_T41 (SEQ ID NO: 71) | 497 | 499 |
| HSTNFR1A_T5 (SEQ ID NO: 60) | 691 | 693 |
| HSTNFR1A_T6 (SEQ ID NO: 61) | 691 | 693 |
| HSTNFR1A_T8 (SEQ ID NO: 62) | 691 | 693 |

Segment cluster HSTNFR1A_N56 (SEQ ID NO:88) according to the present invention is supported by 156 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 92 below describes the starting and ending position of this segment on each transcript.

TABLE 92
Segment location on transcripts

| | Segment | Segment |
|---|---|---|
| Transcript name | starting position | ending position |
| HSTNFR1A_T12 (SEQ ID NO: 63) | 776 | 854 |
| HSTNFR1A_T14 (SEQ ID NO: 64) | 776 | 854 |
| HSTNFR1A_T19 (SEQ ID NO: 65) | 776 | 854 |
| HSTNFR1A_T20 (SEQ ID NO: 66) | 776 | 854 |
| HSTNFR1A_T23 (SEQ ID NO: 67) | 801 | 879 |
| HSTNFR1A_T24 (SEQ ID NO: 68) | 778 | 856 |
| HSTNFR1A_T35 (SEQ ID NO: 69) | 890 | 968 |
| HSTNFR1A_T41 (SEQ ID NO: 71) | 582 | 660 |
| HSTNFR1A_T5 (SEQ ID NO: 60) | 776 | 854 |
| HSTNFR1A_T6 (SEQ ID NO: 61) | 776 | 854 |
| HSTNFR1A_T8 (SEQ ID NO: 62) | 776 | 854 |

Segment cluster HSTNFR1A_N59 (SEQ ID NO:89) according to the present invention is supported by 13 libraries. The number of libraries was determined as previously described. This segment can be found in the following transcript(s): HSTNFR1A_T12 (SEQ ID NO:63), HSTNFR1A_T19 (SEQ ID NO:65), HSTNFR1A_T5 (SEQ ID NO:60) and HSTNFR1A_T6 (SEQ ID NO:61). Table 93 below describes the starting and ending position of this segment on each transcript.

TABLE 93
Segment location on transcripts

| | Segment | Segment |
|---|---|---|
| Transcript name | starting position | ending position |
| HSTNFR1A_T12 (SEQ ID NO: 63) | 855 | 956 |
| HSTNFR1A_T19 (SEQ ID NO: 65) | 855 | 956 |
| HSTNFR1A_T5 (SEQ ID NO: 60) | 855 | 956 |
| HSTNFR1A_T6 (SEQ ID NO: 61) | 855 | 956 |

Segment cluster HSTNFR1A_N60 (SEQ ID NO:90) according to the present invention is supported by 10 libraries. The number of libraries was determined as previously described. This segment can be found in the following transcript(s): HSTNFR1A_T12 (SEQ ID NO:63), HSTNFR1A_T5 (SEQ ID NO:60) and HSTNFR1A_T6 (SEQ ID NO:61). Table 94 below describes the starting and ending position of this segment on each transcript.

TABLE 94
Segment location on transcripts

| | Segment | Segment |
|---|---|---|
| Transcript name | starting position | ending position |
| HSTNFR1A_T12 (SEQ ID NO: 63) | 957 | 972 |
| HSTNFR1A_T5 (SEQ ID NO: 60) | 957 | 972 |
| HSTNFR1A_T6 (SEQ ID NO: 61) | 957 | 972 |

Segment cluster HSTNFR1A_N61 (SEQ ID NO:91) according to the present invention is supported by 4 libraries. The number of libraries was determined as previously described. This segment can be found in the following transcript(s): HSTNFR1A_T12 (SEQ ID NO:63) and HSTNFR1A_T5 (SEQ ID NO:60). Table 95 below describes the starting and ending position of this segment on each transcript.

TABLE 95
Segment location on transcripts

| | Segment | Segment |
|---|---|---|
| Transcript name | starting position | ending position |
| HSTNFR1A_T12 (SEQ ID NO: 63) | 973 | 976 |
| HSTNFR1A_T5 (SEQ ID NO: 60) | 973 | 976 |

Segment cluster HSTNFR1A_N63 (SEQ ID NO:92) according to the present invention is supported by 142 libraries. The number of libraries was determined as previously described. This segment can be found in the following transcript(s): HSTNFR1A_T12 (SEQ ID NO:63), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 96 below describes the starting and ending position of this segment on each transcript.

TABLE 96
Segment location on transcripts

| | Segment | Segment |
|---|---|---|
| Transcript name | starting position | ending position |
| HSTNFR1A_T12 (SEQ ID NO: 63) | 1922 | 1995 |
| HSTNFR1A_T20 (SEQ ID NO: 66) | 855 | 928 |
| HSTNFR1A_T23 (SEQ ID NO: 67) | 880 | 953 |
| HSTNFR1A_T24 (SEQ ID NO: 68) | 857 | 930 |
| HSTNFR1A_T35 (SEQ ID NO: 69) | 969 | 1042 |
| HSTNFR1A_T41 (SEQ ID NO: 71) | 661 | 734 |
| HSTNFR1A_T5 (SEQ ID NO: 60) | 1922 | 1995 |
| HSTNFR1A_T6 (SEQ ID NO: 61) | 973 | 1046 |
| HSTNFR1A_T8 (SEQ ID NO: 62) | 855 | 928 |

Segment cluster HSTNFR1A_N65 (SEQ ID NO:93) according to the present invention is supported by 117 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T23 (SEQ ID NO:67), HSTNFR1A_T24 (SEQ ID NO:68), HSTNFR1A_T35 (SEQ ID NO:69), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 97 below describes the starting and ending position of this segment on each transcript.

TABLE 97
Segment location on transcripts

| | Segment | Segment |
|---|---|---|
| Transcript name | starting position | ending position |
| HSTNFR1A_T12 (SEQ ID NO: 63) | 2137 | 2159 |
| HSTNFR1A_T14 (SEQ ID NO: 64) | 855 | 877 |
| HSTNFR1A_T23 (SEQ ID NO: 67) | 954 | 976 |
| HSTNFR1A_T24 (SEQ ID NO: 68) | 931 | 953 |
| HSTNFR1A_T35 (SEQ ID NO: 69) | 1043 | 1065 |
| HSTNFR1A_T41 (SEQ ID NO: 71) | 735 | 757 |
| HSTNFR1A_T5 (SEQ ID NO: 60) | 1996 | 2018 |
| HSTNFR1A_T6 (SEQ ID NO: 61) | 1047 | 1069 |
| HSTNFR1A_T8 (SEQ ID NO: 62) | 1070 | 1092 |

Segment cluster HSTNFR1A_N75 (SEQ ID NO:94) according to the present invention is supported by 111 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T23 (SEQ ID NO:67), HSTNFR1A_T24 (SEQ ID NO:68), HSTNFR1A_T35 (SEQ ID NO:69), HSTNFR1A_T38 (SEQ ID NO:70), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 98 below describes the starting and ending position of this segment on each transcript.

TABLE 98
Segment location on transcripts

| | Segment | Segment |
|---|---|---|
| Transcript name | starting position | ending position |
| HSTNFR1A_T12 (SEQ ID NO: 63) | 2344 | 2368 |
| HSTNFR1A_T14 (SEQ ID NO: 64) | 1062 | 1086 |
| HSTNFR1A_T19 (SEQ ID NO: 65) | 957 | 981 |
| HSTNFR1A_T23 (SEQ ID NO: 67) | 1161 | 1185 |
| HSTNFR1A_T24 (SEQ ID NO: 68) | 1138 | 1162 |
| HSTNFR1A_T35 (SEQ ID NO: 69) | 1250 | 1274 |
| HSTNFR1A_T38 (SEQ ID NO: 70) | 524 | 548 |
| HSTNFR1A_T41 (SEQ ID NO: 71) | 942 | 966 |
| HSTNFR1A_T5 (SEQ ID NO: 60) | 2203 | 2227 |
| HSTNFR1A_T6 (SEQ ID NO: 61) | 1254 | 1278 |
| HSTNFR1A_T8 (SEQ ID NO: 62) | 1277 | 1301 |

Segment cluster HSTNFR1A_N82 (SEQ ID NO:95) according to the present invention is supported by 104 libraries. The number of libraries was determined as previously described. This segment can be found in the following transcript(s): HSTNFR1A_T12 (SEQ ID NO:63), HSTNFR1A_T14 (SEQ ID NO:64), HSTNFR1A_T19 (SEQ ID NO:65), HSTNFR1A_T20 (SEQ ID NO:66), HSTNFR1A_T23 (SEQ ID NO:67), HSTNFR1A_T24 (SEQ ID NO:68), HSTNFR1A_T35 (SEQ ID NO:69), HSTNFR1A_T38 (SEQ ID NO:70), HSTNFR1A_T41 (SEQ ID NO:71), HSTNFR1A_T5 (SEQ ID NO:60), HSTNFR1A_T6 (SEQ ID NO:61) and HSTNFR1A_T8 (SEQ ID NO:62). Table 99 below describes the starting and ending position of this segment on each transcript.

TABLE 99
Segment location on transcripts

| | Segment | Segment |
|---|---|---|
| Transcript name | starting position | ending position |
| HSTNFR1A_T12 (SEQ ID NO: 63) | 2447 | 2477 |
| HSTNFR1A_T14 (SEQ ID NO: 64) | 1165 | 1195 |
| HSTNFR1A_T19 (SEQ ID NO: 65) | 1060 | 1090 |
| HSTNFR1A_T20 (SEQ ID NO: 66) | 929 | 959 |
| HSTNFR1A_T23 (SEQ ID NO: 67) | 1264 | 1294 |
| HSTNFR1A_T24 (SEQ ID NO: 68) | 1241 | 1271 |
| HSTNFR1A_T35 (SEQ ID NO: 69) | 1353 | 1383 |
| HSTNFR1A_T38 (SEQ ID NO: 70) | 627 | 657 |
| HSTNFR1A_T41 (SEQ ID NO: 71) | 1045 | 1075 |
| HSTNFR1A_T5 (SEQ ID NO: 60) | 2306 | 2336 |
| HSTNFR1A_T6 (SEQ ID NO: 61) | 1357 | 1387 |
| HSTNFR1A_T8 (SEQ ID NO: 62) | 1380 | 1410 |

### Variant Protein Alignment to the Previously Known Protein:

Alignment of: HSTNFR1A_P26 (SEQ ID NO:102)×known protein(s) TNR1A_HUMAN (SEQ ID NO: 96) and NP_{—}001056 (SEQ ID NO: 96):
Total length: 607
Matching length: 442

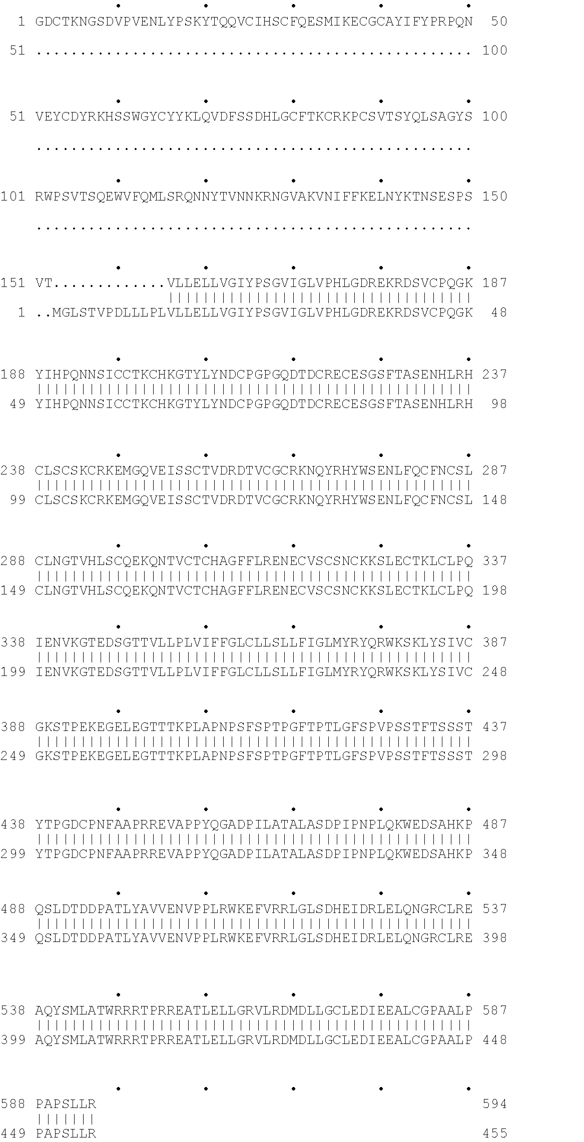

Alignment of: HSTNFR1A_P27 (SEQ ID NO:103)×known protein(s) TNR1A_HUMAN (SEQ ID NO: 96) and NP_{—}001056 (SEQ ID NO: 96):
Total length: 455
Matching length: 251

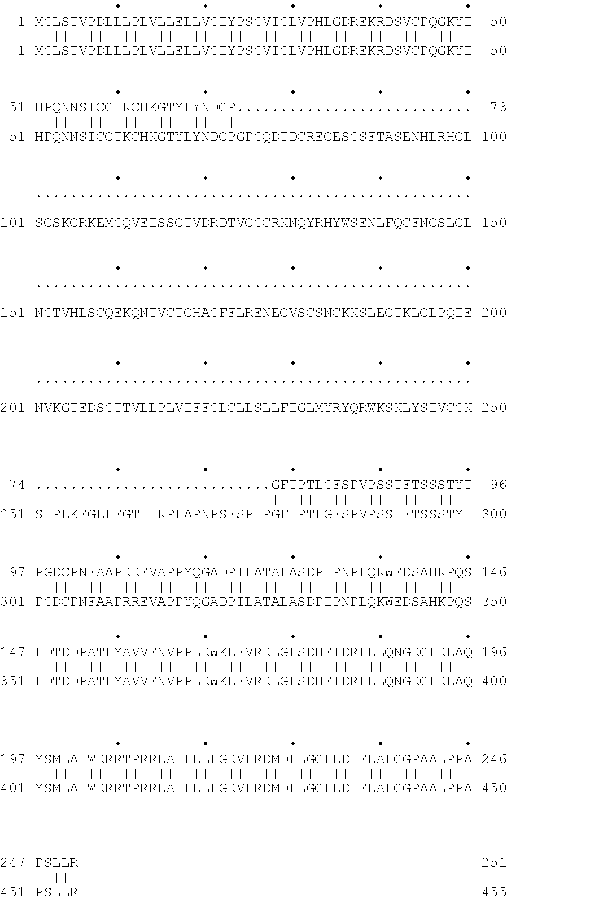

Expression of Homo sapiens TNFRFS1A Cluster Using MED Discovery Engine:

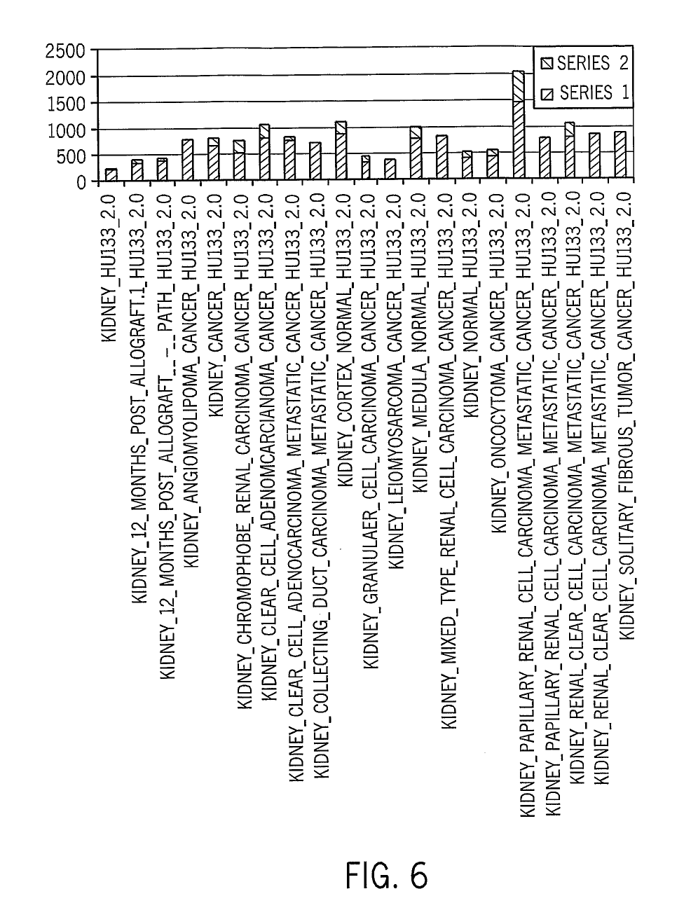

MED discovery engine, described in Example 4 herein, was used to assess the expression of TNFRFS1A transcripts. Expression data for Affymetrix probe set 207643_s_at representing TNFRFS1A from HG-U133 family data is shown in FIGS. 6. As is evident from the scatter plot, presented in FIG. 6, the expression of TNFRFS1A transcripts detectable with the above probe set in some diseased samples was higher than in the normal samples. FIG. 6 presents the results on kidney panel, showing over expression in Kidney Papillary Renal Cell Carcinoma.

### Description for Cluster Z18303

Cluster Z18303 (internal ID 13314805) features 7 transcript(s) and 11 segment(s) of interest, the names for which are given in Tables 100 and 101. The selected protein variants are given in table 102.

TABLE 100
Transcripts of interest
Transcript Name

Z18303_T3 (SEQ ID NO: 109)
Z18303_T4 (SEQ ID NO: 110)
Z18303_T6 (SEQ ID NO: 111)
Z18303_T7 (SEQ ID NO: 112)
Z18303_T11 (SEQ ID NO: 113)
Z18303_T13 (SEQ ID NO: 114)
Z18303_T22 (SEQ ID NO: 115)

TABLE 101
Segments of interest
Segment Name

Z18303_N28 (SEQ ID NO: 116)
Z18303_N29 (SEQ ID NO: 117)
Z18303_N30 (SEQ ID NO: 118)
Z18303_N57 (SEQ ID NO: 119)
Z18303_N87 (SEQ ID NO: 120)
Z18303_N19 (SEQ ID NO: 121)
Z18303_N20 (SEQ ID NO: 122)
Z18303_N47 (SEQ ID NO: 123)
Z18303_N75 (SEQ ID NO: 124)
Z18303_N85 (SEQ ID NO: 125)
Z18303_N88 (SEQ ID NO: 126)

TABLE 102
Proteins of interest

| Protein Name | Corresponding Transcript(s) |
|---|---|
| Z18303_P3 (SEQ ID NO: 129) | Z18303_T3 (SEQ ID NO: 109) |
| Z18303_P4 (SEQ ID NO: 130) | Z18303_T4 (SEQ ID NO: 110) |
| Z18303_P6 (SEQ ID NO: 131) | Z18303_T6 (SEQ ID NO: 111) |
| Z18303_P7 (SEQ ID NO: 132) | Z18303_T7 (SEQ ID NO: 112) |
| Z18303_P10 (SEQ ID NO: 133) | Z18303_T11 (SEQ ID NO: 113) |
| Z18303_P12 (SEQ ID NO: 134) | Z18303_T13 (SEQ ID NO: 114) |
| Z18303_P19 (SEQ ID NO: 135) | Z18303_T22 (SEQ ID NO: 115) |

These sequences are variants of the known protein Myosin-binding protein C (SEQ ID NO:127), cardiac-type (SwissProt accession identifier MYPC_HUMAN (SEQ ID NO: 127); known also according to the synonyms Cardiac MyBP-C; C-protein, cardiac muscle isoform), referred to herein as the previously known protein.

Protein Myosin-binding protein C (SEQ ID NO:127), cardiac-type is known or believed to have the following function(s): Thick filament-associated protein located in the crossbridge region of vertebrate striated muscle a bands. In vitro it binds MHC, F-actin and native thin filaments, and modifies the activity of actin-activated myosin ATPase. It may modulate muscle contraction or may play a more structural role. Known polymorphisms for this sequence are as shown in Table 103.

TABLE 103
Amino acid mutations for Known Protein

| SNP position(s) | |
|---|---|
| on amino | |
| acid sequence | Comment |
| 257 | H -> P (in CMH4). /FTId = VAR_019889 |
| 258 | E -> K (in CMH4). /FTId = VAR_019890 |
| 278 | G -> E (in CMH4). /FTId = VAR_019891 |
| 382 | R -> W. /FTId = VAR_020570 |
| 383 | L -> V. /FTId = VAR_020571 |
| 811 | K -> R (in CMH4). /FTId = VAR_019897 |
| 833 | A -> V (in CMH4). /FTId = VAR_019898 |
| 1048 | R -> C. /FTId = VAR_020575 |
| 820 | R -> Q |
| 158 | V -> M (in dbSNP: 3729986). /FTId = VAR_020085 |
| 281 | R -> Q. /FTId = VAR_020569 |
| 326 | R -> Q (in CMH4). /FTId = VAR_019893 |
| 504 | Missing (in CMH4). /FTId = VAR_019896 |
| 1194 | A -> T (in CMH4). /FTId = VAR_019900 |
| 502 | R -> W (in CMH4). /FTId = VAR_019895 |
| 522 | A -> T. /FTId = VAR_020573 |
| 755 | N -> K (in CMH4). /FTId = VAR_003919 |
| 302-303 | RD -> SS |
| 352 | L -> P (in CMH4). /FTId = VAR_019894 |
| 248 | D -> E |
| 408-409 | SK -> R |
| 536 | A -> R |
| 189 | V -> I. /FTId = VAR_020568 |
| 542 | E -> Q (in CMH4). /FTId = VAR_003917 |
| 998 | Q -> E. /FTId = VAR_020574 |
| 279 | G -> A (in CMH4). /FTId = VAR_019892 |
| 520 | Missing (in 522). /FTId = VAR_020572 |
| 654 | R -> H (in CMH4; dbSNP: 1800565). |
| | /FTId = VAR_003918 |
| 896 | V -> M (in CMH4; uncertain pathological significance; |
| | may act as a phenotype modifier). |
| | /FTId = VAR_019899 |
| 236 | S -> G (in dbSNP: 3729989). /FTId = VAR_020086 |
| 1255 | A -> T (in CMH4). /FTId = VAR_019901 |

The following GO Annotation(s) apply to the previously known protein. The following annotation(s) were found: regulation of striated muscle contraction; muscle contraction, which are annotation(s) related to Biological Process; and structural constituent of muscle, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

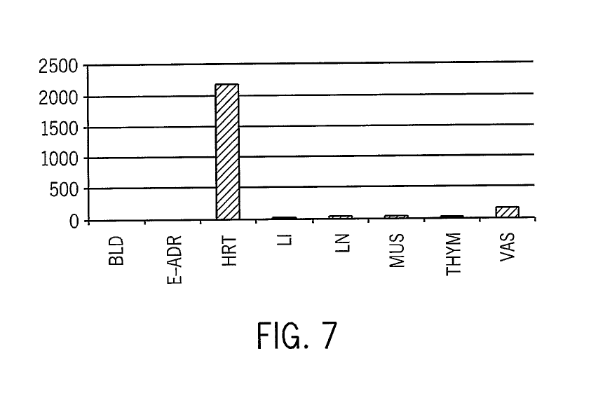

The heart-selective diagnostic marker prediction engine provided the following results with regard to cluster Z18303. Predictions were made for selective expression of transcripts of this contig in heart tissue, according to the previously described methods. The numbers on the y-axis of FIG. 7 refer to weighted expression of ESTs in each category, as “parts per million” (ratio of the expression of ESTs for a particular cluster to the expression of all ESTs in that category, according to parts per million).

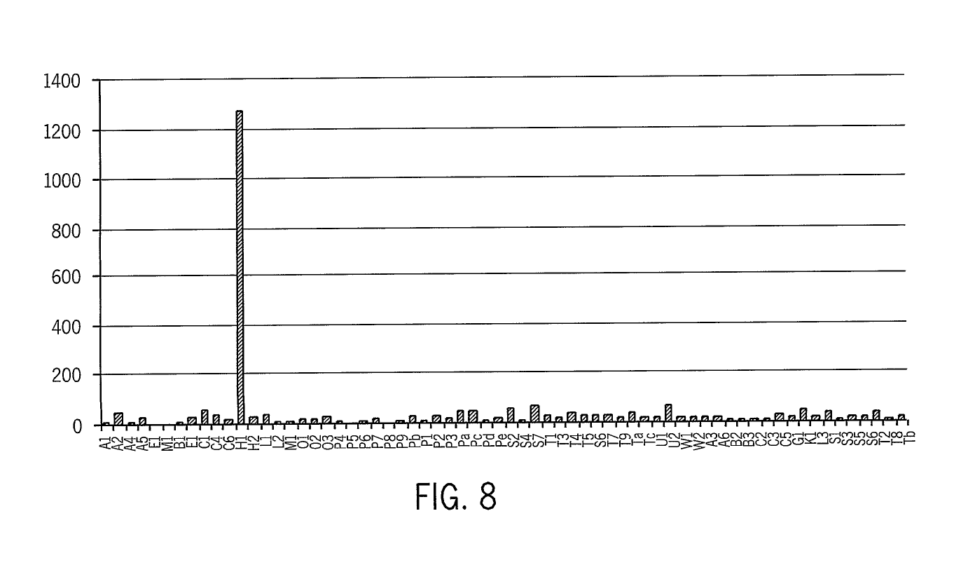

Overall, the following results were obtained as shown with regard to the histogram in FIG. 7, concerning the number of heart-specific clones in libraries/sequences; as well as with regard to the histogram in FIG. 8, concerning the actual expression of oligonucleotides in various tissues, including heart. FIG. 8 demonstrates Expression of oligonucleotides in various tissues, prob 208040_s_at (SEQ ID NO:150).

This cluster was found to be selectively expressed in heart for the following reasons: in a comparison of the ratio of expression of the cluster in heart specific ESTs to the overall expression of the cluster in non-heart ESTs, which was found to be 27.2; the ratio of expression of the cluster in heart specific ESTs to the overall expression of the cluster in muscle-specific ESTs which was found to be 58.7; and fisher exact test P-values were computed both for library and weighted clone counts to check that the counts are statistically significant, and were found to be 1.30E-61.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 27.2, which clearly supports specific expression in heart tissue.

As noted above, cluster Z18303 features 7 transcript(s), which were listed in Table 100 above. These transcript(s) encode for protein(s) which are variant(s) of protein Myosin-binding protein C (SEQ ID NO:127), cardiac-type. A description of each variant protein according to the present invention is now provided.

Variant protein Z18303_P3 (SEQ ID NO:129) according to the present invention is encoded by transcript(s) Z18303_T3 (SEQ ID NO:109).

The localization of the variant protein was determined according to results from a number of different software programs and analyses, including analyses from SignalP and other specialized programs. The variant protein is believed to be located as follows with regard to the cell: intracellularly.

Comparison Report Between Z18303 P3 and Known Protein(s) NP_{—}000247:

A. An isolated chimeric polypeptide encoding for Z18303_P3, comprising a first amino acid sequence being at least 90% homologous to MPEPGICKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247, which also corresponds to amino acids 1-247 of Z18303_P3, a bridging amino acid D corresponding to amino acid 248 of Z18303_P3, a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVTDLRGMLKRLKGMRRDEKKST corresponding to amino acids 249-363 of known protein(s) NP_{—}000247, which also corresponds to amino acids 249-363 of Z18303_P3, and a third amino acid G corresponding to amino acids 364-364 of Z18303_P3, wherein said first amino acid sequence, bridging amino acid, second amino acid sequence and third amino acid are contiguous and in a sequential order.

Variant protein Z18303_P3 (SEQ ID NO:129) also has the following non-silent SNPs (Single Nucleotide Polymorphisms) as listed in Table 104, (given according to their position(s) on the amino acid sequence, with the alternative amino acid(s) listed; the last column indicates whether the SNP is known or not; the presence of known SNPs in variant protein Z18303_P3 (SEQ ID NO:129) sequence provides support for the deduced sequence of this variant protein according to the present invention).

TABLE 104
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |

The phosphorylation sites of variant protein Z18303_P3 (SEQ ID NO:129), as compared to the known protein, are described in Table 105 (given according to their position(s) on the amino acid sequence in the first column; the second column indicates whether the phosphorylation site is present in the variant protein; and the last column indicates whether the position is different on the variant protein).

TABLE 105
Phosphorylation site(s)

| Position(s) on | | |
|---|---|---|
| known amino | Present in | Position(s) on |
| acid sequence | variant protein? | variant protein |
| 275 | Yes | 275 |
| 284 | Yes | 284 |
| 304 | Yes | 304 |

Variant protein Z18303_P3 (SEQ ID NO:129) is encoded by the following transcript(s): Z18303_T3 (SEQ ID NO:109). The coding portion of transcript Z18303_T3 (SEQ ID NO:109) starts at position 56 and ends at position 1147. The transcript also has the following SNPs as listed in Table 106 (given according to their position on the nucleotide sequence, with the alternative nucleic acid listed; the last column indicates whether the SNP is known or not; the presence of known SNPs in variant protein Z18303_P3 (SEQ ID NO:129) sequence provides support for the deduced sequence of this variant protein according to the present invention).

TABLE 106
Nucleic acid SNPs

| Polymorphism | SNP position(s) on nucleotide sequence |
|---|---|
| T -> C | 88, 3627, 6561 |
| G -> T | 263, 1646 |
| C -> T | 427, 547, 592, 841, 2792, 3502, 3523, 3689, 3704, 3781, |
| | 5135, 5184, 5238, 5641, 5779 |
| G -> A | 527, 620, 897, 1532, 2994, 3697, 4201, 4598, 5096, |
| | 5908, 5925, 6531, 6698 |
| T -> A | 726 |
| A -> G | 761, 3680, 6487 |
| C -> A | 799, 2808, 4700, 5047 |
| G -> C | 1327 |
| A -> C | 1605, 3097, 3235, 3525 |
| C -> G | 3682, 3784, 5629 |
| C -> | 6674 |

Variant protein Z18303_P4 (SEQ ID NO:130) according to the present invention is encoded by transcript(s) Z18303_T4 (SEQ ID NO:110). An alignment is given to the known protein (Myosin-binding protein C (SEQ ID NO:127), cardiac-type) at the end of the application. One or more alignments to one or more previously published protein sequences are given at the end of the application. A brief description of the relationship of the variant protein according to the present invention to each such aligned protein is as follows:

### 1. Comparison Report Between Z18303_P4 (SEQ ID NO:130) and Known Protein(s) MYPC_HUMAN (SEQ ID NO: 127) and Q9UM53_HUMAN (SEQ ID NO: 127):

A. An isolated chimeric polypeptide comprising Z18303_P4 (SEQ ID NO:130), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR ISDSHEDTGILDFSSLLKKRDSFRTPRDSKLEAPAEEDVWEILRQAPPSEYERIAFQYGVTDLRGMLKRLKG IVIRRDEKKSTAFQKKLEPAYQVSKGHKIRLTVELADHDAEVKWLKNGQEIQMSGSKYIFESIGAKRTLTISQ CSLADDAAYQCVVGGEKCSTELFVKEPPVLITRPLED QLVMVGQRVEFECEVSEEGAQVKWLKDGVELTR EETFKYRFKKDGQRHHLIINEAMLEDAGHYALCTSGGQALAELIVQEKKLEVYQSIADLMVGAKDQAVFK CEVSDENVRGVWLKNGKELVPDSRIKVSHIGRVHKLTIDDVTPADEADYSFVPEGFACNLSAKLHFMEVKI DFVPRQEPPKIHLDCPGRIPDTIVVVAGNKLRLDVPISGDPAPTVIWQKAITQGNKAPARPAPDAPEDTGDS DEWVFDKKLLCETEGRVRVETTKDRSIFTVEGAEKEDEGVYTVTVKNPVGEDQVNLTVKVI corresponding to amino acids 1-769 of known protein(s) MYPC_HUMAN (SEQ ID NO: 127) and Q9UM53_HUMAN (SEQ ID NO: 127), which also corresponds to amino acids 1-769 of Z18303_P4 (SEQ ID NO:130), and a second amino acid sequence being at least 70%, optionally at least 80%, preferably at least 85%, more preferably at least 90% and most preferably at least 95% homologous to a polypeptide having the sequence GEAGRGPSWRTQRGSPKEGPSVRSFSHSTNITGMLPEPSGNRTGFSAQKEPRSGGDGGRCIQQISCDTAVHE PCPECRGSTEQRAIPVPGGDAFRRLRR (SEQ ID NO: 147) corresponding to amino acids 770-868 of Z18303_P4 (SEQ ID NO:130), wherein said first amino acid sequence and second amino acid sequence are contiguous and in a sequential order.

B. An isolated polypeptide comprising an edge portion of Z18303_P4 (SEQ ID NO:130), comprising an amino acid sequence being at least 70%, optionally at least about 80%, preferably at least about 85%, more preferably at least about 90% and most preferably at least about 95% homologous to the sequence GEAGRGPSWRTQRGSPKEGPSVRSFSHSTNITGMLPEPSGNRTGFSAQKEPRSGGDGGRCIQQISCDTAVHE PCPECRGSTEQRAIPVPGGDAFRRLRR (SEQ ID NO: 147) of Z18303_P4 (SEQ ID NO:130).

### 2. Comparison Report Between Z18303_P4 (SEQ ID NO:130) and Known Protein(s) NP_{—}000247 (SEQ ID NO: 128):

A. An isolated chimeric polypeptide comprising Z18303_P4 (SEQ ID NO:130), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247 (SEQ ID NO: 128), which also corresponds to amino acids 1-247 of Z18303_P4 (SEQ ID NO:130), a bridging amino acid D corresponding to amino acid 248 of Z18303_P4 (SEQ ID NO:130), a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVTDLRGMLKRLKGMRRDEKKSTAFQKKLEPAYQVSKGHKIRLTVELADH DAEVKWLKNGQEIQMSGSKYIFESIGAKRTLTISQCSLADDAAYQCVVGGEKCSTELFVKEPPVLITRPLED QLVMVGQRVEFECEVSEEGAQVKWLICDGVELTREETFKYRFKKDGQRHHLIINEAMLEDAGHYALCTSG GQAL corresponding to amino acids 249-535 of known protein(s) NP_{—}000247 (SEQ ID NO: 128), which also corresponds to amino acids 249-535 of Z18303_P4 (SEQ ID NO:130), a bridging amino acid A corresponding to amino acid 536 of Z18303_P4 (SEQ ID NO:130), a third amino acid sequence being at least 90% homologous to ELIVQEKKLEVYQSIADLMVGAKDQAVFKCEVSDENVRGVWLKNGKELVPDSRIKVSHIGRVHKLTIDDV TPADEADYSFVPEGFACNLSAKLHFMEVKIDFVPRQEPPKIHLDCPGRIPDTIVVVAGNKLRLDVPISGDPAP TVIWQKAITQGNKAPARPAPDAPEDTGDSDEWVFDKKLLCETEGRVRVETTKDRSIFTVEGAEKEDEGVY TVTVKNPVGEDQVNLTVKVI corresponding to amino acids 537-769 of known protein(s) NP_{—}000247 (SEQ ID NO: 128), which also corresponds to amino acids 537-769 of Z18303_P4 (SEQ ID NO:130), and a fourth amino acid sequence being at least 70%, optionally at least 80%, preferably at least 85%, more preferably at least 90% and most preferably at least 95% homologous to a polypeptide having the sequence GEAGRGPSWRTQRGSPKEGPSVRSFSHSTNITGMLPEPSGNRTGFSAQKEPRSGGDGGRCIQQISCDTAVHE PCPECRGSTEQRAIPVPGGDAFRRLRR (SEQ ID NO: 147) corresponding to amino acids 770-868 of Z18303_P4 (SEQ ID NO:130), wherein said first amino acid sequence, bridging amino acid, second amino acid sequence, bridging amino acid, third amino acid sequence and fourth amino acid sequence are contiguous and in a sequential order.

B. An isolated polypeptide comprising an edge portion of Z18303_P4 (SEQ ID NO:130), comprising an amino acid sequence being at least 70%, optionally at least about 80%, preferably at least about 85%, more preferably at least about 90% and most preferably at least about 95% homologous to the sequence GEAGRGPSWRTQRGSPKEGPSVRSFSHSTNITGMLPEPSGNRTGFSAQKEPRSGGDGGRCIQQISCDTAVHE PCPECRGSTEQRAIPVPGGDAFRRLRR (SEQ ID NO: 147) of Z18303_P4 (SEQ ID NO:130).

The localization of the variant protein was determined according to results from a number of different software programs and analyses, including analyses from SignalP and other specialized programs. The variant protein is believed to be located as follows with regard to the cell: intracellularly.

Variant protein Z18303_P4 (SEQ ID NO:130) also has the following non-silent SNPs (Single Nucleotide Polymorphisms) as listed in Table 107, (given according to their position(s) on the amino acid sequence, with the alternative amino acid(s) listed; the last column indicates whether the SNP is known or not; the presence of known SNPs in variant protein Z18303_P4 (SEQ ID NO:130) sequence provides support for the deduced sequence of this variant protein according to the present invention).

TABLE 107
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |
| 382 | R -> W |
| 383 | L -> V |
| 522 | A -> T |
| 654 | R -> H |
| 688 | T -> K |
| 776 | P -> A |
| 801 | T -> M |
| 809 | G -> S |

The phosphorylation sites of variant protein Z18303_P4 (SEQ ID NO:130), as compared to the known protein, are described in Table 108 (given according to their position(s) on the amino acid sequence in the first column; the second column indicates whether the phosphorylation site is present in the variant protein; and the last column indicates whether the position is different on the variant protein).

TABLE 108
Phosphorylation site(s)

| Position(s) on | | |
|---|---|---|
| known amino | Present in | Position(s) on |
| acid sequence | variant protein? | variant protein |
| 275 | Yes | 275 |
| 284 | Yes | 284 |
| 304 | Yes | 304 |

Variant protein Z18303_P4 (SEQ ID NO:130) is encoded by the following transcript(s): Z18303_T4 (SEQ ID NO:110). The coding portion of transcript Z18303_T4 (SEQ ID NO:110) starts at position 56 and ends at position 2659. The transcript also has the following SNPs as listed in Table 109 (given according to their position on the nucleotide sequence, with the alternative nucleic acid listed; the last column indicates whether the SNP is known or not; the presence of known SNPs in variant protein Z18303_P4 (SEQ ID NO:130) sequence provides support for the deduced sequence of this variant protein according to the present invention).

TABLE 109
Nucleic acid SNPs

| Polymorphism | SNP position(s) on nucleotide sequence |
|---|---|
| T -> C | 88, 4277 |
| G -> T | 263 |
| C -> T | 427, 547, 592, 841, 1199, 2457, 2851, 2900, 2954, 3357, |
| | 3495 |
| G -> A | 527, 620, 897, 1619, 2016, 2480, 2812, 3624, 3641, |
| | 4247, 4414 |
| T -> A | 726 |
| A -> G | 761, 4203 |
| C -> A | 799, 2118, 2763 |
| C -> G | 1202, 2381, 3345 |
| A -> T | 2581 |
| C -> | 4390 |

Variant protein Z18303_P6 (SEQ ID NO:131) according to the present invention is encoded by transcript(s) Z18303_T6 (SEQ ID NO:111).

The localization of the variant protein was determined according to results from a number of different software programs and analyses, including analyses from SignalP and other specialized programs. The variant protein is believed to be located as follows with regard to the cell: intracellularly.

Comparison Report Between Z18303_P6 and Known Protein(s) MYPC_HUMAN and Q9UM53_HUMAN:

A. An isolated chimeric polypeptide encoding for Z18303_P6, comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR ISDSHEDTGILDFSSLLKKRDSFRTPRDSKLEAPAEEDVWEILRQAPPSEYERIAFQYGVTDLRGMLKRLKG MRRDEKKSTAFQKKLEPAYQVSKGHKIRLTVELADHDAEVKWLKNGQEIQMSGSKYIFESIGAKRTLTISQ CSLADDAAYQCVVGGEKCSTELFVKEPPVLITRPLEDQLVMVGQRVEFECEVSEEGAQVKWLKDGVELTR EETFKYRFKKDGQRHHLIINEAMLEDAGHYALCTSGGQALAELIVQEKKLEVYQSIADLMVGAKDQAVFK CEVSDENVRGVWLKNGKELVPDSRIKVSHIGRVHKLTIDDVTPADEADYSFVPEGFACNLSAKLHFM corresponding to amino acids 1-632 of known protein(s) MYPC_HUMAN and Q9UM53_HUMAN, which also corresponds to amino acids 1-632 of Z18303_P6, and a second amino acid sequence being at least 70%, optionally at least 80%, preferably at least 85%, more preferably at least 90% and most preferably at least 95% homologous to a polypeptide having the sequence GEPAPG corresponding to amino acids 633-638 of Z18303_P6, wherein said first amino acid sequence and second amino acid sequence are contiguous and in a sequential order.

B. An isolated polypeptide encoding for an edge portion of Z18303_P6, comprising an amino acid sequence being at least 70%, optionally at least about 80%, preferably at least about 85%, more preferably at least about 90% and most preferably at least about 95% homologous to the sequence GEPAPG of Z18303_P6.

### 2. Comparison Report Between Z18303_P6 and Known Protein(s) NP_{—}000247:

A. An isolated chimeric polypeptide encoding for Z18303_P6, comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247, which also corresponds to amino acids 1-247 of Z18303_P6, a bridging amino acid D corresponding to amino acid 248 of Z18303_P6, a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVIDLRGMLICRLKGMRRDEKKSTAFQKKLEPAYQVSKGHKIRLTVELADH DAEVKWLKNGQEIQMSGSKYIFESIGAKRTLTISQCSLADDAAYQCVVGGEKCSTELFVKEPPVLITRPLED QLVMVGQRVEFECEVSEEGAQVKWLICDGVELTREETFKYRFKKDGQRHHLIINEAMLEDAGHYALCTSG GQAL corresponding to amino acids 249-535 of known protein(s) NP_{—}000247, which also corresponds to amino acids 249-535 of Z18303_P6, a bridging amino acid A corresponding to amino acid 536 of Z18303_P6, a third amino acid sequence being at least 90% homologous to ELIVQEKKLEVYQSIADLMVGAKDQAVFKCEVSDENVRGVWLKNGKELVPDSRIKVSHIGRVHKLTIDDV TPADEADYSFVPEGFACNLSAKLHFM corresponding to amino acids 537-632 of known protein(s) NP_{—}000247, which also corresponds to amino acids 537-632 of Z18303_P6, and a fourth amino acid sequence being at least 70%, optionally at least 80%, preferably at least 85%, more preferably at least 90% and most preferably at least 95% homologous to a polypeptide having the sequence GEPAPG corresponding to amino acids 633-638 of Z18303_P6, wherein said first amino acid sequence, bridging amino acid, second amino acid sequence, bridging amino acid, third amino acid sequence and fourth amino acid sequence are contiguous and in a sequential order.

B. An isolated polypeptide encoding for an edge portion of Z18303_P6, comprising an amino acid sequence being at least 70%, optionally at least about 80%, preferably at least about 85%, more preferably at least about 90% and most preferably at least about 95% homologous to the sequence GEPAPG of Z18303_P6.

Variant protein Z18303_P6 (SEQ ID NO:131) also has the following non-silent SNPs (Single Nucleotide Polymorphisms) as listed in Table 110, (given according to their position(s) on the amino acid sequence, with the alternative amino acid(s) listed; the last column indicates whether the SNP is known or not; the presence of known SNPs in variant protein Z18303_P6 (SEQ ID NO:131) sequence provides support for the deduced sequence of this variant protein according to the present invention).

TABLE 110
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |
| 382 | R -> W |
| 383 | L -> V |
| 522 | A -> T |

The phosphorylation sites of variant protein Z18303_P6 (SEQ ID NO:131), as compared to the known protein, are described in Table 111 (given according to their position(s) on the amino acid sequence in the first column; the second column indicates whether the phosphorylation site is present in the variant protein; and the last column indicates whether the position is different on the variant protein).

TABLE 111
Phosphorylation site(s)

| Position(s) on | | |
|---|---|---|
| known amino | Present in | Position(s) on |
| acid sequence | variant protein? | variant protein |
| 275 | Yes | 275 |
| 284 | Yes | 284 |
| 304 | Yes | 304 |

Variant protein Z18303_P6 (SEQ ID NO:131) is encoded by the following transcript(s): Z18303_T6 (SEQ ID NO:111). The coding portion of transcript Z18303_T6 (SEQ ID NO:111) starts at position 56 and ends at position 1969. The transcript also has the following SNPs as listed in Table 112 (given according to their position on the nucleotide sequence, with the alternative nucleic acid listed; the last column indicates whether the SNP is known or not; the presence of known SNPs in variant protein Z18303_P6 (SEQ ID NO:131) sequence provides support for the deduced sequence of this variant protein according to the present invention).

TABLE 112
Nucleic acid SNPs

| Polymorphism | SNP position(s) on nucleotide sequence |
|---|---|
| T -> C | 88, 4084 |
| G -> T | 263 |
| C -> T | 427, 547, 592, 841, 1199, 2658, 2707, 2761, 3164, 3302 |
| G -> A | 527, 620, 897, 1619, 1999, 2121, 2619, 3431, 3448, |
| | 4054, 4221 |
| T -> A | 726 |
| A -> G | 761, 4010 |
| C -> A | 799, 2223, 2570 |
| C -> G | 1202, 3152 |
| C -> | 4197 |

Variant protein Z18303_P7 (SEQ ID NO:132) according to the present invention is encoded by transcript(s) Z18303_T7 (SEQ ID NO:112).

The localization of the variant protein was determined according to results from a number of different software programs and analyses, including analyses from SignalP and other specialized programs. The variant protein is believed to be located as follows with regard to the cell: intracellularly.

Comparison report between Z18303_P7 and known protein(s) MYPC_HUMAN and Q9UM53_HUMAN:

A. An isolated chimeric polypeptide encoding for Z18303_P7, comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR IS corresponding to amino acids 1-284 of known protein(s) MYPC_HUMAN and Q9UM53_HUMAN, which also corresponds to amino acids 1-284 of Z18303_P7, and a second amino acid sequence being at least 70%, optionally at least 80%, preferably at least 85%, more preferably at least 90% and most preferably at least 95% homologous to a polypeptide having the sequence PSLQPHWGSLSIVIAMRTLGFWTSAHC corresponding to amino acids 285-311 of Z18303_P7, wherein said first amino acid sequence and second amino acid sequence are contiguous and in a sequential order.

B. An isolated polypeptide encoding for an edge portion of Z18303_P7, comprising an amino acid sequence being at least 70%, optionally at least about 80%, preferably at least about 85%, more preferably at least about 90% and most preferably at least about 95% homologous to the sequence PSLQPHWGSLSIVIAMRTLGFWTSAHC of Z18303_P7.

### 2. Comparison Report Between Z18303_P7 and Known Protein(s) NP_{—}000247:

A. An isolated chimeric polypeptide encoding for Z18303_P7, comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247, which also corresponds to amino acids 1-247 of Z18303_P7, a bridging amino acid D corresponding to amino acid 248 of Z18303_P7, a second amino acid sequence being at least 90% homologous to CSNFNLTVHEAMGTGDLDLLSAFRRTSLAGGGRRIS corresponding to amino acids 249-284 of known protein(s) NP_{—}000247, which also corresponds to amino acids 249-284 of Z18303_P7, and a third amino acid sequence being at least 70%, optionally at least 80%, preferably at least 85%, more preferably at least 90% and most preferably at least 95% homologous to a polypeptide having the sequence PSLQPHWGSLSIVIAMRTLGFWTSAHC corresponding to amino acids 285-311 of Z18303_P7, wherein said first amino acid sequence, bridging amino acid, second amino acid sequence and third amino acid sequence are contiguous and in a sequential order.

B. An isolated polypeptide encoding for an edge portion of Z18303_P7, comprising an amino acid sequence being at least 70%, optionally at least about 80%, preferably at least about 85%, more preferably at least about 90% and most preferably at least about 95% homologous to the sequence PSLQPHWGSLSIVIAMRTLGFWTSAHC of Z18303_P7.

Variant protein Z18303_P7 (SEQ ID NO:132) also has the following non-silent SNPs (Single Nucleotide Polymorphisms) as listed in Table 113, (given according to their position(s) on the amino acid sequence, with the alternative amino acid(s) listed; the last column indicates whether the SNP is known or not; the presence of known SNPs in variant protein Z18303_P7 (SEQ ID NO:132) sequence provides support for the deduced sequence of this variant protein according to the present invention).

TABLE 113
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |

The phosphorylation sites of variant protein Z18303_P7 (SEQ ID NO:132), as compared to the known protein, are described in Table 114 (given according to their position(s) on the amino acid sequence in the first column; the second column indicates whether the phosphorylation site is present in the variant protein; and the last column indicates whether the position is different on the variant protein).

TABLE 114
Phosphorylation site(s)

| Position(s) on | | |
|---|---|---|
| known amino | Present in | Position(s) on |
| acid sequence | variant protein? | variant protein |
| 275 | Yes | 275 |
| 284 | Yes | 284 |
| 304 | No | |

Variant protein Z18303_P7 (SEQ ID NO:132) is encoded by the following transcript(s): Z18303_T7 (SEQ ID NO:112). The coding portion of transcript Z18303_T7 (SEQ ID NO:112) starts at position 56 and ends at position 988. The transcript also has the following SNPs as listed in Table 115 (given according to their position on the nucleotide sequence, with the alternative nucleic acid listed; the last column indicates whether the SNP is known or not; the presence of known SNPs in variant protein Z18303_P7 (SEQ ID NO:132) sequence provides support for the deduced sequence of this variant protein according to the present invention).

TABLE 115
Nucleic acid SNPs

| Polymorphism | SNP position(s) on nucleotide sequence |
|---|---|
| T -> C | 88, 4273 |
| G -> T | 263 |
| C -> T | 427, 547, 592, 841, 1493, 2847, 2896, 2950, 3353, 3491 |
| G -> A | 527, 620, 897, 1215, 1913, 2310, 2808, 3620, 3637, |
| | 4243, 4410 |
| T -> A | 726 |
| A -> G | 761, 4199 |
| C -> A | 799, 1044, 2412, 2759 |
| G -> C | 1139 |
| C -> G | 1496, 3341 |
| C -> | 4386 |

Variant protein Z18303_P10 (SEQ ID NO:133) according to the present invention is encoded by transcript(s) Z18303_T11 (SEQ ID NO:113). An alignment is given to the known protein (Myosin-binding protein C (SEQ ID NO:127), cardiac-type) at the end of the application. One or more alignments to one or more previously published protein sequences are given at the end of the application. A brief description of the relationship of the variant protein according to the present invention to each such aligned protein is as follows:

### 1. Comparison Report Between Z18303_P10 (SEQ ID NO:133) and Known Protein(s) MYPC_HUMAN (SEQ ID NO: 127) and Q9UM53_HUMAN (SEQ ID NO: 127):

A. An isolated chimeric polypeptide comprising Z18303_P10 (SEQ ID NO:133), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR ISDSHEDTGILDFSSLLKKRDSFRTPRDSKLEAPAEEDVWEILRQAPPSEYERIAFQYGVTDLRGMLKRLKG MRRDEKKSTAFQKKLEPAYQVSKGHKIRLTVELADHDAEVKWLKNGQEIQMSGSKYIFESIGAKRTLTISQ CSLADDAAYQCVVGGEKCSTELFVKEPPVLITRPLED QLVMVGQRVEFECEVSEEGAQVKWLKDGVELTR EETFKYRFKKDGQRHHLIINEAMLEDAGHYALCTSGGQALAELIVQEKKLEVYQSIADLMVGAKDQAVFK CEVSDENVRGVWLKNGKELVPDSRIKVSHIGRVHKLTIDDVTPADEADYSFVPEGFACNLSAKLHFMEVKI DFVPRQEPPKIRLDCPGRIPDTIVVVAGNKLRLDVPISGDPAPTVIWQKAITQGNKAPARPAPDAPEDTGDS DEWVFDKKLLCETEGRVRVETTKDRSIFTVEGAEKEDEGVYTVTVKNPVGEDQVNLTVKVIDVPDAPAAP KISNVGEDSCTVQWEPPAYDGGQPILGYILERKKKKSYRWMRLNFDLIQELSHEARRMIEGVVYEMRVYA VNAIGMSRPSPASQPFMPIGPPSEPTHLAVEDVSDTTVSLKWRPPERVGAGGLDGYSVEYCPEGCSEWVAA LQGLTEHTSILVKDLPTGARLLFRVRAHNMAGPGAPVTTTEPVTVQEILQRPRLQLPRHLRQTIQKKVGEPV NLLIPFQGKPRPQVTWTKEGQPLAGEEVSIRNSPTDTILFIRAARRVHSGTYQVTVRIENMEDKATLVLQVV DKPSPPQDLRVTDAWGLNVALEWKPPQDVGNTELWGYTVQKADKKTMEWFTVLEHYRRTHCVVPELIIG NGYYFRVFSQNMVGFSDRAATTKEPVFIPRPGITYEPPNYKALDFSEAPSFTQPLVNRSVIAGYTAMLCCAV RGSPKPKISWFKNGLDLGEDARFRMFSKQGVLTLEIRKPCPFDGGIYVCRATNLQGEARCECRLEVR corresponding to amino acids 1-1271 of known protein(s) MYPC_HUMAN (SEQ ID NO: 127) and Q9UM53_HUMAN (SEQ ID NO: 127), which also corresponds to amino acids 1-1271 of Z18303_P10 (SEQ ID NO:133), and a second amino acid sequence being at least 70%, optionally at least 80%, preferably at least 85%, more preferably at least 90% and most preferably at least 95% homologous to a polypeptide having the sequence GEEPSGPGPGRWEERAAVGTLGFAPLSSAKHLPWLQGHGPCHAQMGNSFQKAGRTQ (SEQ ID NO: 148) corresponding to amino acids 1272-1327 of Z18303_P10 (SEQ ID NO:133), wherein said first amino acid sequence and second amino acid sequence are contiguous and in a sequential order.

B. An isolated polypeptide comprising an edge portion of Z18303_P10 (SEQ ID NO:133), comprising an amino acid sequence being at least 70%, optionally at least about 80%, preferably at least about 85%, more preferably at least about 90% and most preferably at least about 95% homologous to the sequence GEEPSGPGPGRWEERAAVGTLGFAPLSSAKHLPWLQGHGPCHAQMGNSFQKAGRTQ (SEQ ID NO: 148) of Z18303_P10 (SEQ ID NO:133).

### 2. Comparison Report Between Z18303_P10 (SEQ ID NO:133) and Known Protein(s) NP_{—}000247 (SEQ ID NO: 128):

A. An isolated chimeric polypeptide comprising Z18303_P10 (SEQ ID NO:133), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247 (SEQ ID NO: 128), which also corresponds to amino acids 1-247 of Z18303_P10 (SEQ ID NO:133), a bridging amino acid D corresponding to amino acid 248 of Z18303_P10 (SEQ ID NO:133), a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVTDLRGMLKRLKGMRRDEKKSTAFQKKLEPAYQVSKGHKIRLTVELADH DAEVKWLKNGQEIQMSGSKYIFESIGAKRTLTISQCSLADDAAYQCVVGGEKCSTELFVKEPPVLITRPLED QLVMVGQRVEFECEVSEEGAQVKWLKDGVELTREETFKYRFKKDGQRHHLIINEAMLEDAGHYALCTSG GQAL corresponding to amino acids 249-535 of known protein(s) NP_{—}000247 (SEQ ID NO: 128), which also corresponds to amino acids 249-535 of Z18303_P10 (SEQ ID NO:133), a bridging amino acid A corresponding to amino acid 536 of Z18303_P10 (SEQ ID NO:133), a third amino acid sequence being at least 90% homologous to ELIVQEKKLEVYQSIADLMVGAKDQAVFKCEVSDENVRGVWLKNGKELVPDSRIKVSHIGRVHKLTIDDV TPADEADYSFVPEGFACNLSAKLHFMEVKIDFVPRQEPPKIHLDCPGRIPDTIVVVAGNKLRLDVPISGDPAP TVFWQKAITQGNKAPARPAPDAPEDTGDSDEWVFDKKLLCETEGRVRVETTKDRSIFTVEGAEKEDEGVY TVTVKNPVGEDQVNLTVKVIDVPDAPAAPKISNVGEDSCTVQWEPPAYDGGQPILGYILERKKKKSYRWM corresponding to amino acids 537-819 of known protein(s) NP_{—}000247 (SEQ ID NO: 128), which also corresponds to amino acids 537-819 of Z18303_P10 (SEQ ID NO:133), a bridging amino acid R corresponding to amino acid 820 of Z18303_P10 (SEQ ID NO:133), a fourth amino acid sequence being at least 90% homologous to LNFDLIQELSHEARRMIEGVVYEMRVYAVNAIGMSRPSPASQPFMPIGPPSEPTHLAVEDVSDTTVSLKWRP PERVGAGGLDGYSVEYCPEGCSEWVAALQGLTEHTSILVKDLPTGARLLFRVRAHNMAGPGAPVTTTEPV TVQEILQRPRLQLPRHLRQTIQKKVGEPVNLLIPFQGKPRPQVTWTKEGQPLAGEEVSIRNSPTDTILFIRAA RRVHSGTYQVTVRIENMEDKATLVLQVVDKPSPPQDLRVTDAWGLNVALEWKPPQDVGNTELWGYTVQ KADKKTMEWFTVLEHYRRTHCVVPELIIGNGYYFRVFSQNMVGFSDRAATTKEPVFIPRPGITYEPPNYKA LDFSEAPSFTQPLVNRSVIAGYTAMLCCAVRGSPKPKISWFKNGLDLGEDARFRMFSKQGVLTLEIRKPCPF DGGIYVCRATNLQGEARCECRLEVR corresponding to amino acids 821-1271 of known protein(s) N_P000247 (SEQ ID NO: 128), which also corresponds to amino acids 821-1271 of Z18303_P10 (SEQ ID NO:133), and a fifth amino acid sequence being at least 70%, optionally at least 80%, preferably at least 85%, more preferably at least 90% and most preferably at least 95% homologous to a polypeptide having the sequence GEEPSGPGPGRWEERAAVGTLGFAPLSSAKHLPWLQGHGPCHAQMGNSFQKAGRTQ (SEQ ID NO: 148) corresponding to amino acids 1272-1327 of Z18303_P10 (SEQ ID NO:133), wherein said first amino acid sequence, bridging amino acid, second amino acid sequence, bridging amino acid, third amino acid sequence, bridging amino acid, fourth amino acid sequence and fifth amino acid sequence are contiguous and in a sequential order.

B. An isolated polypeptide comprising an edge portion of Z18303_P10 (SEQ ID NO:133), comprising an amino acid sequence being at least 70%, optionally at least about 80%, preferably at least about 85%, more preferably at least about 90% and most preferably at least about 95% homologous to the sequence GEEPSGPGPGRWEERAAVGTLGFAPLSSAKHLPWLQGHGPCHAQMGNSFQKAGRTQ (SEQ ID NO: 148) of Z18303_P10 (SEQ ID NO:133).

The localization of the variant protein was determined according to results from a number of different software programs and analyses, including analyses from SignalP and other specialized programs. The variant protein is believed to be located as follows with regard to the cell: intracellularly.

Variant protein Z18303_P10 (SEQ ID NO:133) also has the following non-silent SNPs (Single Nucleotide Polymorphisms) as listed in Table 116, (given according to their position(s) on the amino acid sequence, with the alternative amino acid(s) listed; the last column indicates whether the SNP is known or not; the presence of known SNPs in variant protein Z18303_P10 (SEQ ID NO:133) sequence provides support for the deduced sequence of this variant protein according to the present invention).

TABLE 116
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |
| 382 | R -> W |
| 383 | L -> V |
| 522 | A -> T |
| 654 | R -> H |
| 688 | T -> K |
| 804 | L -> M |
| 820 | R -> Q |
| 833 | A -> V |
| 998 | Q -> E |
| 1002 | R -> W |
| 1048 | R -> C |
| 1091 | D -> N |
| 1305 | W -> * |
| 1322 | K -> N |

The phosphorylation sites of variant protein Z18303_P10 (SEQ ID NO:133), as compared to the known protein, are described in Table 117 (given according to their position(s) on the amino acid sequence in the first column; the second column indicates whether the phosphorylation site is present in the variant protein; and the last column indicates whether the position is different on the variant protein).

TABLE 117
Phosphorylation site(s)

| Position(s) | | |
|---|---|---|
| on known | Present | Position(s) |
| amino acid | in variant | on variant |
| sequence | protein? | protein |
| 275 | Yes | 275 |
| 284 | Yes | 284 |
| 304 | Yes | 304 |

Variant protein Z18303_P10 (SEQ ID NO:133) is encoded by the following transcript(s): Z18303_T11 (SEQ ID NO:113). The coding portion of transcript Z18303_T11 (SEQ ID NO:113) starts at position 56 and ends at position 4036. The transcript also has the following SNPs as listed in Table 118 (given according to their position on the nucleotide sequence, with the alternative nucleic acid listed; the last column indicates whether the SNP is known or not; the presence of known SNPs in variant protein Z18303_P10 (SEQ ID NO:133) sequence provides support for the deduced sequence of this variant protein according to the present invention).

TABLE 118
Nucleic acid SNPs

| Polymorphism | SNP position(s) on nucleotide sequence |
|---|---|
| T -> C | 88, 4169 |
| G -> T | 263, 4021 |
| C -> T | 427, 547, 592, 841, 1199, 2553, 2602, 2656, 3059, 3197, |
| | 3994 |
| G -> A | 527, 620, 897, 1619, 2016, 2514, 3326, 3343, 3969, |
| | 4139, 4306 |
| T -> A | 726 |
| A -> G | 761, 4095 |
| C -> A | 799, 2118, 2465 |
| C -> G | 1202, 3047 |
| C -> | 4282 |

Variant protein Z18303_P12 (SEQ ID NO:134) according to the present invention is encoded by transcript(s) Z18303_T13 (SEQ ID NO:114). An alignment is given to the known protein (Myosin-binding protein C (SEQ ID NO:127), cardiac-type) at the end of the application. One or more alignments to one or more previously published protein sequences are given at the end of the application. A brief description of the relationship of the variant protein according to the present invention to each such aligned protein is as follows:

### 1. Comparison Report Between Z18303_P12 (SEQ ID NO:134) and Known Protein(s) MYPC_HUMAN (SEQ ID NO: 127) and Q9UM53_HUMAN (SEQ ID NO: 127):

A. An isolated chimeric polypeptide comprising Z18303_P12 (SEQ ID NO:134), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR. ISDSHEDTGILDFSSLLKKRDSFRTPRDSKLEAPAEEDVWEILRQAPPSEYERIAFQYGVTDLRGMLKRLKG MRRDEKKSTAFQICKLEPAYQVSKGHKIRLTVELADHDAEVKWLKNGQEIQMSGSKYIFESIGAKRTLTISQ CSLADDAAYQCVVGGEKCSTELFVKEPPVLITRPLEDQLVMVGQRVEFECEVSEEGAQVKWLKDGVELTR EETFKYRFKKDGQRHHLIINEAMLEDAGHYALCTSGGQALAELIVQEKKLEVYQSIADLMVGAKDQAVFK CEVSDENVRGVWLKNGKELVPDSRIKVSHIGRVHKLTIDDVTPADEADYSFVPEGFACNLSAKLHFMEVKI DFVPRQEPPKIHLDCPGRIPDTIVVVAGNKLRLDVPISGDPAPTVIWQKAITQGNKAPARPAPDAPEDTGDS DEWVFDKKLLCETEGRVRVETTKDRSIFTVEGAEKEDEGVYTVTVKNPVGEDQVNLTVKVIDVPDAPAAP KISNVGEDSCTVQWEPPAYDGGQPILGYILERKKKKSYRWMRLNFDLIQELSHEARRMIEGVVYEMRVYA VNAIGMSRPSPASQPFMPIGPPSEPTHLAVEDVSDTTVSLKWRPPERVGAGGLDGYSVEYCPEGCSEWVAA LQGLTEHTSILVKDLPTGARLLFRVRAHNMAGPGAPVTTTEPVTVQEILQRPRLQLPRHLRQTIQKKVGEPV NLLIPFQGKPRPQVTWTKEGQPLAGEEVSIRNSPTDTILFIRAARRVHSGTYQVTVRIENMEDKATLVLQVV DKPSPPQDLRVTDAWGLNVALEWKPPQDVGNTELWGYTVQKADKKTMEWFTVLEHYRRTHCVVPELIIG NGYYFRVFSQNMVGFSDRAATTKEPVFIPRPGITYEPPNYKALDFSEAPSFTQPLVNRSVIAGYTAMLCCAV RGSPKPKISWFKNGLDLGEDARFRMFSKQGVLTLEIRKPCPFDGGIYVCRATNLQGEARCECRLE corresponding to amino acids 1-1269 of known protein(s) MYPC_HUMAN (SEQ ID NO: 127) and Q9UM53_HUMAN (SEQ ID NO: 127), which also corresponds to amino acids 1-1269 of Z18303_P12 (SEQ ID NO:134), and a second amino acid sequence being at least 70%, optionally at least 80%, preferably at least 85%, more preferably at least 90% and most preferably at least 95% homologous to a polypeptide having the sequence CLSDQAGSWGWPGTTGCQPRARSLEGSWGNPSLLLDVCVTSVSPVLRWGISRAVVGQS (SEQ ID NO: 149) corresponding to amino acids 1270-1327 of Z18303_P12 (SEQ ID NO:134), wherein said first amino acid sequence and second amino acid sequence are contiguous and in a sequential order.

B. An isolated polypeptide comprising an edge portion of Z18303_P12 (SEQ ID NO:134), comprising an amino acid sequence being at least 70%, optionally at least about 80%, preferably at least about 85%, more preferably at least about 90% and most preferably at least about 95% homologous to the sequence CLSDQAGSWGWPGTTGCQPRARSLEGSWGNPSLLLDVCVTSVSPVLRWGISRAVVGQS (SEQ ID NO: 149) of Z18303_P12 (SEQ ID NO:134).

### 2. Comparison Report Between Z18303_P12 (SEQ ID NO:134) and Known Protein(s) NP_{—}000247 (SEQ ID NO: 128):

A. An isolated chimeric polypeptide comprising Z18303_P12 (SEQ ID NO:134), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247 (SEQ ID NO: 128), which also corresponds to amino acids 1-247 of Z18303_P12 (SEQ ID NO:134), a bridging amino acid D corresponding to amino acid 248 of Z18303_P12 (SEQ ID NO:134), a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVTDLRGMLKRLKGMRRDEKKSTAFQKKLEPAYQVSKGHKIRLTVELADH DAEVKWLKNGQEIQMSGSKYIFESIGAKRTLTISQCSLADDAAYQCVVGGEKCSTELFVKEPPVLITRPLED QLVMVGQRVEFECEVSEEGAQVKWLKDGVELTREETFKYRFKKDGQRHHLIINEAMLEDAGHYALCTSG GQAL corresponding to amino acids 249-535 of known protein(s) NP_{—}000247 (SEQ ID NO: 128), which also corresponds to amino acids 249-535 of Z18303_P12 (SEQ ID NO:134), a bridging amino acid A corresponding to amino acid 536 of Z18303_P12 (SEQ ID NO:134), a third amino acid sequence being at least 90% homologous to ELIVQEKKLEVYQSIADLMVGAICDQAVFKCEVSDENVRGVWLKNGKELVPDSRIKVSHIGRVHKLTIDDV TPADEADYSFVPEGFACNLSAKLHFMEVKIDFVPRQEPPKIHLDCPGRIPDTIVVVAGNKLRLDVPISGDPAP TVIWQKAITQGNKAPARPAPDAPEDTGDSDEWVFDKKLLCETEGRVRVETTKDRSIFTVEGAEKEDEGVY TVTVKNPVGEDQVNLTVKVIDVPDAPAAPKISNVGEDSCTVQWEPPAYDGGQPILGYILERKKKKSYRWM corresponding to amino acids 537-819 of known protein(s) NP_{—}000247 (SEQ ID NO: 128), which also corresponds to amino acids 537-819 of Z18303_P12 (SEQ ID NO:134), a bridging amino acid R corresponding to amino acid 820 of Z18303_P12 (SEQ ID NO:134), a fourth amino acid sequence being at least 90% homologous to LNFDLIQELSHEARRMIEGVVYEMRVYAVNAIGMSR SPASQPFMPIGPPSEPTHLAVEDVSDTTVSLKWRP PERVGAGGLDGYSVEYCPEGCSEWVAALQGLTEHTSILVKDLPTGARLLFRVRAHNMAGPGAPVTTTEPV TVQEILQRPRLQLPRHLRQTIQKKVGEPVNLLIPFQGKPRPQVTWTKEGQPLAGEEVSIRNSPTDTILFIRAA RRVHSGTYQVTVRIENMEDKATLVLQVVDKPSPPQDLRVTDAWGLNVALEWKPPQDVGNTELWGYTVQ KADKKTMEWFTVLEHYRRTHCVVPELIIGNGYYFRVFSQNMVGFSDRAATTKEPVFIPRPGITYEPPNYKA LDFSEAPSFTQPLVNRSVIAGYTAMLCCAVRGSPKPKISWFKNGLDLGEDARFRMFSKQGVLTLEIRKPCPF DGGIYVCRATNLQGEARCECRLE corresponding to amino acids 821-1269 of known protein(s) NP_{—}000247 (SEQ ID NO: 128), which also corresponds to amino acids 821-1269 of Z18303_P12 (SEQ ID NO:134), and a fifth amino acid sequence being at least 70%, optionally at least 80%, preferably at least 85%, more preferably at least 90% and most preferably at least 95% homologous to a polypeptide having the sequence CLSDQAGSWGWPGTTGCQPRARSLEGSWGNPSLLLDVCVTSVSPVLRWGISRAVVGQS (SEQ ID NO: 149) corresponding to amino acids 1270-1327 of Z18303_P12 (SEQ ID NO:134), wherein said first amino acid sequence, bridging amino acid, second amino acid sequence, bridging amino acid, third amino acid sequence, bridging amino acid, fourth amino acid sequence and fifth amino acid sequence are contiguous and in a sequential order.

B. An isolated polypeptide comprising an edge portion of Z18303_P12 (SEQ ID NO:134), comprising an amino acid sequence being at least 70%, optionally at least about 80%, preferably at least about 85%, more preferably at least about 90% and most preferably at least about 95% homologous to the sequence CLSDQAGSWGWPGTTGCQPRARSLEGSWGNPSLLLDVCVTSVSPVLRWGISRAVVGQS (SEQ ID NO: 149) of Z18303_P12 (SEQ ID NO:134).

The localization of the variant protein was determined according to results from a number of different software programs and analyses, including analyses from SignalP and other specialized programs. The variant protein is believed to be located as follows with regard to the cell: intracellularly.

Variant protein Z18303_P12 (SEQ ID NO:134) also has the following non-silent SNPs (Single Nucleotide Polymorphisms) as listed in Table 119, (given according to their position(s) on the amino acid sequence, with the alternative amino acid(s) listed; the last column indicates whether the SNP is known or not; the presence of known SNPs in variant protein Z18303_P12 (SEQ ID NO:134) sequence provides support for the deduced sequence of this variant protein according to the present invention).

TABLE 119
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |
| 382 | R -> W |
| 383 | L -> V |
| 522 | A -> T |
| 654 | R -> H |
| 688 | T -> K |
| 804 | L -> M |
| 820 | R -> Q |
| 833 | A -> V |
| 998 | Q -> E |
| 1002 | R -> W |
| 1048 | R -> C |
| 1091 | D -> N |
| 1296 | S -> N |
| 1306 | V -> A |

The phosphorylation sites of variant protein Z18303_P12 (SEQ ID NO:134), as compared to the known protein, are described in Table 120 (given according to their position(s) on the amino acid sequence in the first column; the second column indicates whether the phosphorylation site is present in the variant protein; and the last column indicates whether the position is different on the variant protein).

TABLE 120
Phosphorylation site(s)

| Position(s) | | |
|---|---|---|
| on known | Present | Position(s) |
| amino acid | in variant | on variant |
| sequence | protein? | protein |
| 275 | Yes | 275 |
| 284 | Yes | 284 |
| 304 | Yes | 304 |

Variant protein Z18303_P12 (SEQ ID NO:134) is encoded by the following transcript(s): Z18303_T13 (SEQ ID NO:114). The coding portion of transcript Z18303_T13 (SEQ ID NO:114) starts at position 56 and ends at position 4036. The transcript also has the following SNPs as listed in Table 121 (given according to their position on the nucleotide sequence, with the alternative nucleic acid listed; the last column indicates whether the SNP is known or not; the presence of known SNPs in variant protein Z18303_P12 (SEQ ID NO:134) sequence provides support for the deduced sequence of this variant protein according to the present invention).

TABLE 121
Nucleic acid SNPs

| Polymorphism | SNP position(s) on nucleotide sequence |
|---|---|
| T -> C | 88, 3972 |
| G -> T | 263 |
| C -> T | 427, 547, 592, 841, 1199, 2553, 2602, 2656, 3059, 3197 |
| G -> A | 527, 620, 897, 1619, 2016, 2514, 3326, 3343, 3942, |
| | 4109 |
| T -> A | 726 |
| A -> G | 761, 3898 |
| C -> A | 799, 2118, 2465 |
| C -> G | 1202, 3047 |
| C -> | 4085 |

Variant protein Z18303_P19 (SEQ ID NO:135) according to the present invention is encoded by transcript(s) Z18303_T22 (SEQ ID NO:115). An alignment is given to the known protein (Myosin-binding protein C (SEQ ID NO:127), cardiac-type) at the end of the application. One or more alignments to one or more previously published protein sequences are given at the end of the application. A brief description of the relationship of the variant protein according to the present invention to each such aligned protein is as follows:

### 1. Comparison Report Between Z18303_P19 (SEQ ID NO:135) and Known Protein(s) MYPC_HUMAN (SEQ ID NO: 127) and Q9UM53_HUMAN (SEQ ID NO: 127):

A. An isolated chimeric polypeptide comprising Z18303_P19 (SEQ ID NO:135), comprising a amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKFDCSNFNLTVHEAMGTGDLDLLSAFRRTSLAGGGRR ISDSHEDTGILDFSSLLICKRDSFRTPRDSKLEAPAEEDVWEILRQAPPSEYERIAFQYGVTDLRGMLICRLKG MRRDEKKSTAFQKKLEPAYQVSKGHKIRLTVELADHDAEVKWLKNGQEIQMSGSKYIFESIGAKRTLTISQ CSLADDAAYQCVVGGEKCSTELFVKEPPVLITRPLEDQLVMVGQRVEFECEVSEEGAQVKWLKDGVELTR EETFKYRFKKDGQRHHLIINEAMLEDAGHYALCTSGGQALAELIVQEKKLEVYQSIADLMVGAKDQAVFK CEVSDENVRGVWLKNGKELVPDSRIKVSHIGRVHKLTIDDVTPADEADYSFVPEGFACNLSAKLHFMEVKI DFVPRQEPPKIHLDCPGRIPDTIVVVAGNKLRLDVPISGDPAPTVIWQKAITQGNKAPARPAPDAPEDTGDS DEWVFDKKLLCETEGRVRVETTKDRSIFTVEGAEKEDEGVYTVTVKNPVGEDQVNLTVKVIDVPDAPAAP KISNVGEDSCTVQWEPPAYDGGQPILGYILERKKKKSYRWMRLNFDLIQELSHEARRMIEGVVYEMRVYA VNAIGMSRPSPASQPFMPIGPPSEPTHLAVEDVSDTIVSLKWRPPERVGAGGLDGYSVEYCPEGCSEWVAA LQGLTEHTSILVKDLPTGARLLFRVRAHNMAGPGAPVTTTEPVTVQEILQRPRLQLPRHLRQTIQKKVGEPV NLLIPFQGKPRPQVTWTKEGQPLAGEEVSIRNSPTDTILFIRAARRVHSGTYQVTVRIENMEDKATLVLQVV DKPSPPQDLRVTDAWGLNVALEWKPPQDVGNTELWGYTVQKADKKTM corresponding to amino acids 1-1110 of known protein(s) MYPC_HUMAN (SEQ ID NO: 127) and Q9UM53_HUMAN (SEQ ID NO: 127), which also corresponds to amino acids 1-1110 of Z18303_P19 (SEQ ID NO:135).

### 2. Comparison Report Between Z18303_P19 (SEQ ID NO:135) and Known Protein(s) NP_{—}000247 (SEQ ID NO: 128):

A. An isolated chimeric polypeptide comprising Z18303_P19 (SEQ ID NO:135), comprising a first amino acid sequence being at least 90% homologous to MPEPGKKPVSAFSKKPRSVEVAAGSPAVFEAETERAGVKVRWQRGGSDISASNKYGLATEGTRHTLTVRE VGPADQGSYAVIAGSSKVKFDLKVIEAEKAEPMLAPAPAPAEATGAPGEAPAPAAELGESAPSPKGSSSAA LNGPTPGAPDDPIGLFVMRPQDGEVTVGGSITFSARVAGASLLKPPVVKWFKGKWVDLSSKVGQHLQLHD SYDRASKVYLFELHITDAQPAFTGSYRCEVSTKDKF corresponding to amino acids 1-247 of known protein(s) NP_{—}000247 (SEQ ID NO: 128), which also corresponds to amino acids 1-247 of Z18303_P19 (SEQ ID NO:135), a bridging amino acid D corresponding to amino acid 248 of Z18303_P19 (SEQ ID NO:135), a second amino acid sequence being at least 90% homologous to CSNFNLTVHEAMGTGDLDLLSAFRRTSLAGGGRRISDSHEDTGILDFSSLLKKRDSFRTPRDSKLEAPAEED VWEILRQAPPSEYERIAFQYGVTDLRGMLKRLKGMRRDEKKSTAFQKKLEPAYQVSKGHKIRLTVELADH DAEVKWLKNGQEIQMSGSKYIFESIGAKRTLTISQCSLADDAAYQCVVGGEKCSTELFVKEPPVLITRPLED QLVMVGQRVEFECEVSEEGAQVKWLKDGVELTREETFKYRFKKDGQRHHLIINEAMLEDAGHYALCTSG GQAL corresponding to amino acids 249-535 of known protein(s) NP_{—}000247 (SEQ ID NO: 128), which also corresponds to amino acids 249-535 of Z18303_P19 (SEQ ID NO:135), a bridging amino acid A corresponding to amino acid 536 of Z18303_P19 (SEQ ID NO:135), a third amino acid sequence being at least 90% homologous to ELIVQEKKLEVYQSIADLMVGAKDQAVFKCEVSDENVRGVWLKNGKELVPDSRIKVSHIGRVHKLTIDDV TPADEADYSFVPEGFACNLSAKLHFMEVKIDFVPRQEPPKIHLDCPGRIPDTIVVVAGNKLRLDVPISGDPAP TVIWQKAITQGNKAPARPAPDAPEDTGDSDEWVFDKKLLCETEGRVRVETTKDRSIFTVEGAEKEDEGVY TVTVKNPVGEDQVNLTVKVIDVPDAPAAPKISNVGEDSCTVQWEPPAYDGGQPILGYILERKKKKSYRWM corresponding to amino acids 537-819 of known protein(s) NP_{—}000247 (SEQ ID NO: 128), which also corresponds to amino acids 537-819 of Z18303_P19 (SEQ ID NO:135), a bridging amino acid R corresponding to amino acid 820 of Z18303_P19 (SEQ ID NO:135), and a fourth amino acid sequence being at least 90% homologous to LNFDLIQELSHEARRMIEGVVYEMRVYAVNAIGMSRPSPASQPFMPIGPPSEPTHLAVEDVSDTTVSLKWRP PERVGAGGLDGYSVEYCPEGCSEWVAALQGLTEHTSILVKDLPTGARLLFRVRAHNMAGPGAPVTTTEPV TVQEILQRPRLQLPRHLRQTIQKKVGEPVNLLIPFQGKPRPQVTWTKEGQPLAGEEVSIRNSPTDTILFIRAA RRVHSGTYQVTVRIENMEDKATLVLQVVDKPSPPQDLRVTDAWGLNVALEWKPPQDVGNTELWGYTVQ KADKKTM corresponding to amino acids 821-1110 of known protein(s) NP_{—}000247 (SEQ ID NO: 128), which also corresponds to amino acids 821-1110 of Z18303_P19 (SEQ ID NO:135), wherein said first amino acid sequence, bridging amino acid, second amino acid sequence, bridging amino acid, third amino acid sequence, bridging amino acid and fourth amino acid sequence are contiguous and in a sequential order.

The localization of the variant protein was determined according to results from a number of different software programs and analyses, including analyses from SignalP and other specialized programs. The variant protein is believed to be located as follows with regard to the cell: intracellularly.

Variant protein Z18303_P19 (SEQ ID NO:135) also has the following non-silent SNPs (Single Nucleotide Polymorphisms) as listed in Table 122, (given according to their position(s) on the amino acid sequence, with the alternative amino acid(s) listed; the last column indicates whether the SNP is known or not; the presence of known SNPs in variant protein Z18303_P19 (SEQ ID NO:135) sequence provides support for the deduced sequence of this variant protein according to the present invention).

TABLE 122
Amino acid mutations

| SNP position(s) | |
|---|---|
| on amino acid | Alternative |
| sequence | amino acid(s) |
| 70 | E -> * |
| 158 | V -> M |
| 189 | V -> I |
| 224 | L -> Q |
| 236 | S -> G |
| 248 | D -> E |
| 281 | R -> Q |
| 382 | R -> W |
| 383 | L -> V |
| 522 | A -> T |
| 654 | R -> H |
| 688 | T -> K |
| 804 | L -> M |
| 820 | R -> Q |
| 833 | A -> V |
| 998 | Q -> E |
| 1002 | R -> W |
| 1048 | R -> C |
| 1091 | D -> N |

The phosphorylation sites of variant protein Z18303_P19 (SEQ ID NO:135), as compared to the known protein, are described in Table 123 (given according to their position(s) on the amino acid sequence in the first column; the second column indicates whether the phosphorylation site is present in the variant protein; and the last column indicates whether the position is different on the variant protein).

TABLE 123
Phosphorylation site(s)

| Position(s) | | |
|---|---|---|
| on known | Present | Position(s) |
| amino acid | in variant | on variant |
| sequence | protein? | protein |
| 275 | Yes | 275 |
| 284 | Yes | 284 |
| 304 | Yes | 304 |

Variant protein Z18303_P19 (SEQ ID NO:135) is encoded by the following transcript(s): Z18303_T22 (SEQ ID NO:115). The coding portion of transcript Z18303_T22 (SEQ ID NO:115) starts at position 56 and ends at position 3385. The transcript also has the following SNPs as listed in Table 124 (given according to their position on the nucleotide sequence, with the alternative nucleic acid listed; the last column indicates whether the SNP is known or not; the presence of known SNPs in variant protein Z18303_P19 (SEQ ID NO:135) sequence provides support for the deduced sequence of this variant protein according to the present invention).

TABLE 124
Nucleic acid SNPs

| Polymorphism | SNP position(s) on nucleotide sequence |
|---|---|
| T -> C | 88 |
| G -> T | 263 |
| C -> T | 427, 547, 592, 841, 1199, 2553, 2602, 2656, 3059, 3197 |
| G -> A | 527, 620, 897, 1619, 2016, 2514, 3326, 3343 |
| T -> A | 726 |
| A -> G | 761 |
| C -> A | 799, 2118, 2465 |
| C -> G | 1202, 3047 |

As noted above, cluster Z18303 features 11 segment(s), which were listed in Table 101. These segment(s) are portions of nucleic acid sequence(s) which are described herein separately because they are of particular interest. A description of each segment according to the present invention is now provided.

Segment cluster Z18303_N28 (SEQ ID NO:116) according to the present invention is supported by 18 libraries. The number of libraries was determined as previously described. This segment can be found in the following transcript(s): Z18303_T11 (SEQ ID NO:113), Z18303_T13 (SEQ ID NO:114), Z18303_T22 (SEQ ID NO:115), Z18303_T3 (SEQ ID NO:109), Z18303_T4 (SEQ ID NO:110), Z18303_T6 (SEQ ID NO:111) and Z18303_T7 (SEQ ID NO:112). Table 125 below describes the starting and ending position of this segment on each transcript.

TABLE 125
Segment location on transcripts

| | Segment | Segment |
|---|---|---|
| Transcript name | starting position | ending position |
| Z18303_T11 (SEQ ID NO: 113) | 982 | 1145 |
| Z18303_T13 (SEQ ID NO: 114) | 982 | 1145 |
| Z18303_T22 (SEQ ID NO: 115) | 982 | 1145 |
| Z18303_T3 (SEQ ID NO: 109) | 982 | 1145 |
| Z18303_T4 (SEQ ID NO: 110) | 982 | 1145 |
| Z18303_T6 (SEQ ID NO: 111) | 982 | 1145 |
| Z18303_T7 (SEQ ID NO: 112) | 1276 | 1439 |

Segment cluster Z18303_N29 (SEQ ID NO:117) according to the present invention is supported by 18 libraries. The number of libraries was determined as previously described. This segment can be found in the following transcript(s): Z18303_T3 (SEQ ID NO:109). Table 126 below describes the starting and ending position of this segment on each transcript.

TABLE 126
Segment location on transcripts

| | Segment | Segment |
|---|---|---|
| Transcript name | starting position | ending position |
| Z18303_T3 (SEQ ID NO: 109) | 1146 | 3600 |

Segment cluster Z18303_N30 (SEQ ID NO:118) according to the present invention is supported by 5 libraries. The number of libraries was determined as previously described. This segment can be found in the following transcript(s): Z18303_T3 (SEQ ID NO:109). Table 127 below describes the starting and ending position of this segment on each transcript.

TABLE 27
Segment location on transcripts

| | Segment | Segment |
|---|---|---|
| Transcript name | starting position | ending position |
| Z18303_T3 (SEQ ID NO: 109) | 3601 | 3727 |

Segment cluster Z18303_N57 (SEQ ID NO:119) according to the present invention is supported by 6 libraries. The number of libraries was determined as previously described. This segment can be found in the following transcript(s): Z18303_T4 (SEQ ID NO:110). Table 128 below describes the starting and ending position of this segment on each transcript.

TABLE 128
Segment location on transcripts

| | Segment | Segment |
|---|---|---|
| Transcript name | starting position | ending position |
| Z18303_T4 (SEQ ID NO: 110) | 2364 | 2661 |

Segment cluster Z18303_N87 (SEQ ID NO:120) according to the present invention is supported by 8 libraries. The number of libraries was determined as previously described. This segment can be found in the following transcript(s): Z18303_T11 (SEQ ID NO:113). Table 129 below describes the starting and ending position of this segment on each transcript.

TABLE 129
Segment location on transcripts

| | Segment | Segment |
|---|---|---|
| Transcript name | starting position | ending position |
| Z18303_T11 (SEQ ID NO: 113) | 3870 | 4059 |

According to an optional embodiment of the present invention, short segments related to the above cluster are also provided. These segments are up to about 120 by in length, and so are included in a separate description.

Segment cluster Z18303_N19 (SEQ ID NO:121) according to the present invention is supported by 2 libraries. The number of libraries was determined as previously described. This segment can be found in the following transcript(s): Z18303_T7 (SEQ ID NO:112). Table 130 below describes the starting and ending position of this segment on each transcript.

TABLE 130
Segment location on transcripts

| | Segment | Segment |
|---|---|---|
| Transcript name | starting position | ending position |
| Z18303_T7 (SEQ ID NO: 112) | 907 | 944 |

Segment cluster Z18303_N120 (SEQ ID NO:122) according to the present invention is supported by 16 libraries. The number of libraries was determined as previously described. This segment can be found in the following transcript(s): Z18303_T11 (SEQ ID NO:113), Z18303_T13 (SEQ ID NO:114), Z18303_T22 (SEQ ID NO:115), Z18303_T13 (SEQ ID NO:109), Z18303_T4 (SEQ ID NO:110), Z18303_T6 (SEQ ID NO:111) and Z18303_T7 (SEQ ID NO:112). Table 131 below describes the starting and ending position of this segment on each transcript.

TABLE 131
Segment location on transcripts

| | Segment | Segment |
|---|---|---|
| Transcript name | starting position | ending position |
| Z18303_T11 (SEQ ID NO: 113) | 907 | 960 |
| Z18303_T13 (SEQ ID NO: 114) | 907 | 960 |
| Z18303_T22 (SEQ ID NO: 115) | 907 | 960 |
| Z18303_T3 (SEQ ID NO: 109) | 907 | 960 |
| Z18303_T4 (SEQ ID NO: 110) | 907 | 960 |
| Z18303_T6 (SEQ ID NO: 111) | 907 | 960 |
| Z18303_T7 (SEQ ID NO: 112) | 945 | 998 |

Segment cluster Z18303_N47 (SEQ ID NO:123) according to the present invention is supported by 2 libraries. The number of libraries was determined as previously described. This segment can be found in the following transcript(s): Z18303_T6 (SEQ ID NO:111). Table 132 below describes the starting and ending position of this segment on each transcript.

TABLE 132
Segment location on transcripts

| | Segment | Segment |
|---|---|---|
| Transcript name | starting position | ending position |
| Z18303_T6 (SEQ ID NO: 111) | 1953 | 2057 |

Segment cluster Z18303_N75 (SEQ ID NO:124) according to the present invention is supported by 1 libraries. The number of libraries was determined as previously described. This segment can be found in the following transcript(s): Z18303_T22 (SEQ ID NO:115). Table 133 below describes the starting and ending position of this segment on each transcript.

TABLE 133
Segment location on transcripts

| | Segment | Segment |
|---|---|---|
| Transcript name | starting position | ending position |
| Z18303_T22 (SEQ ID NO: 115) | 3386 | 3451 |

Segment cluster Z18303_N85 (SEQ ID NO:125) according to the present invention is supported by 36 libraries. The number of libraries was determined as previously described. This segment can be found in the following transcript(s): Z18303_T11 (SEQ ID NO:113), Z18303_T13 (SEQ ID NO:114), Z18303_T3 (SEQ ID NO:109), Z18303_T4 (SEQ ID NO:110), Z18303_T6 (SEQ ID NO:111) and Z18303_T7 (SEQ ID NO:112). Table 134 below describes the starting and ending position of this segment on each transcript.

TABLE 134
Segment location on transcripts

| | Segment | Segment |
|---|---|---|
| Transcript name | starting position | ending position |
| Z18303_T11 (SEQ ID NO: 113) | 3817 | 3862 |
| Z18303_T13 (SEQ ID NO: 114) | 3817 | 3862 |
| Z18303_T3 (SEQ ID NO: 109) | 6399 | 6444 |
| Z18303_T4 (SEQ ID NO: 110) | 4115 | 4160 |
| Z18303_T6 (SEQ ID NO: 111) | 3922 | 3967 |
| Z18303_T7 (SEQ ID NO: 112) | 4111 | 4156 |

Segment cluster Z18303_N88 (SEQ ID NO:126) according to the present invention is supported by 35 libraries. The number of libraries was determined as previously described. This segment can be found in the following transcript(s): Z18303_T11 (SEQ ID NO:113), Z18303_T13 (SEQ ID NO:114), Z18303_T3 (SEQ ID NO:109), Z18303_T4 (SEQ ID NO:110), Z18303_T6 (SEQ ID NO:111) and Z18303_T7 (SEQ ID NO:112). Table 135 below describes the starting and ending position of this segment on each transcript.

TABLE 135
Segment location on transcripts

| | Segment | Segment |
|---|---|---|
| Transcript name | starting position | ending position |
| Z18303_T11 (SEQ ID NO: 113) | 4060 | 4096 |
| Z18303_T13 (SEQ ID NO: 114) | 3863 | 3899 |
| Z18303_T3 (SEQ ID NO: 109) | 6452 | 6488 |
| Z18303_T4 (SEQ ID NO: 110) | 4168 | 4204 |
| Z18303_T6 (SEQ ID NO: 111) | 3975 | 4011 |
| Z18303_T7 (SEQ ID NO: 112) | 4164 | 4200 |

Variant Protein Alignment to the Previously Known Protein:

Alignment of: Z18303_P4 (SEQ ID NO:130)×known protein(s) MYPC_HUMAN (SEQ ID NO: 127) and Q9UM53_HUMAN (SEQ ID NO: 127):
Total length: 1373
Matching length: 769

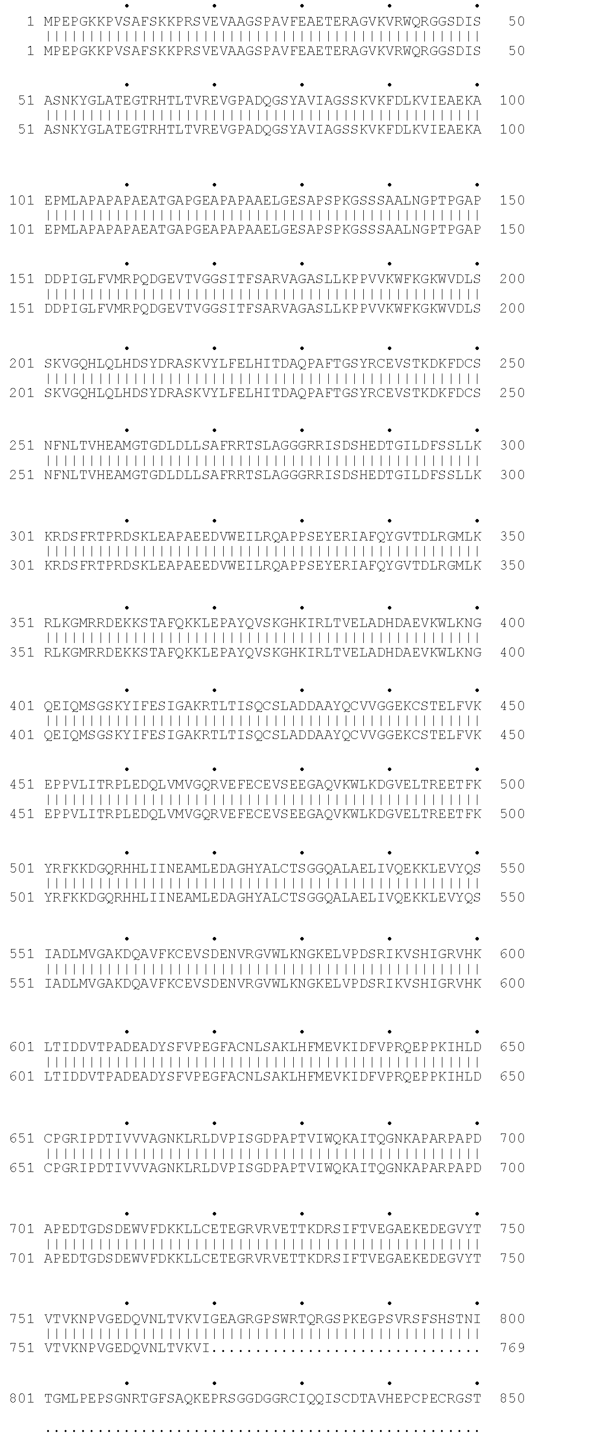

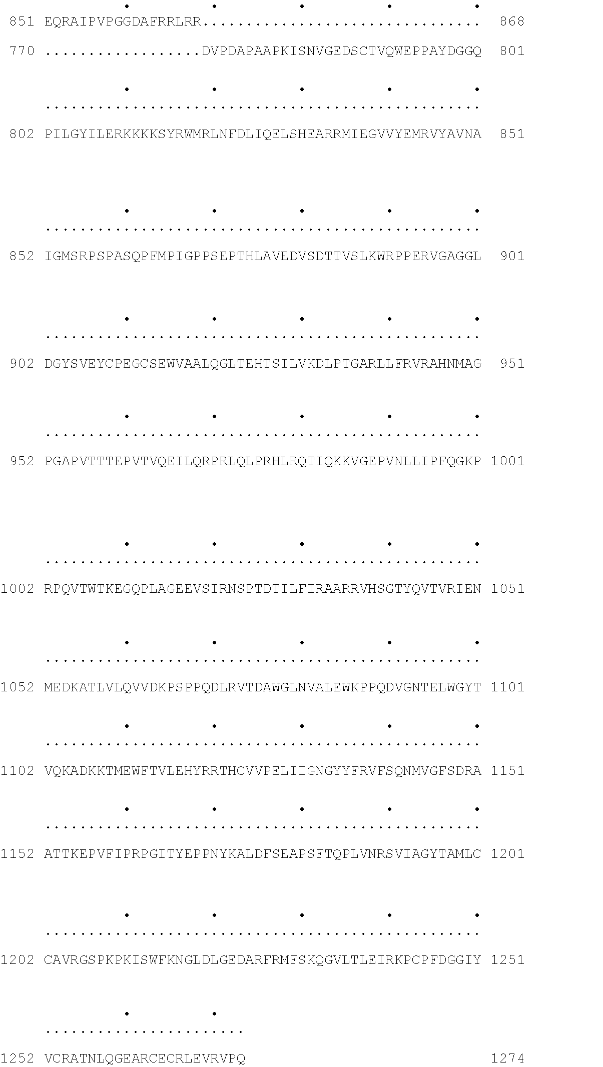

Alignment of: Z18303_P4 (SEQ ID NO:130)×known protein(s) NP_{—}000247 (SEQ ID NO: 128):
Total length: 1373
Matching length: 769

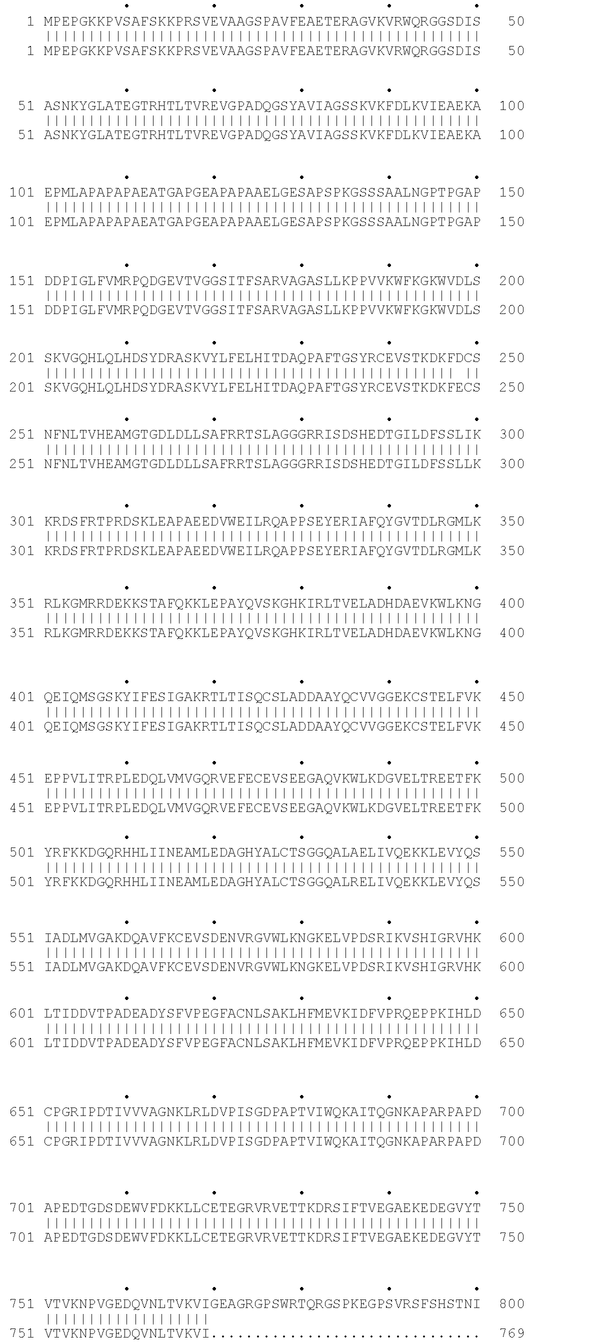

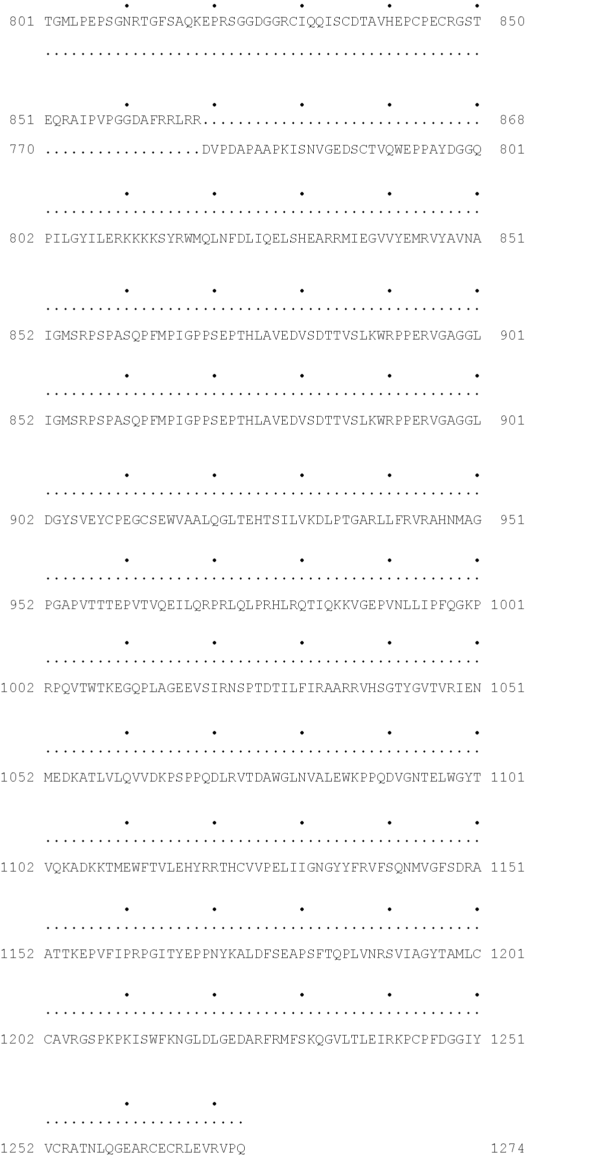

Alignment of: Z18303_P10 (SEQ ID NO:133)×known protein(s) MYPC_HUMAN (SEQ ID NO: 127) and Q9UM53_HUMAN (SEQ ID NO: 127):
Total length: 1330
Matching length: 1271

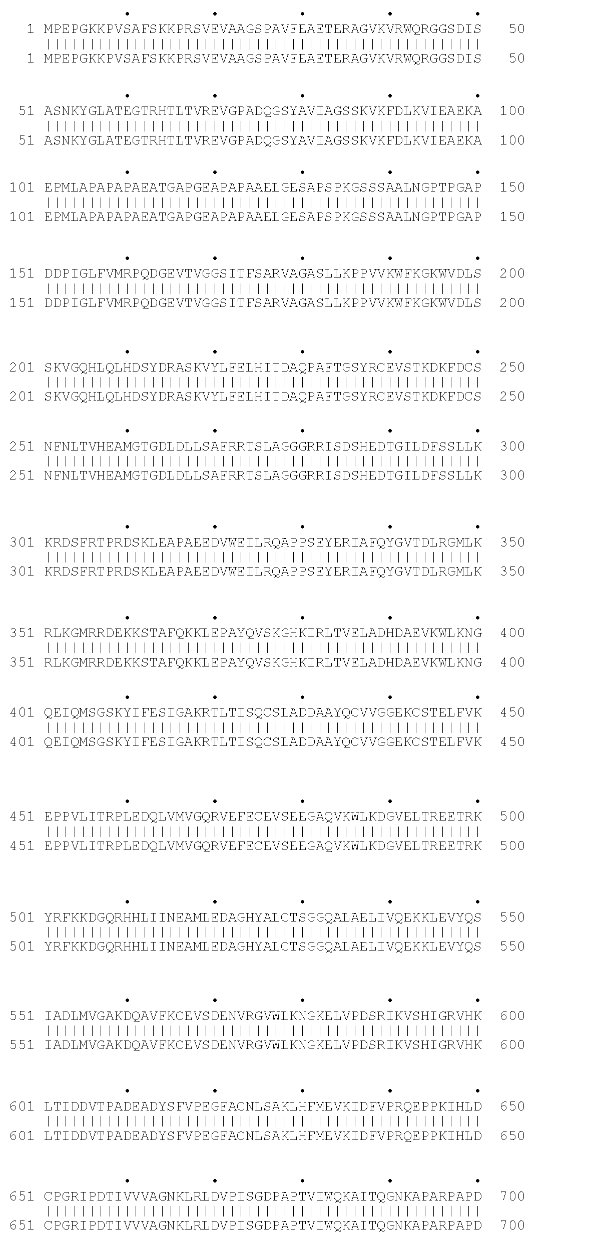

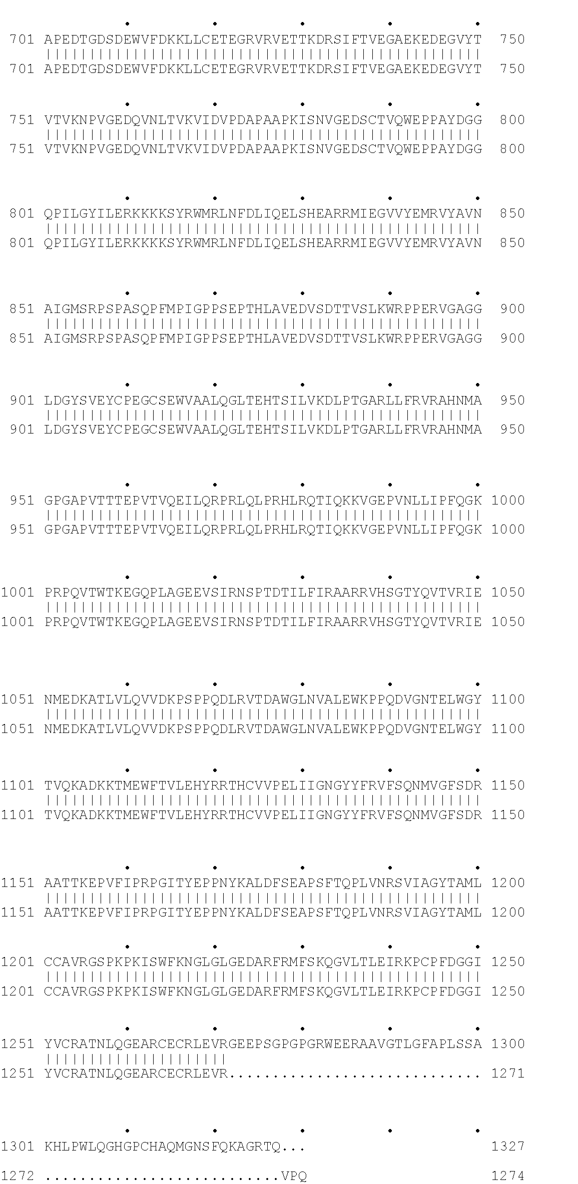

Alignment of: Z18303_P10 (SEQ ID NO:133)×known protein(s) NP_{—}000247 (SEQ ID NO: 128):
Total length: 1330
Matching length: 1271

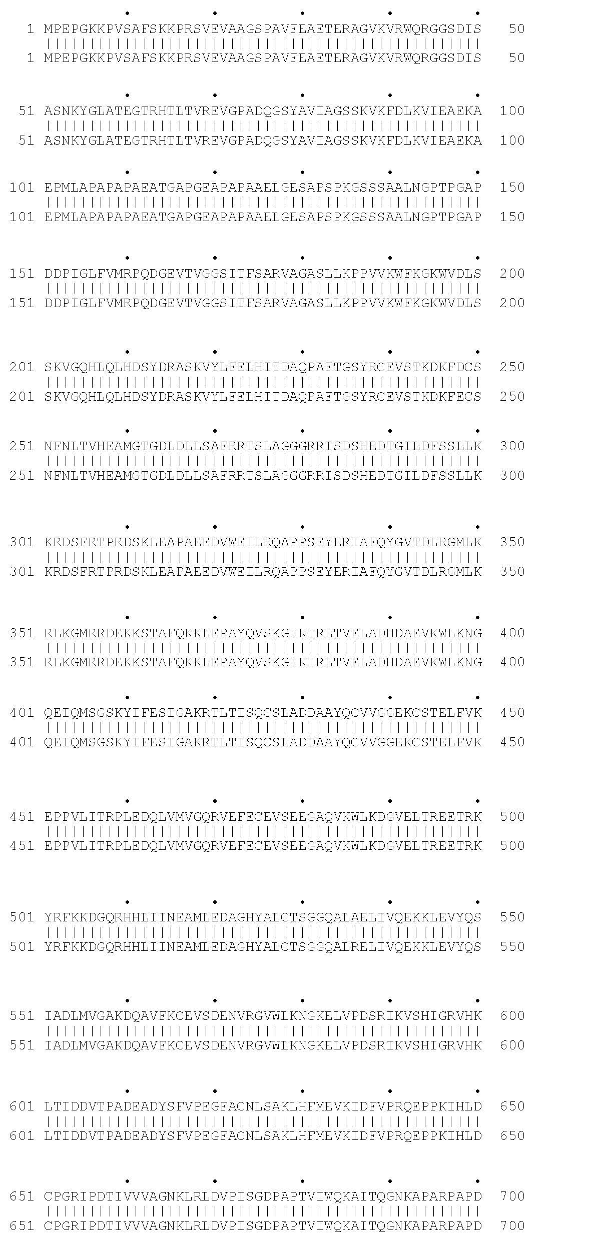

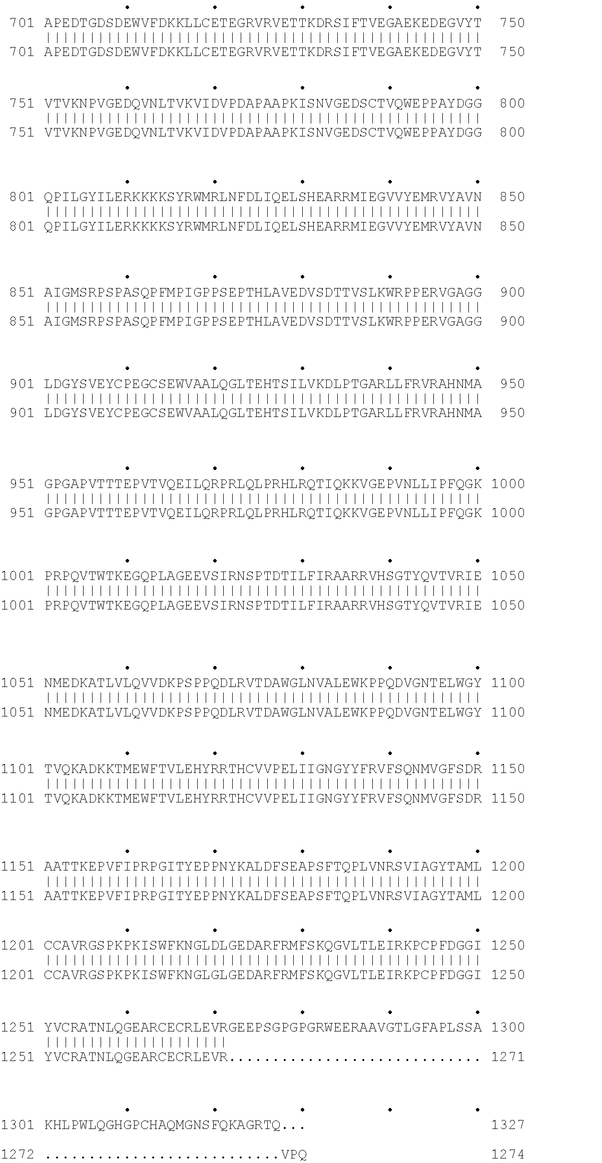

Alignment of: Z18303_P12 (SEQ ID NO:134)×known protein(s) MYPC_HUMAN (SEQ ID NO: 127) and Q9UM53_HUMAN (SEQ ID NO: 127):
Total length: 1332
Matching length: 1269

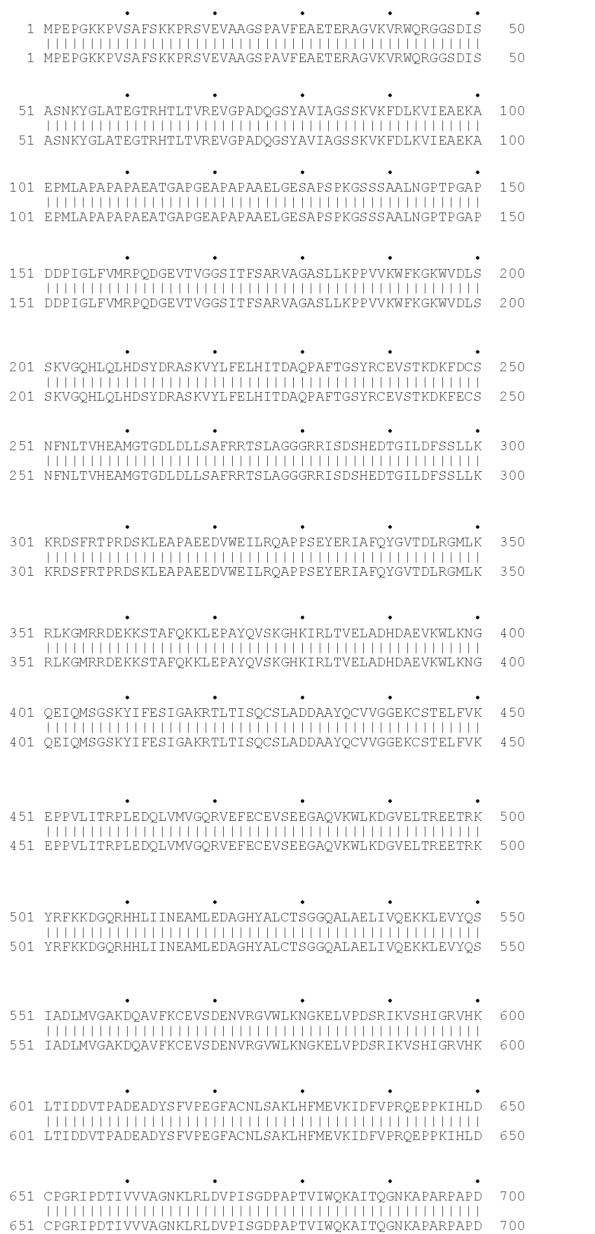

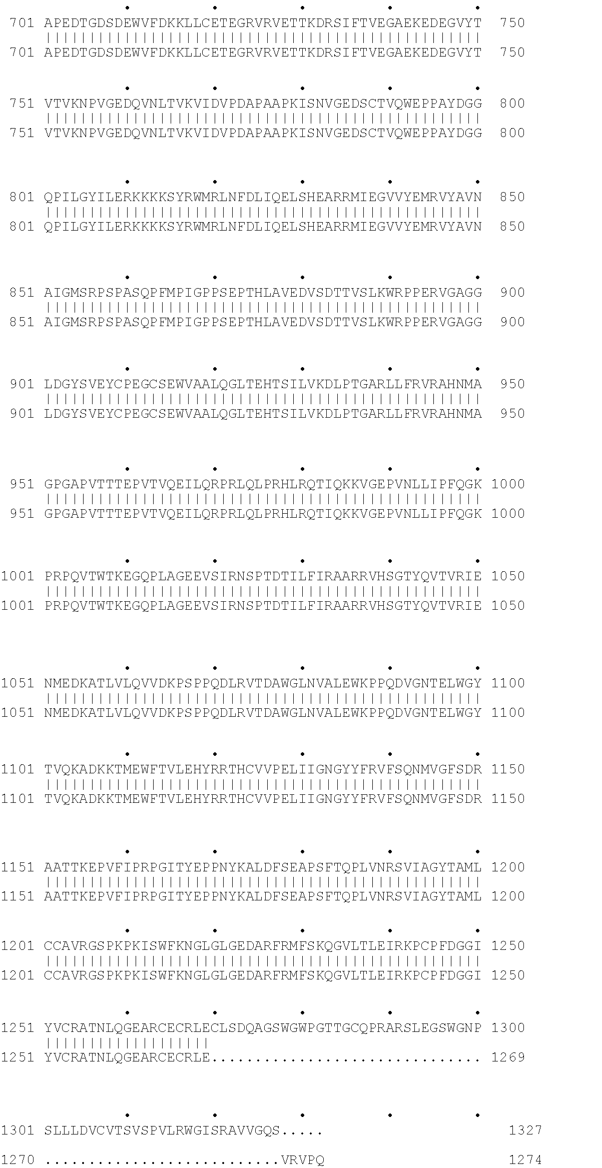

Alignment of: Z18303_P12 (SEQ ID NO:134)×known protein(s) NP_{—}000247 (SEQ ID NO: 128):
Total length: 1332
Matching length: 1269

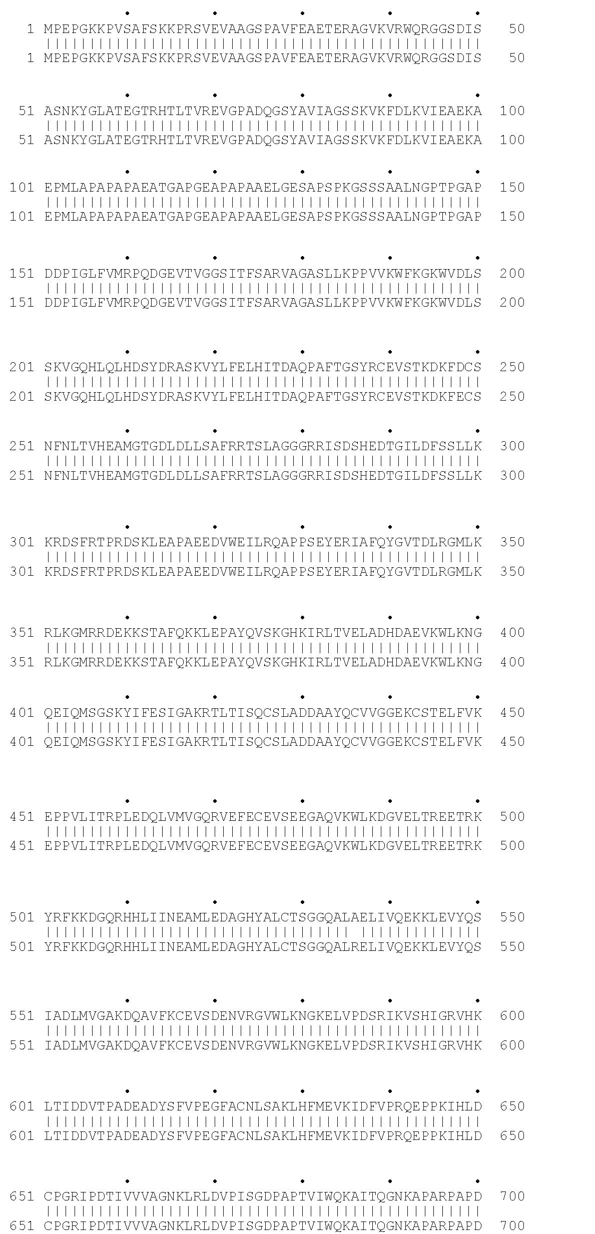

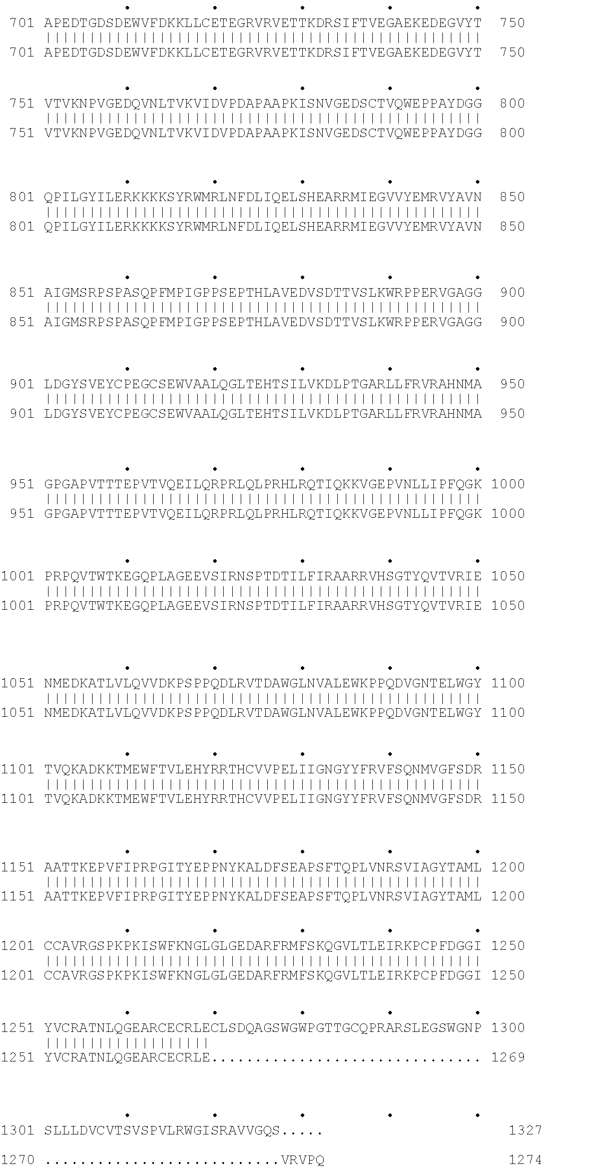

Alignment of: Z18303_P19 (SEQ ID NO:135)×known protein(s) MYPC_HUMAN (SEQ ID NO: 127) and Q9UM53_HUMAN (SEQ ID NO: 127):
Total length: 1274
Matching length: 1110

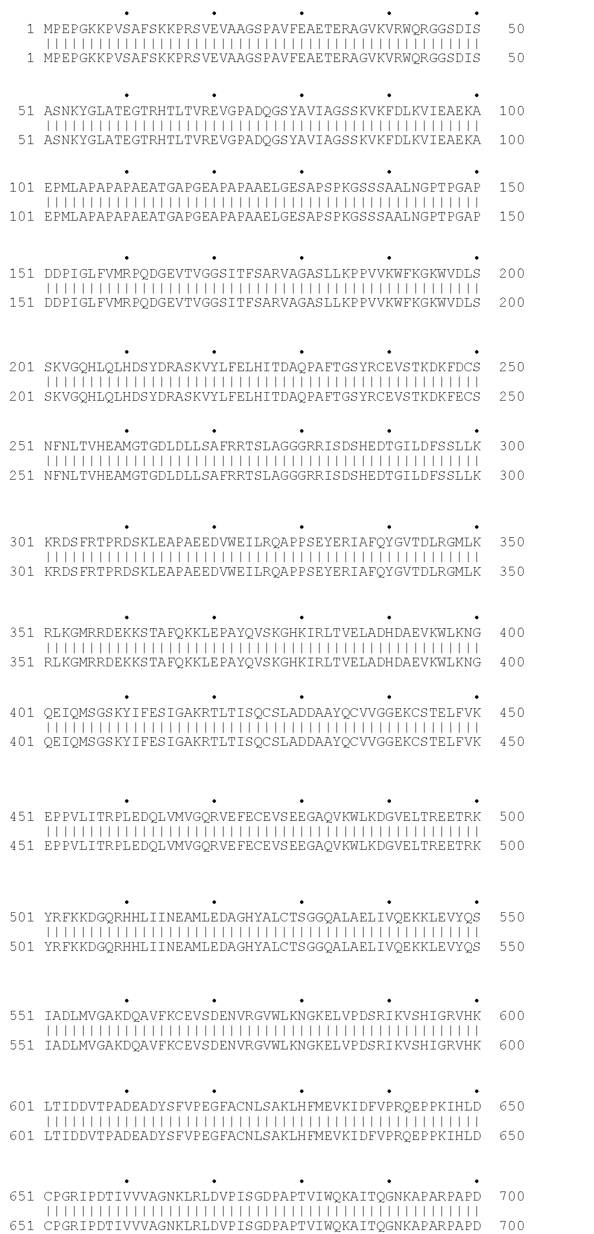

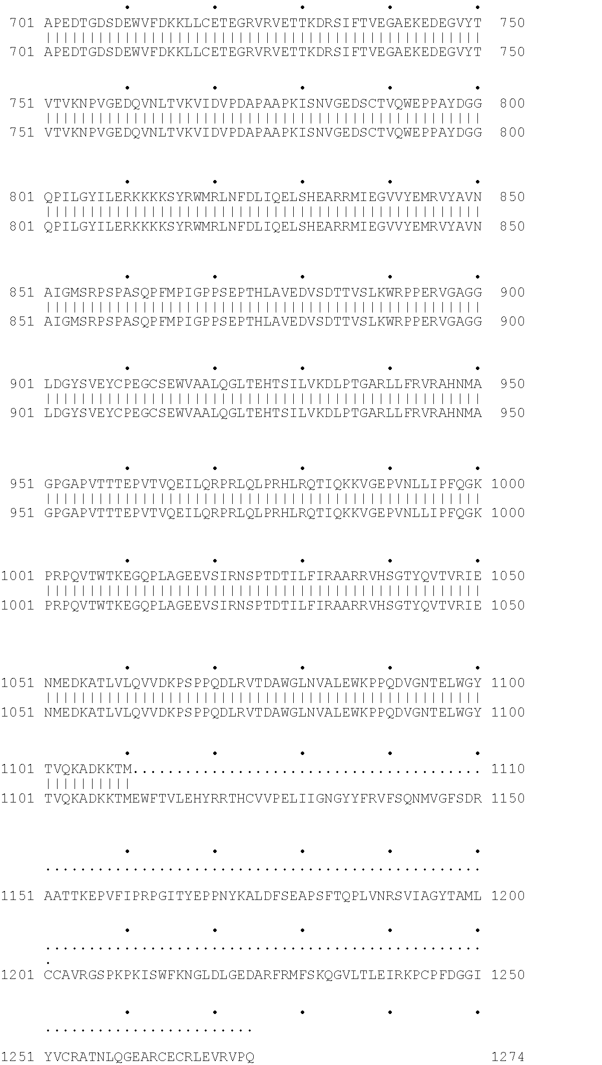

Alignment of: Z18303_P19 (SEQ ID NO:135)×known protein(s) NP_{—}000247 (SEQ ID NO: 128):
Total length: 1274
Matching length: 1110

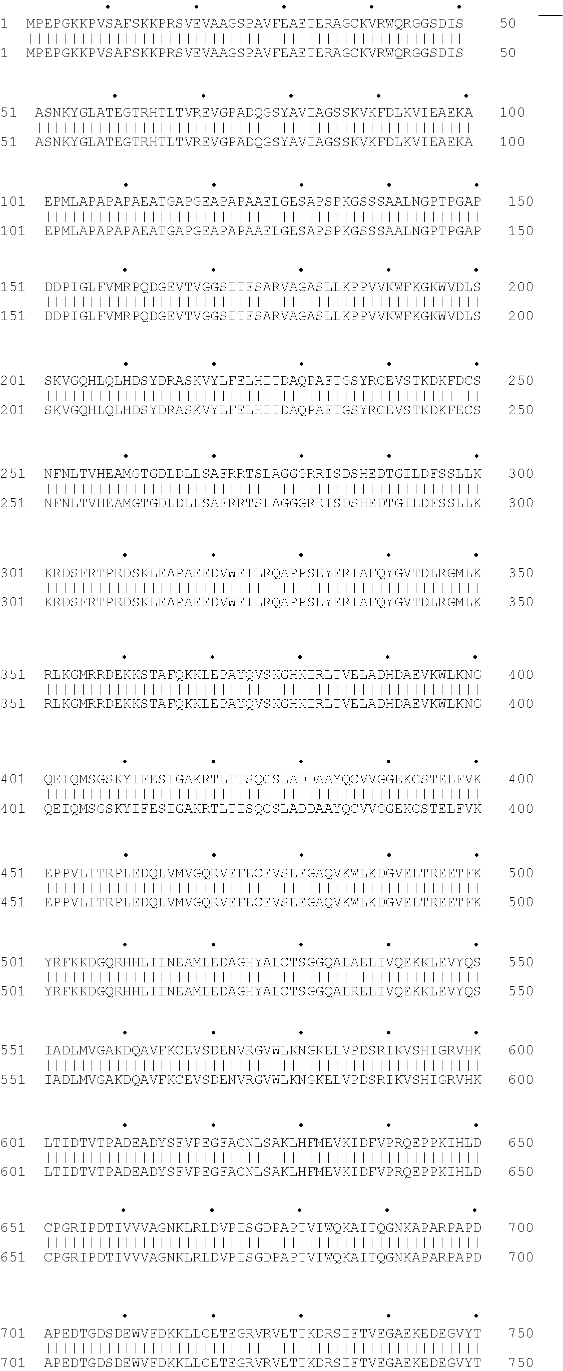

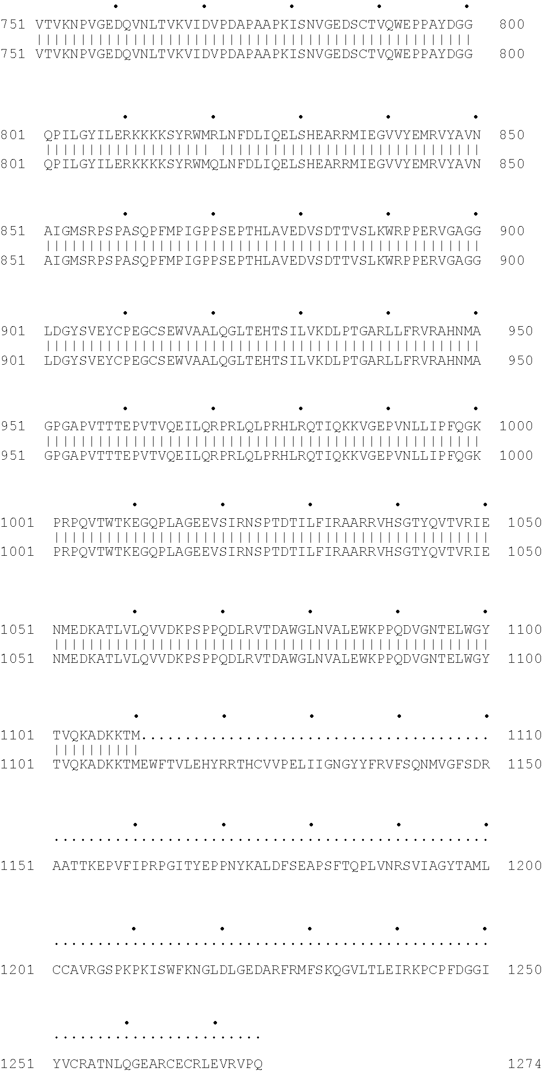

Expression of MYBPC3-Myosin Binding Protein C218303 Transcripts which are Detectable by Amplicon as Depicted in Sequence Name Z18303_seg19-20 (SEQ ID NO: 138) Specifically in Heart Tissue

Expression of MYBPC3-myosin binding protein C transcripts detectable by or according to seg19-20-Z18303_seg19-20 (SEQ ID NO: 138) amplicon and primers Z18303_seg19-20F (SEQ ID NO:136) and Z18303_seg19-20R (SEQ ID NO:137) was measured by real time PCR. Non-detected samples (sample(s) no. 16, 36, 45 and 68) were assigned Ct value of 41 and were calculated accordingly. In parallel the expression of several housekeeping genes—SDHA (GenBank Accession No. NM_{—}004168 (SEQ ID NO: 31); amplicon—SDHA-amplicon (SEQ ID NO: 34)), Ubiquitin (GenBank Accession No. BC000449 (SEQ ID NO: 27); amplicon—Ubiquitin-amplicon (SEQ ID NO: 30)), and TATA box (GenBank Accession No. NM_{—}003194 (SEQ ID NO: 23); TATA amplicon (SEQ ID NO: 26)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the heart samples (sample numbers 65, 66 and 67, Table 1_{—}5 above), to obtain a value of relative expression for each sample relative to median of the heart samples.

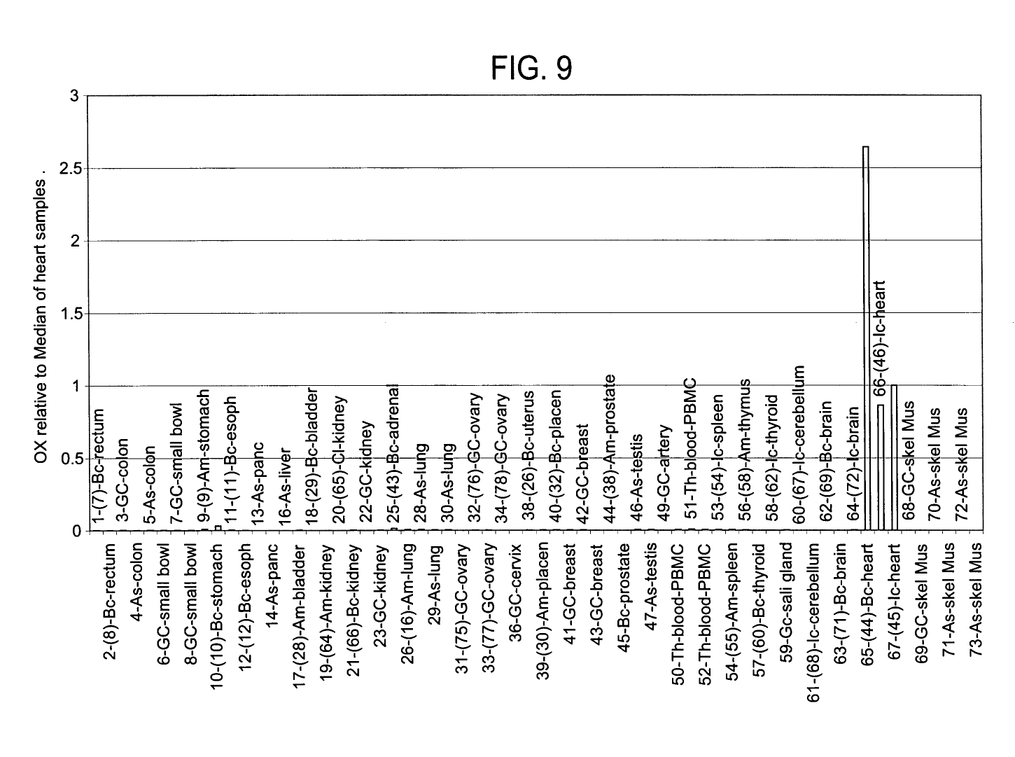

FIG. 9 is a histogram showing relative expression of the above-indicated MYBPC3-myosin binding protein C transcripts in heart tissue samples as opposed to other tissues.

As is evident from FIG. 9, the expression of MYBPC3-myosin binding protein C transcripts detectable by the above amplicon in heart tissue samples was significantly higher than in the other samples.

Primer pairs are also optionally and preferably encompassed within the present invention; for example, for the above experiment, the following primer pair was used as a non-limiting illustrative example only of a suitable primer pair: Z18303_seg19-20F (SEQ ID NO:136) forward primer; and Z18303_seg19-20R (SEQ ID NO:137) reverse primer.

The present invention also preferably encompasses any amplicon obtained through the use of any suitable primer pair; for example, for the above experiment, the following amplicon was obtained as a non-limiting illustrative example only of a suitable amplicon: Z18303_seg19-20 (SEQ ID NO: 138).

Forward Primer (Z18303_seg19-20F (SEQ ID NO:
136)):
TCCCTCACTGCAGCCTCAC
Reverse Primer (Z18303_seg19-20R (SEQ ID NO:
137)):
CTCTTTTTCAGCAGTGAGCTGAAGTC
Amplicon (Z18303_seg19-20) (SEQ ID NO: 138) :
TCCCTCACTGCAGCCTCACTGGGGGTCCCTCTCCATAGTGATAGCCATGA
GGACACTGGGATTCTGGACTTCAGCTCACTGCTGAAAAAGAG

Expression of Myosin Binding Protein C, Cardiac (MYBPC3) Z18303 Transcripts which are Detectable by Amplicon as Depicted in Sequence Name Z18303_seg28-29 (SEQ ID NO:141) Specifically in Heart Tissue

Expression of myosin binding protein C, cardiac (MYBPC3) transcripts detectable by or according to seg28-29-Z18303_seg28-29 (SEQ ID NO:141) amplicon and primers Z18303_seg28-29F (SEQ ID NO:139) and Z18303_seg28-29R (SEQ ID NO:140) was measured by real time PCR. Non-detected sample (sample no. 20) was assigned Ct value of 41 and were calculated accordingly. In parallel the expression of several housekeeping genes —SDHA (GenBank Accession No. NM_{—}004168 (SEQ ID NO: 31); amplicon—SDHA-amplicon (SEQ ID NO: 34)), Ubiquitin (GenBank Accession No. BC000449 (SEQ ID NO: 27); amplicon—Ubiquitin-amplicon (SEQ ID NO: 30)), and TATA box (GenBank Accession No. NM_{—}003194 (SEQ ID NO: 23); TATA amplicon (SEQ ID NO: 26)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the heart samples (sample numbers 65, 66 and 67, Table 1_{—}5 above), to obtain a value of relative expression for each sample relative to median of the heart samples.

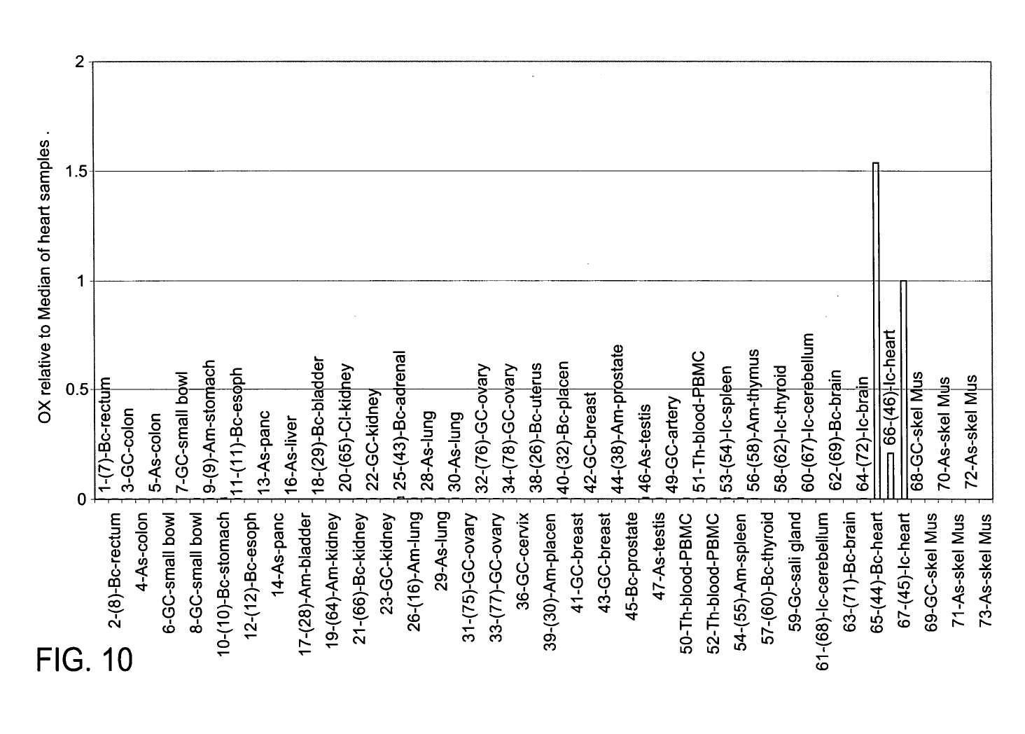

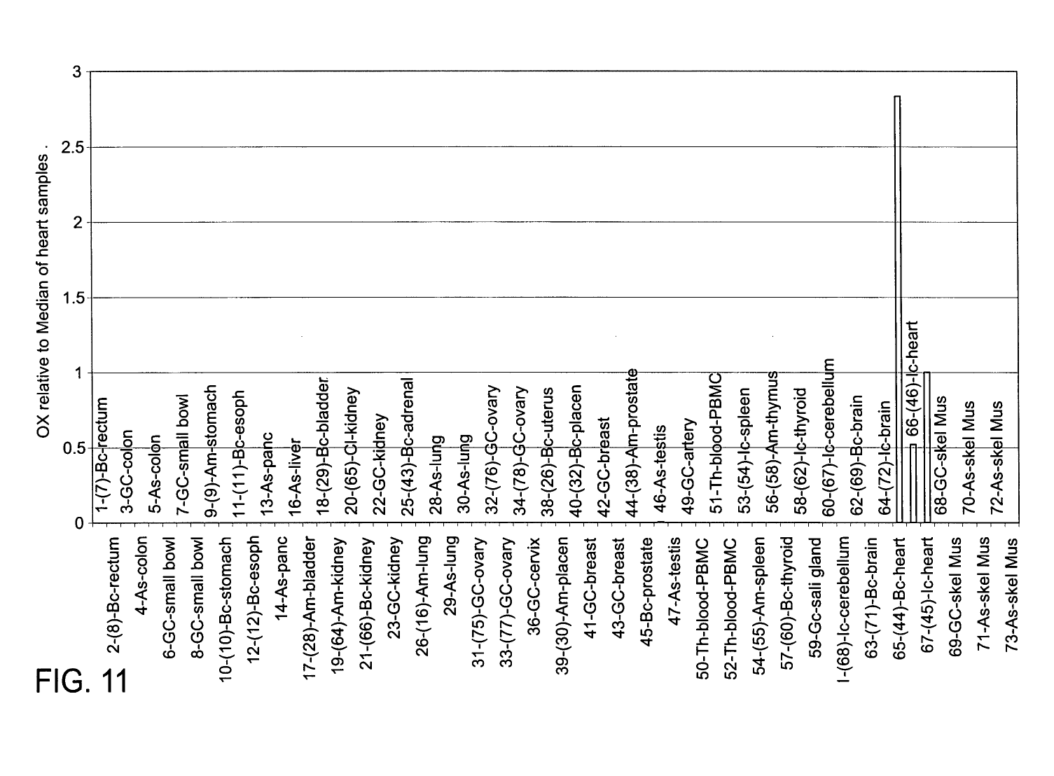

FIG. 10 is a histogram showing relative expression of the above-indicated myosin binding protein C, cardiac (MYBPC3) transcripts in heart tissue samples as opposed to other tissues.

As is evident from FIG. 10, the expression of myosin binding protein C, cardiac (MYBPC3) transcripts detectable by the above amplicon in heart tissue samples was significantly higher than in the other samples.

Primer pairs are also optionally and preferably encompassed within the present invention; for example, for the above experiment, the following primer pair was used as a non-limiting illustrative example only, of a suitable primer pair: Z18303_seg28-29F (SEQ ID NO:139) forward primer; and Z18303_seg28-29R (SEQ ID NO:140) reverse primer.

The present invention also preferably encompasses any amplicon obtained through the use of any suitable primer pair; for example, for the above experiment, the following amplicon was obtained as a non-limiting illustrative example only of a suitable amplicon: Z18303_seg28-29 (SEQ ID NO:141).

Forward Primer (Z18303_seg28-29F (SEQ ID NO:
139)):
TGAGGCGCGATGAGAAGAA
Reverse Primer (Z18303_seg28-29R (SEQ ID NO:
140)):
CATGTATGTGGACGAGGTGGG
Amplicon (Z18303_seg28-29 (SEQ ID NO: 141)):
TGAGGCGCGATGAGAAGAAGAGCACAGGTTAGCCCTTCCTCAGAGGGGAG
AGGAGAGGGCACAGGCTAGCCCTTCCCTACACAGGAGAGGAGAGGGCATA
GGTTAGCCCTTCCCCACAGGGGAGAGGAGAGGGCACAGGTTAGCCCCCCA
CCTCGTCCACATACATG

Expression of MYBPC3-Myosin Binding Protein C Z18303 Transcripts which are Detectable by Amplicon as Depicted in Sequence Name Z18303_seg71F2R2_WT (SEQ ID NO: 144) Specifically in Heart Tissue

Expression of MYBPC3-myosin binding protein C transcripts detectable by or according to seg71F2R2-Z18303_seg71F2R2_WT (SEQ ID NO:144) amplicon and primers Z18303_seg71F2 (SEQ ID NO:142) and Z18303_seg71R2 (SEQ ID NO:143) was measured by real time PCR. Non-detected samples (sample(s) no. 14) were assigned Ct value of 41 and were calculated accordingly. In parallel the expression of several housekeeping genes—SDHA (GenBank Accession No. NM_{—}004168 (SEQ ID NO: 31); amplicon—SDHA-amplicon (SEQ ID NO: 34)), Ubiquitin (GenBank Accession No. BC000449 (SEQ ID NO: 27); amplicon—Ubiquitin-amplicon (SEQ ID NO: 30)), and TATA box (GenBank Accession No. NM_{—}003194 (SEQ ID NO: 23); TATA amplicon (SEQ ID NO: 26)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the heart samples (sample numbers 65, 66 and 67, Table 1_{—}5 above), to obtain a value of relative expression for each sample relative to median of the heart samples.

FIG. 11 is a histogram showing relative expression of the above-indicated MYBPC3-myosin binding protein C transcripts in cancerous heart tissue samples as opposed to other tissues.

As is evident from FIG. 11, the expression of MYBPC3-myosin binding protein C transcripts detectable by the above amplicon in heart tissue samples was significantly higher than in the other samples.

Primer pairs are also optionally and preferably encompassed within the present invention; for example, for the above experiment, the following primer pair was used as a non-limiting illustrative example only of a suitable primer pair: Z18303_seg71F2 (SEQ ID NO:142) forward primer; and Z18303_seg71R2 (SEQ ID NO:143) reverse primer.

The present invention also preferably encompasses any amplicon obtained through the use of any suitable primer pair; for example, for the above experiment, the following amplicon was obtained as a non-limiting illustrative example only of a suitable amplicon: Z18303_seg71F2R2_WT (SEQ ID NO:144).

Forward Primer (Z18303_seg71F2 (SEQ ID NO: 142)):
AGGCGAGGAGGTGAGCATC
Reverse Primer (Z18303_seg71R2 (SEQ ID NO: 143)):
TCCTCCATGTTCTCAATGCG
Amplicon (Z18303_seg71F2R2_WT (SEQ ID NO: 144)):
AGGCGAGGAGGTGAGCATCCGCAACAGCCCCACAGACACCATCCTGTTCA
TCCGGGCCGCTCGCCGCGTGCATTCAGGCACTTACCAGGTGACGGTGCGC
ATTGAGAACATGGAGGA

### Description for Cluster HUMCA1XIA

Cluster HUMCA1XIA features 1 transcript(s) and 1 segment(s) of interest, the names for which are given in Tables 136 and 137, respectively. The selected protein variants are given in table 138.

TABLE 136
Transcripts of interest
Transcript Name

HUMCA1XIA_T3 (SEQ ID NO: 153)

TABLE 137
Segments of interest
Segment Name

HUMCA1XIA_N15 (SEQ ID NO: 154)

TABLE 138
Proteins of interest

| Protein Name | Corresponding Transcript(s) |
|---|---|
| HUMCA1XIA_P26 | HUMCA1XIA_T3 (SEQ ID NO: 153) |
| (SEQ ID NO: 162) | |

These sequences are variants of the known protein Collagen alpha 1 (SEQ ID NO:155) (SwissProt accession identifier COBA1_HUMAN), referred to herein as the previously known protein.

Protein Collagen alpha 1 (SEQ ID NO:155) is known or believed to have the following function(s): May play an important role in fibrillogenesis by controlling lateral growth of collagen II fibrils. Known polymorphisms for this sequence are as shown in Table 139.

TABLE 139
Amino acid mutations for Known Protein

| SNP position(s) | |
|---|---|
| on amino acid | |
| sequence | Comment |
| 625 | G -> V (in STL2). /FTId = VAR_013583 |
| 986 | Y -> H |
| 1074 | R -> P |
| 1516 | G -> V (in STL2; overlapping phenotype |
| | with Marshall syndrome). |
| | /FTId = VAR_013587 |
| 1786 | S -> N |
| 1758 | T -> A |
| 1313-1315 | Missing (in STL2; overlapping phenotype |
| | with Marshall syndrome). |
| | /FTId = VAR_013586 |
| 1218 | M -> W |
| 676 | G -> R (in STL2; overlapping phenotype |
| | with Marshall syndrome). |
| | /FTId = VAR_013584 |
| 921-926 | Missing (in STL2; overlapping phenotype |
| | with Marshall syndrome). |
| | /FTId = VAR_013585 |
| 941-944 | KDGL -> RMGC |
| 1142 | G -> D |

The following GO Annotation(s) apply to the previously known protein. The following annotation(s) were found: extracellular matrix organization and biogenesis; visual perception; cartilage condensation; cell-cell adhesion, which are annotation(s) related to Biological Process; extracellular matrix structural constituent; protein binding, bridging, which are annotation(s) related to Molecular Function; and collagen type XI; extracellular matrix (sensu Metazoa), which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

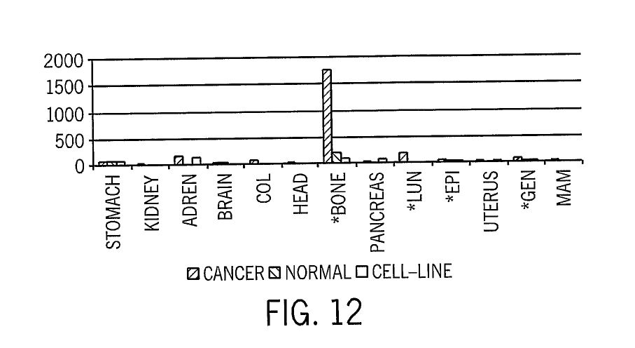

Cluster HUMCA1XIA can be used as a diagnostic marker according to overexpression of transcripts of this cluster in cancer. Expression of such transcripts in normal tissues is also given according to the previously described methods. The term “number” in the left hand column of the table and the numbers on the y-axis of the FIG. 12 below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 12 and Table 140. This cluster is overexpressed (at least at a minimum level) in the following pathological conditions: bone malignant tumors, lung malignant tumors, epithelial malignant tumors and a mixture of malignant tumors from different tissues.

TABLE 140
Normal tissue distribution

| Name of Tissue | Number |
|---|---|
| stomach | 72 |
| kidney | 0 |
| adrenal | 0 |
| brain | 12 |
| colon | 0 |
| head and neck | 0 |
| bone | 202 |
| pancreas | 0 |
| lung | 0 |
| epithelial | 11 |
| uterus | 8 |
| general | 11 |
| breast | 8 |

TABLE 141
P values and ratios for expression in cancerous tissue

| Name of Tissue | P1 | P2 | SP1 | R3 | SP2 | R4 |
|---|---|---|---|---|---|---|
| stomach | 4.6e−01 | 5.5e−01 | 6.9e−01 | 1.0 | 6.7e−01 | 0.8 |
| kidney | 5.7e−01 | 6.4e−01 | 3.3e−01 | 2.5 | 4.8e−01 | 1.9 |
| adrenal | 3.8e−01 | 1.6e−01 | 9.5e−02 | 3.4 | 8.0e−02 | 3.6 |
| brain | 3.0e−01 | 5.4e−01 | 1.6e−01 | 2.3 | 5.1e−01 | 1.1 |
| colon | 1.2e−02 | 2.4e−02 | 2.3e−01 | 3.0 | 3.5e−01 | 2.4 |
| head and neck | 1.2e−01 | 2.1e−01 | N/A | N/A | N/A | N/A |
| bone | 2.4e−01 | 6.3e−01 | 1.0e−09 | 4.2 | 5.5e−03 | 1.6 |
| pancreas | 3.1e−01 | 1.6e−01 | 4.2e−01 | 2.4 | 1.5e−01 | 3.7 |
| lung | 4.2e−02 | 7.5e−02 | 5.3e−05 | 7.4 | 4.8e−03 | 4.1 |
| epithelial | 3.8e−04 | 3.0e−03 | 1.3e−03 | 2.3 | 1.8e−02 | 1.8 |
| uterus | 6.8e−01 | 6.6e−01 | 6.6e−01 | 1.1 | 6.4e−01 | 1.1 |
| general | 4.0e−05 | 1.1e−03 | 1.9e−16 | 4.5 | 1.5e−09 | 2.8 |
| breast | 4.3e−01 | 6.0e−01 | 6.9e−01 | 1.4 | 8.2e−01 | 1.1 |

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster but not other segments/transcripts below, shown in Table 142.

TABLE 142
Oligonucleotides related to this cluster

| | Overexpressed | Chip |
|---|---|---|
| Oligonucleotide name | in cancers | reference |
| HUMCA1XIA_0_18_0 (SEQ ID NO: 166) | colorectal | Colon |
| | cancer | |
| HUMCA1XIA_0_18_0 (SEQ ID NO: 166) | breast | BRS |
| | malignant | |
| | tumors | |
| HUMCA1XIA_0_18_0 (SEQ ID NO: 166) | lung malignant | LUN |
| | tumors | |

The sequence of the HUMCA1XIA_{—}0_{—}18_{—}0 oligonucleotide is (SEQ ID NO:166) TTCAGAACTGTTAACATCGCTGACGGGAAGTGGCATCGGGTAGCAATCAG

As noted above, cluster HUMCA1XIA features 1 transcript(s), which were listed in Table 136 above. These transcript(s) encode for protein(s) which are variant(s) of protein Collagen alpha 1 (SEQ ID NO:155). A description of each variant protein according to the present invention is now provided.

Variant protein HUMCA1XIA_P26 (SEQ ID NO:162) according to the present invention is encoded by transcript(s) HUMCA1XIA_T3 (SEQ ID NO:153). An alignment is given to the known protein (Collagen alpha 1 (SEQ ID NO:155)) at the end of the application. One or more alignments to one or more previously published protein sequences are given at the end of the application. A brief description of the relationship of the variant protein according to the present invention to each such aligned protein is as follows:

### 1. Comparison Report Between HUMCA1XIAP26 (SEQ ID NO:162) and COBA1_HUMAN (SEQ ID NO:155):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDE corresponding to amino acids 1-260 of COBA1_HUMAN (SEQ ID NO:155), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKMRTVATKSKEKSKKFTPPKSEKFSSKKKKSYQASAKAKLGVKVAKKKQSRSILDKLEDL (SEQ ID NO:167) corresponding to amino acids 261-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167) of HUMCA1XIA_P26 (SEQ ID NO:162).

### 2. Comparison Report Between HUMCA1XIA_P26 (SEQ ID NO:162) and P12107-2 (SEQ ID NO:159):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDEKKKSNFKKKMRTVATKSKEKSKKFTPP KSEKFSSKKKKSYQASAKAKLGVK corresponding to amino acids 1-311 of P12107-2 (SEQ ID NO:159), which also corresponds to amino acids 1-311 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence VAKKKQSRSILDKLEDL (SEQ ID NO:168) corresponding to amino acids 312-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence VAKKKQSRSILDKLEDL (SEQ ID NO:168) of HUMCA1XIA_P26 (SEQ ID NO:162).

### 3. Comparison Report Between HUMCA1XIA_P26 (SEQ ID NO:162) and NP_{—}542196 (SEQ ID NO:157):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDEKKKSNFKKKMRTVATKSKEKSKKFTPP KSEKFSSKKKKSYQASAKAKLGVK corresponding to amino acids 1-311 of NP_{—}542196 (SEQ ID NO:157), which also corresponds to amino acids 1-311 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence VAKKKQSRSILDKLEDL (SEQ ID NO:168) corresponding to amino acids 312-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence VAKKKQSRSILDKLEDL (SEQ ID NO:168) of HUMCA1XIA_P26 (SEQ ID NO:162).

### 4. Comparison Report Between HUMCA1XIA_P26 (SEQ ID NO:162) and P12107-3 (SEQ ID NO:161):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDE corresponding to amino acids 1-260 of P12107-3 (SEQ ID NO:161), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKMRTVATKSKEKSKKFTPPKSEKFSSKKKKSYQASAKAKLGVKVAKKKQSRSILDKLEDL (SEQ ID NO:167)corresponding to amino acids 261-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167) of HUMCA1XIA_P26 (SEQ ID NO:162).

### 5. Comparison Report Between HUMCA1XIAP26 (SEQ ID NO:162) and NP_{—}542197 (SEQ ID NO:158):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDE corresponding to amino acids 1-260 of NP_{—}542197 (SEQ ID NO:158), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKCMRTVATKSKEKSKKFTPPKSEKFSSKKKKSYQASAKAKLGVKVAKKKQSRSILDKLEDL (SEQ ID NO:167) corresponding to amino acids 261-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167) of HUMCA1XIA_P26 (SEQ ID NO:162).

### 6. Comparison Report Between HUMCA1XIA_P26 (SEQ ID NO:162) and Q5VT31_HUMAN (SEQ ID NO:156):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDE corresponding to amino acids 1-260 of Q5VT31_HUMAN (SEQ ID NO:156), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKMRTVATKSKEKSKKFTPPKSEKFSSKKKKSYQASAKAKLGVKVAKKKQSRSILDKLEDL (SEQ ID NO:167) corresponding to amino acids 261-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167) of HUMCA1XIA P26 (SEQ ID NO:162).

### 7. Comparison Report Between HUMCA1XIA_P26 (SEQ ID NO:162) and NP_{—}001845 (SEQ ID NO:160):

A. An isolated chimeric polypeptide comprising HUMCA1XIA_P26 (SEQ ID NO:162), comprising a first amino acid sequence being at least 90% homologous to MEPWSSRWKTKRWLWDFTVTTLALTFLFQAREVRGAAPVDVLKALDFHNSPEGISKTTGFCTNRKNSKGS DTAYRVSKQAQLSAPTKQLFPGGTFPEDFSILFTVKPKKGIQSFLLSIYNEHGIQQIGVEVGRSPVFLFEDHTG KPAPEDYPLFRTVNIADGKWHRVAISVEKKTVTMIVDCKKKTTKPLDRSERAIVDTNGITVFGTRILDEEVF EGDIQQFLITGDPKAAYDYCEHYSPDCDSSAPKAAQAQEPQIDE corresponding to amino acids 1-260 of NP_{—}001845 (SEQ ID NO:160), which also corresponds to amino acids 1-260 of HUMCA1XIA_P26 (SEQ ID NO:162), and a second amino acid sequence being at least 70%, optionally at least 80%, preferably at least 85%, more preferably at least 90% and most preferably at least 95% homologous to a polypeptide having the sequence KKKSNFKKKMRTVATKSKEKSKKFTPPKSEKFSSKKKKSYQASAKAKLGVKVAKKKQSRSILDKLEDL (SEQ ID NO:167) corresponding to amino acids 261-328 of HUMCA1XIA_P26 (SEQ ID NO:162), wherein said first amino acid sequence and second amino acid sequence are contiguous and in a sequential order.

B. An isolated polypeptide comprising an edge portion of HUMCA1XIA_P26 (SEQ ID NO:162), comprising an amino acid sequence being at least 70%, optionally at least about 80%, preferably at least about 85%, more preferably at least about 90% and most preferably at least about 95% homologous to the sequence KKKSNFKKKMRTVATKSKEKSKKFTPPKSEKFSSKKKKSYQASAKAKLGVKVAKKKQSRSILDKLEDL (SEQ ID NO:167) of HUMCA1XIA_P26 (SEQ ID NO:162).

The localization of the variant protein was determined according to results from a number of different software programs and analyses, including analyses from SignalP and other specialized programs. The variant protein is believed to be located as follows with regard to the cell: secreted.

Variant protein HUMCA1XIA_P26 (SEQ ID NO:162) also has the following non-silent SNPs (Single Nucleotide Polymorphisms) as listed in Table 143, (given according to their position(s) on the amino acid sequence, with the alternative amino acid(s) listed; the last column indicates whether the SNP is known or not; the presence of known SNPs in variant protein HUMCA1XIA_P26 (SEQ ID NO:162) sequence provides support for the deduced sequence of this variant protein according to the present invention).

TABLE 143
Amino acid mutations

| SNP position(s) on amino acid | |
|---|---|
| sequence | Alternative amino acid(s) |
| 46 | D -> E |
| 276 | K -> N |

The glycosylation sites of variant protein HUMCA1XIA_P26 (SEQ ID NO:162), as compared to the known protein Collagen alpha 1 (SEQ ID NO:155), are described in Table 144 (given according to their position(s) on the amino acid sequence in the first column; the second column indicates whether the glycosylation site is present in the variant protein; and the last column indicates whether the position is different on the variant protein).

TABLE 144
Glycosylation site(s)

| Position(s) on known amino acid | | |
|---|---|---|
| sequence | | Present in variant protein? |
| | 1640 | No |

The phosphorylation sites of variant protein HUMCA1XIA_P26 (SEQ ID NO:162), as compared to the known protein, are described in Table 145 (given according to their position(s) on the amino acid sequence in the first column; the second column indicates whether the phosphorylation site is present in the variant protein; and the last column indicates whether the position is different on the variant protein).

TABLE 145
Phosphorylation site(s)

| Position(s) on known amino acid | | |
|---|---|---|
| sequence | | Present in variant protein? |
| | 612 | No |
| | 1452 | No |

Variant protein HUMCA1XIA_P26 (SEQ ID NO:162) is encoded by the following transcript(s): HUMCA1XIA_T3 (SEQ ID NO:153). The coding portion of transcript HUMCA1XIA T3 (SEQ ID NO:153) starts at position 319 and ends at position 1302. The transcript also has the following SNPs as listed in Table 146 (given according to their position on the nucleotide sequence, with the alternative nucleic acid listed; the last column indicates whether the SNP is known or not; the presence of known SNPs in variant protein HUMCA1XIA_P26 (SEQ ID NO:162) sequence provides support for the deduced sequence of this variant protein according to the present invention).

TABLE 146
Nucleic acid SNPs

| SNP position(s) on nucleotide | |
|---|---|
| sequence | Alternative nucleic acid(s) |
| 157 | A -> G |
| 456 | T -> G |
| 1146 | A -> C |
| 2215 | G -> A |
| 3034 | G -> T |
| 3496 | C -> T |
| 3915 | T -> C |
| 4065 | G -> C |
| 4189 | C -> |
| 4194 | -> G |

As noted above, cluster HUMCA1XIA features 1 segment(s), which were listed in Table 137. These segment(s) are portions of nucleic acid sequence(s) which are described herein separately because they are of particular interest. A description of each segment according to the present invention is now provided.

Segment cluster HUMCA1XIA_N15 (SEQ ID NO:154) according to the present invention is supported by 1 libraries. The number of libraries was determined as previously described. This segment can be found in the following transcript(s): HUMCA1XIA_T3 (SEQ ID NO:153). Table 147 below describes the starting and ending position of this segment on each transcript.

TABLE 147
Segment location on transcripts

| | | Segment |
|---|---|---|
| | Segment | ending |
| Transcript name | starting position | position |
| HUMCA1XIA_T3 (SEQ ID NO: 153) | 1252 | 1437 |

### Variant Protein Alignment to the Previously Known Protein:

### Alignment of: HUMCA1XIA_P26 (SEQ ID NO:162)×COBA1_HUMAN (SEQ ID NO:155)

Total length: 1874
Matching length: 260

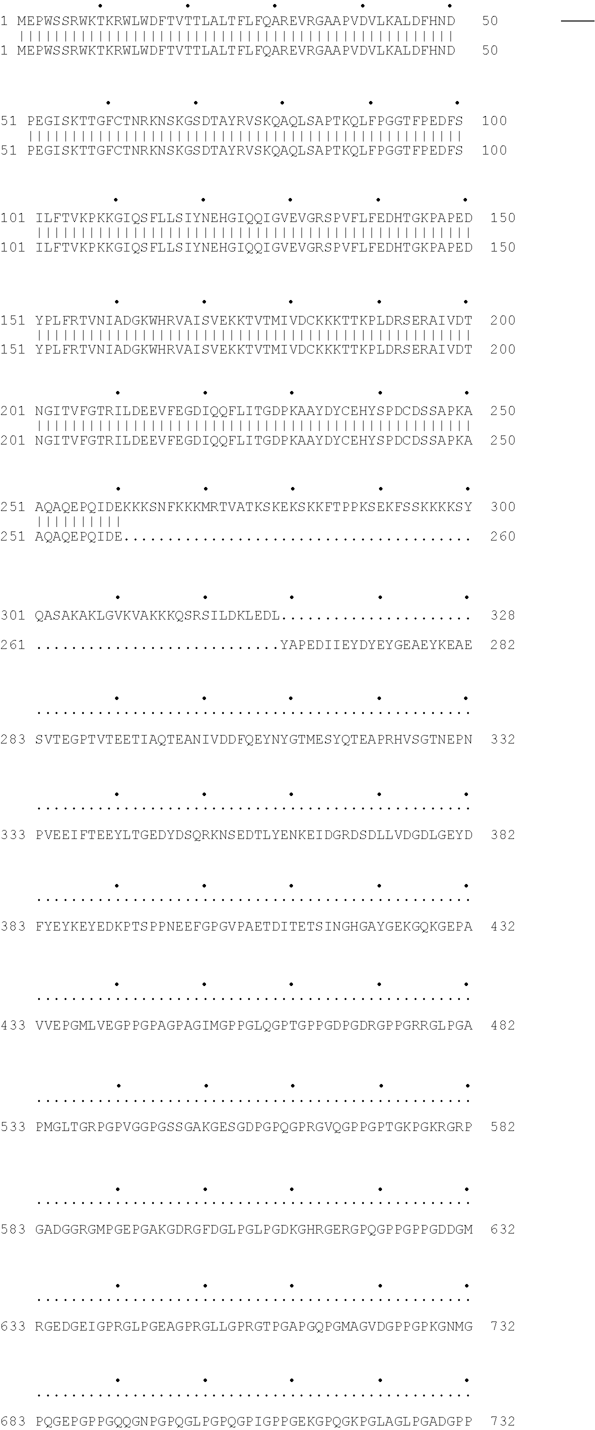

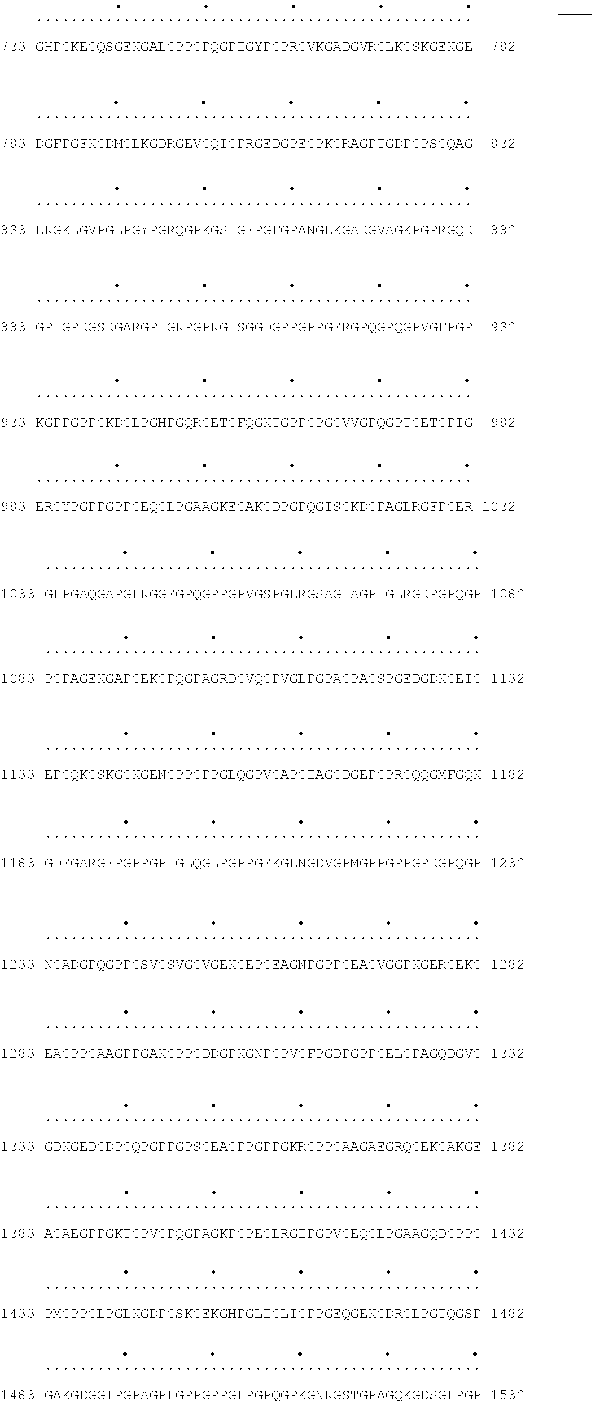

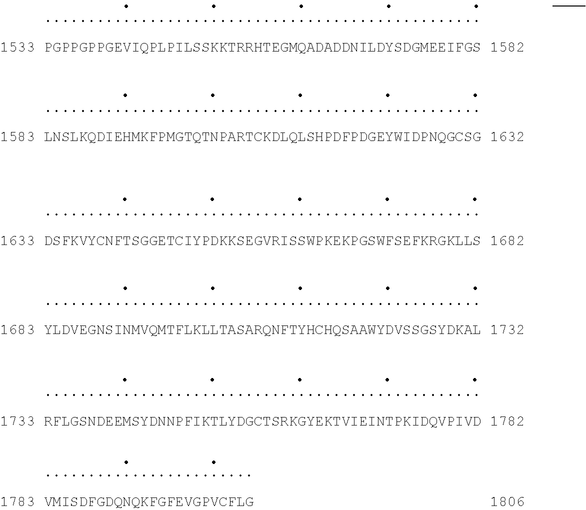

### Alignment of : HUMCA1XIA_P26 (SEQ ID NO:162)×P12107-2 (SEQ ID NO:159):

Total length: 1835
Matching length: 311

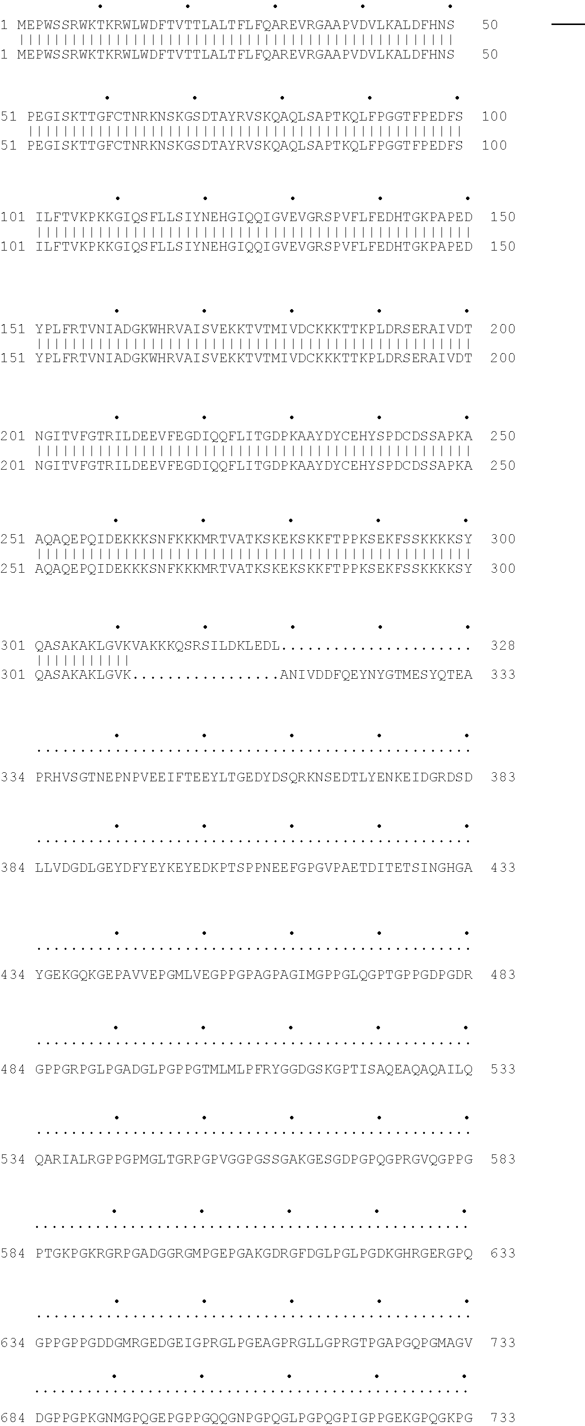

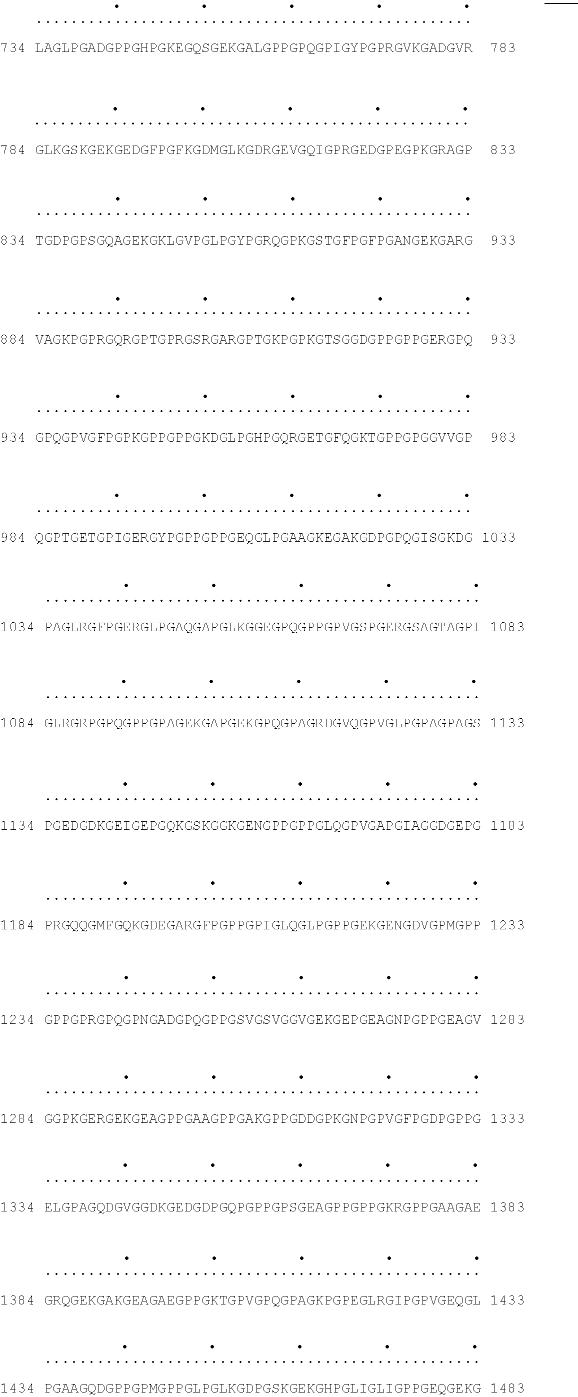

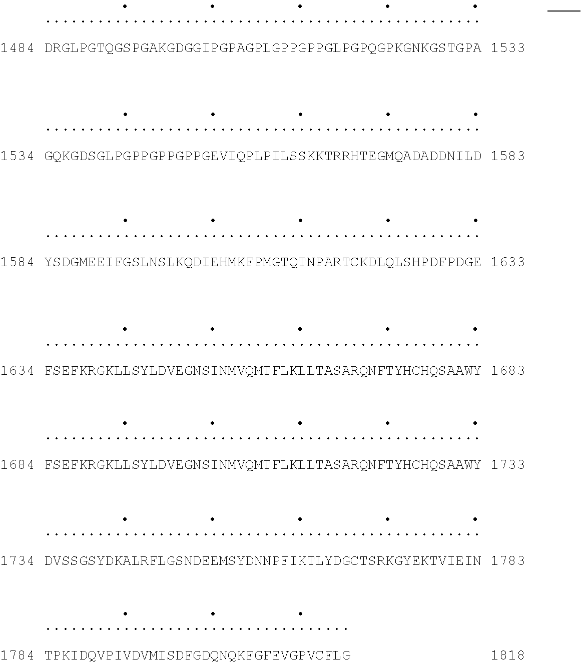

### Alignment of: HUMCA1XIA_P26 (SEQ ID NO:162)×NP_{—}542196 (SEQ ID NO:157):

Total length: 1835
Matching length: 311

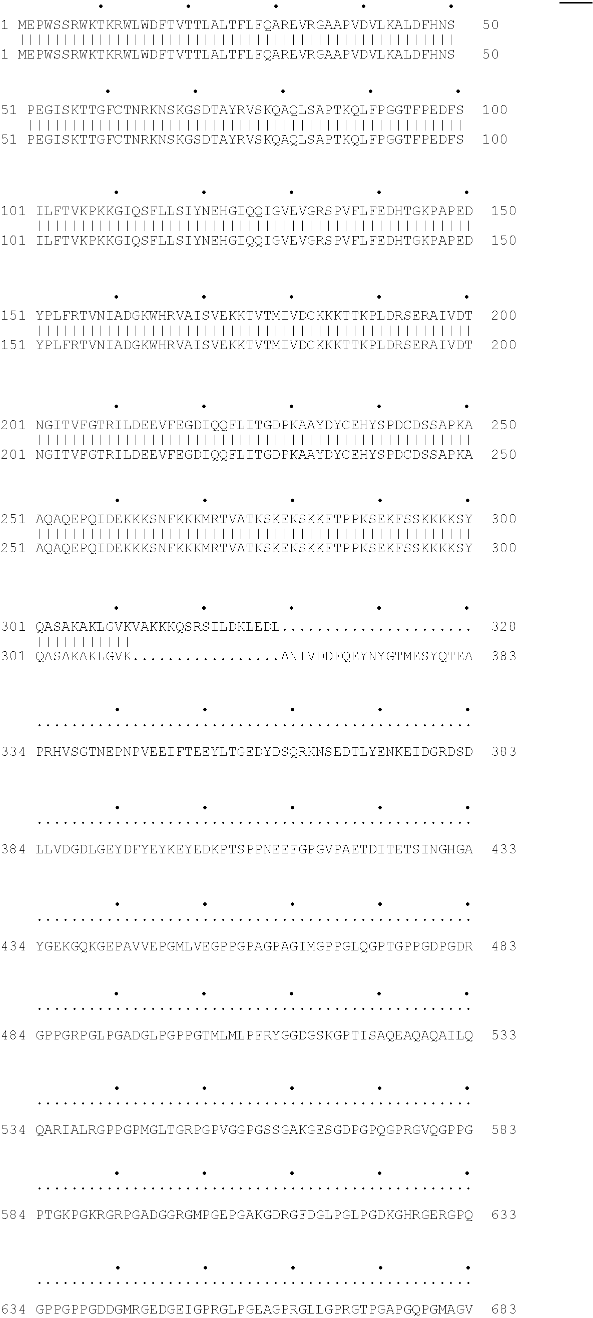

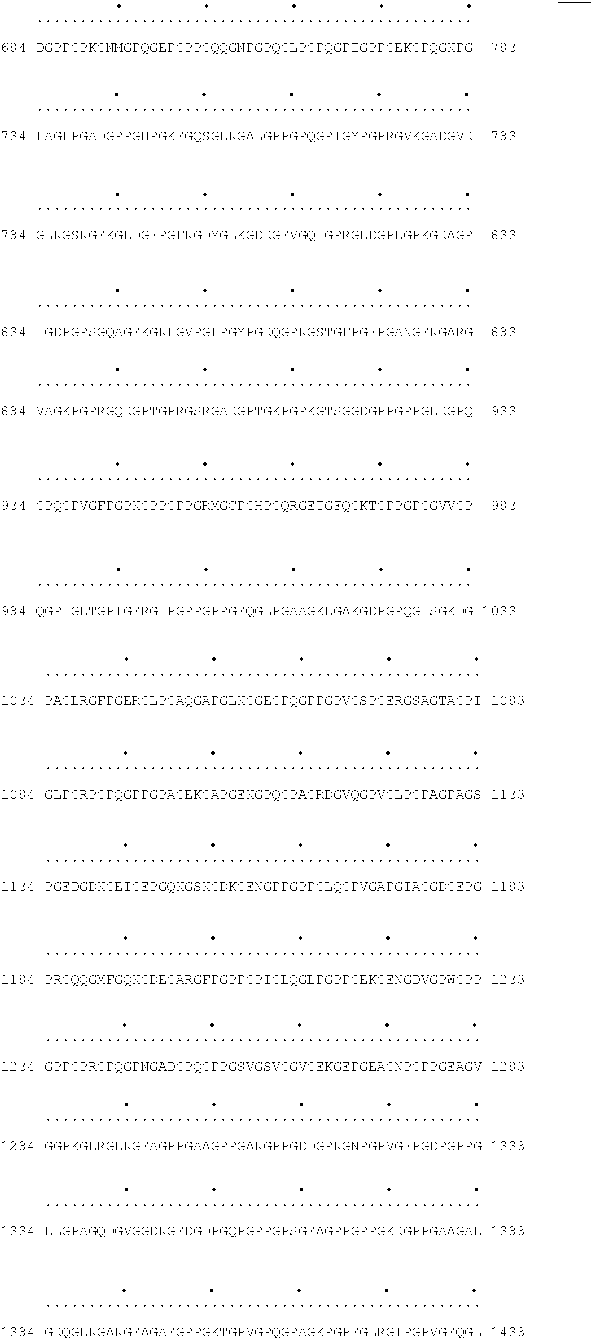

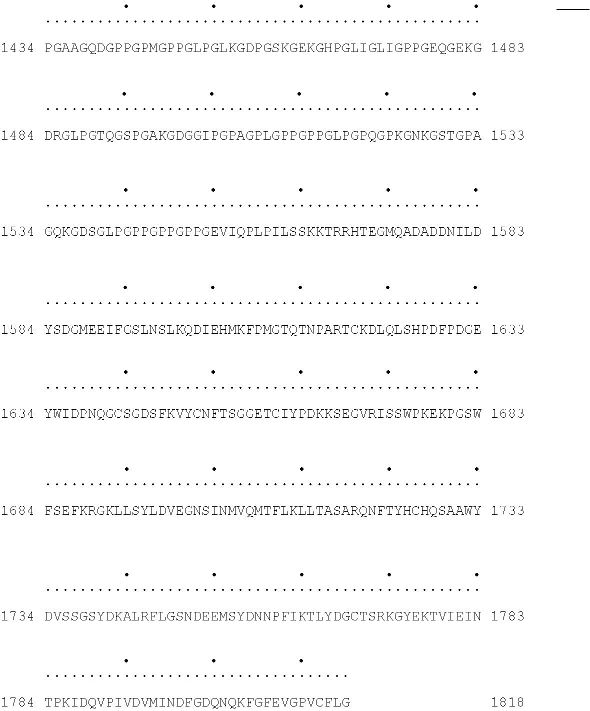

### Alignment of: HUMCA1XIA_P26 (SEQ ID NO:162)×P12107-3 (SEQ ID NO:161):

Total length: 1835
Matching length: 260

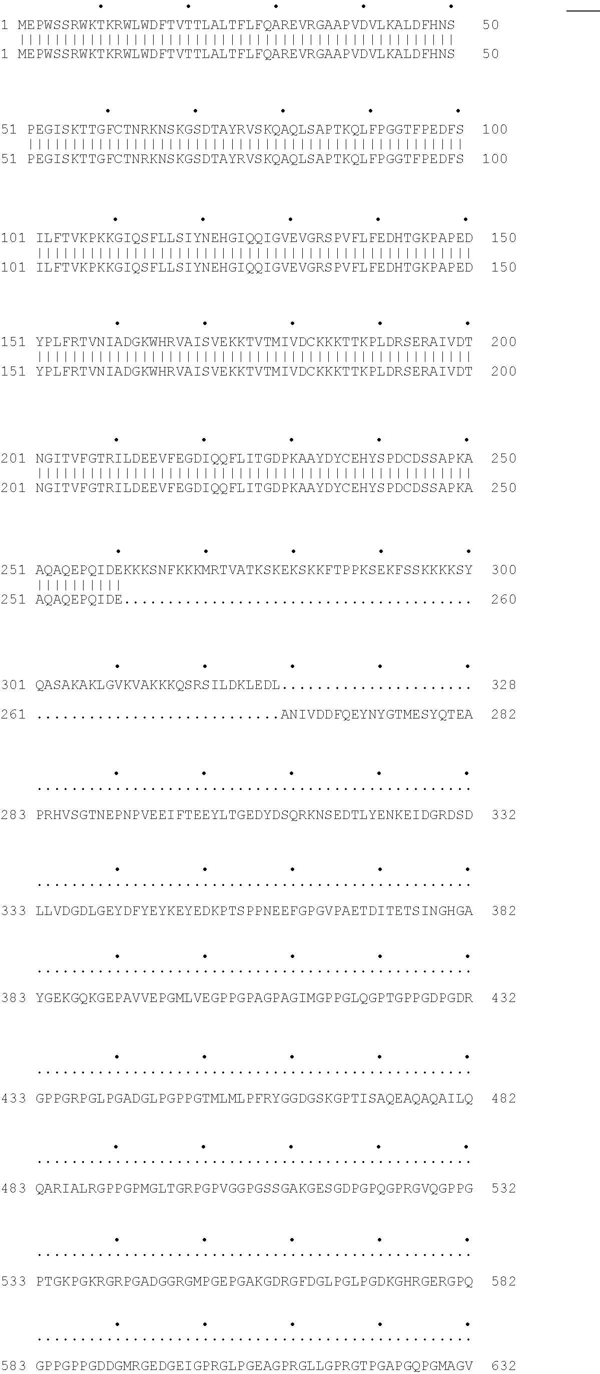

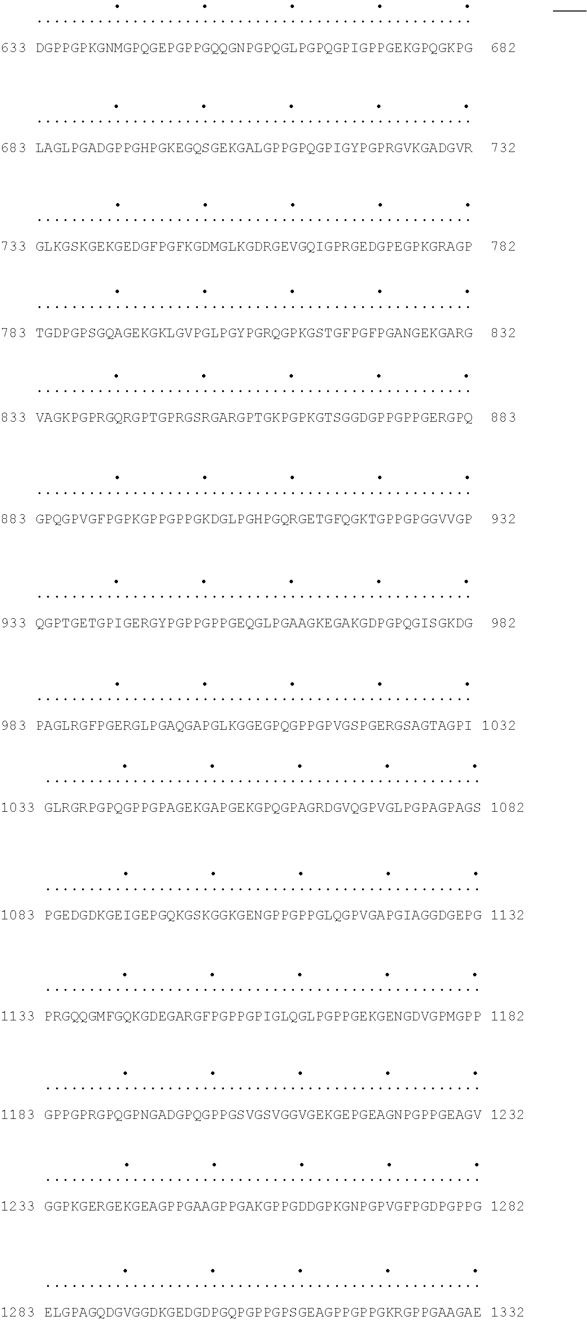

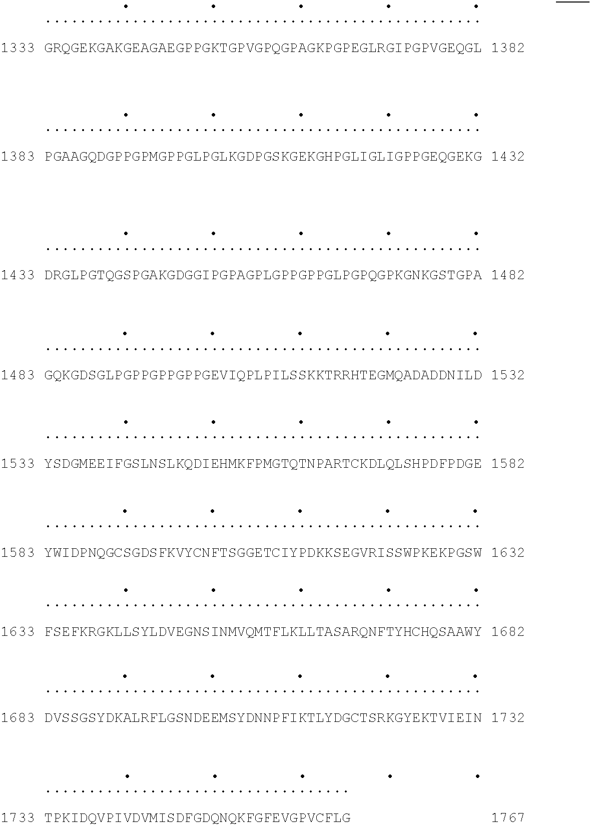

### Alignment of: HUMCA1XIA_P26 (SEQ ID NO:162)×NP_{—}542197 (SEQ ID NO:158):

Total length: 1835
Matching length: 260

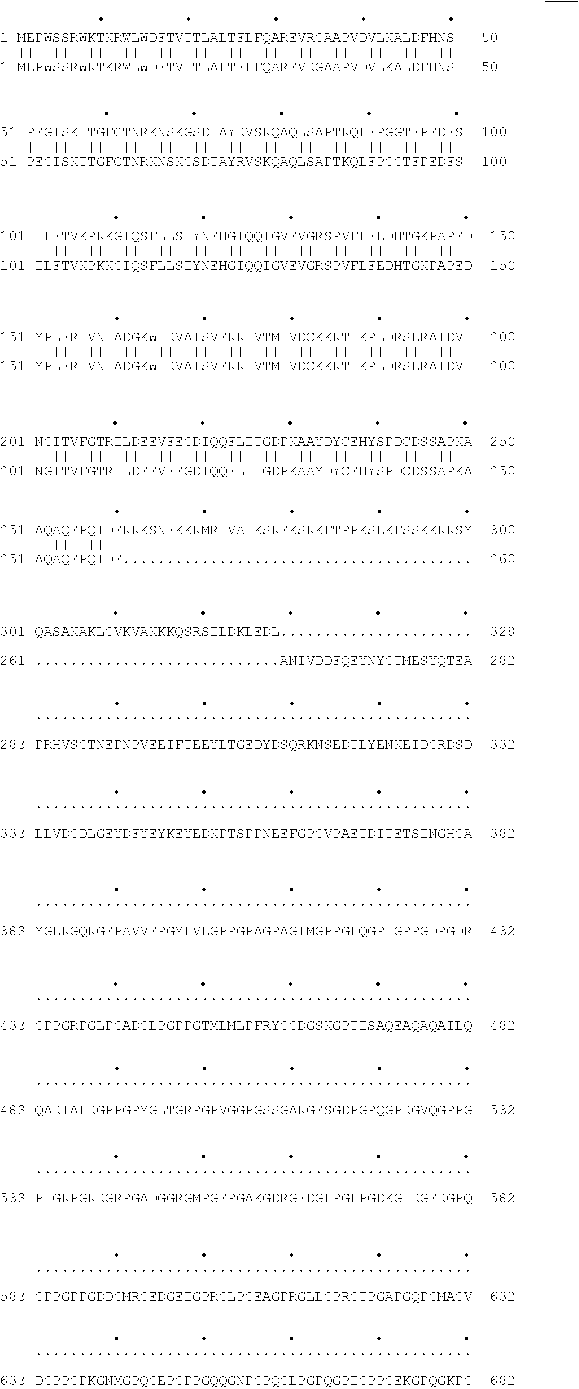

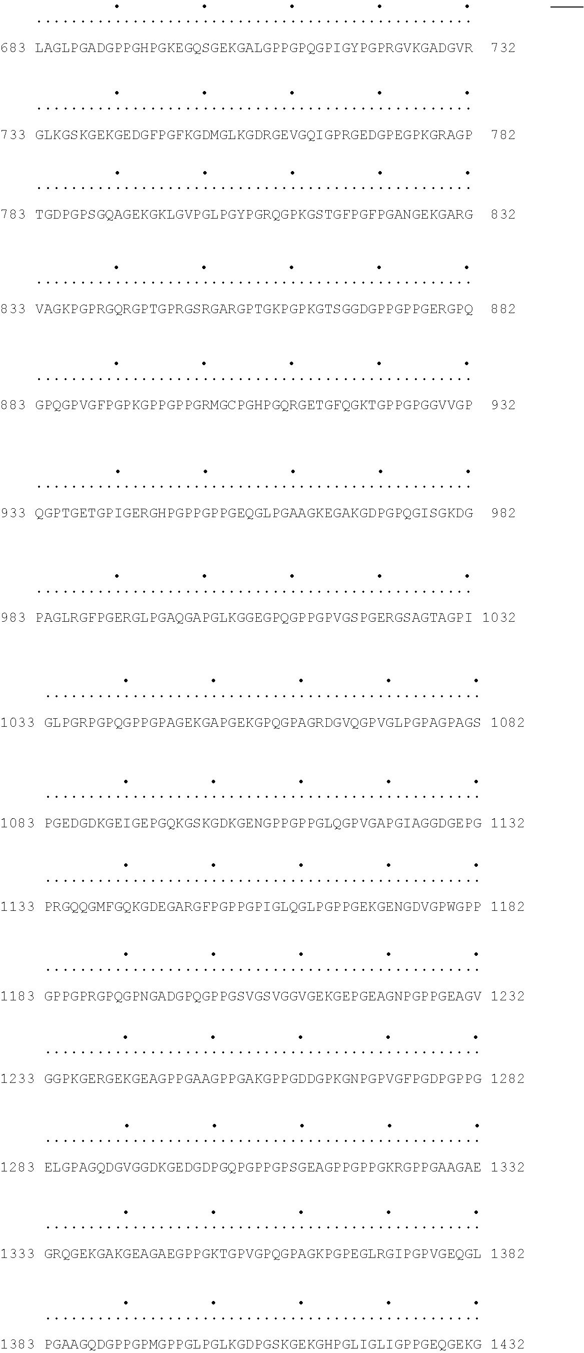

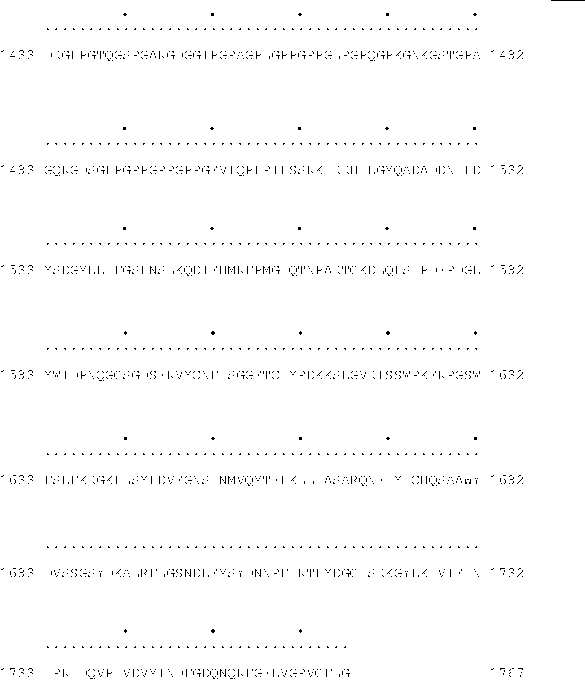

### Alignment of: HUMCA1XIA_P26 (SEQ ID NO:162)×Q5VT31_HUMAN (SEQ ID NO:156)

Total length: 1874
Matching length: 260

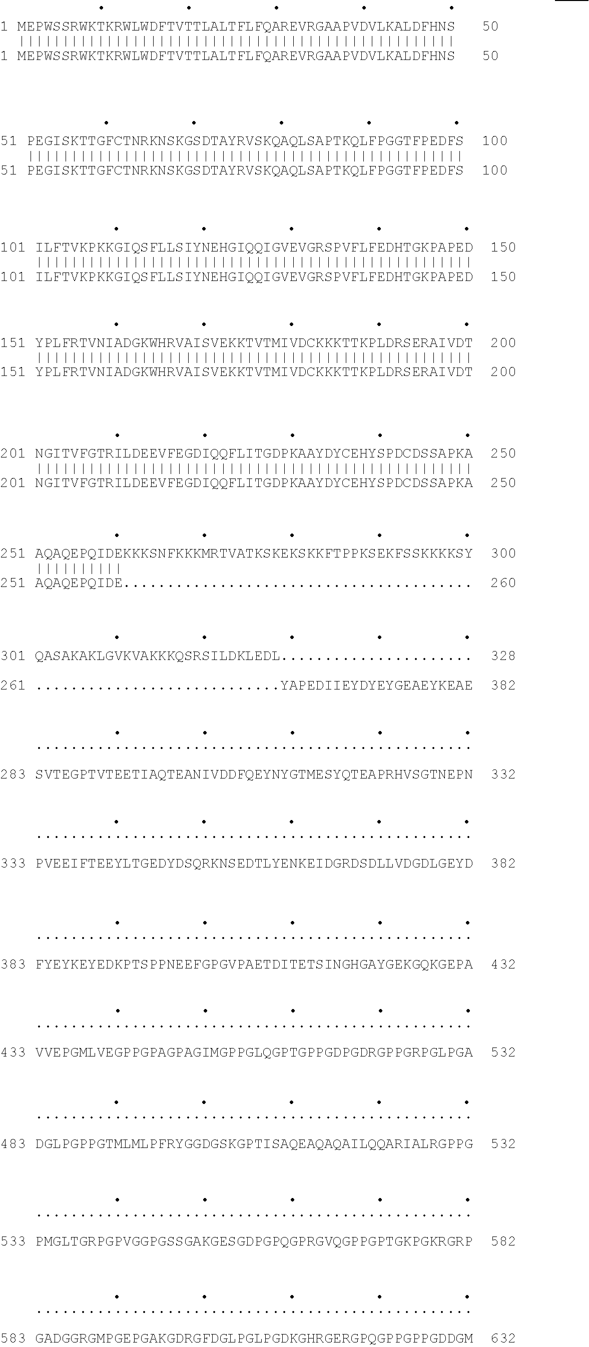

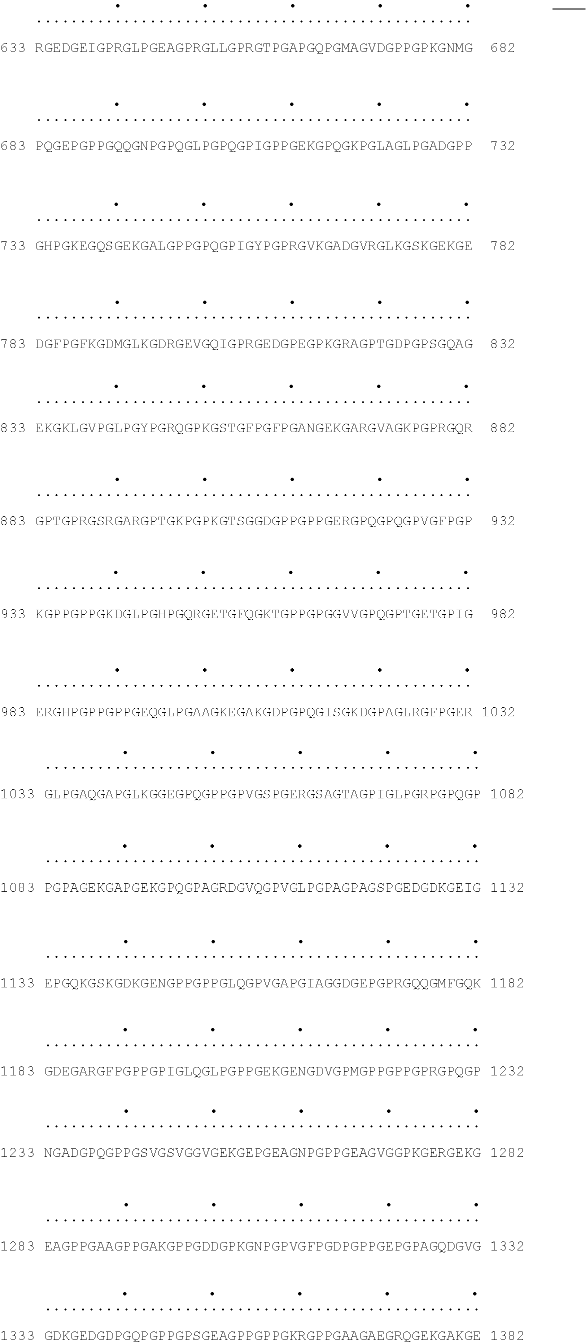

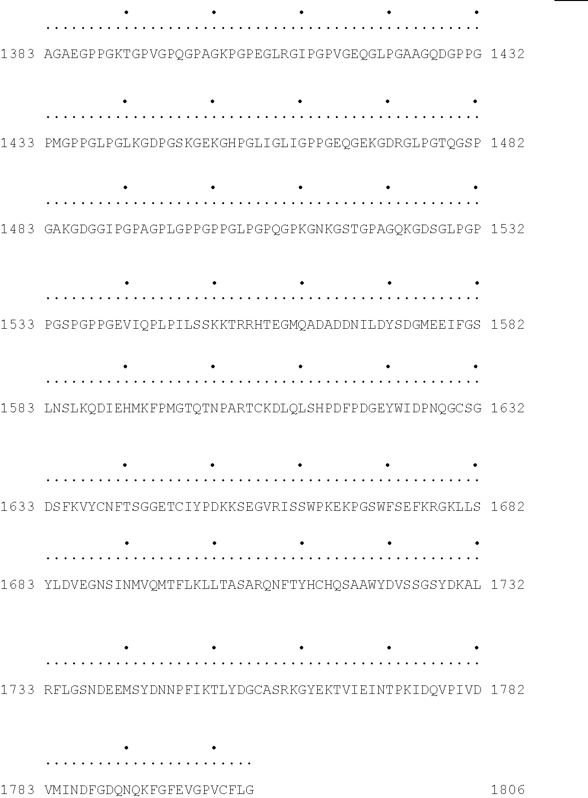

### Alignment of: HUMCA1XIA_P26 (SEQ ID NO:162)×NP_{—}001845 (SEQ ID NO:160):

Total length: 1874
Matching length: 260

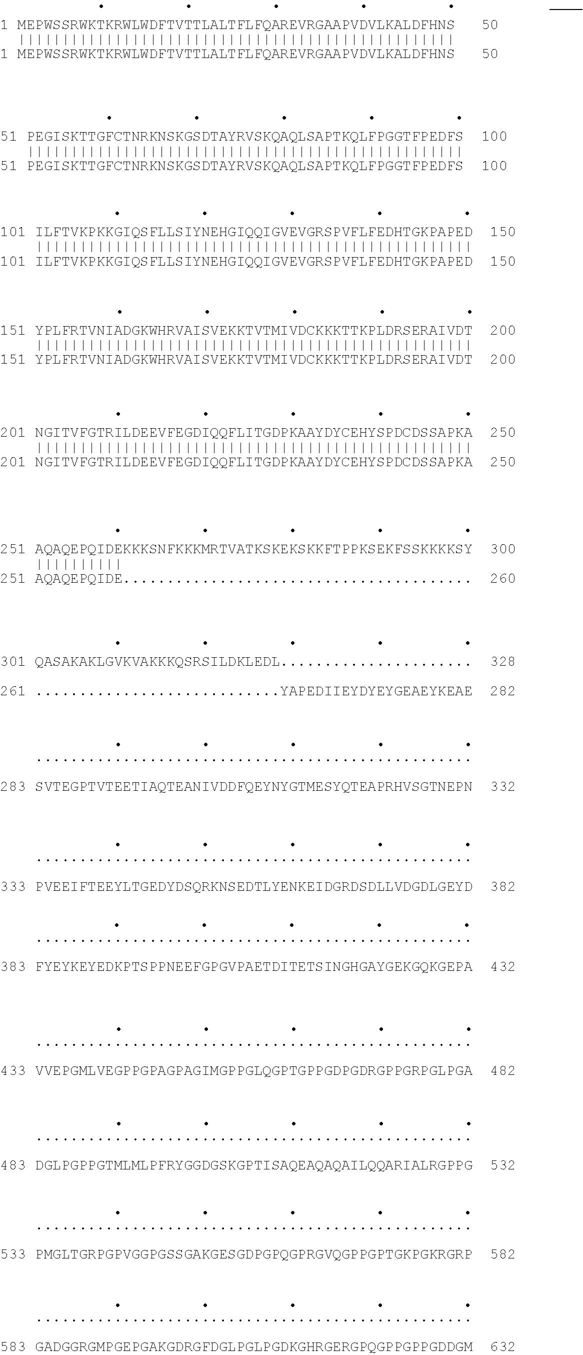

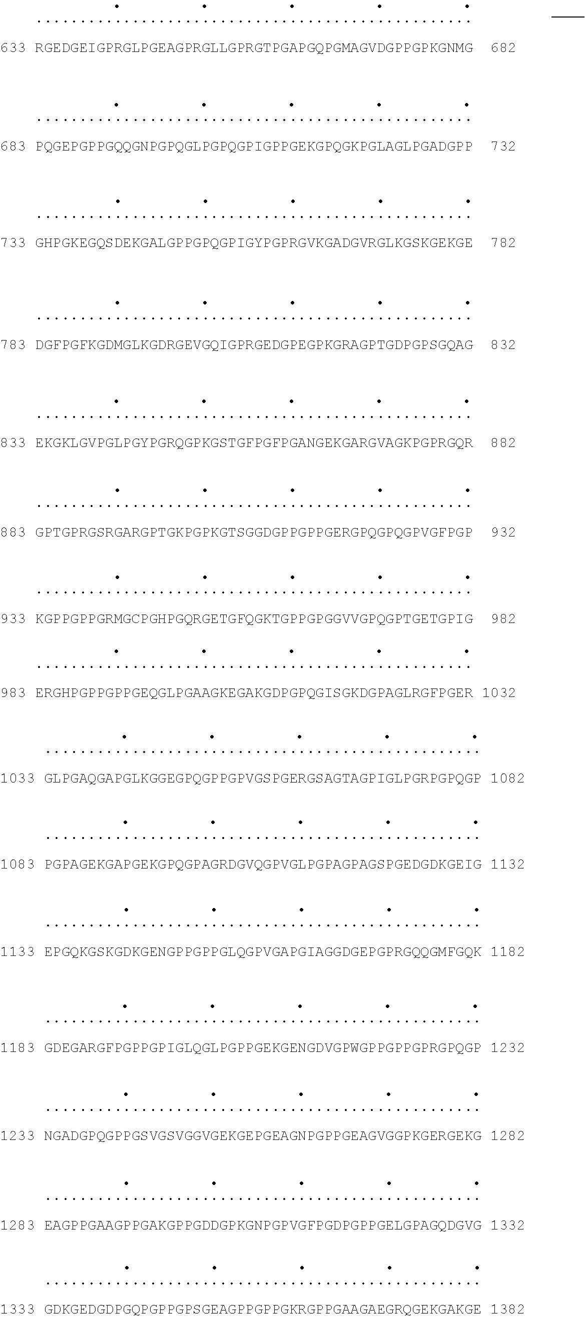

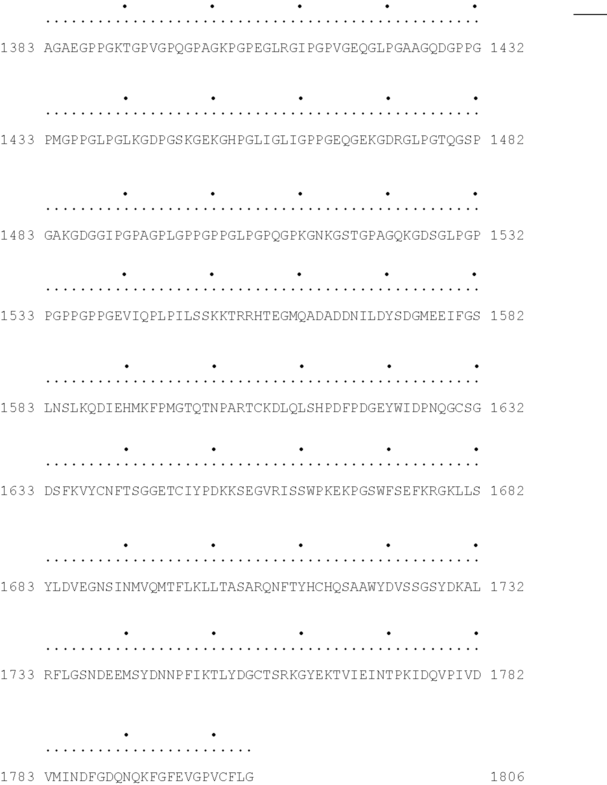

Expression of Homo sapiens Collagen, Type XI, Alpha 1 (COL11A1) HUMCA1XIA Transcripts which are Detectable by Amplicon as Depicted in Sequence Name HUMCA1XIA_seg15 (SEQ ID NO:165) in Normal and Cancerous Colon Tissues

Expression of Homo sapiens collagen, type XI, alpha 1 (COL11A1) transcripts detectable by or according to seg15—HUMCA1XIA_seg15 (SEQ ID NO:165) amplicon and primers HUMCA1XIA_seg15F (SEQ ID NO:163) and HUMCA1XIA_seg15R (SEQ ID NO:164) was measured by real time PCR. In parallel the expression of several housekeeping genes—HPRT1 (GenBank Accession No. NM_{—}000194 (SEQ ID NO:5); amplicon—HPRT1—amplicon (SEQ ID NO:8)), PBGD (GenBank Accession No. BC019323 (SEQ ID NO:1); amplicon—PBGD-amplicon (SEQ ID NO:4)), and G6PD (GenBank Accession No. NM_{—}000402 (SEQ ID NO:13); G6PD amplicon (SEQ ID NO:151) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the normal samples (sample numbers 42, 43, 44, 45, 46, 47, 48, 49, 50, 51, 52, 53, 54 and 55, Table 1_{—}4 above), to obtain a value of fold up-regulation for each sample relative to median of the normal samples.

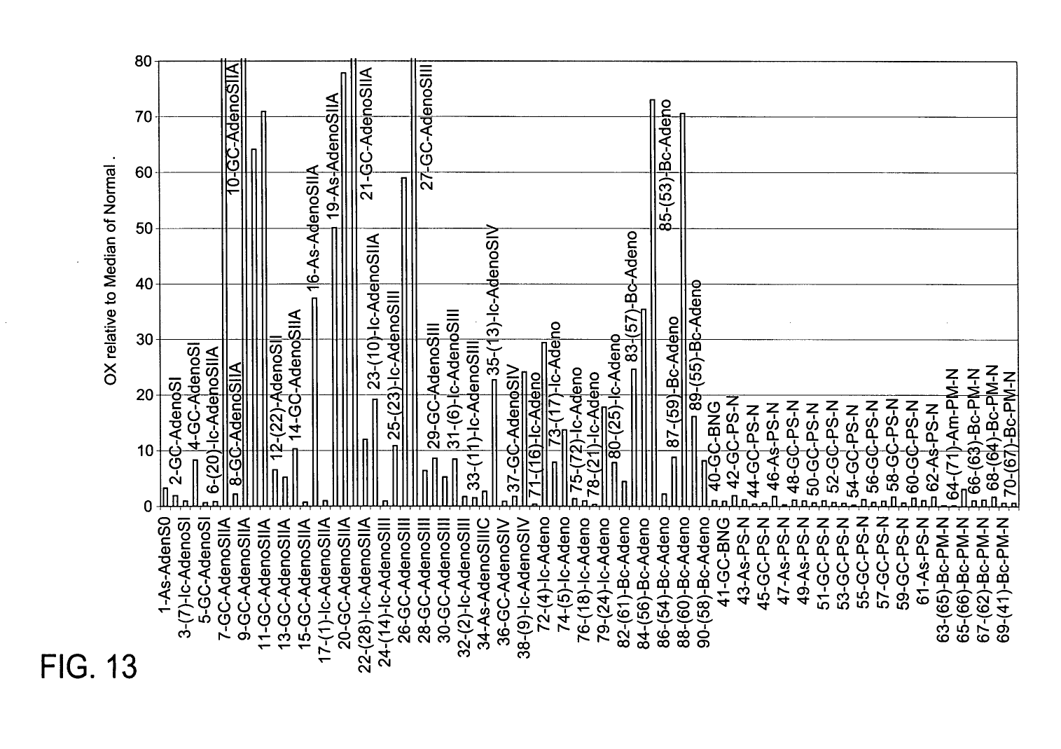

FIG. 13 is a histogram showing over expression of the above-indicated Homo sapiens collagen, type XI, alpha 1 (COL11A1) transcripts in cancerous Colon samples relative to the normal samples.

As is evident from FIG. 13, the expression of Homo sapiens collagen, type XI, alpha 1 (COL11A1) transcripts detectable by the above amplicon in cancer samples was significantly higher than in the non-cancerous samples (sample numbers 42, 43, 44, 45, 46, 47, 48, 49, 50, 51, 52, 53, 54 and 55, Table 1_{—}4 above). Notably an over-expression of at least 5 fold was found in 35 out of 55 adenocarcinoma samples.

Statistical analysis was applied to verify the significance of these results, as described below.

The P value for the difference in the expression levels of Homo sapiens collagen, type XI, alpha 1 (COL11A1) transcripts detectable by the above amplicon in Colon cancer samples versus the normal tissue samples was determined by T test as 2.49E-06.

Threshold of 5 fold over expression was found to differentiate between cancer and normal samples with P value of 9.58E-10 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg15F (SEQ ID NO:163) forward primer; and HUMCA1XIA_seg15R (SEQ ID NO:164) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg15 (SEQ ID NO:165).

Forward Primer (HUMCA1XIA_seg15F (SEQ ID NO:
163)):
AATCAAGATCTATCCTAGATAAGCTGGAAG
Reverse Primer (HUMCA1XIA_seg15R (SEQ ID NO:
164)):
AATTGGGTTATAGTATTAGCCAAGTGAGTA
Amplicon (HUMCA1XIA_seg15 (SEQ ID NO: 165)):
AATCAAGATCTATCCTAGATAAGCTGGAAGATCTCTGATCATGAATCATA
GTCTTAACCCAACTAGATAAATACTCACTTGGCTAATACTATAACCCAAT
T

Expression of Homo sapiens Collagen, Type XI, Alpha 1 (COL11A1) HUMCA1XIA Transcripts which are Detectable by Amplicon as Depicted in Sequence Name HUMCA1XIA_seg15 (SEQ ID NO:165) in Normal and Cancerous Ovary Tissues

Expression of Homo sapiens collagen, type XI, alpha 1 (COL11A1) transcripts detectable by or according to seg15—HUMCA1XIA_seg15 (SEQ ID NO:165) amplicon and primers HUMCA1XIA_seg15F (SEQ ID NO:163) and HUMCA1XIA_seg15R (SEQ ID NO:164) was measured by real time PCR. In parallel the expression of several housekeeping genes—SDHA (GenBank Accession No. NM_{—}004168 (SEQ ID NO:31); amplicon—SDHA-amplicon (SEQ ID NO:34)), HPRT1 (GenBank Accession No. NM_{—}000194 (SEQ ID NO:5); amplicon—HPRT1—amplicon (SEQ ID NO:8)) and G6PD (GenBank Accession No. NM_{—}000402 (SEQ ID NO:13); G6PD amplicon (SEQ ID NO:151)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the normal samples (sample numbers 52, 53, 54, 55, 56, 57, 58, 59, 60, 61, 62, 63, 64 and 65, Table 1_{—}1 above), to obtain a value of fold up-regulation for each sample relative to median of the normal samples.

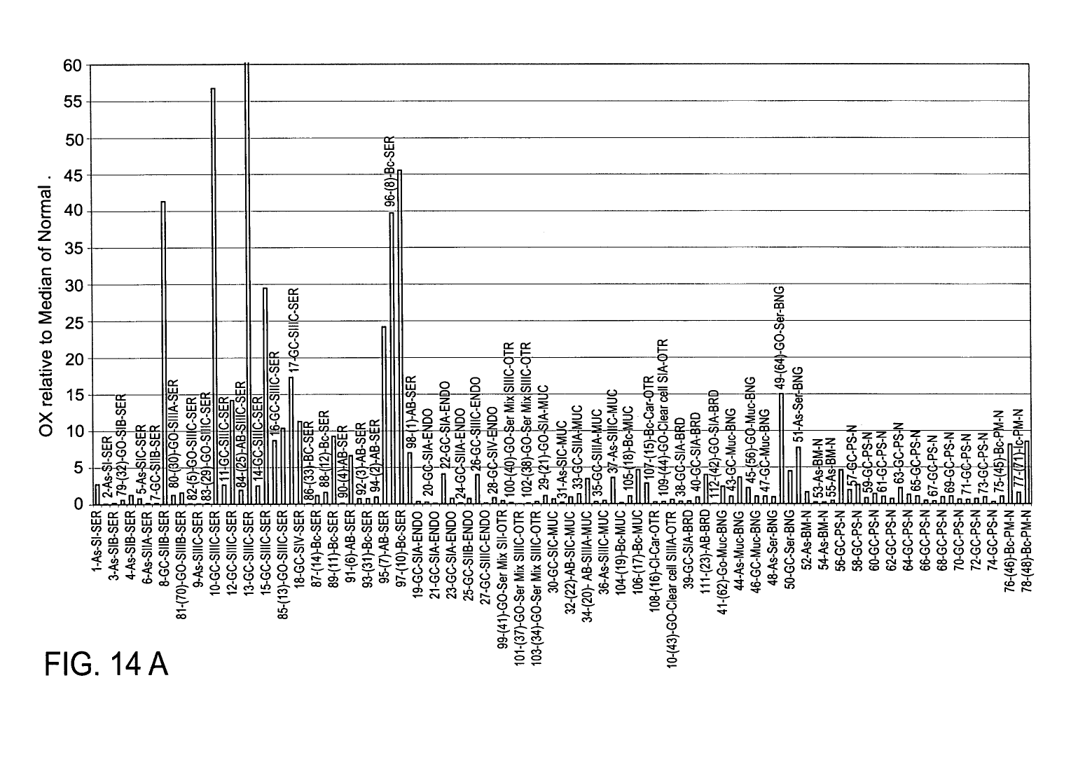

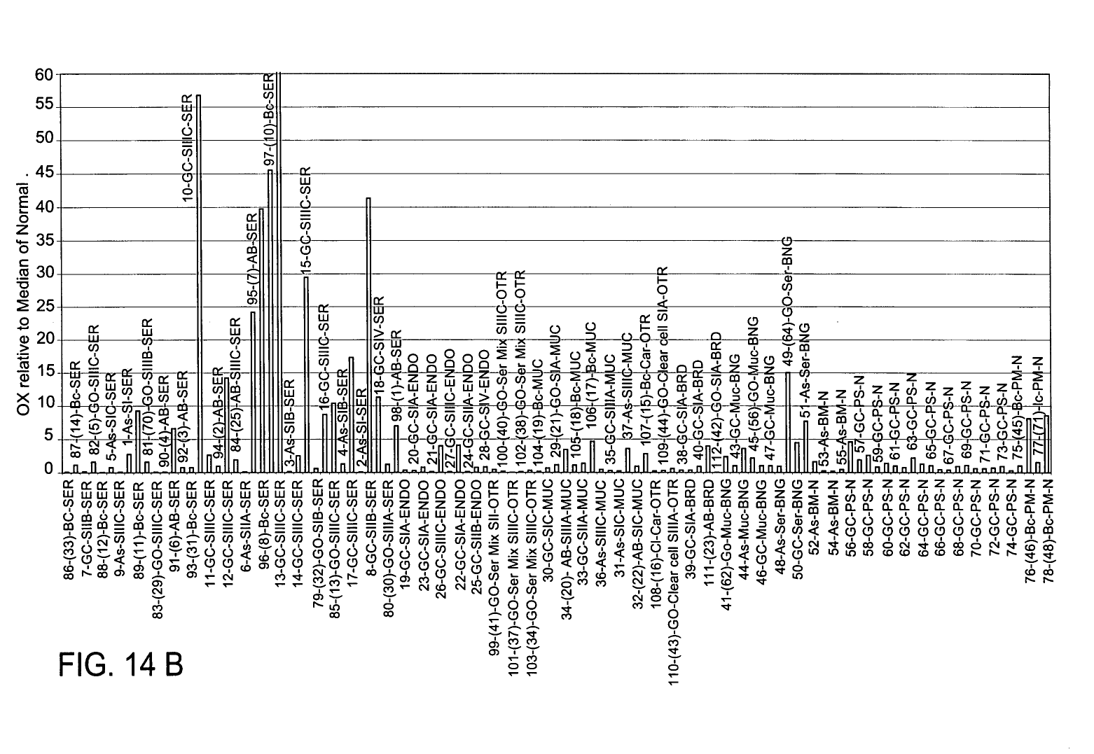

FIGS. 14A and 14B are a histograms showing over expression of the above-indicated Homo sapiens collagen, type XI, alpha 1 (COL11A1) transcripts in cancerous Ovary samples relative to the normal samples. In figure A samples are arranged according to the disease stage, and in figure B samples are arranged according to the patient's age.

As is evident from FIGS. 14A and 14B, the expression of Homo sapiens collagen, type XI, alpha 1 (COL11A1) transcripts detectable by the above amplicon in serous carcinoma samples was significantly higher than in the non-cancerous samples (sample numbers 52, 53, 54, 55, 56, 57, 58, 59, 60, 61, 62, 63, 64 and 65, Table 1_{—}1 above). Notably an over-expression of at least 5 fold was found in 15 out of 38 serous carcinoma samples. Over expression was observed especially in patients in stages 3-4 (FIG. 14A) with age above 53 (FIG. 14B).

Statistical analysis was applied to verify the significance of these results, as described below.

The P value for the difference in the expression levels of Homo sapiens collagen, type XI, alpha 1 (COL11A1) transcripts detectable by the above amplicon in Ovary serous carcinoma samples versus the normal tissue samples was determined by T test as 1.03E-02.

Threshold of 5 fold over expression was found to differentiate between serous carcinoma and normal samples with P value of 3.24E-03 as checked by exact Fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg15F (SEQ ID NO:163) forward primer; and HUMCA1XIA_seg15R (SEQ ID NO:164) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg15 (SEQ ID NO:165).

Forward Primer (HUMCA1XIA_seg15F (SEQ ID NO:
163)):
AATCAAGATCTATCCTAGATAAGCTGGAAG
Reverse Primer (HUMCA1XIA_seg15R (SEQ ID NO:
164)):
AATTGGGTTATAGTATTAGCCAAGTGAGTA
Amplicon (HUMCA1XIA_seg15 (SEQ ID NO: 165)):
AATCAAGATCTATCCTAGATAAGCTGGAAGATCTCTGATCATGAATCATA
GTCTTAACCCAACTAGATAAATACTCACTTGGCTAATACTATAACCCAAT
T

Expression of Homo sapiens Collagen, Type XI, Alpha 1 (COL11A1) HUMCA1XIA Transcripts which are Detectable by Amplicon as Depicted in Sequence Name HUMCA1XIA_seg15 (SEQ ID NO:165) in Normal and Cancerous Breast Tissues

Expression of Homo sapiens collagen, type XI, alpha 1 (COL11A1) transcripts detectable by or according to seg15—HUMCA1XIA_seg15 (SEQ ID NO:165) amplicon and primers HUMCA1XIA_seg15F (SEQ ID NO:163) and HUMCA1XIA_seg15R (SEQ ID NO:164) was measured by real time PCR. In parallel the expression of several housekeeping genes—G6PD (GenBank Accession No. NM_{—}000402 (SEQ ID NO:13); G6PD amplicon (SEQ ID NO:151)), RPL19 (GenBank Accession No. NM_{—}000981 (SEQ ID NO:19); RPL19 amplicon (SEQ ID NO:22)), PBGD (GenBank Accession No. BC019323 (SEQ ID NO:1); amplicon—PBGD-amplicon (SEQ ID NO:4)) and SDHA (GenBank Accession No. NM_{—}004168 (SEQ ID NO:31); amplicon—SDHA-amplicon (SEQ ID NO:34)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the normal samples (sample numbers 43, 45, 46, 47, 48, 49, 50, 51, 52, 54, 56, 58, 59 and 60, Table 1_{—}3 above), to obtain a value of fold up-regulation for each sample relative to median of the normal samples.

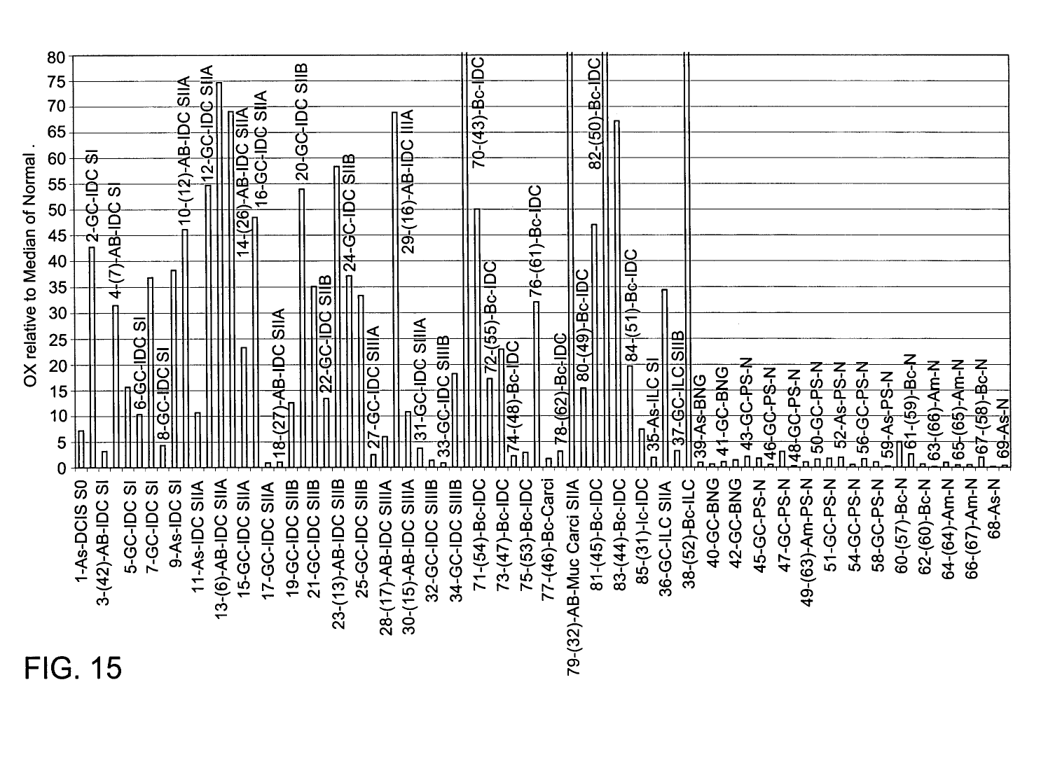

FIG. 15 is a histogram showing over expression of the above-indicated Homo sapiens collagen, type XI, alpha 1 (COL11A1) transcripts in cancerous Breast samples relative to the normal samples.

As is evident from FIG. 15, the expression of Homo sapiens collagen, type XI, alpha 1 (COL11A1) transcripts detectable by the above amplicon in cancer samples was significantly higher than in the non-cancerous samples (sample numbers 43, 45, 46, 47, 48, 49, 50, 51, 52, 54, 56, 58, 59 and 60, Table 1_{—}3 above). Notably an over-expression of at least 5 fold was found in 39 out of 53 adenocarcinoma samples.

Statistical analysis was applied to verify the significance of these results, as described below.

The P value for the difference in the expression levels of Homo sapiens collagen, type XI, alpha 1 (COL11A1) transcripts detectable by the above amplicon in Breast cancer samples versus the normal tissue samples was determined by T test as 7.89E-08.

Threshold of 5 fold over expression was found to differentiate between cancer and normal samples with P value of 3.58E-10 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg15F (SEQ ID NO:163) forward primer; and HUMCA1XIA_seg15R (SEQ ID NO:164) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg15 (SEQ ID NO:165).

Forward Primer (HUMCA1XIA_seg15F (SEQ ID NO:
163)):
AATCAAGATCTATCCTAGATAAGCTGGAAG
Reverse Primer (HUMCA1XIA_seg15R (SEQ ID NO:
164)):
AATTGGGTTATAGTATTAGCCAAGTGAGTA
Amplicon (HUMCA1XIA_seg15 (SEQ ID NO: 165)):
AATCAAGATCTATCCTAGATAAGCTGGAAGATCTCTGATCATGAATCATA
GTCTTAACCCAACTAGATAAATACTCACTTGGCTAATACTATAACCCAAT
T

Expression of Homo sapiens Collagen, Type XI, Alpha 1 (COL11A1) HUMCA1XIA Transcripts which are Detectable by Amplicon as Depicted in Sequence Name HUMCA1XIA_seg15 (SEQ ID NO:165) in Normal and Cancerous Lung Tissues

Expression of Homo sapiens collagen, type XI, alpha 1 (COL11A1) transcripts detectable by or according to seg15—HUMCA1XIA_seg15 (SEQ ID NO:165) amplicon and primers HUMCA1XIA_seg15F (SEQ ID NO:163) and HUMCA1XIA_seg15R (SEQ ID NO:164) was measured by real time PCR. In parallel the expression of several housekeeping genes—HPRT1 (GenBank Accession No. NM_{—}000194 (SEQ ID NO:5); amplicon—HPRT1—amplicon (SEQ ID NO:8)), PBGD (GenBank Accession No. BC019323 (SEQ ID NO:1); amplicon—PBGD-amplicon (SEQ ID NO:4)), SDHA (GenBank Accession No. NM_{—}004168 (SEQ ID NO:31); amplicon—SDHA-amplicon (SEQ ID NO:34)) and Ubiquitin (GenBank Accession No. BC000449 (SEQ ID NO:27); amplicon—Ubiquitin-amplicon (SEQ ID NO:30)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the normal samples (sample numbers 51, 52, 53, 54, 55, 56, 57, 58, 59, 60, 61, 62, 63 and 64, Table 12 above), to obtain a value of fold up-regulation for each sample relative to median of the normal samples.

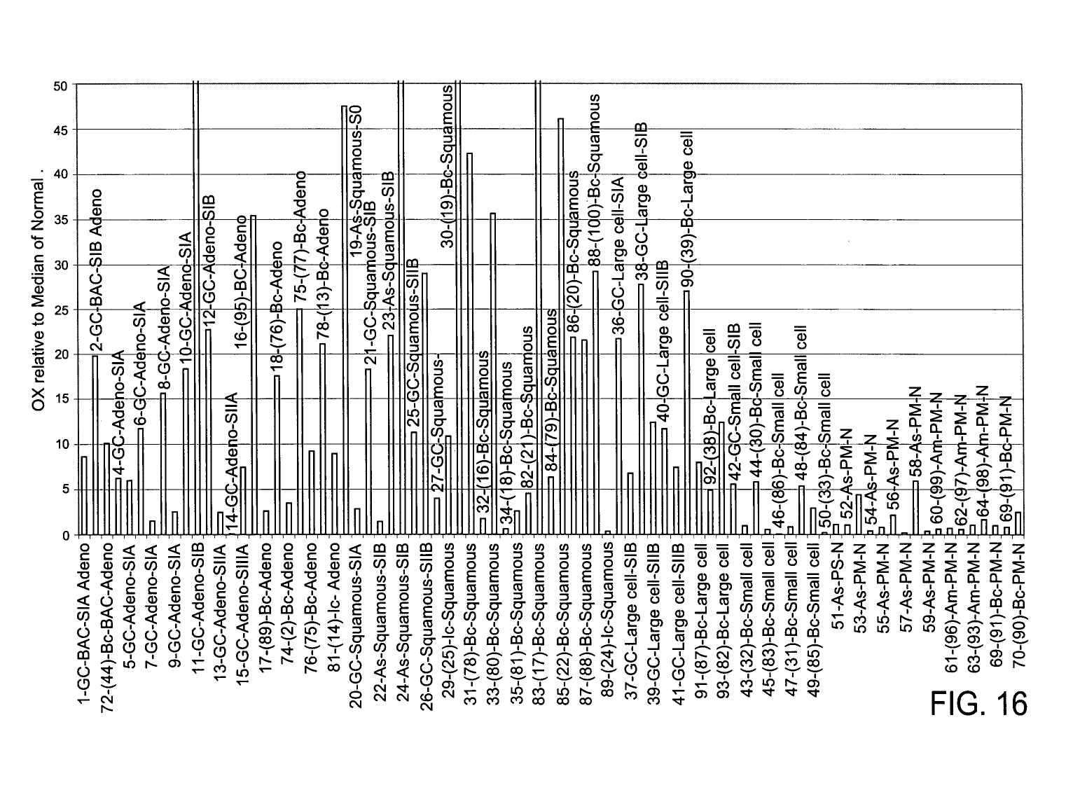

FIG. 16 is a histogram showing over expression of the above-indicated Homo sapiens collagen, type XI, alpha 1 (COL11A1) transcripts in cancerous Lung samples relative to the normal samples.

As is evident from FIG. 16, the expression of Homo sapiens collagen, type XI, alpha 1 (COL11A1) transcripts detectable by the above amplicon in non-small cell carcinoma samples was significantly higher than in the non-cancerous samples (sample numbers 51, 52, 53, 54, 55, 56, 57, 58, 59, 60, 61, 62, 63 and 64, Table 12 above). Notably an over-expression of at least 5 fold was found in 42 out of 57 non-small cell carcinoma samples (17 out of 23 adenocarcinoma samples, in 16 out of 24 squamous cell carcinoma samples, in 9 out of 10 large cell carcinoma samples).

Statistical analysis was applied to verify the significance of these results, as described below.

The P value for the difference in the expression levels of Homo sapiens collagen, type XI, alpha 1 (COL11A1) transcripts detectable by the above amplicon in Lung non-small cell carcinoma samples versus the normal tissue samples was determined by T test as 7.79e-008 (Lung adenocarcinoma, squamous cell carcinoma, and large cell carcinoma samples versus the normal tissue samples was determined by T test as 4.85e-004, 2.55e-004 and 5.52e-004 respectively).

Threshold of 5 fold over expression was found to differentiate between non-small cell carcinoma and normal samples with P value of 5.74e-007 as checked by exact Fisher test. Threshold of 5 fold over expression was found to differentiate between adenocarcinoma, squamous cell carcinoma and large cell carcinoma and normal samples with value of 1.54e-005, 8.45e-005 and 2.02e-005 was checked by exact Fisher test respectively. The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg15F (SEQ ID NO:163) forward primer; and HUMCA1XIA_seg15R (SEQ ID NO:164) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg15 (SEQ ID NO:165).

Forward Primer (HUMCA1XIA_seg15F (SEQ ID NO:
163)):
AATCAAGATCTATCCTAGATAAGCTGGAAG
Reverse Primer (HUMCA1XIA_seg15R (SEQ ID NO:
164)):
AATTGGGTTATAGTATTAGCCAAGTGAGTA
Amplicon (HUMCA1XIA_seg15 (SEQ ID NO: 165)):
AATCAAGATCTATCCTAGATAAGCTGGAAGATCTCTGATCATGAATCATA
GTCTTAACCCAACTAGATAAATACTCACTTGGCTAATACTATAACCCAAT
T

Expression of Homo sapiens Collagen, Type XI, Alpha 1 (COL11A1) HUMCA1XIA Transcripts which are Detectable by Amplicon as Depicted in Sequence Name HUMCA1XIA_seg15 (SEQ ID NO:165) in Different Normal Tissues

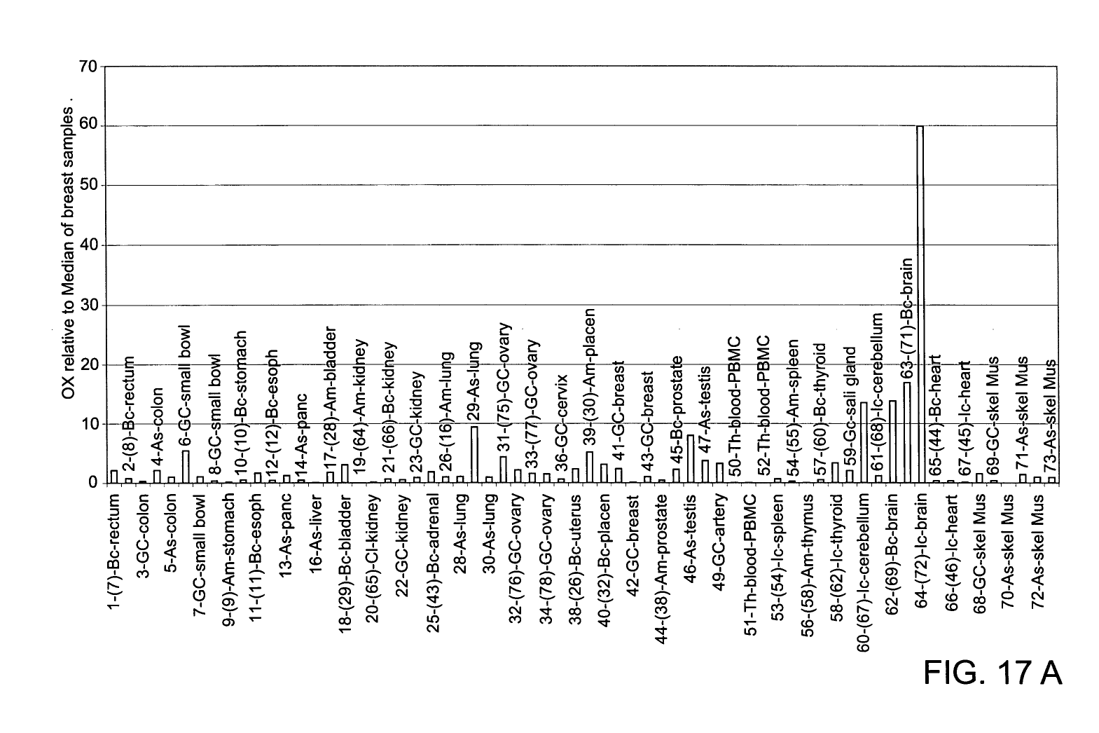

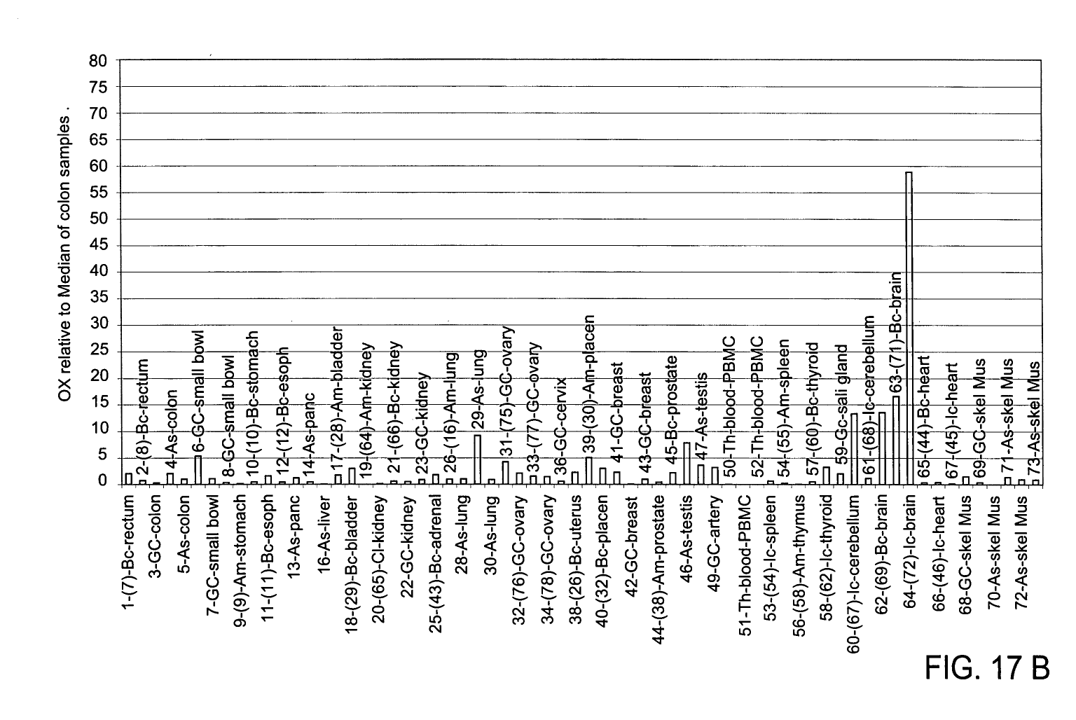

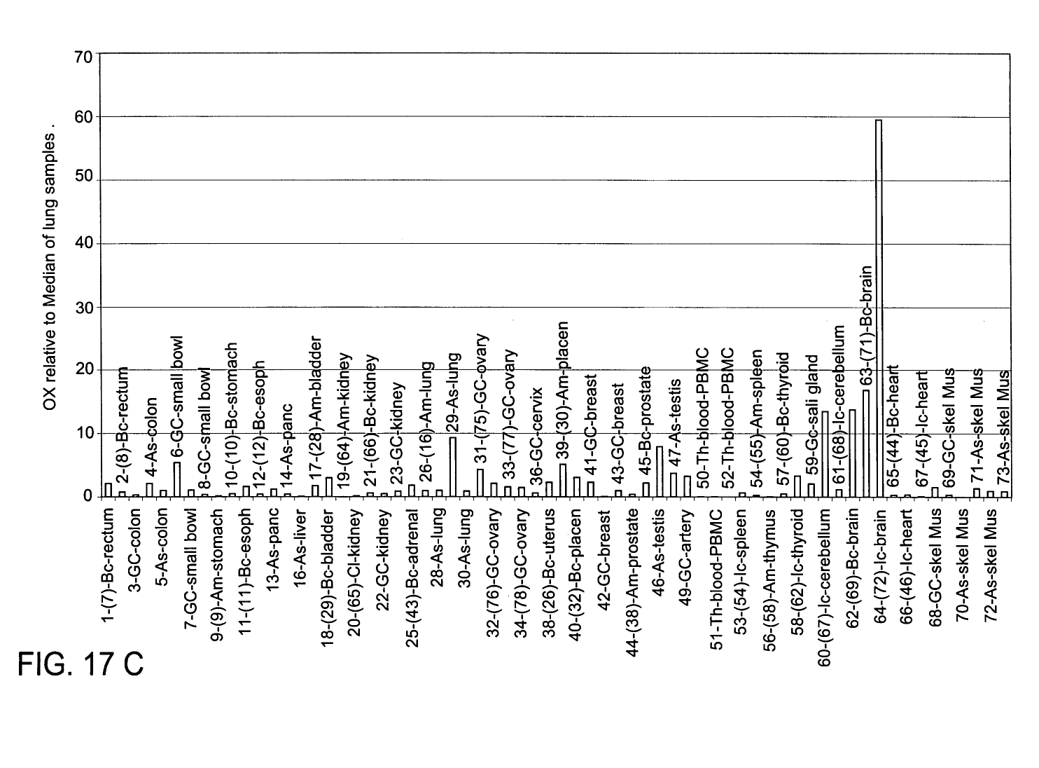

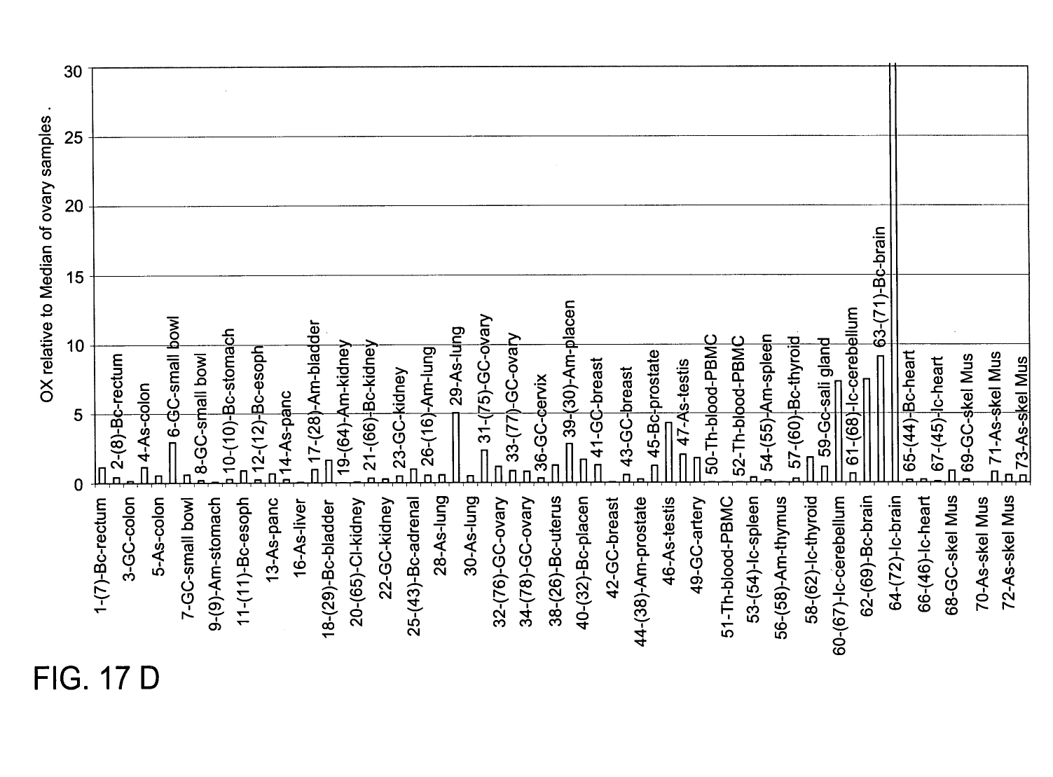

Expression of Homo sapiens collagen, type XI, alpha 1 (COL11A1) transcripts detectable by or according to seg15—HUMCA1XIA_seg15 (SEQ ID NO:165) amplicon and primers HUMCA1XIA_seg15F (SEQ ID NO:163) and HUMCA1XIA_seg15R (SEQ ID NO:164) was measured by real time PCR. Non-detected samples (sample(s) no. 16, 19, 42, 50, 51, 52, 56 and 70) were assigned Ct value of 41 and were calculated accordingly. In parallel the expression of several housekeeping genes—SDHA (GenBank Accession No. NM_{—}004168 (SEQ ID NO:31); amplicon—SDHA-amplicon (SEQ ID NO:34)), Ubiquitin (GenBank Accession No. BC000449 (SEQ ID NO:27); amplicon—Ubiquitin-amplicon (SEQ ID NO:30)), and TATA box (GenBank Accession No. NM_{—}003194 (SEQ ID NO:23); TATA amplicon (SEQ ID NO:26)) was measured similarly. For each RT sample, the expression of the above amplicon was normalized to the normalization factor calculated from the expression of these house keeping genes as described in the “materials and methods” section. The normalized quantity of each RT sample was then divided by the median of the quantities of the breast samples (sample numbers 41, 42 and 43, Table 1_{—}5 above), to obtain a value of relative expression of each sample relative to median of the breast samples, as presented in FIG. 17A, or to the median of the quantities of the colon samples (sample numbers 3, 4 and 5, Table 1_{—}5 above), to obtain a value of relative expression of each sample relative to median of the colon samples, as presented in FIG. 17B, or to the median of the quantities of the lung samples (sample numbers 26, 28, 29 and 30, Table 15 above), to obtain a value of relative expression of each sample relative to median of the lung samples, as presented in FIG. 17C, or to the median of the quantities of the ovary samples (sample numbers 31, 32, 33 and 34, Table 1_{—}5 above), to obtain a value of relative expression of each sample relative to median of the ovary samples, as presented in FIG. 17D.

Forward Primer (HUMCA1XIA_seg15F (SEQ ID NO:
163)):
AATCAAGATCTATCCTAGATAAGCTGGAAG
Reverse Primer (HUMCA1XIA_seg15R (SEQ ID NO:
164)):
AATTGGGTTATAGTATTAGCCAAGTGAGTA
Amplicon (HUMCA1XIA_seg15 (SEQ ID NO: 165)):
AATCAAGATCTATCCTAGATAAGCTGGAAGATCTCTGATCATGAATCATA
GTCTTAACCCAACTAGATAAATACTCACTTGGCTAATACTATAACCCAAT
T

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-59. (canceled)

60. An isolated polynucleotide comprising a nucleic acid sequence at least 95% homologous to SEQ ID NO:121.

61. The isolated polynucleotide of claim 60, comprising a nucleic acid sequence as set forth in SEQ ID NO:121.

62. The isolated polynucleotide of claim 60, comprising a nucleic acid sequence as set forth in SEQ ID NO:112.

63. An isolated polypeptide comprising an amino acid sequence at least 85% homologous to SEQ ID NO:170.

64. The polypeptide of claim 63, wherein the amino acid sequence is at least 95% homologous.

65. The polypeptide of claim 64, wherein the amino acid sequence is SEQ ID NO:170.

66. The isolated polypeptide of claim 63, comprising an amino acid sequence as set forth in SEQ ID NO:132.

67. An isolated amplicon having a nucleic acid sequence as set forth in SEQ ID NO:138.

68. A primer pair, comprising a pair of isolated oligonucleotides capable of amplifying a polynucleotide, wherein the polynucleotide comprises a nucleic acid sequence that is at least 95% homologous to SEQ ID NOs:112, 121, or 138.

69. The primer pair of claim 68, wherein the nucleic acid sequence is SEQ ID NOs:112, 121, or 138.

70. The primer pair of claim 68, comprising a pair of isolated oligonucleotides having a nucleic acid sequence as set forth in SEQ ID NO:136 and SEQ ID NO:137.

71. An antibody capable of specifically binding to at least one epitope of any one of a polypeptide comprising an amino acid sequence at least 85% homologous to the amino acid sequence set forth in SEQ ID NOs:132 or 170, wherein the epitope is not present within known proteins having the amino acid sequence set forth in anyone of SEQ ID NOs:127-128.

72. The polypeptide of claim 71, wherein the amino acid sequence is at least 95% homologous.

73. The polypeptide of claim 72, wherein the amino acid sequence is SEQ ID NOs:132 or 170.

74. The antibody of claim 71, wherein said antibody is capable of specifically binding to at least one epitope of an amino acid sequence selected from the group consisting of SEQ ID NOs:132 and 170.

75. A kit for detecting a disease, comprising a marker capable of detecting a polynucleotide comprising a nucleic acid sequence as set forth in any one of SEQ ID NOs:112, 121, 138.

76. The kit of claim 75, wherein said kit comprises at least one nucleotide probe or primer.

77. The kit of claim 76, wherein said kit comprises at least one primer pair capable of selectively hybridizing to a nucleic acid sequence as set forth in SEQ ID NO:138.

78. The kit of claim 77, wherein said primer pair has a nucleic acid sequence as set forth in SEQ ID NOs: 136 and SEQ ID NO:137.

79. The kit of claim 75, wherein said kit further comprises means for detecting expression of polynucleotide using a NAT (nucleic acid technology)-based assay.

80. The kit of claim 79, wherein the NAT-based assay is selected from the group consisting of a PCR, Real-Time PCR, LCR, Self-Sustained Synthetic Reaction, Q-Beta Replicase, Cycling probe reaction, Branched DNA, RFLP analysis, DGGE/TGGE, Single-Strand Conformation Polymorphism, Dideoxy fingerprinting, microarrays, Fluorescense In Situ Hybridization and Comparative Genomic Hybridization.

81. A kit for detecting a disease, comprising a marker capable of detecting any one of a polypeptide comprising an amino acid sequence at least 85% homologous to the amino acid sequence set forth in SEQ ID NOs:132 or 170.

82. The polypeptide of claim 81, wherein the amino acid sequence is at least 95% homologous.

83. The polypeptide of claim 82, wherein the amino acid sequence is SEQ ID NOs:132 or 170.

84. The kit of claim 81, wherein said kit comprises an antibody capable of specifically binding to at least one epitope of any one of a polypeptide comprising an amino acid sequence at least 85% homologous to the amino acid sequence set forth in SEQ ID NOs:132 or 170, wherein the epitope is not present within known proteins having the amino acid sequence set forth in anyone of SEQ ID NOs:127-128

85. The kit of claim 81, wherein said kit further comprises at least one reagent for performing an immunoassay.

86. The kit of claim 84, wherein said immunoassay is selected from the group consisting of an ELISA, an RIA, a slot blot, immunohistochemical assay, FACS, a radio-imaging assay or a Western blot.

87. A method for screening for a disease, disorder or condition in a subject, comprising detecting in the subject or in a sample obtained from said subject a polypeptide comprising an amino acid sequence at least 85% homologous to the amino acid sequence set forth in SEQ ID NOs:132 or 170.

88. The polypeptide of claim 87, wherein the amino acid sequence is at least 95% homologous.

89. The polypeptide of claim 88, wherein the amino acid sequence is SEQ ID NOs:132 or 170.

90. The method of claim 87, wherein screening for a disease comprises detecting the presence or severity of the disease, disorder or condition, or prognosis of the subject, or treatment selection for said subject, or treatment monitoring of said subject.

91. The method of claim 90, wherein the detection is conducted by immunoassay.

92. A method for screening for a disease, disorder or condition in a subject, comprising detecting in the subject or in a sample obtained from said subject any one of a polynucleotide comprising a nucleic acid sequence at least 95% homologous to SEQ ID NOs:112, 121, or 138.

93. The primer pair of claim 92, wherein the nucleic acid sequence is SEQ ID NOs:112, 121, or 138.

94. The method of claim 92, wherein screening for a disease comprises detecting the presence or severity of the disease, disorder or condition, or prognosis of the subject, or treatment selection for said subject, or treatment monitoring of said subject.

95. The method of claim 92, wherein the disease is cardiovascular disease selected from the group consisting of myocardial infarct, acute coronary syndrome, coronary artery disease, angina pectoris (stable and unstable), cardiomyopathy, myocarditis, congestive heart failure or any type of heart failure and reinfarction.

96. The method of claim 92, wherein the detection is performed using an oligonucleotide primer pair capable of hybridizing to at least a portion of the polynucleotide.

97. The method of claim 96, wherein the primer pair has a nucleic acid sequence as set forth in SEQ ID NOs: 136 and SEQ ID NO:137.

98. The method of claim 92, wherein the sample is selected from the group consisting of blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, lung tissue, isolated cells or tissues from a human organ, a sample obtained by lavage, and samples of cell culture constituents.
